SEQUENCE LISTING (990 sequences)

SEQ ID NO: 1 - protein, 20 aa, synthetic construct
KTRGVEEVAEGVVLLRRRGN

SEQ ID NO: 2 - protein, 180 aa, synthetic construct
SVEELLRKLEEVLRKIREENERSLKELRDRAREIVKRNRETNRELEEVIKELEKRLSGADKEKVEELVRRIRRIVERVVEEDRRTVEEIEKIAREVVKRDRDSADRVRRTVEDVLRKATGSEDIVRKIERIVETIEREVRESVKKVEEIARDIRRKVDESVKNVEKLLRDVDKKARDRKK

SEQ ID NO: 3 - protein, 174 aa, synthetic construct
SESDDVIRKLRELLEELRTHVEKSIRDLRKILEDSTRHAKRSIEELERLLEEVRKKPGDEEVRKTVEEISRRVAENVKRLEDLYRRMEEEVKKNLDRLRKRVEDIIREVEEARKKGVDEEKLKDLIRKLRDILRRAAEAHKKLIDDARESLERAKREHEKLIDRLKKILEELER

SEQ ID NO: 4 - protein, 182 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 5 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 6 - protein, 247 aa, synthetic construct
SEAEDLEELIKELAELLKDVIRKLEKINRRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEAERRRRELKDKLDRLREEHEEVKRRLEEELTRLRETHKKIEKELREALKRVRDRST

SEQ ID NO: 7 - protein, 247 aa, synthetic construct; identical to SEQ ID NO: 6

SEQ ID NO: 8 - protein, 247 aa, synthetic construct
DEDRIIEEIARLLEELLRELLELIKKLIETNRRLNEEHERAVRELARLLEELLDRLVKKGISDEKLKRIRERLKRALDDLERLHREINKRLEDLVRELEKLVREILKELKDALEELRRASARAGGEEVLRRLEEIVKKLLDLVRRILERLKEIHKDNVRLLRELNERLTRIVEDLVRLIREILREAGVDEKIAEEIERELEELRRMIKRLHEDLERKLKESEDELREIEARLEEKIRRLEKLERKRR

SEQ ID NO: 9 - protein, 241 aa, synthetic construct
SKKELLEEVVRRAIELLKRHLEKLKRILEEIVRLLEEHLEKVERVLEAILSLLDDLLRRGGDERAIRTLEDVKRRLREILERLADENAKAIKRLADLLDKLEKRNKEAIERLEEILEELKRVRRDEELLRVLETLLKIIEDILRENTKVLEDLLRLVEEILEANLRVVEELLRLAREILTEIVGDEDKLKEIEDELRRLLEELRRLDKAIKDRLRELKKDLDEANRRIKETLKKLLREVEK

SEQ ID NO: 10 - protein, 233 aa, synthetic construct
SKEEKLKDDVRAVLEDLDRVLKELEKLSEDNLRELKRVLDRITDLHRRILDELRKGIGSEELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERGDSKAAEDILRVLEKLVKVSREAIKLILELSEHHVRVSTRIARLLLDVARKLAEVIKEAER

SEQ ID NO: 11 - protein, 249 aa, synthetic construct
DAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIVREVERIVRELAKRSDEILKKLEDIVEKLRE

SEQ ID NO: 12 - protein, 249 aa, synthetic construct; identical to SEQ ID NO: 11

SEQ ID NO: 13 - protein, 243 aa, synthetic construct
DEEETLRRLLERKVELAKEYLDVSKEVIDRTTKLLDEYLKTSKRIVDATVELLERGDLGPDELIKRLAEELERSLRELEEEIKRLKRELEESLKKLKEIIDRLAEEAEKLLAVLKRGEGSEEEALRALASLVRELIEVLRENDERLRDVLRRLIEALRKNNEILERVLRKLVRAAEERGRDESSREALEEARRRLEELLRELNEITKDLEAKLEKLLRDLNELTKALEEELKRLLDELKKRTD

SEQ ID NO: 14 - protein, 180 aa, synthetic construct
SVEELLRKLEEVLRKIREENERSLKELRDRAREIVKRNRETNRELEEVIKELEKRLSGADKEKVEELVRRIRRIVERVVEEDRRTVEEIEKIAREVVKRDRDSADRVRRTVEDVLRKATGKTRGVEEVAEGVVLLRRRGNESVKKVEEIARDIRRKVDESVKNVEKLLRDVDKKARDRKK

SEQ ID NO: 15 - protein, 180 aa, synthetic construct
SVEELLRKLEEVLRKIREENERSLKELRDRAREIVKRNRETNRELEEVIKELEKRLSGADKEKVEELVRRIRRIVERVVEEDRRTVEEIEKIAREVVKRDRDSADRVRRTVEDVLRKATGSEDIKTRGVEEVAEGVVLLRRRGNKVEEIARDIRRKVDESVKNVEKLLRDVDKKARDRKK

SEQ ID NO: 16 - protein, 180 aa, synthetic construct
SVEELLRKLEEVLRKIREENERSLKELRDRAREIVKRNRETNRELEEVIKELEKRLSGADKEKVEELVRRIRRIVERVVEEDRRTVEEIEKIAREVVKRDRDSADRVRRTVEDVLRKATGSEDIVRKKTRGVEEVAEGVVLLRRRGNEIARDIRRKVDESVKNVEKLLRDVDKKARDRKK

SEQ ID NO: 17 - protein, 180 aa, synthetic construct
SKTRGVEEVAEGVVLLRRRGNRSLKELRDRAREIVKRNRETNRELEEVIKELEKRLSGADKEKVEELVRRIRRIVERVVEEDRRTVEEIEKIAREVVKRDRDSADRVRRTVEDVLRKATGSEDIVRKIERIVETIEREVRESVKKVEEIARDIRRKVDESVKNVEKLLRDVDKKARDRKK

SEQ ID NO: 18 - protein, 180 aa, synthetic construct
SVEELKTRGVEEVAEGVVLLRRRGNELRDRAREIVKRNRETNRELEEVIKELEKRLSGADKEKVEELVRRIRRIVERVVEEDRRTVEEIEKIAREVVKRDRDSADRVRRTVEDVLRKATGSEDIVRKIERIVETIEREVRESVKKVEEIARDIRRKVDESVKNVEKLLRDVDKKARDRKK

SEQ ID NO: 19 - protein, 174 aa, synthetic construct
SESDDVIRKLRELLEELRTHVEKSIRDLRKILEDSTRHAKRSIEELERLLEEVRKKPGDEEVRKTVEEISRRVAENVKRLEDLYRRMEEEVKKNLDRLRKRVEDIIREVEEARKKGVDEEKLKDLIRKLRDILRRAAEAHKKLIDDARESLERKTRGVEEVAEGVVLLRRRGNR

SEQ ID NO: 20 - protein, 174 aa, synthetic construct
SESDDVIRKLRELLEELRTHVEKSIRDLRKILEDKTRGVEEVAEGVVLLRRRGNKKPGDEEVRKTVEEISRRVAENVKRLEDLYRRMEEEVKKNLDRLRKRVEDIIREVEEARKKGVDEEKLKDLIRKLRDILRRAAEAHKKLIDDARESLERAKREHEKLIDRLKKILEELER

SEQ ID NO: 21 - protein, 182 aa, synthetic construct
SELEEIEKKKTRGVEEVAEGVVLLRRRGNELIETNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 22 - protein, 182 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLKTRGVEEVAEGVVLLRRRGNDIKRALDELLKELEKSVRESEERLRRLLEEVKRVIEEVAT

SEQ ID NO: 23 - protein, 182 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKAKTRGVEEVAEGVVLLRRRGNALDELLKELEKSVRESEERLRRLLEEVKRVIEEVAT

SEQ ID NO: 24 - protein, 182 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKALEEKTRGVEEVAEGVVLLRRRGNELLKELEKSVRESEERLRRLLEEVKRVIEEVAT

SEQ ID NO: 25 - protein, 182 aa, synthetic construct
SELEEIEKKVRELKTRGVEEVAEGVVLLRRRGNTNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 26 - protein, 182 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSKTRGVEEVAEGVVLLRRRGNEVAT

SEQ ID NO: 27 - protein, 182 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESKTRGVEEVAEGVVLLRRRGN

SEQ ID NO: 28 - protein, 182 aa, synthetic construct
SELEEIEKKVRELTKRKTRGVEEVAEGVVLLRRRGNRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 29 - protein, 182 aa, synthetic construct
SELEEIEKKVRELTKRHRELKTRGVEEVAEGVVLLRRRGNTLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 30 - protein, 182 aa, synthetic construct
SELEEIEKKVRELTKRHRELVERVRKTKTRGVEEVAEGVVLLRRRGNRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 31 - protein, 182 aa, synthetic construct
SEKTRGVEEVAEGVVLLRRRGNRVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 32 - protein, 182 aa, synthetic construct
SELEEIEKKVRELTKRHRELVERVRKTVKELKTRGVEEVAEGVVLLRRRGNEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 33 - protein, 182 aa, synthetic construct
SELEEIEKKVRELTKRHRELVERVRKTVKELIETKTRGVEEVAEGVVLLRRRGNDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 34 - protein, 182 aa, synthetic construct
SELEEIEKKVRELTKRHRELVERVRKTVKELIETNRRLKTRGVEEVAEGVVLLRRRGNRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 35 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGKTRGVEEVAEGVVLLRRRGNESMRRVKEDADRVSEANKEVLDRVREEVKRLIEEVRETLR

SEQ ID NO: 36 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDKTRGVEEVAEGVVLLRRRGNRVKEDADRVSEANKEVLDRVREEVKRLIEEVRETLR

SEQ ID NO: 37 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRKTRGVEEVAEGVVLLRRRGNEDADRVSEANKEVLDRVREEVKRLIEEVRETLR

SEQ ID NO: 38 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERKTRGVEEVAEGVVLLRRRGNRVSEANKEVLDRVREEVKRLIEEVRETLR

SEQ ID NO: 39 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREKTRGVEEVAEGVVLLRRRGNEANKEVLDRVREEVKRLIEEVRETLR

SEQ ID NO: 40 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVKTRGVEEVAEGVVLLRRRGNANKEVLDRVREEVKRLIEEVRETLR

SEQ ID NO: 41 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESKTRGVEEVAEGVVLLRRRGNRVREEVKRLIEEVRETLR

SEQ ID NO: 42 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRKTRGVEEVAEGVVLLRRRGNEEVKRLIEEVRETLR

SEQ ID NO: 43 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKTRGVEEVAEGVVLLRRRGNEVKRLIEEVRETLR

SEQ ID NO: 44 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDKTRGVEEVAEGVVLLRRRGNRLIEEVRETLR

SEQ ID NO: 45 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVKTRGVEEVAEGVVLLRRRGNEVRETLR

SEQ ID NO: 46 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKTRGVEEVAEGVVLLRRRGNTLR

SEQ ID NO: 47 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEKTRGVEEVAEGVVLLRRRGNR

SEQ ID NO: 48 - protein, 184 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVKTRGVEEVAEGVVLLRRRGN

SEQ ID NO: 49 - protein, 184 aa, synthetic construct
DEIRKVVKEITDLLKASNDKKTRGVEEVAEGVVLLRRRGNELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 50 - protein, 184 aa, synthetic construct
DEIRKVVKEITDLLKASNDKNRKKTRGVEEVAEGVVLLRRRGNERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 51 - protein, 184 aa, synthetic construct
DEIRKVVKEITDLLKASNDKNRKVKTRGVEEVAEGVVLLRRRGNRLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 52 - protein, 184 aa, synthetic construct
DEIRKVVKEITDLLKASNDKNRKVVEEIRDLLRKSKKLKTRGVEEVAEGVVLLRRRGNKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 53 - protein, 184 aa, synthetic construct
DEIRKKTRGVEEVAEGVVLLRRRGNEEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 54 - protein, 184 aa, synthetic construct
DEIRKVKTRGVEEVAEGVVLLRRRGNEIRDLLRKSKKLADELVERLRALVEDLRRRIDKSGDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 55 - protein, 247 aa, synthetic construct
SEAEDLEELIKELAELLKDVIRKLEKINRRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKKTRGVEEVAEGVVLLRRRGNHEEVKRRLEEELTRLRETHKKIEKELREALKRVRDRST

SEQ ID NO: 56 - protein, 247 aa, synthetic construct
SEAEDLEELIKELAELLKDVIRKLEKINRRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEKTRGVEEVAEGVVLLRRRGNEVKRRLEEELTRLRETHKKIEKELREALKRVRDRST

SEQ ID NO: 57 - protein, 247 aa, synthetic construct
SEAEDLEELKTRGVEEVAEGVVLLRRRGNRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEAERRRRELKDKLDRLREEHEEVKRRLEEELTRLRETHKKIEKELREALKRVRDRST

SEQ ID NO: 58 - protein, 247 aa, synthetic construct
SEAEDLEELIKELAELLKDVIRKLEKINRRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEAERRRREKTRGVEEVAEGVVLLRRRGNEELTRLRETHKKIEKELREALKRVRDRST

SEQ ID NO: 59 - protein, 247 aa, synthetic construct
SEAEDLEELIKELAELLKDVIRKLEKINRRLVKIKTRGVEEVAEGVVLLRRRGNKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEAERRRRELKDKLDRLREEHEEVKRRLEEELTRLRETHKKIEKELREALKRVRDRST

SEQ ID NO: 60 - protein, 247 aa, synthetic construct
SEAEDKTRGVEEVAEGVVLLRRRGNKINRRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEAERRRRELKDKLDRLREEHEEVKRRLEEELTRLRETHKKIEKELREALKRVRDRST

SEQ ID NO: 61 - protein, 248 aa, synthetic construct
DEDRIIEEIARLLEELLRELLELIKKLIETNRRLNEEHERAVRELARLLEELLDRLVKKGISDEKLKRIRERLKRALDDLERLHREINKRLEDLVRELEKLVREILKELKDALEELRRASARAGGEEVLRRLEEIVKKLLDLVRRILERLKEIHKDNVRLLRELNERLTRIVEDLVRLIREILREAGVDEKIAEEIERELEELRRMIKRLHEDLERKKTRGVEEVAEGVVLLRRRGNRLEEKLERKRR

SEQ ID NO: 62 - protein, 248 aa, synthetic construct
DEKTRGVEEVAEGVVLLRRRGNLIKKLIETNRRLNEEHERAVRELARLLEELLDRLVKKGISDEKLKRIRERLKRALDDLERLHREINKRLEDLVRELEKLVREILKELKDALEELRRASARAGGEEVLRRLEEIVKKLLDLVRRILERLKEIHKDNVRLLRELNERLTRIVEDLVRLIREILREAGVDEKIAEEIERELEELRRMIKRLHEDLERKLKESEDELREIEARLEEKIRRLEEKLERKRR

SEQ ID NO: 63 - protein, 241 aa, synthetic construct
SKKELLEEVVRRAIELLKRHLEKLKRILEEIVRLLEEHLEKVERVLEAILSLLDDLLRRGGDERAIRTLEDVKRRLREILERLADENAKAIKRLADLLDKLEKRNKEAIERLEEILEELKRVRRDEELLRVLETLLKIIEDILRENTKVLEDLLRLVEEILEANLRVVEELLRLAREILTEIVGDEDKLKEIEDELRRLKTRGVEEVAEGVVLLRRRGNDLDEANRRIKETLKKLLREVEK

SEQ ID NO: 64 - protein, 233 aa, synthetic construct
SKKTRGVEEVAEGVVLLRRRGNELEKLSEDNLRELKRVLDRITDLHRRILDELRKGIGSEELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERGDSKAAEDILRVLEKLVKVSREAIKLILELSEHHVRVSTRIARLLLDVARKLAEVIKEAER

SEQ ID NO: 65 - protein, 233 aa, synthetic construct
SKEEKLKDDVRAVLEDLDRVLKELEKLSEDNLRELKRVLDRITDLHRRILDELRKGIGSEELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERGDSKAAEDILRVLEKLVKVSREAIKLILELSEHHVRVSTRIKTRGVEEVAEGVVLLRRRGN

SEQ ID NO: 66 - protein, 233 aa, synthetic construct
SKEEKKTRGVEEVAEGVVLLRRRGNKLSEDNLRELKRVLDRITDLHRRILDELRKGIGSEELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERGDSKAAEDILRVLEKLVKVSREAIKLILELSEHHVRVSTRIARLLLDVARKLAEVIKEAER

SEQ ID NO: 67 - protein, 233 aa, synthetic construct
SKEEKLKDDVRAKTRGVEEVAEGVVLLRRRGNRELKRVLDRITDLHRRILDELRKGIGSEELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERGDSKAAEDILRVLEKLVKVSREAIKLILELSEHHVRVSTRIARLLLDVARKLAEVIKEAER

SEQ ID NO: 68 - protein, 249 aa, synthetic construct
DAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIKTRGVEEVAEGVVLLRRRGNKLEDIVEKLRE

SEQ ID NO: 69 - protein, 249 aa, synthetic construct
DAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIVREKTRGVEEVAEGVVLLRRRGNDIVEKLRE

SEQ ID NO: 70 - protein, 249 aa, synthetic construct
DAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLKTRGVEEVAEGVVLLRRRGNDVKKASEDIVREVERIVRELAKRSDEILKKLEDIVEKLRE

SEQ ID NO: 71 - protein, 249 aa, synthetic construct
DAEEVVKRLADVKTRGVEEVAEGVVLLRRRGNENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIVREVERIVRELAKRSDEILKKLEDIVEKLRE

SEQ ID NO: 72 - protein, 249 aa, synthetic construct
DAEEVKTRGVEEVAEGVVLLRRRGNDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIVREVERIVRELAKRSDEILKKLEDIVEKLRE

SEQ ID NO: 73 - protein, 249 aa, synthetic construct
DAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDKTRGVEEVAEGVVLLRRRGNKASEDIVREVERIVRELAKRSDEILKKLEDIVEKLRE

SEQ ID NO: 74 - protein, 249 aa, synthetic construct
DAEEVVKRLADVLRENDETIRKVVEDLVRIAEENDRLKTRGVEEVAEGVVLLRRRGNRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIVREVERIVRELAKRSDEILKKLEDIVEKLRE

SEQ ID NO: 75 - protein, 249 aa, synthetic construct; identical to SEQ ID NO: 71

SEQ ID NO: 76 - protein, 243 aa, synthetic construct
DEEETLRRLLERKVELAKEYLDVSKEVIDRTTKLLDEYLKTSKRIVDATVELLERGDLGPDELIKRLAEELERSLRELEEEIKRLKRELEESLKKLKEIIDRLAEEAEKLLAVLKRGEGSEEEALRALASLVRELIEVLRENDERLRDVLRRLIEALRKNNEILERVLRKLVRAAEERGRDESSREALEEARRRLEELLRELNEIKTRGVEEVAEGVVLLRRRGNALEEELKRLLDELKKRTD

SEQ ID NO: 77 - protein, 59 aa, synthetic construct
EDIVRKIERIVETIEREVRESVKKVEEIARDIRRKVDESVKNVEKLLRDVDKKARDRKK

SEQ ID NO: 78 - protein, 59 aa, synthetic construct
SVEELLRKLEEVLRKIREENERSLKELRDRAREIVKRNRETNRELEEVIKELEKRLSGA

SEQ ID NO: 79 - protein, 56 aa, synthetic construct
EEKLKDLIRKLRDILRRAAEAHKKLIDDARESLERAKREHEKLIDRLKKILEELER

SEQ ID NO: 80 - protein, 55 aa, synthetic construct
SESDDVIRKLRELLEELRTHVEKSIRDLRKILEDSTRHAKRSIEELERLLEEVRK

SEQ ID NO: 81 - protein, 59 aa, synthetic construct
DKALEEAKRVLAENNEVIKDIKRALDELLKELEKSVRESEERLRRLLEEVKRVIEEVAT

SEQ ID NO: 82 - protein, 59 aa, synthetic construct
SELEEIEKKVRELTKRHRELVERVRKTVKELIETNRRLLETLTERIKRVLEEVRDLERR

SEQ ID NO: 83 - protein, 59 aa, synthetic construct
SEDLKRVEERAREVSRRNEESMRRVKEDADRVSEANKEVLDRVREEVKRLIEEVRETLR

SEQ ID NO: 84 - protein, 59 aa, synthetic construct
DEIRKVVKEITDLLKASNDKNRKVVEEIRDLLRKSKKLADELVERLRALVEDLRRRIDK

SEQ ID NO: 85 - protein, 59 aa, synthetic construct
KDEAERRRRELKDKLDRLREEHEEVKRRLEEELTRLRETHKKIEKELREALKRVRDRST

SEQ ID NO: 86 - protein, 56 aa, synthetic construct
SEAEDLEELIKELAELLKDVIRKLEKINRRLVKILEDIIRRLKEISKEAEEELRKG

SEQ ID NO: 87 - protein, 60 aa, synthetic construct
SDKEEIKRRVEKTARDLETEHDKIKKRLEDTVRDIKRELDELLEKYERVLRKIEKTLREG

SEQ ID NO: 88 - protein, 59 aa, synthetic construct
EKIAEEIERELEELRRMIKRLHEDLERKLKESEDELREIEARLEEKIRRLEEKLERKRR

SEQ ID NO: 89 - protein, 60 aa, synthetic construct
DEDRIIEEIARLLEELLRELLELIKKLIETNRRLNEEHERAVRELARLLEELLDRLVKKG

SEQ ID NO: 90 - protein, 56 aa, synthetic construct
EDKLKEIEDELRRLLEELRRLDKAIKDRLRELKKDLDEANRRIKETLKKLLREVEK

SEQ ID NO: 91 - protein, 59 aa, synthetic construct
SKAAEDILRVLEKLVKVSREAIKLILELSEHHVRVSTRIARLLLDVARKLAEVIKEAER

SEQ ID NO: 92 - protein, 57 aa, synthetic construct
SKEEKLKDDVRAVLEDLDRVLKELEKLSEDNLRELKRVLDRITDLHRRILDELRKGI

SEQ ID NO: 93 - protein, 59 aa, synthetic construct
EDVKRALEELVSRLRKLLEDVKKASEDIVREVERIVRELAKRSDEILKKLEDIVEKLRE

SEQ ID NO: 94 - protein, 59 aa, synthetic construct
DAEEVVKRLADVLRENDETIRKVVEDLVRIAEENDRLWKKLVEDIAEILRRIVELLRRG

SEQ ID NO: 95 - protein, 59 aa, synthetic construct; identical to SEQ ID NO: 94

SEQ ID NO: 96 - protein, 60 aa, synthetic construct
SREALEEARRRLEELLRELNEITKDLEAKLEKLLRDLNELTKALEEELKRLLDELKKRTD

SEQ ID NO: 97 - protein, 646 aa, synthetic construct
MVLQTQVFISLLLWISGAYGQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSSELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGLDDKALEEAKRVLAENNEVIKDIKRALDELLKELEKSVRESKTRGVEEVAEGVVLLRRRGN

SEQ ID NO: 98 - protein, 1938 aa, synthetic construct
ATGGTCTTGCAGACTCAAGTATTTATATCCCTTTTGCTCTGGATCTCTGGAGCTTACGGCCAGGAACAGCTCGTCGAAAGCGGCGGCAGACTGGTGACACCTGGCGGCAGCCTGACCCTGAGCTGCAAGGCCAGCGGCTTCGACTTCAGCGCCTACTACATGAGCTGGGTCCGCCAGGCCCCTGGCAAGGGACTGGAATGGATCGCCACCATCTACCCCAGCAGCGGCAAGACCTACTACGCCACCTGGGTGAACGGACGGTTCACCATCTCCAGCGACAACGCCCAGAACACCGTGGACCTGCAGATGAACAGCCTGACAGCCGCCGACCGGGCCACCTACTTTTGCGCTCGGGACAGCTACGCCGACGACGGCGCCCTGTTCAACATCTGGGGCCCTGGCACCCTGGTGACAATCTCTAGCGGCGGAGGCGGATCTGGTGGCGGAGGAAGTGGCGGCGGAGGATCTGAGCTGGTGCTGACCCAGAGCCCCTCTGTGTCTGCTGCCCTGGGAAGCCCTGCCAAGATCACCTGTACCCTGAGCAGCGCCCACAAGACCGACACCATCGACTGGTATCAGCAGCTGCAGGGCGAGGCCCCCAGATACCTGATGCAGGTGCAGAGCGACGGCAGCTACACCAAGAGGCCAGGCGTGCCCGACAGGTTCAGCGGATCTAGCTCTGGCGCCGACCGCTACCTGATCATCCCCAGCGTGCAGGCCGATGACGAGGCCGATTACTACTGTGGCGCCGACTACATCGGCGGCTACGTGTTCGGCGGAGGCACCCAGCTGACCGTGACCGGTGAAAGCAAATACGGACCGCCCTGCCCCCCTTGCCCTATGTTCTGGGTGCTGGTGGTGGTCGGAGGCGTGCTGGCCTGCTACAGCCTGCTGGTCACCGTGGCCTTCATCATCTTTTGGGTGAAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTGCGGGTGAAGTTCAGCAGAAGCGCCGACGCCCCTGCCTACCAGCAGGGCCAGAATCAGCTGTACAACGAGCTGAACCTGGGCAGAAGGGAAGAGTACGACGTCCTGGATAAGCGGAGAGGCCGGGACCCTGAGATGGGCGGCAAGCCTCGGCGGAAGAACCCCCAGGAAGGCCTGTATAACGAACTGCAGAAAGACAAGATGGCCGAGGCCTACAGCGAGATCGGCATGAAGGGCGAGCGGAGGCGGGGCAAGGGCCACGACGGCCTGTATCAGGGCCTGTCCACCGCCACCAAGGATACCTACGACGCCCTGCACATGCAGGCCCTGCCCCCAAGAGGATCCTCCGAGCTGGAGGAGATTGAGAAGAAGGTGAGGGAGCTGACCAAGCGCCACCGCGAACTGGTGGAGCGAGTGCGCAAGACCGTGAAGGAGCTCATTGAGACTAACCGCCGGCTGCTCGAGACCCTGACGGAGCGTATCAAGCGTGTCCTCGAGGAGGTGCGCGACCTGGAGCGGCGCGGGGAGCCCAAGGAAGATCTCAAGGCCCGCCTGGAAGACATCATCCGCCGCCTGGCAGAGGACATTGAGCGCGTCAAGCGCGAGACCGACAAGCTGTTGGAAGAGCTTGCTCGCATCATCGAGGAGGTGGAGAAAGAGATGCGTGAAACCAACAAGGAGTTGGATAAAGAGATCAAAGACGGCGGCCTTGATGACAAGGCCCTGGAGGAGGCCAAGCGCGTGCTGGCCGAGAACAACGAGGTGATTAAGGACATCAAGAGAGCTCTGGACGAGCTGCTGAAGGAGCTGGAGAAGTCCGTGAGGGAGAGCAAGACTCGCGGTGTGGAGGAAGTGGCGGAGGGCGTGGTCCTGCTGCGCCGTCGCGGCAAC

SEQ ID NO: 99 - protein, 713 aa, synthetic construct
MVLQTQVFISLLLWISGAYGQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSDAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDKTRGVEEVAEGVVLLRRRGNKASEDIVREVERIVRELAKRSDEILKKLEDIVEKLRE

SEQ ID NO: 100 - DNA, 2139 bp, synthetic construct
  source 1..2139: mol_type = other DNA
atggtcttgcagactcaagtatttatatcccttttgctctggatctctggagcttacggccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgctcgggacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgacaggttcagcggatctagctctggcgccgaccgctacctgatcatccccagcgtgcaggccgatgacgaggccgattactactgtggcgccgactacatcggcggctacgtgttcggcggaggcacccagctgaccgtgaccggtgaaagcaaatacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagaggatccgacgccgaggaggtggtcaagcgtctagctgatgtgctgcgagagaacgacgagactatccgcaaggtggtcgaagatctggtgcgcatcgctgaggagaatgacaggctttggaagaagctggtagaagacatcgccgagattctgcgtcgcatcgtggagctgcttcgccgcggcggcgtgcccgaggagctgctggaccgcctggcgaaagtggttaagtctatcgtggagaaagccgagaagatactggagcgcctgaaccgcgtgtcgaaggccatcgcggaaaagctcaagaccatcgtcgacgagctcaacgaggtgtccaaagagatcgtaaagcgcgccgaagacatcctgcgcaagggcaaggacaaggagaccgtgctgcgcgcgctgcgcaccctggtgaaggagtacgcggacctgtccaaggaggtattggagcgagtggagcgcattgtcagggagtacgtgaagctttccgacgaggtggtgaagagcctggcagagatcgtggaggaactcatccgtatcattgaggacctgctgcgcaaaggcaacctggatgaagataaaacccgcggtgtggaagaggtggcggagggcgtcgtcctgctccgtcgtcggggcaacaaggcttccgaggacattgtgagggaggtggagcggatcgtgcgcgagctggccaagcgcagcgatgagatcctgaagaagctggaggacatcgtggagaagctgcgggag

SEQ ID NO: 101 - protein, 5 aa, synthetic construct
GSGGS

SEQ ID NO: 102 - protein, 4 aa, synthetic construct
GGGS

SEQ ID NO: 103 - protein, 4 aa, synthetic construct
GGSG

SEQ ID NO: 104 - protein, 5 aa, synthetic construct
GGSGG

SEQ ID NO: 105 - protein, 5 aa, synthetic construct
GSGSG

SEQ ID NO: 106 - protein, 5 aa, synthetic construct
GSGGG

SEQ ID NO: 107 - protein, 5 aa, synthetic construct
GGGSG

SEQ ID NO: 108 - protein, 5 aa, synthetic construct
GSSSG

SEQ ID NO: 109 - protein, 5 aa, synthetic construct
GSSSG

SEQ ID NO: 110 - protein, 4 aa, synthetic construct
RRRG

SEQ ID NO: 111 - protein, 5 aa, synthetic construct
RRRGN

SEQ ID NO: 112 - protein, 5 aa, synthetic construct
LRRRG

SEQ ID NO: 113 - protein, 6 aa, synthetic construct
LRRRGN

SEQ ID NO: 114 - protein, 6 aa, synthetic construct
LLRRRG

SEQ ID NO: 115 - protein, 7 aa, synthetic construct
LLRRRGN

SEQ ID NO: 116 - protein, 7 aa, synthetic construct
VLLRRRG

SEQ ID NO: 117 - protein, 8 aa, synthetic construct
VLLRRRGN

SEQ ID NO: 118 - protein, 8 aa, synthetic construct
VVLLRRRG

SEQ ID NO: 119 - protein, 9 aa, synthetic construct
VVLLRRRGN

SEQ ID NO: 120 - protein, 9 aa, synthetic construct
GVVLLRRRG

SEQ ID NO: 121 - protein, 10 aa, synthetic construct
GVVLLRRRGN

SEQ ID NO: 122 - protein, 10 aa, synthetic construct
EGVVLLRRRG

SEQ ID NO: 123 - protein, 11 aa, synthetic construct
EGVVLLRRRGN

SEQ ID NO: 124 - protein, 11 aa, synthetic construct
AEGVVLLRRRG

SEQ ID NO: 125 - protein, 12 aa, synthetic construct
AEGVVLLRRRGN

SEQ ID NO: 126 - protein, 12 aa, synthetic construct
VAEGVVLLRRRG

SEQ ID NO: 127 - protein, 13 aa, synthetic construct
VAEGVVLLRRRGN

SEQ ID NO: 128 - protein, 13 aa, synthetic construct
EVAEGVVLLRRRG

SEQ ID NO: 129 - protein, 14 aa, synthetic construct
EVAEGVVLLRRRGN

SEQ ID NO: 130 - protein, 14 aa, synthetic construct
EEVAEGVVLLRRRG

SEQ ID NO: 131 - protein, 15 aa, synthetic construct
EEVAEGVVLLRRRGN

SEQ ID NO: 132 - protein, 15 aa, synthetic construct
VEEVAEGVVLLRRRG

SEQ ID NO: 133 - protein, 16 aa, synthetic construct
VEEVAEGVVLLRRRGN

SEQ ID NO: 134 - protein, 16 aa, synthetic construct
GVEEVAEGVVLLRRRG

SEQ ID NO: 135 - protein, 17 aa, synthetic construct
GVEEVAEGVVLLRRRGN

SEQ ID NO: 136 - protein, 17 aa, synthetic construct
RGVEEVAEGVVLLRRRG

SEQ ID NO: 137 - protein, 18 aa, synthetic construct
RGVEEVAEGVVLLRRRGN

SEQ ID NO: 138 - protein, 18 aa, synthetic construct
TRGVEEVAEGVVLLRRRG

SEQ ID NO: 139 - protein, 19 aa, synthetic construct
TRGVEEVAEGVVLLRRRGN

SEQ ID NO: 140 - protein, 19 aa, synthetic construct
KTRGVEEVAEGVVLLRRRG

SEQ ID NO: 141 - protein, 20 aa, synthetic construct
KTRGVEEVAEGVVLLRRRGN

SEQ ID NO: 142 - protein, 120 aa, synthetic construct
KEKVEELVRRIRRIVERVVEEDRRTVEEIEKIAREVVKRDRDSADRVRRTVEDVLRKATGSEDIVRKIERIVETIEREVRESVKKVEEIARDIRRKVDESVKNVEKLLRDVDKKARDRKK

SEQ ID NO: 143 - protein, 120 aa, synthetic construct
SVEELLRKLEEVLRKIREENERSLKELRDRAREIVKRNRETNRELEEVIKELEKRLSGADKEKVEELVRRIRRIVERVVEEDRRTVEEIEKIAREVVKRDRDSADRVRRTVEDVLRKATG

SEQ ID NO: 144 - protein, 114 aa, synthetic construct
EVRKTVEEISRRVAENVKRLEDLYRRMEEEVKKNLDRLRKRVEDIIREVEEARKKGVDEEKLKDLIRKLRDILRRAAEAHKKLIDDARESLERAKREHEKLIDRLKKILEELER

SEQ ID NO: 145 - protein, 114 aa, synthetic construct
SESDDVIRKLRELLEELRTHVEKSIRDLRKILEDSTRHAKRSIEELERLLEEVRKKPGDEEVRKTVEEISRRVAENVKRLEDLYRRMEEEVKKNLDRLRKRVEDIIREVEEARK

SEQ ID NO: 146 - protein, 122 aa, synthetic construct
EPKEDLKARLEDIIRRLAEDIERVKRETDKLLEELARIIEEVEKEMRETNKELDKEIKDGGLDDKALEEAKRVLAENNEVIKDIKRALDELLKELEKSVRESEERLRRLLEEVKRVIEEVAT

SEQ ID NO: 147 - protein, 122 aa, synthetic construct
SELEEIEKKVRELTKRHRELVERVRKTVKELIETNRRLLETLTERIKRVLEEVRDLERRGEPKEDLKARLEDIIRRLAEDIERVKRETDKLLEELARIIEEVEKEMRETNKELDKEIKDGGL

SEQ ID NO: 148 - protein, 124 aa, synthetic construct
GDKETAEDIVRRIIEELKRILKEIEDLARRINREIERLVEEVERDNRDVNRAIEELLKDIARRGGSEDLKRVEERAREVSRRNEESMRRVKEDADRVSEANKEVLDRVREEVKRLIEEVRETLR

SEQ ID NO: 149 - protein, 124 aa, synthetic construct
DEIRKVVKEITDLLKASNDKNRKVVEEIRDLLRKSKKLADELVERLRALVEDLRRRIDKSGDKETAEDIVRRIIEELKRILKEIEDLARRINREIERLVEEVERDNRDVNRAIEELLKDIARRG

SEQ ID NO: 150 - protein, 187 aa, synthetic construct
KDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGKDKDEAERRRRELKDKLDRLREEHEEVKRRLEEELTRLRETHKKIEKELREALKRVRDRST

SEQ ID NO: 151 - protein, 187 aa, synthetic construct
SEAEDLEELIKELAELLKDVIRKLEKINRRLVKILEDIIRRLKEISKEAEEELRKGTVEDKDILRDLERRLREILEESDRLLEELKRRLEEILRKSKELLRRLEEVLREILKRAEEVKRSNLPKEELIKEIVKLLEELLRVIEKILEDNIRLLEELVEVIKEILEKHLRLLEELVRVIERILREVGK

SEQ ID NO: 152 - protein, 187 aa, synthetic construct; identical to SEQ ID NO: 150

SEQ ID NO: 153 - protein, 187 aa, synthetic construct; identical to SEQ ID NO: 151

SEQ ID NO: 154 - protein, 187 aa, synthetic construct
ISDEKLKRIRERLKRALDDLERLHREINKRLEDLVRELEKLVREILKELKDALEELRRASARAGGEEVLRRLEEIVKKLLDLVRRILERLKEIHKDNVRLLRELNERLTRIVEDLVRLIREILREAGVDEKIAEEIERELEELRRMIKRLHEDLERKLKESEDELREIEARLEEKIRRLEKLERKRR

SEQ ID NO: 155 - protein, 187 aa, synthetic construct
DEDRIIEEIARLLEELLRELLELIKKLIETNRRLNEEHERAVRELARLLEELLDRLVKKGISDEKLKRIRERLKRALDDLERLHREINKRLEDLVRELEKLVREILKELKDALEELRRASARAGGEEVLRRLEEIVKKLLDLVRRILERLKEIHKDNVRLLRELNERLTRIVEDLVRLIREILREAG

SEQ ID NO: 156 - protein, 181 aa, synthetic construct
GDERAIRTLEDVKRRLREILERLADENAKAIKRLADLLDKLEKRNKEAIERLEEILEELKRVRRDEELLRVLETLLKIIEDILRENTKVLEDLLRLVEEILEANLRVVEELLRLAREILTEIVGDEDKLKEIEDELRRLLEELRRLDKAIKDRLRELKKDLDEANRRIKETLKKLLREVEK

SEQ ID NO: 157 - protein, 181 aa, synthetic construct
SKKELLEEVVRRAIELLKRHLEKLKRILEEIVRLLEEHLEKVERVLEAILSLLDDLLRRGGDERAIRTLEDVKRRLREILERLADENAKAIKRLADLLDKLEKRNKEAIERLEEILEELKRVRRDEELLRVLETLLKIIEDILRENTKVLEDLLRLVEEILEANLRVVEELLRLAREILTE

SEQ ID NO: 158 - protein, 173 aa, synthetic construct
ELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERGDSKAAEDILRVLEKLVKVSREAIKLILELSEHHVRVSTRIARLLLDVARKLAEVIKEAER

SEQ ID NO: 159 - protein, 173 aa, synthetic construct
SKEEKLKDDVRAVLEDLDRVLKELEKLSEDNLRELKRVLDRITDLHRRILDELRKGIGSEELLRRVEKVLKDNLDLLRKLVEEHKESSERDLKRVEDLVREIKEVLRKLLELEDRGTDIRKIEEEIERLLRKIRKAVEESKDLNRRNSERIEEVARRSEELARRLLKEIRERG

SEQ ID NO: 160 - protein, 189 aa, synthetic construct
VPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNLDEDVKRALEELVSRLRKLLEDVKKASEDIVREVERIVRELAKRSDEILKKLEDIVEKLRE

SEQ ID NO: 161 - protein, 189 aa, synthetic construct
DAEEVVKRLADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKEIVKRAEDILRKGKDKETVLRALRTLVKEYADLSKEVLERVERIVREYVKLSDEVVKSLAEIVEELIRIIEDLLRKGNL

SEQ ID NO: 162 - protein, 189 aa, synthetic construct; identical to SEQ ID NO: 160

SEQ ID NO: 163 - protein, 189 aa, synthetic construct; identical to SEQ ID NO: 161

SEQ ID NO: 164 - protein, 183 aa, synthetic construct
DELIKRLAEELERSLRELEEEIKRLKRELEESLKKLKEIIDRLAEEAEKLLAVLKRGEGSEEEALRALASLVRELIEVLRENDERLRDVLRRLIEALRKNNEILERVLRKLVRAAEERGRDESSREALEEARRRLEELLRELNEITKDLEAKLEKLLRDLNELTKALEEELKRLLDELKKRTD

SEQ ID NO: 165 - protein, 183 aa, synthetic construct
DEEETLRRLLERKVELAKEYLDVSKEVIDRTTKLLDEYLKTSKRIVDATVELLERGDLGPDELIKRLAEELERSLRELEEEIKRLKRELEESLKKLKEIIDRLAEEAEKLLAVLKRGEGSEEEALRALASLVRELIEVLRENDERLRDVLRRLIEALRKNNEILERVLRKLVRAAEERGRDES

SEQ ID NO: 166 - protein, 60 aa, synthetic construct
SVEELLRKLEEVLRKIREENERSLKELRDRAREIVKRNRETNRELEEVIKELEKRLSGAD

SEQ ID NO: 167 - protein, 60 aa, synthetic construct
SEDIVRKIERIVETIEREVRESVKKVEEIARDIRRKVDESVKNVEKLLRDVDKKARDRKK

SEQ ID NO: 168 - protein, 60 aa, synthetic construct
SESDDVIRKLRELLEELRTHVEKSIRDLRKILEDSTRHAKRSIEELERLLEEVRKKPGDE

SEQ ID NO: 169 - protein, 60 aa, synthetic construct
KGVDEEKLKDLIRKLRDILRRAAEAHKKLIDDARESLERAKREHEKLIDRLKKILEELER

SEQ ID NO: 170 - protein, 60 aa, synthetic construct
SELEEIEKKVRELTKRHRELVERVRKTVKELIETNRRLLETLTERIKRVLEEVRDLERRG

SEQ ID NO: 171 - protein, 60 aa, synthetic construct
DDKALEEAKRVLAENNEVIKDIKRALDELLKELEKSVRESEERLRRLLEEVKRVIEEVAT

SEQ ID NO: 172 - protein, 60 aa, synthetic construct
DEIRKVVKEITDLLKASNDKNRKVVEEIRDLLRKSKKLADELVERLRALVEDLRRRIDKS

SEQ ID NO: 173 - protein, 60 aa, synthetic construct
GSEDLKRVEERAREVSRRNEESMRRVKEDADRVSEANKEVLDRVREEVKRLIEEVRETLR

SEQ ID NO: 174 - protein, 60 aa, synthetic construct
SEAEDLEELIKELAELLKDVIRKLEKINRRLVKILEDIIRRLKEISKEAEEELRKGTVED

SEQ ID NO: 175 - protein, 60 aa, synthetic construct
DKDEAERRRRELKDKLDRLREEHEEVKRRLEEELTRLRETHKKIEKELREALKRVRDRST

SEQ ID NO: 176 - protein, 60 aa, synthetic construct; identical to SEQ ID NO: 174

SEQ ID NO: 177 - protein, 60 aa, synthetic construct; identical to SEQ ID NO: 175

SEQ ID NO: 178 - protein, 60 aa, synthetic construct; identical to SEQ ID NO: 89

SEQ ID NO: 179 - protein, 60 aa, synthetic construct
VDEKIAEEIERELEELRRMIKRLHEDLERKLKESEDELREIEARLEEKIRRLEKLERKRR

SEQ ID NO: 180 - protein, 60 aa, synthetic construct
SKKELLEEVVRRAIELLKRHLEKLKRILEEIVRLLEEHLEKVERVLEAILSLLDDLLRRG

SEQ ID NO: 181 - protein, 60 aa, synthetic construct
IVGDEDKLKEIEDELRRLLEELRRLDKAIKDRLRELKKDLDEANRRIKETLKKLLREVEK

SEQ ID NO: 182 - protein, 60 aa, synthetic construct
SKEEKLKDDVRAVLEDLDRVLKELEKLSEDNLRELKRVLDRITDLHRRILDELRKGIGSE

SEQ ID NO: 183 - protein, 60 aa, synthetic construct
DSKAAEDILRVLEKLVKVSREAIKLILELSEHHVRVSTRIARLLLDVARKLAEVIKEAER

SEQ ID NO: 184 - protein, 60 aa, synthetic construct
DAEEVVKRLADVLRENDETIRKVVEDLVRIAEENDRLWKKLVEDIAEILRRIVELLRRGG

SEQ ID NO: 185 - protein, 60 aa, synthetic construct
DEDVKRALEELVSRLRKLLEDVKKASEDIVREVERIVRELAKRSDEILKKLEDIVEKLRE

SEQ ID NO: 186 - protein, 60 aa, synthetic construct; identical to SEQ ID NO: 184

SEQ ID NO: 187 - protein, 60 aa, synthetic construct; identical to SEQ ID NO: 185

SEQ ID NO: 188 - protein, 60 aa, synthetic construct
DEEETLRRLLERKVELAKEYLDVSKEVIDRTTKLLDEYLKTSKRIVDATVELLERGDLGP

SEQ ID NO: 189 - protein, 60 aa, synthetic construct; identical to SEQ ID NO: 96

SEQ ID NO: 190 - protein, 57 aa, synthetic construct
EELLRKLEEVLRKIREENERSLKELRDRAREIVKRNRETNRELEEVIKELEKRLSGA

SEQ ID NO: 191 - protein, 57 aa, synthetic construct
EKVEELVRRIRRIVERVVEEDRRTVEEIEKIAREVVKRDRDSADRVRRTVEDVLRKA

SEQ ID NO: 192 - protein, 58 aa, synthetic construct
DIVRKIERIVETIEREVRESVKKVEEIARDIRRKVDESVKNVEKLLRDVDKKARDRKK

SEQ ID NO: 193 - protein, 53 aa, synthetic construct
SDDVIRKLRELLEELRTHVEKSIRDLRKILEDSTRHAKRSIEELERLLEEVRK

SEQ ID NO: 194 - protein, 55 aa, synthetic construct
EVRKTVEEISRRVAENVKRLEDLYRRMEEEVKKNLDRLRKRVEDIIREVEEARKK

SEQ ID NO: 195 - protein, 55 aa, synthetic construct
EKLKDLIRKLRDILRRAAEAHKKLIDDARESLERAKREHEKLIDRLKKILEELER

SEQ ID NO: 196 - protein, 57 aa, synthetic construct
LEEIEKKVRELTKRHRELVERVRKTVKELIETNRRLLETLTERIKRVLEEVRDLERR

SEQ ID NO: 197 - protein, 57 aa, synthetic construct
EDLKARLEDIIRRLAEDIERVKRETDKLLEELARIIEEVEKEMRETNKELDKEIKDG

SEQ ID NO: 198 - protein, 58 aa, synthetic construct
KALEEAKRVLAENNEVIKDIKRALDELLKELEKSVRESEERLRRLLEEVKRVIEEVAT

SEQ ID NO: 199 - protein, 57 aa, synthetic construct
IRKVVKEITDLLKASNDKNRKVVEEIRDLLRKSKKLADELVERLRALVEDLRRRIDK

SEQ ID NO: 200 - protein, 61 aa, synthetic construct
ETAEDIVRRIIEELKRILKEIEDLARRINREIERLVEEVERDNRDVNRAIEELLKDIARRG

SEQ ID NO: 201 - protein, 58 aa, synthetic construct
EDLKRVEERAREVSRRNEESMRRVKEDADRVSEANKEVLDRVREEVKRLIEEVRETLR

SEQ ID NO: 202 - protein, 54 aa, synthetic construct
AEDLEELIKELAELLKDVIRKLEKINRRLVKILEDIIRRLKEISKEAEEELRKG

SEQ ID NO: 203 - protein, 59 aa, synthetic construct
DILRDLERRLREILEESDRLLEELKRRLEEILRKSKELLRRLEEVLREILKRAEEVKRS

SEQ ID NO: 204 - protein, 62 aa, synthetic construct
EELIKEIVKLLEELLRVIEKILEDNIRLLEELVEVIKEILEKHLRLLEELVRVIERILREVG

SEQ ID NO: 205 - protein, 58 aa, synthetic construct
DEAERRRRELKDKLDRLREEHEEVKRRLEEELTRLRETHKKIEKELREALKRVRDRST

SEQ ID NO: 206 - protein, 54 aa, synthetic construct; identical to SEQ ID NO: 202

SEQ ID NO: 207 - protein, 59 aa, synthetic construct; identical to SEQ ID NO: 203

SEQ ID NO: 208 - protein, 62 aa, synthetic construct; identical to SEQ ID NO: 204

SEQ ID NO: 209 - protein, 58 aa, synthetic construct; identical to SEQ ID NO: 205

SEQ ID NO: 210 - protein, 58 aa, synthetic construct
DRIIEEIARLLEELLRELLELIKKLIETNRRLNEEHERAVRELARLLEELLDRLVKKG

SEQ ID NO: 211 - protein, 58 aa, synthetic construct
KLKRIRERLKRALDDLERLHREINKRLEDLVRELEKLVREILKELKDALEELRRASAR

SEQ ID NO: 212 - protein, 61 aa, synthetic construct
EVLRRLEEIVKKLLDLVRRILERLKEIHKDNVRLLRELNERLTRIVEDLVRLIREILREAG

SEQ ID NO: 213 - protein, 58 aa, synthetic construct
KIAEEIERELEELRRMIKRLHEDLERKLKESEDELREIEARLEEKIRRLEEKLERKRR

SEQ ID NO: 214 - protein, 58 aa, synthetic construct
KELLEEVVRRAIELLKRHLEKLKRILEEIVRLLEEHLEKVERVLEAILSLLDDLLRRG

SEQ ID NO: 215 - protein, 59 aa, synthetic construct
RAIRTLEDVKRRLREILERLADENAKAIKRLADLLDKLEKRNKEAIERLEEILEELKRV

SEQ ID NO: 216 - protein, 58 aa, synthetic construct
EELLRVLETLLKIIEDILRENTKVLEDLLRLVEEILEANLRVVEELLRLAREILTEIV

SEQ ID NO: 217 - protein, 55 aa, synthetic construct
DKLKEIEDELRRLLEELRRLDKAIKDRLRELKKDLDEANRRIKETLKKLLREVEK

SEQ ID NO: 218 - protein, 55 aa, synthetic construct
EEKLKDDVRAVLEDLDRVLKELEKLSEDNLRELKRVLDRITDLHRRILDELRKGI

SEQ ID NO: 219 - protein, 55 aa, synthetic construct
ELLRRVEKVLKDNLDLLRKLVEEHKESSERDLKRVEDLVREIKEVLRKLLELEDR

SEQ ID NO: 220 - protein, 54 aa, synthetic construct
RKIEEEIERLLRKIRKAVEESKDLNRRNSERIEEVARRSEELARRLLKEIRERG

SEQ ID NO: 221 - protein, 58 aa, synthetic construct
KAAEDILRVLEKLVKVSREAIKLILELSEHHVRVSTRIARLLLDVARKLAEVIKEAER

SEQ ID NO: 222 - protein, 57 aa, synthetic construct
EEVVKRLADVLRENDETIRKVVEDLVRIAEENDRLWKKLVEDIAEILRRIVELLRRG

SEQ ID NO: 223 - protein, 60 aa, synthetic construct
ELLDRLAKVVKSIVEKAEKILERLNRVSKAIAEKLKTIVDELNEVSKEIVKRAEDILRKG

SEQ ID NO: 224 - protein, 62 aa, synthetic construct
ETVLRALRTLVKEYADLSKEVLERVERIVREYVKLSDEVVKSLAEIVEELIRIIEDLLRKGN

SEQ ID NO: 225 - protein, 58 aa, synthetic construct
DVKRALEELVSRLRKLLEDVKKASEDIVREVERIVRELAKRSDEILKKLEDIVEKLRE

SEQ ID NO: 226 - protein, 57 aa, synthetic construct; identical to SEQ ID NO: 222

SEQ ID NO: 227 - protein, 60 aa, synthetic construct; identical to SEQ ID NO: 223

SEQ ID NO: 228 - protein, 61 aa, synthetic construct
ETVLRALRTLVKEYADLSKEVLERVERIVREYVKLSDEVVKSLAEIVEELIRIIEDLLRKG

SEQ ID NO: 229 - protein, 58 aa, synthetic construct; identical to SEQ ID NO: 225

SEQ ID NO: 230 - protein, 55 aa, synthetic construct
EETLRRLLERKVELAKEYLDVSKEVIDRTTKLLDEYLKTSKRIVDATVELLERGD

SEQ ID NO: 231 - protein, 56 aa, synthetic construct
ELIKRLAEELERSLRELEEEIKRLKRELEESLKKLKEIIDRLAEEAEKLLAVLKRG

SEQ ID NO: 232 - protein, 58 aa, synthetic construct
EEALRALASLVRELIEVLRENDERLRDVLRRLIEALRKNNEILERVLRKLVRAAEERG

SEQ ID NO: 233 - protein, 59 aa, synthetic construct
REALEEARRRLEELLRELNEITKDLEAKLEKLLRDLNELTKALEEELKRLLDELKKRTD

SEQ ID NO: 234 - protein, 57 aa, synthetic construct
EELLRKLKTRGVEEVAEGVVLLRRRGNRAREIVKRNRETNRELEEVIKELEKRLSGA

SEQ ID NO: 235 - protein, 57 aa, synthetic construct; identical to SEQ ID NO: 191

SEQ ID NO: 236 - protein, 58 aa, synthetic construct; identical to SEQ ID NO: 192

SEQ ID NO: 237 - protein, 57 aa, synthetic construct; identical to SEQ ID NO: 190

SEQ ID NO: 238 - protein, 57 aa, synthetic construct; identical to SEQ ID NO: 191

SEQ ID NO: 239 - protein, 58 aa, synthetic construct
RGVEEVAEGVVLLRRRGNESVKKVEEIARDIRRKVDESVKNVEKLLRDVDKKARDRKK

SEQ ID NO: 240 - protein, 57 aa, synthetic construct; identical to SEQ ID NO: 190

SEQ ID NO: 241 - protein, 57 aa, synthetic construct; identical to SEQ ID NO: 191

SEQ ID NO: 242 - protein, 58 aa, synthetic construct
TRGVEEVAEGVVLLRRRGNSVKKVEEIARDIRRKVDESVKNVEKLLRDVDKKARDRKK

SEQ ID NO: 243 - protein, 57 aa, synthetic construct; identical to SEQ ID NO: 190

SEQ ID NO: 244 - protein, 57 aa, synthetic construct; identical to SEQ ID NO: 191

SEQ ID NO: 245 - protein, 58 aa, synthetic construct
DKTRGVEEVAEGVVLLRRRGNKKVEEIARDIRRKVDESVKNVEKLLRDVDKKARDRKK

SEQ ID NO: 246 - protein, 57 aa, synthetic construct; identical to SEQ ID NO: 190

SEQ ID NO: 247 - protein, 57 aa, synthetic construct; identical to SEQ ID NO: 191

SEQ ID NO: 248 - protein, 58 aa, synthetic construct
DIKTRGVEEVAEGVVLLRRRGNKVEEIARDIRRKVDESVKNVEKLLRDVDKKARDRKK

SEQ ID NO: 249 - protein, 57 aa, synthetic construct; identical to SEQ ID NO: 190

SEQ ID NO: 250 - protein, 57 aa, synthetic construct; identical to SEQ ID NO: 191

SEQ ID NO: 251 - protein, 58 aa, synthetic construct
DIVRKKTRGVEEVAEGVVLLRRRGNEIARDIRRKVDESVKNVEKLLRDVDKKARDRKK

SEQ ID NO: 252 - protein, 57 aa, synthetic construct
EELLRKLEEVKTRGVEEVAEGVVLLRRRGNEIVKRNRETNRELEEVIKELEKRLSGA

SEQ ID NO: 253 - protein, 57 aa, synthetic construct; identical to SEQ ID NO: 191

SEQ ID NO: 254 - protein, 58 aa, synthetic construct; identical to SEQ ID NO: 192

SEQ ID NO: 255 - protein, 57 aa, synthetic construct; identical to SEQ ID NO: 190

SEQ ID NO: 256 - protein, 57 aa, synthetic construct; identical to SEQ ID NO: 191

SEQ ID NO: 257 - protein, 58 aa, synthetic construct
DIVRKIERIVETIEREKTRGVEEVAEGVVLLRRRGNESVKNVEKLLRDVDKKARDRKK

SEQ ID NO: 258 - protein, 57 aa, synthetic construct; identical to SEQ ID NO: 190

SEQ ID NO: 259 - protein, 57 aa, synthetic construct; identical to SEQ ID NO: 191

SEQ ID NO: 260 - protein, 58 aa, synthetic construct
DIVRKIERIVETIEREVRESVKKVEEIARDIRRKKTRGVEEVAEGVVLLRRRGNDRKK

SEQ ID NO: 261 - protein, 57 aa, synthetic construct
TRGVEEVAEGVVLLRRRGNRSLKELRDRAREIVKRNRETNRELEEVIKELEKRLSGA

SEQ ID NO: 262 - protein, 57 aa, synthetic construct; identical to SEQ ID NO: 191

SEQ ID NO: 263 - protein, 58 aa, synthetic construct; identical to SEQ ID NO: 192

SEQ ID NO: 264 - protein, 57 aa, synthetic construct
EELLRKLEEVLRKIREENERSKTRGVEEVAEGVVLLRRRGNELEEVIKELEKRLSGA

SEQ ID NO: 265 - protein, 57 aa, synthetic construct; identical to SEQ ID NO: 191

SEQ ID NO: 266 - protein, 58 aa, synthetic construct; identical to SEQ ID NO: 192

SEQ ID NO: 267 - protein, 57 aa, synthetic construct
EELKTRGVEEVAEGVVLLRRRGNELRDRAREIVKRNRETNRELEEVIKELEKRLSGA

SEQ ID NO: 268 - protein, 57 aa, synthetic construct; identical to SEQ ID NO: 191

SEQ ID NO: 269 - protein, 58 aa, synthetic construct; identical to SEQ ID NO: 192

SEQ ID NO: 270 - protein, 53 aa, synthetic construct
SDDVIRKKTRGVEEVAEGVVLLRRRGNKILEDSTRHAKRSIEELERLLEEVRK

SEQ ID NO: 271 - protein, 55 aa, synthetic construct; identical to SEQ ID NO: 194

SEQ ID NO: 272 - protein, 55 aa, synthetic construct; identical to SEQ ID NO: 195

SEQ ID NO: 273 - protein, 53 aa, synthetic construct; identical to SEQ ID NO: 193

SEQ ID NO: 274 - protein, 55 aa, synthetic construct; identical to SEQ ID NO: 194

SEQ ID NO: 275 - protein, 55 aa, synthetic construct
RGVEEVAEGVVLLRRRGNEAHKKLIDDARESLERAKREHEKLIDRLKKILEELER

SEQ ID NO: 276 - protein, 53 aa, synthetic construct; identical to SEQ ID NO: 193

SEQ ID NO: 277 - protein, 55 aa, synthetic construct; identical to SEQ ID NO: 194

SEQ ID NO: 278 - protein, 55 aa, synthetic construct
EKLKDLIRKLKTRGVEEVAEGVVLLRRRGNSLERAKREHEKLIDRLKKILEELER

SEQ ID NO: 279 - protein, 53 aa, synthetic construct; identical to SEQ ID NO: 193

SEQ ID NO: 280 - protein, 55 aa, synthetic construct; identical to SEQ ID NO: 194

SEQ ID NO: 281 - protein, 55 aa, synthetic construct
EKLKDLIRKLRDIKTRGVEEVAEGVVLLRRRGNRAKREHEKLIDRLKKILEELER

SEQ ID NO: 282 - protein, 53 aa, synthetic construct; identical to SEQ ID NO: 193

SEQ ID NO: 283 - protein, 55 aa, synthetic construct; identical to SEQ ID NO: 194

SEQ ID NO: 284 - protein, 55 aa, synthetic construct
EKLKDLIRKLRDILRRAAEAKTRGVEEVAEGVVLLRRRGNKLIDRLKKILEELER

SEQ ID NO: 285 - protein, 53 aa, synthetic construct; identical to SEQ ID NO: 193

SEQ ID NO: 286 - protein, 55 aa, synthetic construct; identical to SEQ ID NO: 194

SEQ ID NO: 287 - protein, 55 aa, synthetic construct
EKLKDLIRKLRDILRRAAEAHKKLIDDKTRGVEEVAEGVVLLRRRGNKILEELER

SEQ ID NO: 288 - protein, 53 aa, synthetic construct; identical to SEQ ID NO: 193

SEQ ID NO: 289 - protein, 55 aa, synthetic construct; identical to SEQ ID NO: 194

SEQ ID NO: 290 - protein, 55 aa, synthetic construct
EKLKDLIRKLRDILRRAAEAHKKLIDDARESKTRGVEEVAEGVVLLRRRGNELER

SEQ ID NO: 291 - protein, 53 aa, synthetic construct; identical to SEQ ID NO: 193

SEQ ID NO: 292 - protein, 55 aa, synthetic construct; identical to SEQ ID NO: 194

SEQ ID NO: 293 - protein, 55 aa, synthetic construct
EKLKDLIRKLRDILRRAAEAHKKLIDDARESLERKTRGVEEVAEGVVLLRRRGNR

SEQ ID NO: 294 - protein, 53 aa, synthetic construct
SDDVIRKLRELLEELRTHVEKSKTRGVEEVAEGVVLLRRRGNELERLLEEVRK

SEQ ID NO: 295 - protein, 55 aa, synthetic construct; identical to SEQ ID NO: 194

SEQ ID NO: 296 - protein, 55 aa, synthetic construct; identical to SEQ ID NO: 195

SEQ ID NO: 297 - protein, 53 aa, synthetic construct
SDDVIRKLRELLEELRTHVEKSIRDKTRGVEEVAEGVVLLRRRGNRLLEEVRK

SEQ ID NO: 298 - protein, 55 aa, synthetic construct; identical to SEQ ID NO: 194

SEQ ID NO: 299 - protein, 55 aa, synthetic construct; identical to SEQ ID NO: 195

SEQ ID NO: 300 - protein, 53 aa, synthetic construct
SDDVIRKLRELLEELRTHVEKSIRDLRKIKTRGVEEVAEGVVLLRRRGNEVRK

SEQ ID NO: 301 - protein, 55 aa, synthetic construct; identical to SEQ ID NO: 194

SEQ ID NO: 302 - protein, 55 aa, synthetic construct; identical to SEQ ID NO: 195

SEQ ID NO: 303 - protein, 53 aa, synthetic construct
SDDVIRKLRELLEELRTHVEKSIRDLRKILEDKTRGVEEVAEGVVLLRRRGNK

SEQ ID NO: 304 - protein, 55 aa, synthetic construct; identical to SEQ ID NO: 194

SEQ ID NO: 305 - protein, 55 aa, synthetic construct; identical to SEQ ID NO: 195

SEQ ID NO: 306 - protein, 57 aa, synthetic construct
LEEIEKKKTRGVEEVAEGVVLLRRRGNELIETNRRLLETLTERIKRVLEEVRDLERR

SEQ ID NO: 307 - protein, 57 aa, synthetic construct; identical to SEQ ID NO: 197

SEQ ID NO: 308 - protein, 58 aa, synthetic construct; identical to SEQ ID NO: 198

SEQ ID NO: 309 - protein, 57 aa, synthetic construct
LEEIEKKVKTRGVEEVAEGVVLLRRRGNLIETNRRLLETLTERIKRVLEEVRDLERR

SEQ ID NO: 310 - protein, 57 aa, synthetic construct; identical to SEQ ID NO: 197

SEQ ID NO: 311 - protein, 58 aa, synthetic construct; identical to SEQ ID NO: 198

SEQ ID NO: 312 - protein, 57 aa, synthetic construct; identical to SEQ ID NO: 196

SEQ ID NO: 313 - protein, 58 aa, synthetic construct
EDLKARLEDIIRRLAEDIERVKRETDKLLEELARIIEEVEKEMRETNKELDKEIKDGG

SEQ ID NO: 314 - protein, 58 aa, synthetic construct
RGVEEVAEGVVLLRRRGNDIKRALDELLKELEKSVRESEERLRRLLEEVKRVIEEVAT

SEQ ID NO: 315 - protein, 57 aa, synthetic construct; identical to SEQ ID NO: 196

SEQ ID NO: 316 - protein, 57 aa, synthetic construct; identical to SEQ ID NO: 197

SEQ ID NO: 317 - protein, 58 aa, synthetic construct
KAKTRGVEEVAEGVVLLRRRGNALDELLKELEKSVRESEERLRRLLEEVKRVIEEVAT

SEQ ID NO: 318 - protein, 57 aa, synthetic construct
LEEIEKKVREKTRGVEEVAEGVVLLRRRGNETNRRLLETLTERIKRVLEEVRDLERR

SEQ ID NO: 319 - protein, 57 aa, synthetic construct; identical to SEQ ID NO: 197

SEQ ID NO: 320 - protein, 58 aa, synthetic construct; identical to SEQ ID NO: 198

SEQ ID NO: 321 - protein, 57 aa, synthetic construct; identical to SEQ ID NO: 196

SEQ ID NO: 322 - protein, 58 aa, synthetic construct; identical to SEQ ID NO: 313

SEQ ID NO: 323 - protein, 58 aa, synthetic construct
KALEEKTRGVEEVAEGVVLLRRRGNELLKELEKSVRESEERLRRLLEEVKRVIEEVAT

SEQ ID NO: 324 - protein, 57 aa, synthetic construct
LEEIEKKVRELKTRGVEEVAEGVVLLRRRGNTNRRLLETLTERIKRVLEEVRDLERR

SEQ ID NO: 325 - protein, 57 aa, synthetic construct; identical to SEQ ID NO: 197

SEQ ID NO: 326 - protein, 58 aa, synthetic construct; identical to SEQ ID NO: 198

SEQ ID NO: 327 - protein, 57 aa, synthetic construct; identical to SEQ ID NO: 196

SEQ ID NO: 328 - protein, 57 aa, synthetic construct; identical to SEQ ID NO: 197

SEQ ID NO: 329 - protein, 58 aa, synthetic construct
KALEEAKRVLAENNEVIKDKTRGVEEVAEGVVLLRRRGNERLRRLLEEVKRVIEEVAT

SEQ ID NO: 330 - protein, 57 aa, synthetic construct; identical to SEQ ID NO: 196

SEQ ID NO: 331 - protein, 57 aa, synthetic construct; identical to SEQ ID NO: 197

SEQ ID NO: 332 - protein, 58 aa, synthetic construct
KALEEAKRVLAENNEVIKDIKRAKTRGVEEVAEGVVLLRRRGNRLLEEVKRVIEEVAT

SEQ ID NO: 333 - protein, 57 aa, synthetic construct; identical to SEQ ID NO: 196

SEQ ID NO: 334 - protein, 57 aa, synthetic construct; identical to SEQ ID NO: 197

SEQ ID NO: 335 - protein, 58 aa, synthetic construct
KALEEAKRVLAENNEVIKDIKRALDELKTRGVEEVAEGVVLLRRRGNEVKRVIEEVAT

SEQ ID NO: 336 - protein, 57 aa, synthetic construct; identical to SEQ ID NO: 196

SEQ ID NO: 337 - protein, 57 aa, synthetic construct; identical to SEQ ID NO: 197

SEQ ID NO: 338 - protein, 58 aa, synthetic construct
KALEEAKRVLAENNEVIKDIKRALDELLKELEKSKTRGVEEVAEGVVLLRRRGNEVAT

SEQ ID NO: 339 - protein, 57 aa, synthetic construct; identical to SEQ ID NO: 196

SEQ ID NO: 340 - protein, 57 aa, synthetic construct; identical to SEQ ID NO: 197

SEQ ID NO: 341 - protein, 58 aa, synthetic construct
KALEEAKRVLAENNEVIKDIKRALDELLKELEKSVKTRGVEEVAEGVVLLRRRGNVAT

SEQ ID NO: 342 - protein, 57 aa, synthetic construct; identical to SEQ ID NO: 196

SEQ ID NO: 343 - protein, 57 aa, synthetic construct; identical to SEQ ID NO: 197

SEQ ID NO: 344 - protein, 58 aa, synthetic construct
KALEEAKRVLAENNEVIKDIKRALDELLKELEKSVREKTRGVEEVAEGVVLLRRRGNT

SEQ ID NO: 345 - protein, 57 aa, synthetic construct; identical to SEQ ID NO: 196

SEQ ID NO: 346 - protein, 57 aa, synthetic construct; identical to SEQ ID NO: 197

SEQ ID NO: 347 - protein, 58 aa, synthetic construct
KALEEAKRVLAENNEVIKDIKRALDELLKELEKSVRESKTRGVEEVAEGVVLLRRRGN

SEQ ID NO: 348 - protein, 57 aa, synthetic construct
LEEIEKKVRELTKRKTRGVEEVAEGVVLLRRRGNRLLETLTERIKRVLEEVRDLERR

SEQ ID NO: 349 - protein, 57 aa, synthetic construct; identical to SEQ ID NO: 197

SEQ ID NO: 350 - protein, 58 aa, synthetic construct; identical to SEQ ID NO: 198

SEQ ID NO: 351 - protein, 57 aa, synthetic construct
LEEIEKKVRELTKRHRELKTRGVEEVAEGVVLLRRRGNTLTERIKRVLEEVRDLERR

SEQ ID NO: 352 - protein, 57 aa, synthetic construct; identical to SEQ ID NO: 197

SEQ ID NO: 353 - protein, 58 aa, synthetic construct; identical to SEQ ID NO: 198

SEQ ID NO: 354 - protein, 57 aa, synthetic construct
LEEIEKKVRELTKRHRELVERKTRGVEEVAEGVVLLRRRGNERIKRVLEEVRDLERR

SEQ ID NO: 355 - protein, 57 aa, synthetic construct; identical to SEQ ID NO: 197

SEQ ID NO: 356 - protein, 58 aa, synthetic construct; identical to SEQ ID NO: 198

SEQ ID NO: 357 - protein, 57 aa, synthetic construct
LEEIEKKVRELTKRHRELVERVRKTKTRGVEEVAEGVVLLRRRGNRVLEEVRDLERR

SEQ ID NO: 358 - protein, 57 aa, synthetic construct; identical to SEQ ID NO: 197

SEQ ID NO: 359 - protein, 58 aa, synthetic construct; identical to SEQ ID NO: 198

SEQ ID NO: 360 - protein, 57 aa, synthetic construct
LEEIEKKVRELTKRHRELVERVRKTVKTRGVEEVAEGVVLLRRRGNVLEEVRDLERR

SEQ ID NO: 361 - protein, 57 aa, synthetic construct; identical to SEQ ID NO: 197

SEQ ID NO: 362 - protein, 58 aa, synthetic construct; identical to SEQ ID NO: 198

SEQ ID NO: 363 - protein, 57 aa, synthetic construct
KTRGVEEVAEGVVLLRRRGNRVRKTVKELIETNRRLLETLTERIKRVLEEVRDLERR

SEQ ID NO: 364 - protein, 57 aa, synthetic construct; identical to SEQ ID NO: 197

SEQ ID NO: 365 - protein, 58 aa, synthetic construct; identical to SEQ ID NO: 198

SEQ ID NO: 366 - protein, 57 aa, synthetic construct
LEEIEKKVRELTKRHRELVERVRKTVKELKTRGVEEVAEGVVLLRRRGNEVRDLERR

SEQ ID NO: 367 - protein, 57 aa, synthetic construct; identical to SEQ ID NO: 197

SEQ ID NO: 368 - protein, 58 aa, synthetic construct; identical to SEQ ID NO: 198

SEQ ID NO: 369 - protein, 57 aa, synthetic construct
LEEIEKKVRELTKRHRELVERVRKTVKELIETKTRGVEEVAEGVVLLRRRGNDLERR

SEQ ID NO: 370 - protein, 57 aa, synthetic construct; identical to SEQ ID NO: 197

SEQ ID NO: 371 - protein, 58 aa, synthetic construct; identical to SEQ ID NO: 198

SEQ ID NO: 372 - protein, 57 aa, synthetic construct
LEEIEKKVRELTKRHRELVERVRKTVKELIETNRRLKTRGVEEVAEGVVLLRRRGNR

SEQ ID NO: 373 - protein, 57 aa, synthetic construct; identical to SEQ ID NO: 197

SEQ ID NO: 374 - protein, 58 aa, synthetic construct; identical to SEQ ID NO: 198

SEQ ID NO: 375 - protein, 57 aa, synthetic construct
LEEIKTRGVEEVAEGVVLLRRRGNTVKELIETNRRLLETLTERIKRVLEEVRDLERR

SEQ ID NO: 376 - protein, 57 aa, synthetic construct; identical to SEQ ID NO: 197

SEQ ID NO: 377 - protein, 58 aa, synthetic construct; identical to SEQ ID NO: 198

SEQ ID NO: 378 - protein, 57 aa, synthetic construct
IRKVVKEKTRGVEEVAEGVVLLRRRGNDLLRKSKKLADELVERLRALVEDLRRRIDK

SEQ ID NO: 379 - protein, 61 aa, synthetic construct; identical to SEQ ID NO: 200

SEQ ID NO: 380 - protein, 58 aa, synthetic construct; identical to SEQ ID NO: 201

SEQ ID NO: 381 - protein, 57 aa, synthetic construct; identical to SEQ ID NO: 199

SEQ ID NO: 382 - protein, 61 aa, synthetic construct; identical to SEQ ID NO: 200

SEQ ID NO: 383 - protein, 58 aa, synthetic construct
RGVEEVAEGVVLLRRRGNESMRRVKEDADRVSEANKEVLDRVREEVKRLIEEVRETLR

SEQ ID NO: 384 - protein, 57 aa, synthetic construct; identical to SEQ ID NO: 199

SEQ ID NO: 385 - protein, 61 aa, synthetic construct; identical to SEQ ID NO: 200

SEQ ID NO: 386 - protein, 58 aa, synthetic construct
EDKTRGVEEVAEGVVLLRRRGNRVKEDADRVSEANKEVLDRVREEVKRLIEEVRETLR

SEQ ID NO: 387 - protein, 57 aa, synthetic construct; identical to SEQ ID NO: 199

SEQ ID NO: 388 - protein, 61 aa, synthetic construct; identical to SEQ ID NO: 200

SEQ ID NO: 389 - protein, 58 aa, synthetic construct
EDLKRKTRGVEEVAEGVVLLRRRGNEDADRVSEANKEVLDRVREEVKRLIEEVRETLR

SEQ ID NO: 390 - protein, 57 aa, synthetic construct; identical to SEQ ID NO: 199

SEQ ID NO: 391 - protein, 61 aa, synthetic construct; identical to SEQ ID NO: 200

SEQ ID NO: 392 - protein, 58 aa, synthetic construct
EDLKRVKTRGVEEVAEGVVLLRRRGNDADRVSEANKEVLDRVREEVKRLIEEVRETLR

SEQ ID NO: 393 - protein, 57 aa, synthetic construct; identical to SEQ ID NO: 199

SEQ ID NO: 394 - protein, 61 aa, synthetic construct; identical to SEQ ID NO: 200

SEQ ID NO: 395 - protein, 58 aa, synthetic construct
EDLKRVEEKTRGVEEVAEGVVLLRRRGNDRVSEANKEVLDRVREEVKRLIEEVRETLR

SEQ ID NO: 396 - protein, 57 aa, synthetic construct; identical to SEQ ID NO: 199

SEQ ID NO: 397 - protein, 61 aa, synthetic construct; identical to SEQ ID NO: 200

SEQ ID NO: 398 - protein, 58 aa, synthetic construct
EDLKRVEERKTRGVEEVAEGVVLLRRRGNRVSEANKEVLDRVREEVKRLIEEVRETLR

SEQ ID NO: 399 - protein, 57 aa, synthetic construct; identical to SEQ ID NO: 199

SEQ ID NO: 400 - protein, 61 aa, synthetic construct; identical to SEQ ID NO: 200

SEQ ID NO: 401 - protein, 58 aa, synthetic construct
EDLKRVEERAREKTRGVEEVAEGVVLLRRRGNEANKEVLDRVREEVKRLIEEVRETLR

SEQ ID NO: 402 - protein, 57 aa, synthetic construct; identical to SEQ ID NO: 199

SEQ ID NO: 403 - protein, 61 aa, synthetic construct; identical to SEQ ID NO: 200

SEQ ID NO: 404 - protein, 58 aa, synthetic construct
EDLKRVEERAREVKTRGVEEVAEGVVLLRRRGNANKEVLDRVREEVKRLIEEVRETLR

SEQ ID NO: 405 - protein, 57 aa, synthetic construct; identical to SEQ ID NO: 199

SEQ ID NO: 406 - protein, 61 aa, synthetic construct; identical to SEQ ID NO: 200

SEQ ID NO: 407 - protein, 58 aa, synthetic construct
EDLKRVEERAREVSKTRGVEEVAEGVVLLRRRGNNKEVLDRVREEVKRLIEEVRETLR

SEQ ID NO: 408 - protein, 57 aa, synthetic construct; identical to SEQ ID NO: 199

SEQ ID NO: 409 - protein, 61 aa, synthetic construct; identical to SEQ ID NO: 200

SEQ ID NO: 410 - protein, 58 aa, synthetic construct
EDLKRVEERAREVSRRNKTRGVEEVAEGVVLLRRRGNVLDRVREEVKRLIEEVRETLR

SEQ ID NO: 411 - protein, 57 aa, synthetic construct; identical to SEQ ID NO: 199

SEQ ID NO: 412 - protein, 61 aa, synthetic construct; identical to SEQ ID NO: 200

SEQ ID NO: 413 - protein, 58 aa, synthetic construct
EDLKRVEERAREVSRRNEESKTRGVEEVAEGVVLLRRRGNRVREEVKRLIEEVRETLR

SEQ ID NO: 414 - protein, 57 aa, synthetic construct; identical to SEQ ID NO: 199

SEQ ID NO: 415 - protein, 61 aa, synthetic construct; identical to SEQ ID NO: 200

SEQ ID NO: 416 - protein, 58 aa, synthetic construct
EDLKRVEERAREVSRRNEESMRRKTRGVEEVAEGVVLLRRRGNEEVKRLIEEVRETLR

SEQ ID NO: 417 - protein, 57 aa, synthetic construct; identical to SEQ ID NO: 199

SEQ ID NO: 418 - protein, 61 aa, synthetic construct; identical to SEQ ID NO: 200

SEQ ID NO: 419 - protein, 58 aa, synthetic construct
EDLKRVEERAREVSRRNEESMRRVKTRGVEEVAEGVVLLRRRGNEVKRLIEEVRETLR

SEQ ID NO: 420 - protein, 57 aa, synthetic construct; identical to SEQ ID NO: 199

SEQ ID NO: 421 - protein, 61 aa, synthetic construct; identical to SEQ ID NO: 200

SEQ ID NO: 422 - protein, 58 aa, synthetic construct
EDLKRVEERAREVSRRNEESMRRVKEDKTRGVEEVAEGVVLLRRRGNRLIEEVRETLR

SEQ ID NO: 423 - protein, 57 aa, synthetic construct; identical to SEQ ID NO: 199

SEQ ID NO: 424 - protein, 61 aa, synthetic construct; identical to SEQ ID NO: 200

SEQ ID NO: 425 - protein, 58 aa, synthetic construct
EDLKRVEERAREVSRRNEESMRRVKEDAKTRGVEEVAEGVVLLRRRGNLIEEVRETLR

SEQ ID NO: 426 - protein, 57 aa, synthetic construct; identical to SEQ ID NO: 199

SEQ ID NO: 427 - protein, 61 aa, synthetic construct; identical to SEQ ID NO: 200

SEQ ID NO: 428 - protein, 58 aa, synthetic construct
EDLKRVEERAREVSRRNEESMRRVKEDADRKTRGVEEVAEGVVLLRRRGNEEVRETLR

SEQ ID NO: 429 - protein, 57 aa, synthetic construct; identical to SEQ ID NO: 199

SEQ ID NO: 430 - protein, 61 aa, synthetic construct; identical to SEQ ID NO: 200

SEQ ID NO: 431 - protein, 58 aa, synthetic construct
EDLKRVEERAREVSRRNEESMRRVKEDADRVKTRGVEEVAEGVVLLRRRGNEVRETLR

SEQ ID NO: 432 - protein, 57 aa, synthetic construct; identical to SEQ ID NO: 199

SEQ ID NO: 433 - protein, 61 aa, synthetic construct; identical to SEQ ID NO: 200

SEQ ID NO: 434 - protein, 58 aa, synthetic construct
EDLKRVEERAREVSRRNEESMRRVKEDADRVSEAKTRGVEEVAEGVVLLRRRGNETLR

SEQ ID NO: 435 - protein, 57 aa, synthetic construct; identical to SEQ ID NO: 199

SEQ ID NO: 436 - protein, 61 aa, synthetic construct; identical to SEQ ID NO: 200

SEQ ID NO: 437 - protein, 58 aa, synthetic construct
EDLKRVEERAREVSRRNEESMRRVKEDADRVSEANKTRGVEEVAEGVVLLRRRGNTLR

SEQ ID NO: 438 - protein, 57 aa, synthetic construct; identical to SEQ ID NO: 199

SEQ ID NO: 439 - protein, 61 aa, synthetic construct; identical to SEQ ID NO: 200

SEQ ID NO: 440 - protein, 58 aa, synthetic construct
EDLKRVEERAREVSRRNEESMRRVKEDADRVSEANKEKTRGVEEVAEGVVLLRRRGNR

SEQ ID NO: 441 - protein, 57 aa, synthetic construct; identical to SEQ ID NO: 199

SEQ ID NO: 442 - protein, 61 aa, synthetic construct; identical to SEQ ID NO: 200

SEQ ID NO: 443 - protein, 58 aa, synthetic construct
EDLKRVEERAREVSRRNEESMRRVKEDADRVSEANKEVKTRGVEEVAEGVVLLRRRGN

SEQ ID NO: 444 - protein, 57 aa, synthetic construct
IRKVVKEITDLLKASKTRGVEEVAEGVVLLRRRGNLADELVERLRALVEDLRRRIDK

SEQ ID NO: 445 - protein, 61 aa, synthetic construct; identical to SEQ ID NO: 200

SEQ ID NO: 446 - protein, 58 aa, synthetic construct; identical to SEQ ID NO: 201

SEQ ID NO: 447 - protein, 57 aa, synthetic construct
IRKVVKEITDLLKASNDKKTRGVEEVAEGVVLLRRRGNELVERLRALVEDLRRRIDK

SEQ ID NO: 448 - protein, 61 aa, synthetic construct; identical to SEQ ID NO: 200

SEQ ID NO: 449 - protein, 58 aa, synthetic construct; identical to SEQ ID NO: 201

SEQ ID NO: 450 - protein, 57 aa, synthetic construct
IRKVVKEITDLLKASNDKNRKKTRGVEEVAEGVVLLRRRGNERLRALVEDLRRRIDK

SEQ ID NO: 451 - protein, 61 aa, synthetic construct; identical to SEQ ID NO: 200

SEQ ID NO: 452 - protein, 58 aa, synthetic construct; identical to SEQ ID NO: 201

SEQ ID NO: 453 - protein, 57 aa, synthetic construct
IRKVVKEITDLLKASNDKNRKVKTRGVEEVAEGVVLLRRRGNRLRALVEDLRRRIDK

SEQ ID NO: 454 - protein, 61 aa, synthetic construct; identical to SEQ ID NO: 200

SEQ ID NO: 455 - protein, 58 aa, synthetic construct; identical to SEQ ID NO: 201

SEQ ID NO: 456 - protein, 57 aa, synthetic construct
IRKVVKEITDLLKASNDKNRKVVEEIRDLLRKSKTRGVEEVAEGVVLLRRRGNRIDK

SEQ ID NO: 457 - protein, 61 aa, synthetic construct; identical to SEQ ID NO: 200

SEQ ID NO: 458 - protein, 58 aa, synthetic construct; identical to SEQ ID NO: 201

SEQ ID NO: 459 - protein, 58 aa, synthetic construct
IRKVVKEITDLLKASNDKNRKVVEEIRDLLRKSKKLKTRGVEEVAEGVVLLRRRGNKS

SEQ ID NO: 460 - protein, 61 aa, synthetic construct; identical to SEQ ID NO: 200

SEQ ID NO: 461 - protein, 58 aa, synthetic construct; identical to SEQ ID NO: 201

SEQ ID NO: 462 - protein, 57 aa, synthetic construct
IRKKTRGVEEVAEGVVLLRRRGNEEIRDLLRKSKKLADELVERLRALVEDLRRRIDK

SEQ ID NO: 463 - protein, 61 aa, synthetic construct; identical to SEQ ID NO: 200

SEQ ID NO: 464 - protein, 58 aa, synthetic construct; identical to SEQ ID NO: 201

SEQ ID NO: 465 - protein, 57 aa, synthetic construct
IRKVKTRGVEEVAEGVVLLRRRGNEIRDLLRKSKKLADELVERLRALVEDLRRRIDK

SEQ ID NO: 466 - protein, 61 aa, synthetic construct; identical to SEQ ID NO: 200

SEQ ID NO: 467 - protein, 58 aa, synthetic construct; identical to SEQ ID NO: 201

SEQ ID NO: 468 - protein, 54 aa, synthetic construct
AEDLEELKTRGVEEVAEGVVLLRRRGNRLVKILEDIIRRLKEISKEAEEELRKG

SEQ ID NO: 469 - protein, 59 aa, synthetic construct; identical to SEQ ID NO: 203

SEQ ID NO: 470 - protein, 62 aa, synthetic construct; identical to SEQ ID NO: 204

SEQ ID NO: 471 - protein, 58 aa, synthetic construct; identical to SEQ ID NO: 205

SEQ ID NO: 472 - protein, 54 aa, synthetic construct; identical to SEQ ID NO: 202

SEQ ID NO: 473 - protein, 59 aa, synthetic construct; identical to SEQ ID NO: 203

SEQ ID NO: 474 - protein, 62 aa, synthetic construct; identical to SEQ ID NO: 204

SEQ ID NO: 475 - protein, 58 aa, synthetic construct
KTRGVEEVAEGVVLLRRRGNHEEVKRRLEEELTRLRETHKKIEKELREALKRVRDRST

SEQ ID NO: 476 - protein, 54 aa, synthetic construct; identical to SEQ ID NO: 202

SEQ ID NO: 477 - protein, 59 aa, synthetic construct; identical to SEQ ID NO: 203

SEQ ID NO: 478 - protein, 62 aa, synthetic construct; identical to SEQ ID NO: 204

SEQ ID NO: 479 - protein, 58 aa, synthetic construct
DEKTRGVEEVAEGVVLLRRRGNEVKRRLEEELTRLRETHKKIEKELREALKRVRDRST

SEQ ID NO: 480 - protein, 54 aa, synthetic construct; identical to SEQ ID NO: 202

SEQ ID NO: 481 - protein, 59 aa, synthetic construct; identical to SEQ ID NO: 203

SEQ ID NO: 482 - protein, 62 aa, synthetic construct; identical to SEQ ID NO: 204

SEQ ID NO: 483 - protein, 58 aa, synthetic construct
DEAERRKTRGVEEVAEGVVLLRRRGNRLEEELTRLRETHKKIEKELREALKRVRDRST

SEQ ID NO: 484 - protein, 54 aa, synthetic construct; identical to SEQ ID NO: 202

SEQ ID NO: 485 - protein, 59 aa, synthetic construct; identical to SEQ ID NO: 203

SEQ ID NO: 486 - protein, 62 aa, synthetic construct; identical to SEQ ID NO: 204

SEQ ID NO: 487 - protein, 58 aa, synthetic construct
DEAERRRREKTRGVEEVAEGVVLLRRRGNEELTRLRETHKKIEKELREALKRVRDRST

SEQ ID NO: 488 - protein, 54 aa, synthetic construct; identical to SEQ ID NO: 202

SEQ ID NO: 489 - protein, 59 aa, synthetic construct; identical to SEQ ID NO: 203

SEQ ID NO: 490 - protein, 62 aa, synthetic construct; identical to SEQ ID NO: 204

SEQ ID NO: 491 - protein, 58 aa, synthetic construct
DEAERRRRELKDKKTRGVEEVAEGVVLLRRRGNRLRETHKKIEKELREALKRVRDRST

SEQ ID NO: 492 - protein, 54 aa, synthetic construct; identical to SEQ ID NO: 202

SEQ ID NO: 493 - protein, 59 aa, synthetic construct; identical to SEQ ID NO: 203

SEQ ID NO: 494 - protein, 62 aa, synthetic construct; identical to SEQ ID NO: 204

SEQ ID NO: 495 - protein, 58 aa, synthetic construct
DEAERRRRELKDKLDRLREEKTRGVEEVAEGVVLLRRRGNKIEKELREALKRVRDRST

SEQ ID NO: 496 - protein, 54 aa, synthetic construct; identical to SEQ ID NO: 202

SEQ ID NO: 497 - protein, 59 aa, synthetic construct; identical to SEQ ID NO: 203

SEQ ID NO: 498 - protein, 62 aa, synthetic construct; identical to SEQ ID NO: 204

SEQ ID NO: 499 - protein, 58 aa, synthetic construct
DEAERRRRELKDKLDRLREEHEEVKRRLEEEKTRGVEEVAEGVVLLRRRGNRVRDRST

SEQ ID NO: 500 - protein, 54 aa, synthetic construct; identical to SEQ ID NO: 202

SEQ ID NO: 501 - protein, 59 aa, synthetic construct; identical to SEQ ID NO: 203

SEQ ID NO: 502 - protein, 62 aa, synthetic construct; identical to SEQ ID NO: 204

SEQ ID NO: 503 - protein, 58 aa, synthetic construct
DEAERRRRELKDKLDRLREEHEEVKRRLEEELTRKTRGVEEVAEGVVLLRRRGNDRST

SEQ ID NO: 504 - protein, 54 aa, synthetic construct; identical to SEQ ID NO: 202

SEQ ID NO: 505 - protein, 59 aa, synthetic construct; identical to SEQ ID NO: 203

SEQ ID NO: 506 - protein, 62 aa, synthetic construct; identical to SEQ ID NO: 204

SEQ ID NO: 507 - protein, 58 aa, synthetic construct
DEAERRRRELKDKLDRLREEHEEVKRRLEEELTRLRETKTRGVEEVAEGVVLLRRRGN

SEQ ID NO: 508 - protein, 54 aa, synthetic construct
KTRGVEEVAEGVVLLRRRGNKLEKINRRLVKILEDIIRRLKEISKEAEEELRKG

SEQ ID NO: 509 - protein, 59 aa, synthetic construct; identical to SEQ ID NO: 203

SEQ ID NO: 510 - protein, 62 aa, synthetic construct; identical to SEQ ID NO: 204

SEQ ID NO: 511 - protein, 58 aa, synthetic construct; identical to SEQ ID NO: 205

SEQ ID NO: 512 - protein, 54 aa, synthetic construct
AEDLEELIKELAELLKDVIRKLEKINRRLVKIKTRGVEEVAEGVVLLRRRGNKG

SEQ ID NO: 513 - protein, 59 aa, synthetic construct; identical to SEQ ID NO: 203

SEQ ID NO: 514 - protein, 62 aa, synthetic construct; identical to SEQ ID NO: 204

SEQ ID NO: 515 - protein, 58 aa, synthetic construct; identical to SEQ ID NO: 205

SEQ ID NO: 516 - protein, 54 aa, synthetic construct
AEDKTRGVEEVAEGVVLLRRRGNKINRRLVKILEDIIRRLKEISKEAEEELRKG

SEQ ID NO: 517 - protein, 59 aa, synthetic construct; identical to SEQ ID NO: 203

SEQ ID NO: 518 - protein, 62 aa, synthetic construct; identical to SEQ ID NO: 204

SEQ ID NO: 519 - protein, 58 aa, synthetic construct; identical to SEQ ID NO: 205

SEQ ID NO: 520 - protein, 54 aa, synthetic construct; identical to SEQ ID NO: 202

SEQ ID NO: 521 - protein, 59 aa, synthetic construct; identical to SEQ ID NO: 203

SEQ ID NO: 522 - protein, 62 aa, synthetic construct; identical to SEQ ID NO: 204

SEQ ID NO: 523 - protein, 58 aa, synthetic construct
DEAERRRRELKDKLDRKTRGVEEVAEGVVLLRRRGNETHKKIEKELREALKRVRDRST

SEQ ID NO: 524 - protein, 54 aa, synthetic construct; identical to SEQ ID NO: 516

SEQ ID NO: 525 - protein, 59 aa, synthetic construct; identical to SEQ ID NO: 203

SEQ ID NO: 526 - protein, 62 aa, synthetic construct; identical to SEQ ID NO: 204

SEQ ID NO: 527 - protein, 58 aa, synthetic construct; identical to SEQ ID NO: 205

SEQ ID NO: 528 - protein, 58 aa, synthetic construct; identical to SEQ ID NO: 210

SEQ ID NO: 529 - protein, 58 aa, synthetic construct; identical to SEQ ID NO: 211

SEQ ID NO: 530 - protein, 61 aa, synthetic construct; identical to SEQ ID NO: 212

SEQ ID NO: 531 - protein, 58 aa, synthetic construct
KIAEEIERELEELRRMIKRLHEDLERKKTRGVEEVAEGVVLLRRRGNRLEEKLERKRR

SEQ ID NO: 532 - protein, 58 aa, synthetic construct
KTRGVEEVAEGVVLLRRRGNLIKKLIETNRRLNEEHERAVRELARLLEELLDRLVKKG

SEQ ID NO: 533 - protein, 58 aa, synthetic construct; identical to SEQ ID NO: 211

SEQ ID NO: 534 - protein, 61 aa, synthetic construct; identical to SEQ ID NO: 212

SEQ ID NO: 535 - protein, 58 aa, synthetic construct; identical to SEQ ID NO: 213

SEQ ID NO: 536 - protein, 58 aa, synthetic construct; identical to SEQ ID NO: 214

SEQ ID NO: 537 - protein, 59 aa, synthetic construct; identical to SEQ ID NO: 215

SEQ ID NO: 538 - protein, 58 aa, synthetic construct; identical to SEQ ID NO: 216

SEQ ID NO: 539 - protein, 55 aa, synthetic construct
DKLKEIEDEKTRGVEEVAEGVVLLRRRGNELKKDLDEANRRIKETLKKLLREVEK

SEQ ID NO: 540 - protein, 58 aa, synthetic construct; identical to SEQ ID NO: 214

SEQ ID NO: 541 - protein, 59 aa, synthetic construct; identical to SEQ ID NO: 215

SEQ ID NO: 542 - protein, 58 aa, synthetic construct; identical to SEQ ID NO: 216

SEQ ID NO: 543 - protein, 55 aa, synthetic construct
DKLKEIEDELRRLKTRGVEEVAEGVVLLRRRGNDLDEANRRIKETLKKLLREVEK

SEQ ID NO: 544 - protein, 58 aa, synthetic construct; identical to SEQ ID NO: 214

SEQ ID NO: 545 - protein, 59 aa, synthetic construct; identical to SEQ ID NO: 215

SEQ ID NO: 546 - protein, 58 aa, synthetic construct; identical to SEQ ID NO: 216

SEQ ID NO: 547 - protein, 55 aa, synthetic construct
DKLKEIEDELRRLLEEKTRGVEEVAEGVVLLRRRGNEANRRIKETLKKLLREVEK

SEQ ID NO: 548 - protein, 58 aa, synthetic construct; identical to SEQ ID NO: 214

SEQ ID NO: 549 - protein, 59 aa, synthetic construct; identical to SEQ ID NO: 215

SEQ ID NO: 550 - protein, 58 aa, synthetic construct; identical to SEQ ID NO: 216

SEQ ID NO: 551 - protein, 55 aa, synthetic construct
DKLKEIEDELRRLLEELRRLDKAIKDRLRELKTRGVEEVAEGVVLLRRRGNEVEK

SEQ ID NO: 552 - protein, 55 aa, synthetic construct
EEKLKDDKTRGVEEVAEGVVLLRRRGNDNLRELKRVLDRITDLHRRILDELRKGI

SEQ ID NO: 553 - protein, 55 aa, synthetic construct; identical to SEQ ID NO: 219

SEQ ID NO: 554 - protein, 54 aa, synthetic construct; identical to SEQ ID NO: 220

SEQ ID NO: 555 - protein, 58 aa, synthetic construct; identical to SEQ ID NO: 221

SEQ ID NO: 556 - protein, 55 aa, synthetic construct
EEKLKDDVRAKTRGVEEVAEGVVLLRRRGNRELKRVLDRITDLHRRILDELRKGI

SEQ ID NO: 557 - protein, 55 aa, synthetic construct; identical to SEQ ID NO: 219

SEQ ID NO: 558 - protein, 54 aa, synthetic construct; identical to SEQ ID NO: 220

SEQ ID NO: 559 - protein, 58 aa, synthetic construct; identical to SEQ ID NO: 221

SEQ ID NO: 560 - protein, 55 aa, synthetic construct; identical to SEQ ID NO: 218

SEQ ID NO: 561 - protein, 55 aa, synthetic construct; identical to SEQ ID NO: 219

SEQ ID NO: 562 - protein, 54 aa, synthetic construct; identical to SEQ ID NO: 220

SEQ ID NO: 563 - protein, 58 aa, synthetic construct
RGVEEVAEGVVLLRRRGNEAIKLILELSEHHVRVSTRIARLLLDVARKLAEVIKEAER

SEQ ID NO: 564 - protein, 55 aa, synthetic construct; identical to SEQ ID NO: 218

SEQ ID NO: 565 - protein, 55 aa, synthetic construct; identical to SEQ ID NO: 219

SEQ ID NO: 566 - protein, 54 aa, synthetic construct; identical to SEQ ID NO: 220

SEQ ID NO: 567 - protein, 58 aa, synthetic construct
KAAEDILRVLEKLVKVSREAIKLILELSEHHVRVSTRIKTRGVEEVAEGVVLLRRRGN

SEQ ID NO: 568 - protein, 55 aa, synthetic construct
KTRGVEEVAEGVVLLRRRGNELEKLSEDNLRELKRVLDRITDLHRRILDELRKGI

SEQ ID NO: 569 - protein, 55 aa, synthetic construct; identical to SEQ ID NO: 219

SEQ ID NO: 570 - protein, 54 aa, synthetic construct; identical to SEQ ID NO: 220

SEQ ID NO: 571 - protein, 58 aa, synthetic construct; identical to SEQ ID NO: 221

SEQ ID NO: 572 - protein, 55 aa, synthetic construct
EEKKTRGVEEVAEGVVLLRRRGNKLSEDNLRELKRVLDRITDLHRRILDELRKGI

SEQ ID NO: 573 - protein, 55 aa, synthetic construct; identical to SEQ ID NO: 219

SEQ ID NO: 574 - protein, 54 aa, synthetic construct; identical to SEQ ID NO: 220

SEQ ID NO: 575 - protein, 58 aa, synthetic construct; identical to SEQ ID NO: 221

SEQ ID NO: 576 - protein, 57 aa, synthetic construct
EEVVKRLADVKTRGVEEVAEGVVLLRRRGNENDRLWKKLVEDIAEILRRIVELLRRG

SEQ ID NO: 577 - protein, 60 aa, synthetic construct; identical to SEQ ID NO: 223

SEQ ID NO: 578 - protein, 62 aa, synthetic construct; identical to SEQ ID NO: 224

SEQ ID NO: 579 - protein, 58 aa, synthetic construct; identical to SEQ ID NO: 225

SEQ ID NO: 580 - protein, 57 aa, synthetic construct; identical to SEQ ID NO: 222

SEQ ID NO: 581 - protein, 60 aa, synthetic construct; identical to SEQ ID NO: 223

SEQ ID NO: 582 - protein, 62 aa, synthetic construct; identical to SEQ ID NO: 224

SEQ ID NO: 583 - protein, 58 aa, synthetic construct
RGVEEVAEGVVLLRRRGNDVKKASEDIVREVERIVRELAKRSDEILKKLEDIVEKLRE

SEQ ID NO: 584 - protein, 57 aa, synthetic construct; identical to SEQ ID NO: 222

SEQ ID NO: 585 - protein, 60 aa, synthetic construct; identical to SEQ ID NO: 223

SEQ ID NO: 586 - protein, 62 aa, synthetic construct; identical to SEQ ID NO: 224

SEQ ID NO: 587 - protein, 58 aa, synthetic construct
DKTRGVEEVAEGVVLLRRRGNKASEDIVREVERIVRELAKRSDEILKKLEDIVEKLRE

SEQ ID NO: 588 - protein, 57 aa, synthetic construct; identical to SEQ ID NO: 222

SEQ ID NO: 589 - protein, 60 aa, synthetic construct; identical to SEQ ID NO: 223

SEQ ID NO: 590 - protein, 61 aa, synthetic construct; identical to SEQ ID NO: 228

SEQ ID NO: 591 - protein, 58 aa, synthetic construct
DVKRAKTRGVEEVAEGVVLLRRRGNDIVREVERIVRELAKRSDEILKKLEDIVEKLRE

SEQ ID NO: 592 - protein, 57 aa, synthetic construct; identical to SEQ ID NO: 222

SEQ ID NO: 593 - protein, 60 aa, synthetic construct; identical to SEQ ID NO: 223

SEQ ID NO: 594 - protein, 62 aa, synthetic construct; identical to SEQ ID NO: 224

SEQ ID NO: 595 - protein, 58 aa, synthetic construct
DVKRALEELVSRLRKLLEDKTRGVEEVAEGVVLLRRRGNKRSDEILKKLEDIVEKLRE

SEQ ID NO: 596 - protein, 57 aa, synthetic construct; identical to SEQ ID NO: 222

SEQ ID NO: 597 - protein, 60 aa, synthetic construct; identical to SEQ ID NO: 223

SEQ ID NO: 598 - protein, 62 aa, synthetic construct; identical to SEQ ID NO: 224

SEQ ID NO: 599 - protein, 58 aa, synthetic construct
DVKRALEELVSRLRKLLEDVKKASEDIKTRGVEEVAEGVVLLRRRGNKLEDIVEKLRE

SEQ ID NO: 600 - protein, 57 aa, synthetic construct; identical to SEQ ID NO: 222

SEQ ID NO: 601 - protein, 60 aa, synthetic construct; identical to SEQ ID NO: 223

SEQ ID NO: 602 - protein, 62 aa, synthetic construct; identical to SEQ ID NO: 224

SEQ ID NO: 603 - protein, 58 aa, synthetic construct
DVKRALEELVSRLRKLLEDVKKASEDIVREKTRGVEEVAEGVVLLRRRGNDIVEKLRE

SEQ ID NO: 604 - protein, 57 aa, synthetic construct; identical to SEQ ID NO: 222

SEQ ID NO: 605 - protein, 60 aa, synthetic construct; identical to SEQ ID NO: 223

SEQ ID NO: 606 - protein, 62 aa, synthetic construct; identical to SEQ ID NO: 224

SEQ ID NO: 607 - protein, 58 aa, synthetic construct
DVKRALEELVSRLRKLLEDVKKASEDIVREVERIVREKTRGVEEVAEGVVLLRRRGNE

SEQ ID NO: 608 - protein, 57 aa, synthetic construct; identical to SEQ ID NO: 222

SEQ ID NO: 609 - protein, 60 aa, synthetic construct; identical to SEQ ID NO: 223

SEQ ID NO: 610 - protein, 62 aa, synthetic construct; identical to SEQ ID NO: 224

SEQ ID NO: 611 - protein, 58 aa, synthetic construct
DVKRALEELVSRLRKLLEDVKKASEDIVREVERIVRELKTRGVEEVAEGVVLLRRRGN

SEQ ID NO: 612 - protein, 57 aa, synthetic construct
EEVVKRLADVLRENDETIRKVVEDLVRIKTRGVEEVAEGVVLLRRRGNRIVELLRRG

SEQ ID NO: 613 - protein, 60 aa, synthetic construct; identical to SEQ ID NO: 223

SEQ ID NO: 614 - protein, 62 aa, synthetic construct; identical to SEQ ID NO: 224

SEQ ID NO: 615 - protein, 58 aa, synthetic construct; identical to SEQ ID NO: 225

SEQ ID NO: 616 - protein, 57 aa, synthetic construct
EEVVKRLADVLRENDETIRKVVEDLVRIAEENDRLKTRGVEEVAEGVVLLRRRGNRG

SEQ ID NO: 617 - protein, 60 aa, synthetic construct; identical to SEQ ID NO: 223

SEQ ID NO: 618 - protein, 62 aa, synthetic construct; identical to SEQ ID NO: 224

SEQ ID NO: 619 - protein, 58 aa, synthetic construct; identical to SEQ ID NO: 225

SEQ ID NO: 620 - protein, 57 aa, synthetic construct
EEVKTRGVEEVAEGVVLLRRRGNDLVRIAEENDRLWKKLVEDIAEILRRIVELLRRG

SEQ ID NO: 621 - protein, 60 aa, synthetic construct; identical to SEQ ID NO: 223

SEQ ID NO: 622 - protein, 62 aa, synthetic construct; identical to SEQ ID NO: 224

SEQ ID NO: 623 - protein, 58 aa, synthetic construct; identical to SEQ ID NO: 225

SEQ ID NO: 624 - protein, 57 aa, synthetic construct
EEVVKRKTRGVEEVAEGVVLLRRRGNRIAEENDRLWKKLVEDIAEILRRIVELLRRG

SEQ ID NO: 625 - protein, 60 aa, synthetic construct; identical to SEQ ID NO: 223

SEQ ID NO: 626 - protein, 62 aa, synthetic construct; identical to SEQ ID NO: 224

SEQ ID NO: 627 - protein, 58 aa, synthetic construct; identical to SEQ ID NO: 225

SEQ ID NO: 628 - protein, 57 aa, synthetic construct; identical to SEQ ID NO: 576

SEQ ID NO: 629 - protein, 60 aa, synthetic construct; identical to SEQ ID NO: 223

SEQ ID NO: 630 - protein, 61 aa, synthetic construct; identical to SEQ ID NO: 228

SEQ ID NO: 631 - protein, 58 aa, synthetic construct; identical to SEQ ID NO: 225

SEQ ID NO: 632 - protein, 57 aa, synthetic construct; identical to SEQ ID NO: 222

SEQ ID NO: 633 - protein, 60 aa, synthetic construct; identical to SEQ ID NO: 223

SEQ ID NO: 634 - protein, 61 aa, synthetic construct; identical to SEQ ID NO: 228

SEQ ID NO: 635 - protein, 58 aa, synthetic construct; identical to SEQ ID NO: 583

SEQ ID NO: 636 - protein, 57 aa, synthetic construct
EEVVKRLADVLRENDETKTRGVEEVAEGVVLLRRRGNKLVEDIAEILRRIVELLRRG

SEQ ID NO: 637 - protein, 60 aa, synthetic construct; identical to SEQ ID NO: 223

SEQ ID NO: 638 - protein, 61 aa, synthetic construct; identical to SEQ ID NO: 228

SEQ ID NO: 639 - protein, 58 aa, synthetic construct; identical to SEQ ID NO: 225

SEQ ID NO: 640 - protein, 57 aa, synthetic construct; identical to SEQ ID NO: 222

SEQ ID NO: 641 - protein, 60 aa, synthetic construct; identical to SEQ ID NO: 223

SEQ ID NO: 642 - protein, 61 aa, synthetic construct; identical to SEQ ID NO: 228

SEQ ID NO: 643 - protein, 58 aa, synthetic construct
DVKRALEELKTRGVEEVAEGVVLLRRRGNEVERIVRELAKRSDEILKKLEDIVEKLRE

SEQ ID NO: 644 - protein, 57 aa, synthetic construct; identical to SEQ ID NO: 222

SEQ ID NO: 645 - protein, 60 aa, synthetic construct; identical to SEQ ID NO: 223

SEQ ID NO: 646 - protein, 61 aa, synthetic construct; identical to SEQ ID NO: 228

SEQ ID NO: 647 - protein, 58 aa, synthetic construct; identical to SEQ ID NO: 599

SEQ ID NO: 648 - protein, 55 aa, synthetic construct
EETLRRLLERKTRGVEEVAEGVVLLRRRGNKLLDEYLKTSKRIVDATVELLERGD

SEQ ID NO: 649 - protein, 56 aa, synthetic construct; identical to SEQ ID NO: 231

SEQ ID NO: 650 - protein, 58 aa, synthetic construct; identical to SEQ ID NO: 232

SEQ ID NO: 651 - protein, 59 aa, synthetic construct; identical to SEQ ID NO: 233

SEQ ID NO: 652 - protein, 55 aa, synthetic construct; identical to SEQ ID NO: 230

SEQ ID NO: 653 - protein, 56 aa, synthetic construct; identical to SEQ ID NO: 231

SEQ ID NO: 654 - protein, 58 aa, synthetic construct; identical to SEQ ID NO: 232

SEQ ID NO: 655 - protein, 59 aa, synthetic construct
REAKTRGVEEVAEGVVLLRRRGNDLEAKLEKLLRDLNELTKALEEELKRLLDELKKRTD

SEQ ID NO: 656 - protein, 55 aa, synthetic construct; identical to SEQ ID NO: 230

SEQ ID NO: 657 - protein, 56 aa, synthetic construct; identical to SEQ ID NO: 231

SEQ ID NO: 658 - protein, 58 aa, synthetic construct; identical to SEQ ID NO: 232

SEQ ID NO: 659 - protein, 59 aa, synthetic construct
REALEEARRRLKTRGVEEVAEGVVLLRRRGNLLRDLNELTKALEEELKRLLDELKKRTD

SEQ ID NO: 660 - protein, 55 aa, synthetic construct; identical to SEQ ID NO: 230

SEQ ID NO: 661 - protein, 56 aa, synthetic construct; identical to SEQ ID NO: 231

SEQ ID NO: 662 - protein, 58 aa, synthetic construct; identical to SEQ ID NO: 232

SEQ ID NO: 663 - protein, 59 aa, synthetic construct
REALEEARRRLEELKTRGVEEVAEGVVLLRRRGNDLNELTKALEEELKRLLDELKKRTD

SEQ ID NO: 664 - protein, 55 aa, synthetic construct; identical to SEQ ID NO: 230

SEQ ID NO: 665 - protein, 56 aa, synthetic construct; identical to SEQ ID NO: 231

SEQ ID NO: 666 - protein, 58 aa, synthetic construct; identical to SEQ ID NO: 232

SEQ ID NO: 667 - protein, 59 aa, synthetic construct
REALEEARRRLEELLRELNEIKTRGVEEVAEGVVLLRRRGNALEEELKRLLDELKKRTD

SEQ ID NO: 668 - protein, 55 aa, synthetic construct; identical to SEQ ID NO: 230

SEQ ID NO: 669 - protein, 56 aa, synthetic construct; identical to SEQ ID NO: 231

SEQ ID NO: 670 - protein, 58 aa, synthetic construct; identical to SEQ ID NO: 232

SEQ ID NO: 671 - protein, 59 aa, synthetic construct
REALEEARRRLEELLRELNEITKDLEAKKTRGVEEVAEGVVLLRRRGNRLLDELKKRTD

SEQ ID NO: 672 - protein, 55 aa, synthetic construct; identical to SEQ ID NO: 230

SEQ ID NO: 673 - protein, 56 aa, synthetic construct; identical to SEQ ID NO: 231

SEQ ID NO: 674 - protein, 58 aa, synthetic construct; identical to SEQ ID NO: 232

SEQ ID NO: 675 - protein, 59 aa, synthetic construct
REALEEARRRLEELLRELNEITKDLEAKLEKLKTRGVEEVAEGVVLLRRRGNELKKRTD

SEQ ID NO: 676 - protein, 55 aa, synthetic construct
EETKTRGVEEVAEGVVLLRRRGNEVIDRTTKLLDEYLKTSKRIVDATVELLERGD

SEQ ID NO: 677 - protein, 56 aa, synthetic construct; identical to SEQ ID NO: 231

SEQ ID NO: 678 - protein, 58 aa, synthetic construct; identical to SEQ ID NO: 232

SEQ ID NO: 679 - protein, 59 aa, synthetic construct; identical to SEQ ID NO: 233

SEQ ID NO: 680 - protein, 20 aa, synthetic construct
KTRGVEEVAEGVVLLRRRGN

SEQ ID NO: 681 - protein, 18 aa, synthetic construct
KTRGVEEVAEGVVLLRRR

SEQ ID NO: 682 - protein, 17 aa, synthetic construct
KTRGVEEVAEGVVLLRR

SEQ ID NO: 683 - protein, 16 aa, synthetic construct
KTRGVEEVAEGVVLLR

SEQ ID NO: 684 - protein, 15 aa, synthetic construct
KTRGVEEVAEGVVLL

SEQ ID NO: 685 - protein, 14 aa, synthetic construct
KTRGVEEVAEGVVL

SEQ ID NO: 686 - protein, 13 aa, synthetic construct
KTRGVEEVAEGVV

SEQ ID NO: 687 - protein, 12 aa, synthetic construct
KTRGVEEVAEGV

SEQ ID NO: 688 - protein, 11 aa, synthetic construct
KTRGVEEVAEG

SEQ ID NO: 689 - protein, 10 aa, synthetic construct
KTRGVEEVAE

SEQ ID NO: 690 - protein, 9 aa, synthetic construct
KTRGVEEVA

SEQ ID NO: 691 - protein, 8 aa, synthetic construct
KTRGVEEV

SEQ ID NO: 692 - protein, 7 aa, synthetic construct
KTRGVEE

SEQ ID NO: 693 - protein, 6 aa, synthetic construct
KTRGVE

SEQ ID NO: 694 - protein, 5 aa, synthetic construct
KTRGV

SEQ ID NO: 695 - protein, 4 aa, synthetic construct
KTRG

SEQ ID NO: 696 - protein, 4 aa, synthetic construct
TRGV

SEQ ID NO: 697 - protein, 4 aa, synthetic construct
RGVE

SEQ ID NO: 698 - protein, 4 aa, synthetic construct
GVEE

SEQ ID NO: 699 - protein, 4 aa, synthetic construct
VEEV

SEQ ID NO: 700 - protein, 4 aa, synthetic construct
EEVA

SEQ ID NO: 701 - protein, 4 aa, synthetic construct
VAEG

SEQ ID NO: 702 - protein, 4 aa, synthetic construct
AEGV

SEQ ID NO: 703 - protein, 4 aa, synthetic construct
EGVV

SEQ ID NO: 704 - protein, 4 aa, synthetic construct
GVVL

SEQ ID NO: 705 - protein, 4 aa, synthetic construct
VVLL

SEQ ID NO: 706 - protein, 4 aa, synthetic construct
VLLR

SEQ ID NO: 707 - protein, 4 aa, synthetic construct
LLRR

SEQ ID NO: 708 - protein, 4 aa, synthetic construct
LRRR

SEQ ID NO: 709 - protein, 4 aa, synthetic construct
RRGN

SEQ ID NO: 710 - protein, 5 aa, synthetic construct
TRGVE

SEQ ID NO: 711 - protein, 5 aa, synthetic construct
RGVEE

SEQ ID NO: 712 - protein, 5 aa, synthetic construct
GVEEV

SEQ ID NO: 713 - protein, 5 aa, synthetic construct
VEEVA

SEQ ID NO: 714 - protein, 5 aa, synthetic construct
EEVAE

SEQ ID NO: 715 - protein, 5 aa, synthetic construct
EVAEG

SEQ ID NO: 716 - protein, 5 aa, synthetic construct
VAEGV

SEQ ID NO: 717 - protein, 5 aa, synthetic construct
AEGVV

SEQ ID NO: 718 - protein, 5 aa, synthetic construct
EGVVL

SEQ ID NO: 719 - protein, 5 aa, synthetic construct
GVVLL

SEQ ID NO: 720 - protein, 5 aa, synthetic construct
VVLLR

SEQ ID NO: 721 - protein, 5 aa, synthetic construct
VLLRR

SEQ ID NO: 722 - protein, 5 aa, synthetic construct
LLRRR

SEQ ID NO: 723 - protein, 6 aa, synthetic construct
TRGVEE

SEQ ID NO: 724 - protein, 6 aa, synthetic construct
RGVEEV

SEQ ID NO: 725 - protein, 6 aa, synthetic construct
GVEEVA

SEQ ID NO: 726 - protein, 6 aa, synthetic construct
VEEVAE

SEQ ID NO: 727 - protein, 6 aa, synthetic construct
EEVAEG

SEQ ID NO: 728 - protein, 6 aa, synthetic construct
EVAEGV

SEQ ID NO: 729 - protein, 6 aa, synthetic construct
VAEGVV

SEQ ID NO: 730 - protein, 6 aa, synthetic construct
AEGVVL

SEQ ID NO: 731 - protein, 6 aa, synthetic construct
EGVVLL

SEQ ID NO: 732 - protein, 6 aa, synthetic construct
GVVLLR

SEQ ID NO: 733 - protein, 6 aa, synthetic construct
VVLLRR

SEQ ID NO: 734 - protein, 6 aa, synthetic construct
VLLRRR

SEQ ID NO: 735 - protein, 7 aa, synthetic construct
TRGVEEV

SEQ ID NO: 736 - protein, 7 aa, synthetic construct
RGVEEVA

SEQ ID NO: 737 - protein, 7 aa, synthetic construct
GVEEVAE

SEQ ID NO: 738 - protein, 7 aa, synthetic construct
VEEVAEG

SEQ ID NO: 739 - protein, 7 aa, synthetic construct
EEVAEGV

SEQ ID NO: 740 - protein, 7 aa, synthetic construct
EVAEGVV

SEQ ID NO: 741 - protein, 7 aa, synthetic construct
VAEGVVL

SEQ ID NO: 742 - protein, 7 aa, synthetic construct
AEGVVLL

SEQ ID NO: 743 - protein, 7 aa, synthetic construct
EGVVLLR

SEQ ID NO: 744 - protein, 7 aa, synthetic construct
GVVLLRR

SEQ ID NO: 745 - protein, 7 aa, synthetic construct
VVLLRRR

SEQ ID NO: 746 - protein, 8 aa, synthetic construct
TRGVEEVA

SEQ ID NO: 747 - protein, 8 aa, synthetic construct
RGVEEVAE

SEQ ID NO: 748 - protein, 8 aa, synthetic construct
GVEEVAEG

SEQ ID NO: 749 - protein, 8 aa, synthetic construct
VEEVAEGV

SEQ ID NO: 750 - protein, 8 aa, synthetic construct
EEVAEGVV

SEQ ID NO: 751 - protein, 8 aa, synthetic construct
EVAEGVVL

SEQ ID NO: 752 - protein, 8 aa, synthetic construct
VAEGVVLL

SEQ ID NO: 753 - protein, 8 aa, synthetic construct
AEGVVLLR

SEQ ID NO: 754 - protein, 8 aa, synthetic construct
EGVVLLRR

SEQ ID NO: 755 - protein, 8 aa, synthetic construct
GVVLLRRR

SEQ ID NO: 756 - protein, 9 aa, synthetic construct
TRGVEEVAE

SEQ ID NO: 757 - protein, 9 aa, synthetic construct
RGVEEVAEG

SEQ ID NO: 758 - protein, 9 aa, synthetic construct
GVEEVAEGV

SEQ ID NO: 759 - protein, 9 aa, synthetic construct
VEEVAEGVV

SEQ ID NO: 760 - protein, 9 aa, synthetic construct
EEVAEGVVL

SEQ ID NO: 761 - protein, 9 aa, synthetic construct
EVAEGVVLL

SEQ ID NO: 762 - protein, 9 aa, synthetic construct
VAEGVVLLR

SEQ ID NO: 763 - protein, 9 aa, synthetic construct
AEGVVLLRR

SEQ ID NO: 764 - protein, 9 aa, synthetic construct
EGVVLLRRR

SEQ ID NO: 765 - protein, 10 aa, synthetic construct
TRGVEEVAEG

SEQ ID NO: 766 - protein, 10 aa, synthetic construct
RGVEEVAEGV

SEQ ID NO: 767 - protein, 10 aa, synthetic construct
GVEEVAEGVV

SEQ ID NO: 768 - protein, 10 aa, synthetic construct
VEEVAEGVVL

SEQ ID NO: 769 - protein, 10 aa, synthetic construct
EEVAEGVVLL

SEQ ID NO: 770 - protein, 10 aa, synthetic construct
EVAEGVVLLR

SEQ ID NO: 771 - protein, 10 aa, synthetic construct
VAEGVVLLRR

SEQ ID NO: 772 - protein, 10 aa, synthetic construct
AEGVVLLRRR

SEQ ID NO: 773 - protein, 11 aa, synthetic construct
TRGVEEVAEGV

SEQ ID NO: 774 - protein, 11 aa, synthetic construct
RGVEEVAEGVV

SEQ ID NO: 775 - protein, 11 aa, synthetic construct
GVEEVAEGVVL

SEQ ID NO: 776 - protein, 11 aa, synthetic construct
VEEVAEGVVLL

SEQ ID NO: 777 - protein, 11 aa, synthetic construct
EEVAEGVVLLR

SEQ ID NO: 778 - protein, 11 aa, synthetic construct
EVAEGVVLLRR

SEQ ID NO: 779 - protein, 11 aa, synthetic construct
VAEGVVLLRRR

SEQ ID NO: 780 - protein, 12 aa, synthetic construct
TRGVEEVAEGVV

SEQ ID NO: 781 - protein, 12 aa, synthetic construct
RGVEEVAEGVVL

SEQ ID NO: 782 - protein, 12 aa, synthetic construct
GVEEVAEGVVLL

SEQ ID NO: 783 - protein, 12 aa, synthetic construct
VEEVAEGVVLLR

SEQ ID NO: 784 - protein, 12 aa, synthetic construct
EEVAEGVVLLRR

SEQ ID NO: 785 - protein, 12 aa, synthetic construct
EVAEGVVLLRRR

SEQ ID NO: 786 - protein, 13 aa, synthetic construct
TRGVEEVAEGVVL

SEQ ID NO: 787 - protein, 13 aa, synthetic construct
RGVEEVAEGVVLL

SEQ ID NO: 788 - protein, 13 aa, synthetic construct
GVEEVAEGVVLLR

SEQ ID NO: 789 - protein, 13 aa, synthetic construct
VEEVAEGVVLLRR

SEQ ID NO: 790 - protein, 13 aa, synthetic construct
EEVAEGVVLLRRR

SEQ ID NO: 791 - protein, 14 aa, synthetic construct
TRGVEEVAEGVVLL

SEQ ID NO: 792 - protein, 14 aa, synthetic construct
RGVEEVAEGVVLLR

SEQ ID NO: 793 - protein, 14 aa, synthetic construct
GVEEVAEGVVLLRR

SEQ ID NO: 794 - protein, 14 aa, synthetic construct
VEEVAEGVVLLRRR

SEQ ID NO: 795 - protein, 15 aa, synthetic construct
TRGVEEVAEGVVLLR

SEQ ID NO: 796 - protein, 15 aa, synthetic construct
RGVEEVAEGVVLLRR

SEQ ID NO: 797 - protein, 15 aa, synthetic construct
GVEEVAEGVVLLRRR

SEQ ID NO: 798 - protein, 16 aa, synthetic construct
TRGVEEVAEGVVLLRR

SEQ ID NO: 799 - protein, 16 aa, synthetic construct
RGVEEVAEGVVLLRRR

SEQ ID NO: 800 - protein, 17 aa, synthetic construct
TRGVEEVAEGVVLLRRR

SEQ ID NO: 801 - protein, 21 aa, synthetic construct
KTRGVEEVAEGVVLLRRRGNK

SEQ ID NO: 802 - protein, 17 aa, synthetic construct
KEEVAEGVVLLRRRGNK

SEQ ID NO: 803 - protein, 17 aa, synthetic construct
KTRGVEEVAEGVVLLRK

SEQ ID NO: 804 - protein, 12 aa, synthetic construct
EGVVLLRRRGNK

SEQ ID NO: 805 - protein, 8 aa, synthetic construct
KTRGVEEV

SEQ ID NO: 806 - protein, 12 aa, synthetic construct
KTRGVEEVAEGV

SEQ ID NO: 807 - protein, 179 aa, synthetic construct
KALEEAKRVLAENNEVIKDIKRALDELLKELEKSVRESEERLRRLLEEVKRVIEEVAGSGSPLKARLEDIIRRLAEDIERVKRETDKLLEELARIIEEVEKEMRETNKELDKEIKDAESQNGSPEEIEKKVRELTKRHRELKTRGVEEVAEGVVLLRRRGNTLTERIKRVLEEVRDLER

SEQ ID NO: 808 - protein, 159 aa, synthetic construct
LAENNEVIKDIKRALDELLKELEKSVRESEERLRRLLEEVKRVIEEVATKASKSNDHDLKARLEDIIRRLAEDIERVKRETDKLLEELARIIEEVEKEMRETNKELDGSGSPELTKRHRELKTRGVEEVAEGVVLLRRRGNTLTERIKRVLEEVRDLER

SEQ ID NO: 809 - protein, 148 aa, synthetic construct
KALEEAKRVLAENNEVIKDIKRALDELLKELEKSVRESEERLRRLLEEYKVGSPEDIERVKRETDKLLEELARIIEEVEKEMRETNKELDKEIKDAESQNQSLEEIEKKVRELTKRHRELKTRGVEEVAEGVVLLRRRGNTLTERIKR

SEQ ID NO: 810 - protein, 123 aa, synthetic construct
LAENNEVIKDIKRALDELLKELEKSVRESEERLRRLLEEYKVGSPEDIERVKRETDKLLEELARIIEEVEKEMRETNKELDGSGSPELTKRHRELKTRGVEEVAEGVVLLRRRGNTLTERIKR

SEQ ID NO: 811 - protein, 162 aa, synthetic construct
ARELTKRHRELKTRGVEEVAEGVVLLRRRGNTLTERIKRVLEEVRDLERRGEPKEDLKARLEDIIRRLAEDIERVKRETDKLLEELARIIEEVEKEMRETNKELEEAAKGSPALAENNEVIKDIKRALDELLKELEKSVRESEERLRRLLEEVKRVIEEVAT

SEQ ID NO: 812 - protein, 154 aa, synthetic construct
ATDLLKASNDKNRKVVEEIRDLLRKSKKLADELVERLRALVEDLRRRIDKSGDKETAEDIVRRIIEELKRILKEIEDLARRINREIERLVEEVERDNRDVNRKAGDEEARRRAEESMRRVKEDKTRGVEEVAEGVVLLRRRGNRLIEEVRETLR

SEQ ID NO: 813 - protein, 115 aa, synthetic construct
NRKVVEEMRDLLRKSKKLADELVERLRALVEDLRRRIDKSGDKETAEDIVRRIIEELKRILKEIEDLARRINREIERAGADDSAKTRGVEEVAEGVVLLRRRGNRLIEEVRETLR

SEQ ID NO: 814 - protein, 150 aa, synthetic construct
SELEEIEKKVRELTKRHRELKTRGVEEVAEGVVLLRRRGNTLTEKIKNGKDERKLAEDIERVKRETDKLLEELARIIEEVEKEMRETNKELDKEIKDGGLDDKALEEAKRVLAENNEVIKDIKRALDELLKELEKSVRESEERLRRLLEE

SEQ ID NO: 815 - protein, 104 aa, synthetic construct
TKRIRELKTRGVEEVAEGVVLLRRRGNTLTEKIKNGKDERKLAEDIERVKRETDKLLEELARIIEELKKGNVDDDVSRALDELLKELEKSVRESEERLRRLLEE

SEQ ID NO: 816 - protein, 208 aa, synthetic construct
ADVLRENDETIRKVVEDLVRIAEENDRLKTRGVEEVAEGVVLLRRRGNRGGVPEELLDRLAKVVKSIVEKAEKILERLNRVSKAIAEKLKTIVDELNEVSKSGASQKEFADLSKEVLERVERIVREYVKLSDEVVKSLAEIVEELIRIIEDLLRKGNLDEDVKRALEELVSRLRKLLEDVKKASEDIVREVERIVRELAKRSDEILKK

SEQ ID NO: 817 - protein, 167 aa, synthetic construct
TARKVVEDLVRIAEENDRLKTRGVEEVAEGVVLLRRRGNRGGVPEELLDRLAKVVKSIVEKAEKILERLNRVSKAIAEKLKSTGNKEEVERIVREYVKLSDEVVKSLAEIVEELIRIIEDLLRKGNLDEDVKRALEELVSRLRKLLEDVKKASEDIVREVERIVREL

SEQ ID NO: 818 - protein, 140 aa, synthetic construct
LREEKEEARTHVEKSIRDLRKILEDKTRGVEEVAEGVVLLRRRGNKKPGDEEVRKTVEEISRRVAENVKRLEDLYRRMEEEVKKNLDRLRKEGVDEDILRRAAEAHKKLIDDARESLERAKREHEKLIDRLKKILEELER

SEQ ID NO: 819 - protein, 110 aa, synthetic construct
THVEKSIRDLRKILEDKTRGVEEVAEGVVLLRRRGNKKPGDEEVRKTVEEISRRVAENVKRLEDLYRRMEEEAGSESELADDARESLERAKREHEKLIDRLKKILEELER

SEQ ID NO: 820 - protein, 201 aa, synthetic construct
DEDRIIEEIARLLEELLRELLELIKKLIETNRRLNEEHERAVRELARLGTDKALDDLERLHREINKRLEDLVRELEKLVREILKELKDALEELRRASARAGGEEVLRRLEEIVKKLLDLVRRILERLKEIHKDNVRLLRELNERLTRILGNADEEIRRMVKRLHEDLERKKTRGVEEVAEGVVLLRRRGNRLEEKLERKRR

SEQ ID NO: 821 - protein, 153 aa, synthetic construct
DEDRIIEEIARLLEELLRELLELIKKLIETNRRLAEEANGDSKVNKRLEDLVRELEKLVREILKELKDALEELRRASARAGGEEVLRRLEEIVKKLLDLVRRILERLKEIHKDNSVFGDKRKKTRGVEEVAEGVVLLRRRGNRLEEKLERKRR

SEQ ID NO: 822 - protein, 36 aa, synthetic construct
SGGGGSGGGGSLPMSCAQESGMDRHPAACASARINV

SEQ ID NO: 823 - protein, 179 aa, synthetic construct
KALEEAKRVLAENNEVIKDIKRALDELLKELEKSVRESEERLRRLLEEVKRVIEEVAGSGSPLKARLEDIIRRLAEDIERVKRETDKLLEELARIIEEVEKEMRETNKELDKEIKDAESQNGSPEEIEKKVRELTKRHRELVERVRKTVKELIETNRRLLETLTERIKRVLEEVRDLER

SEQ ID NO: 824 - protein, 159 aa, synthetic construct
LAENNEVIKDIKRALDELLKELEKSVRESEERLRRLLEEVKRVIEEVATKASKSNDHDLKARLEDIIRRLAEDIERVKRETDKLLEELARIIEEVEKEMRETNKELDGSGSPELTKRHRELVERVRKTVKELIETNRRLLETLTERIKRVLEEVRDLER

SEQ ID NO: 825 - protein, 148 aa, synthetic construct
KALEEAKRVLAENNEVIKDIKRALDELLKELEKSVRESEERLRRLLEEYKVGSPEDIERVKRETDKLLEELARIIEEVEKEMRETNKELDKEIKDAESQNQSLEEIEKKVRELTKRHRELVERVRKTVKELIETNRRLLETLTERIKR

SEQ ID NO: 826 - protein, 123 aa, synthetic construct
LAENNEVIKDIKRALDELLKELEKSVRESEERLRRLLEEYKVGSPEDIERVKRETDKLLEELARIIEEVEKEMRETNKELDGSGSPELTKRHRELVERVRKTVKELIETNRRLLETLTERIKR

SEQ ID NO: 827 - protein, 162 aa, synthetic construct
ARELTKRHRELVERVRKTVKELIETNRRLLETLTERIKRVLEEVRDLERRGEPKEDLKARLEDIIRRLAEDIERVKRETDKLLEELARIIEEVEKEMRETNKELEEAAKGSPALAENNEVIKDIKRALDELLKELEKSVRESEERLRRLLEEVKRVIEEVAT

SEQ ID NO: 828 - protein, 150 aa, synthetic construct
SELEEIEKKVRELTKRHRELVERVRKTVKELIETNRRLLETLTEKIKNGKDERKLAEDIERVKRETDKLLEELARIIEEVEKEMRETNKELDKEIKDGGLDDKALEEAKRVLAENNEVIKDIKRALDELLKELEKSVRESEERLRRLLEE

SEQ ID NO: 829 - protein, 104 aa, synthetic construct
TKRIRELVERVRKTVKELIETNRRLLETLTEKIKNGKDERKLAEDIERVKRETDKLLEELARIIEELKKGNVDDDVSRALDELLKELEKSVRESEERLRRLLEE

SEQ ID NO: 830 - protein, 154 aa, synthetic construct
ATDLLKASNDKNRKVVEEIRDLLRKSKKLADELVERLRALVEDLRRRIDKSGDKETAEDIVRRIIEELKRILKEIEDLARRINREIERLVEEVERDNRDVNRKAGDEEARRRAEESMRRVKEDADRVSEANKEVLDRVREEVKRLIEEVRETLR

SEQ ID NO: 831 - protein, 115 aa, synthetic construct
NRKVVEEMRDLLRKSKKLADELVERLRALVEDLRRRIDKSGDKETAEDIVRRIIEELKRILKEIEDLARRINREIERAGADDSAADRVSEANKEVLDRVREEVKRLIEEVRETLR

SEQ ID NO: 832 - protein, 208 aa, synthetic construct
ADVLRENDETIRKVVEDLVRIAEENDRLWKKLVEDIAEILRRIVELLRRGGVPEELLDRLAKVVKSIVEKAEKILERLNRVSKAIAEKLKTIVDELNEVSKSGASQKEFADLSKEVLERVERIVREYVKLSDEVVKSLAEIVEELIRIIEDLLRKGNLDEDVKRALEELVSRLRKLLEDVKKASEDIVREVERIVRELAKRSDEILKK

SEQ ID NO: 833 - protein, 167 aa, synthetic construct
TARKVVEDLVRIAEENDRLWKKLVEDIAEILRRIVELLRRGGVPEELLDRLAKVVKSIVEKAEKILERLNRVSKAIAEKLKSTGNKEEVERIVREYVKLSDEVVKSLAEIVEELIRIIEDLLRKGNLDEDVKRALEELVSRLRKLLEDVKKASEDIVREVERIVREL

SEQ ID NO: 834 - protein, 140 aa, synthetic construct
LREEKEEARTHVEKSIRDLRKILEDSTRHAKRSIEELERLLEEVRKKPGDEEVRKTVEEISRRVAENVKRLEDLYRRMEEEVKKNLDRLRKEGVDEDILRRAAEAHKKLIDDARESLERAKREHEKLIDRLKKILEELER

SEQ ID NO: 835 - protein, 110 aa, synthetic construct
THVEKSIRDLRKILEDSTRHAKRSIEELERLLEEVRKKPGDEEVRKTVEEISRRVAENVKRLEDLYRRMEEEAGSESELADDARESLERAKREHEKLIDRLKKILEELER

SEQ ID NO: 836 - protein, 201 aa, synthetic construct
DEDRIIEEIARLLEELLRELLELIKKLIETNRRLNEEHERAVRELARLGTDKALDDLERLHREINKRLEDLVRELEKLVREILKELKDALEELRRASARAGGEEVLRRLEEIVKKLLDLVRRILERLKEIHKDNVRLLRELNERLTRILGNADEEIRRMVKRLHEDLERKLKESEDELREIEARLEEKIRRLEEKLERKRR

SEQ ID NO: 837 - protein, 153 aa, synthetic construct
DEDRIIEEIARLLEELLRELLELIKKLIETNRRLAEEANGDSKVNKRLEDLVRELEKLVREILKELKDALEELRRASARAGGEEVLRRLEEIVKKLLDLVRRILERLKEIHKDNSVFGDKRKLKESEDELREIEARLEEKIRRLEEKLERKRR

SEQ ID NO: 838 - protein, 132 aa, synthetic construct
KALEEAKRVLAENNEVIKDIKRALDELLKELEKSVRESEERLRRLLEEVKRVIEEVAGSGSPLKARLEDIIRRLAEDIERVKRETDKLLEELARIIEEVEKEMRETNKELDKEIKDAESQNGSPEEIEKKVR

SEQ ID NO: 839 - protein, 47 aa, synthetic construct
ELTKRHRELVERVRKTVKELIETNRRLLETLTERIKRVLEEVRDLER

SEQ ID NO: 840 - protein, 112 aa, synthetic construct
LAENNEVIKDIKRALDELLKELEKSVRESEERLRRLLEEVKRVIEEVATKASKSNDHDLKARLEDIIRRLAEDIERVKRETDKLLEELARIIEEVEKEMRETNKELDGSGSP

SEQ ID NO: 841 - protein, 47 aa, synthetic construct; identical to SEQ ID NO: 839

SEQ ID NO: 842 - protein, 111 aa, synthetic construct
KALEEAKRVLAENNEVIKDIKRALDELLKELEKSVRESEERLRRLLEEYKVGSPEDIERVKRETDKLLEELARIIEEVEKEMRETNKELDKEIKDAESQNQSLEEIEKKVR

SEQ ID NO: 843 - protein, 37 aa, synthetic construct
ELTKRHRELVERVRKTVKELIETNRRLLETLTERIKR

SEQ ID NO: 844 - protein, 86 aa, synthetic construct
LAENNEVIKDIKRALDELLKELEKSVRESEERLRRLLEEYKVGSPEDIERVKRETDKLLEELARIIEEVEKEMRETNKELDGSGSP

SEQ ID NO: 845 - protein, 37 aa, synthetic construct; identical to SEQ ID NO: 843

SEQ ID NO: 846 - protein, 127 aa, synthetic construct
RIKRVLEEVRDLERRGEPKEDLKARLEDIIRRLAEDIERVKRETDKLLEELARIIEEVEKEMRETNKELEEAAKGSPALAENNEVIKDIKRALDELLKELEKSVRESEERLRRLLEEVKRVIEEVAT

SEQ ID NO: 847 - protein, 35 aa, synthetic construct
ARELTKRHRELVERVRKTVKELIETNRRLLETLTE

SEQ ID NO: 848 - protein, 106 aa, synthetic construct
KIKNGKDERKLAEDIERVKRETDKLLEELARIIEEVEKEMRETNKELDKEIKDGGLDDKALEEAKRVLAENNEVIKDIKRALDELLKELEKSVRESEERLRRLLEE

SEQ ID NO: 849 - protein, 44 aa, synthetic construct
SELEEIEKKVRELTKRHRELVERVRKTVKELIETNRRLLETLTE

SEQ ID NO: 850 - protein, 73 aa, synthetic construct
KIKNGKDERKLAEDIERVKRETDKLLEELARIIEELKKGNVDDDVSRALDELLKELEKSVRESEERLRRLLEE

SEQ ID NO: 851 - protein, 31 aa, synthetic construct
TKRIRELVERVRKTVKELIETNRRLLETLTE

SEQ ID NO: 852 - protein, 105 aa, synthetic construct
ATDLLKASNDKNRKVVEEIRDLLRKSKKLADELVERLRALVEDLRRRIDKSGDKETAEDIVRRIIEELKRILKEIEDLARRINREIERLVEEVERDNRDVNRKAG

SEQ ID NO: 853 - protein, 49 aa, synthetic construct
DEEARRRAEESMRRVKEDADRVSEANKEVLDRVREEVKRLIEEVRETLR

SEQ ID NO: 854 - protein, 78 aa, synthetic construct
NRKVVEEMRDLLRKSKKLADELVERLRALVEDLRRRIDKSGDKETAEDIVRRIIEELKRILKEIEDLARRINREIERA

SEQ ID NO: 855 - protein, 37 aa, synthetic construct
GADDSAADRVSEANKEVLDRVREEVKRLIEEVRETLR

SEQ ID NO: 856 - protein, 156 aa, synthetic construct
PEELLDRLAKVVKSIVEKAEKILERLNRVSKAIAEKLKTIVDELNEVSKSGASQKEFADLSKEVLERVERIVREYVKLSDEVVKSLAEIVEELIRIIEDLLRKGNLDEDVKRALEELVSRLRKLLEDVKKASEDIVREVERIVRELAKRSDEILKK

SEQ ID NO: 857 - protein, 52 aa, synthetic construct
ADVLRENDETIRKVVEDLVRIAEENDRLWKKLVEDIAEILRRIVELLRRGGV

SEQ ID NO: 858 - protein, 124 aa, synthetic construct
PEELLDRLAKVVKSIVEKAEKILERLNRVSKAIAEKLKSTGNKEEVERIVREYVKLSDEVVKSLAEIVEELIRIIEDLLRKGNLDEDVKRALEELVSRLRKLLEDVKKASEDIVREVERIVREL

SEQ ID NO: 859 - protein, 43 aa, synthetic construct
TARKVVEDLVRIAEENDRLWKKLVEDIAEILRRIVELLRRGGV

SEQ ID NO: 860 - protein, 93 aa, synthetic construct
PGDEEVRKTVEEISRRVAENVKRLEDLYRRMEEEVKKNLDRLRKEGVDEDILRRAAEAHKKLIDDARESLERAKREHEKLIDRLKKILEELER

SEQ ID NO: 861 - protein, 47 aa, synthetic construct
LREEKEEARTHVEKSIRDLRKILEDSTRHAKRSIEELERLLEEVRKK

SEQ ID NO: 862 - protein, 72 aa, synthetic construct
PGDEEVRKTVEEISRRVAENVKRLEDLYRRMEEEAGSESELADDARESLERAKREHEKLIDRLKKILEELER

SEQ ID NO: 863 - protein, 38 aa, synthetic construct
THVEKSIRDLRKILEDSTRHAKRSIEELERLLEEVRKK

SEQ ID NO: 864 - protein, 152 aa, synthetic construct
DEDRIIEEIARLLEELLRELLELIKKLIETNRRLNEEHERAVRELARLGTDKALDDLERLHREINKRLEDLVRELEKLVREILKELKDALEELRRASARAGGEEVLRRLEEIVKKLLDLVRRILERLKEIHKDNVRLLRELNERLTRILGNA

SEQ ID NO: 865 - protein, 49 aa, synthetic construct
DEEIRRMVKRLHEDLERKLKESEDELREIEARLEEKIRRLEEKLERKRR

SEQ ID NO: 866 - protein, 115 aa, synthetic construct
DEDRIIEEIARLLEELLRELLELIKKLIETNRRLAEEANGDSKVNKRLEDLVRELEKLVREILKELKDALEELRRASARAGGEEVLRRLEEIVKKLLDLVRRILERLKEIHKDNS

SEQ ID NO: 867 - protein, 38 aa, synthetic construct
VFGDKRKLKESEDELREIEARLEEKIRRLEEKLERKRR

SEQ ID NO: 868 - protein, 46 aa, synthetic construct
KALEEAKRVLAENNEVIKDIKRALDELLKELEKSVRESEERLRRLL

SEQ ID NO: 869 - protein, 43 aa, synthetic construct
EEVKRVIEEVAGSGSPLKARLEDIIRRLAEDIERVKRETDKLL

SEQ ID NO: 870 - protein, 43 aa, synthetic construct
EELARIIEEVEKEMRETNKELDKEIKDAESQNGSPEEIEKKVR

SEQ ID NO: 871 - protein, 56 aa, synthetic construct
LAENNEVIKDIKRALDELLKELEKSVRESEERLRRLLEEVKRVIEEVATKASKSND

SEQ ID NO: 872 - protein, 56 aa, synthetic construct
HDLKARLEDIIRRLAEDIERVKRETDKLLEELARIIEEVEKEMRETNKELDGSGSP

SEQ ID NO: 873 - protein, 56 aa, synthetic construct
KALEEAKRVLAENNEVIKDIKRALDELLKELEKSVRESEERLRRLLEEYKVGSPED

SEQ ID NO: 874 - protein, 55 aa, synthetic construct
IERVKRETDKLLEELARIIEEVEKEMRETNKELDKEIKDAESQNQSLEEIEKKVR

SEQ ID NO: 875 - protein, 43 aa, synthetic construct
LAENNEVIKDIKRALDELLKELEKSVRESEERLRRLLEEYKVG

SEQ ID NO: 876 - protein, 43 aa, synthetic construct
SPEDIERVKRETDKLLEELARIIEEVEKEMRETNKELDGSGSP

SEQ ID NO: 877 - protein, 63 aa, synthetic construct
RIKRVLEEVRDLERRGEPKEDLKARLEDIIRRLAEDIERVKRETDKLLEELARIIEEVEKEMR

SEQ ID NO: 878 - protein, 64 aa, synthetic construct
ETNKELEEAAKGSPALAENNEVIKDIKRALDELLKELEKSVRESEERLRRLLEEVKRVIEEVAT

SEQ ID NO: 879 - protein, 53 aa, synthetic construct
KIKNGKDERKLAEDIERVKRETDKLLEELARIIEEVEKEMRETNKELDKEIKD

SEQ ID NO: 880 - protein, 53 aa, synthetic construct
GGLDDKALEEAKRVLAENNEVIKDIKRALDELLKELEKSVRESEERLRRLLEE

SEQ ID NO: 881 - protein, 36 aa, synthetic construct
KIKNGKDERKLAEDIERVKRETDKLLEELARIIEEL

SEQ ID NO: 882 - protein, 37 aa, synthetic construct
KKGNVDDDVSRALDELLKELEKSVRESEERLRRLLEE

SEQ ID NO: 883 - protein, 52 aa, synthetic construct
ATDLLKASNDKNRKVVEEIRDLLRKSKKLADELVERLRALVEDLRRRIDKSG

SEQ ID NO: 884 - protein, 53 aa, synthetic construct
DKETAEDIVRRIIEELKRILKEIEDLARRINREIERLVEEVERDNRDVNRKAG

SEQ ID NO: 885 - protein, 39 aa, synthetic construct
NRKVVEEMRDLLRKSKKLADELVERLRALVEDLRRRIDK

SEQ ID NO: 886 - protein, 39 aa, synthetic construct
SGDKETAEDIVRRIIEELKRILKEIEDLARRINREIERA

SEQ ID NO: 887 - protein, 52 aa, synthetic construct
PEELLDRLAKVVKSIVEKAEKILERLNRVSKAIAEKLKTIVDELNEVSKSGA

SEQ ID NO: 888 - protein, 52 aa, synthetic construct
SQKEFADLSKEVLERVERIVREYVKLSDEVVKSLAEIVEELIRIIEDLLRKG

SEQ ID NO: 889 - protein, 52 aa, synthetic construct
NLDEDVKRALEELVSRLRKLLEDVKKASEDIVREVERIVRELAKRSDEILKK

SEQ ID NO: 890 - protein, 42 aa, synthetic construct
PEELLDRLAKVVKSIVEKAEKILERLNRVSKAIAEKLKSTGN

SEQ ID NO: 891 - protein, 41 aa, synthetic construct
KEEVERIVREYVKLSDEVVKSLAEIVEELIRIIEDLLRKGN

SEQ ID NO: 892 - protein, 41 aa, synthetic construct
LDEDVKRALEELVSRLRKLLEDVKKASEDIVREVERIVREL

SEQ ID NO: 893 - protein, 47 aa, synthetic construct
PGDEEVRKTVEEISRRVAENVKRLEDLYRRMEEEVKKNLDRLRKEGV

SEQ ID NO: 894 - protein, 46 aa, synthetic construct
DEDILRRAAEAHKKLIDDARESLERAKREHEKLIDRLKKILEELER

SEQ ID NO: 895 - protein, 36 aa, synthetic construct
PGDEEVRKTVEEISRRVAENVKRLEDLYRRMEEEAG

SEQ ID NO: 896 - protein, 36 aa, synthetic construct
SESELADDARESLERAKREHEKLIDRLKKILEELER

SEQ ID NO: 897 - protein, 51 aa, synthetic construct
DEDRIIEEIARLLEELLRELLELIKKLIETNRRLNEEHERAVRELARLGTD

SEQ ID NO: 898 - protein, 50 aa, synthetic construct
KALDDLERLHREINKRLEDLVRELEKLVREILKELKDALEELRRASARAG

SEQ ID NO: 899 - protein, 51 aa, synthetic construct
GEEVLRRLEEIVKKLLDLVRRILERLKEIHKDNVRLLRELNERLTRILGNA

SEQ ID NO: 900 - protein, 38 aa, synthetic construct
DEDRIIEEIARLLEELLRELLELIKKLIETNRRLAEEA

SEQ ID NO: 901 - protein, 38 aa, synthetic construct
NGDSKVNKRLEDLVRELEKLVREILKELKDALEELRRA

SEQ ID NO: 902 - protein, 39 aa, synthetic construct
SARAGGEEVLRRLEEIVKKLLDLVRRILERLKEIHKDNS

SEQ ID NO: 903 - protein, 47 aa, synthetic construct; identical to SEQ ID NO: 839

SEQ ID NO: 904 - protein, 47 aa, synthetic construct; identical to SEQ ID NO: 839

SEQ ID NO: 905 - protein, 37 aa, synthetic construct; identical to SEQ ID NO: 843

SEQ ID NO: 906 - protein, 37 aa, synthetic construct; identical to SEQ ID NO: 843

SEQ ID NO: 907 - protein, 35 aa, synthetic construct; identical to SEQ ID NO: 847

SEQ ID NO: 908 - protein, 44 aa, synthetic construct; identical to SEQ ID NO: 849

SEQ ID NO: 909 - protein, 31 aa, synthetic construct; identical to SEQ ID NO: 851

SEQ ID NO: 910 - protein, 49 aa, synthetic construct; identical to SEQ ID NO: 853

SEQ ID NO: 911 - protein, 37 aa, synthetic construct; identical to SEQ ID NO: 855

SEQ ID NO: 912 - protein, 52 aa, synthetic construct; identical to SEQ ID NO: 857

SEQ ID NO: 913 - protein, 43 aa, synthetic construct; identical to SEQ ID NO: 859

SEQ ID NO: 914 - protein, 47 aa, synthetic construct; identical to SEQ ID NO: 861

SEQ ID NO: 915 - protein, 38 aa, synthetic construct; identical to SEQ ID NO: 863

SEQ ID NO: 916 - protein, 49 aa, synthetic construct; identical to SEQ ID NO: 865

SEQ ID NO: 917 - protein, 38 aa, synthetic construct; identical to SEQ ID NO: 867

SEQ ID NO: 918 - protein, 20 aa, synthetic construct
TRGVEEVAEGVVLLRRRGNK

SEQ ID NO: 919 - protein, 19 aa, synthetic construct
RGVEEVAEGVVLLRRRGNK

SEQ ID NO: 920 - protein, 18 aa, synthetic construct
GVEEVAEGVVLLRRRGNK

SEQ ID NO: 921 - protein, 17 aa, synthetic construct
VEEVAEGVVLLRRRGNK

SEQ ID NO: 922 - protein, 16 aa, synthetic construct
EEVAEGVVLLRRRGNK

SEQ ID NO: 923 - protein, 15 aa, synthetic construct
EVAEGVVLLRRRGNK

SEQ ID NO: 924 - protein, 14 aa, synthetic construct
VAEGVVLLRRRGNK

SEQ ID NO: 925 - protein, 13 aa, synthetic construct
AEGVVLLRRRGNK

SEQ ID NO: 926 - protein, 11 aa, synthetic construct
GVVLLRRRGNK

SEQ ID NO: 927 - protein, 10 aa, synthetic construct
VVLLRRRGNK

SEQ ID NO: 928 - protein, 9 aa, synthetic construct
VLLRRRGNK

SEQ ID NO: 929 - protein, 8 aa, synthetic construct
LLRRRGNK

SEQ ID NO: 930 - protein, 7 aa, synthetic construct
LRRRGNK

SEQ ID NO: 931 - protein, 6 aa, synthetic construct
RRRGNK

SEQ ID NO: 932 - protein, 17 aa, synthetic construct
KTEVAEGVVLLRRRGNK

SEQ ID NO: 933 - protein, 17 aa, synthetic construct
KTRGAEGVVLLRRRGNK

SEQ ID NO: 934 - protein, 17 aa, synthetic construct
KTRGVEGVVLLRRRGNK

SEQ ID NO: 935 - protein, 17 aa, synthetic construct
KTRGVEEVVLLRRRGNK

SEQ ID NO: 936 - protein, 17 aa, synthetic construct
KTRGVEEVAELRRRGNK

SEQ ID NO: 937 - protein, 11 aa, synthetic construct
KTVLLRRRGNK

SEQ ID NO: 938 - protein, 11 aa, synthetic construct
KTRGLRRRGNK

SEQ ID NO: 939 - protein, 21 aa, synthetic construct
CTRGVEEVAEGVVLLRRRGNK

SEQ ID NO: 940 - protein, 21 aa, synthetic construct
FTRGVEEVAEGVVLLRRRGNK

SEQ ID NO: 941 - protein, 21 aa, synthetic construct
KIRGVEEVAEGVVLLRRRGNK

SEQ ID NO: 942 - protein, 21 aa, synthetic construct
KLRGVEEVAEGVVLLRRRGNK

SEQ ID NO: 943 - protein, 21 aa, synthetic construct
KNRGVEEVAEGVVLLRRRGNK

SEQ ID NO: 944 - protein, 21 aa, synthetic construct
KRRGVEEVAEGVVLLRRRGNK

SEQ ID NO: 945 - protein, 21 aa, synthetic construct
KTDGVEEVAEGVVLLRRRGNK

SEQ ID NO: 946 - protein, 21 aa, synthetic construct
KTFGVEEVAEGVVLLRRRGNK

SEQ ID NO: 947 - protein, 21 aa, synthetic construct
KTHGVEEVAEGVVLLRRRGNK

SEQ ID NO: 948 - protein, 21 aa, synthetic construct
KTPGVEEVAEGVVLLRRRGNK

SEQ ID NO: 949 - protein, 21 aa, synthetic construct
KTRGKEEVAEGVVLLRRRGNK

SEQ ID NO: 950 - protein, 21 aa, synthetic construct
KTRGMEEVAEGVVLLRRRGNK

SEQ ID NO: 951 - protein, 21 aa, synthetic construct
KTRGTEEVAEGVVLLRRRGNK

SEQ ID NO: 952 - protein, 21 aa, synthetic construct
KTRGVAEVAEGVVLLRRRGNK

SEQ ID NO: 953 - protein, 21 aa, synthetic construct
KTRGVEAVAEGVVLLRRRGNK

SEQ ID NO: 954 - protein, 21 aa, synthetic construct
KTRGVEDVAEGVVLLRRRGNK

SEQ ID NO: 955 - protein, 21 aa, synthetic construct
KTRGVEECAEGVVLLRRRGNK

SEQ ID NO: 956 - protein, 21 aa, synthetic construct
KTRGVEENAEGVVLLRRRGNK

SEQ ID NO: 957 - protein, 21 aa, synthetic construct
KTRGVEETAEGVVLLRRRGNK

SEQ ID NO: 958 - protein, 21 aa, synthetic construct
KTRGVEEVACGVVLLRRRGNK

SEQ ID NO: 959 - protein, 21 aa, synthetic construct
KTRGVEEVAEEVVLLRRRGNK

SEQ ID NO: 960 - protein, 21 aa, synthetic construct
KTRGVEEVAEGCVLLRRRGNK

SEQ ID NO: 961 - protein, 21 aa, synthetic construct
KTRGVEEVAEGVGLLRRRGNK

SEQ ID NO: 962 - protein, 21 aa, synthetic construct
KTRGVEEVAEGVVLLRHRGNK

SEQ ID NO: 963 - protein, 21 aa, synthetic construct
KTRGVEEVAEGVVLLRQRGNK

SEQ ID NO: 964 - protein, 21 aa, synthetic construct
KTRGVEEVAEGVVLLRRRGFK

SEQ ID NO: 965 - protein, 21 aa, synthetic construct
KTRGVEEVAEGVVLLRRRGNH

SEQ ID NO: 966 - protein, 21 aa, synthetic construct
KTRGVEEVAEGVVLTRRRGNK

SEQ ID NO: 967 - protein, 21 aa, synthetic construct
KTRGVEEVAEGWVLLRRRGNK

SEQ ID NO: 968 - protein, 21 aa, synthetic construct
KTRGVEEVAEPVVLLRRRGNK

SEQ ID NO: 969 - protein, 21 aa, synthetic construct
KTRGVEEVAHGVVLLRRRGNK

SEQ ID NO: 970 - protein, 21 aa, synthetic construct
KTRGVEEVAPGVVLLRRRGNK

SEQ ID NO: 971 - protein, 21 aa, synthetic construct
KTRGVEEVAQGVVLLRRRGNK

SEQ ID NO: 972 - protein, 21 aa, synthetic construct
KTRGVEEVATGVVLLRRRGNK

SEQ ID NO: 973 - protein, 21 aa, synthetic construct
KTRGVEEVHEGVVLLRRRGNK

SEQ ID NO: 974 - protein, 21 aa, synthetic construct
KTRGVEEVPEGVVLLRRRGNK

SEQ ID NO: 975 - protein, 21 aa, synthetic construct
KTRGVEEVREGVVLLRRRGNK

SEQ ID NO: 976 - protein, 21 aa, synthetic construct
KTRGVEHVAEGVVLLRRRGNK

SEQ ID NO: 977 - protein, 21 aa, synthetic construct
KTRGVEPVAEGVVLLRRRGNK

SEQ ID NO: 978 - protein, 21 aa, synthetic construct
KTRGVERVAEGVVLLRRRGNK

SEQ ID NO: 979 - protein, 21 aa, synthetic construct
KTRGVEVVAEGVVLLRRRGNK

SEQ ID NO: 980 - protein, 21 aa, synthetic construct
KTRGVQEVAEGVVLLRRRGNK

SEQ ID NO: 981 - protein, 21 aa, synthetic construct
KTRSVEEVAEGVVLLRRRGNK

SEQ ID NO: 982 - protein, 21 aa, synthetic construct
QTRGVEEVAEGVVLLRRRGNK

SEQ ID NO: 983 - protein, 8 aa, synthetic construct
  VARIANT 2: note = replace= T
  VARIANT 4: note = replace= R or H
  VARIANT 7: note = replace= N
  VARIANT 8: note = replace= H
  SITE 1..8: note = Variant residues given in the sequence have no preference with respect to those in the annotations for variant positions
LLRQRGFK

SEQ ID NO: 984 - protein, 7 aa, synthetic construct
  VARIANT 1: note = replace= T or K or C
  VARIANT 3: note = replace= K or R or H or Y
  VARIANT 5: note = replace= G or Y or A or C
  VARIANT 6: note = replace= N or G or Q or P or K or F
  VARIANT 7: note = replace= H or Q or S or W or R
  SITE 1..7: note = Variant residues given in the sequence have no preference with respect to those in the annotations for variant positions
LRQRNLK

SEQ ID NO: 985 - protein, 5 aa, synthetic construct
RRGNK

SEQ ID NO: 986 - protein, 11 aa, synthetic construct
KTRGVERRGNK

SEQ ID NO: 987 - protein, 11 aa, synthetic construct
KTRGVEEVAEK

SEQ ID NO: 988 - protein, 17 aa, synthetic construct
KTRGVEEVAEGVVLGNK

SEQ ID NO: 989 - protein, 11 aa, synthetic construct
KTRGVEEVGNK

SEQ ID NO: 990 - protein, 17 aa, synthetic construct
KTRGVEEVAEGVRRGNK

(19) United States
(12) Patent Application Publication
(10) Pub. No.: US 2023/0159615 A1
(43) Pub. Date: May 25, 2023

(54) CHIMERIC POLYPEPTIDES

(71) Applicant: Outpace Bio, Inc., Seattle, WA (US)

(72) Inventors: Howell MOFFETT, Seattle, WA (US); Robert LANGAN, Seattle, WA (US); Thaddeus DAVENPORT, Seattle, WA (US); Scott BOYKEN, Seattle, WA (US); Marc LAJOIE, Seattle, WA (US)

(21) Appl. No.: 17/812,712

(22) Filed: Jul. 14, 2022

Related U.S. Application Data
(63) Continuation of application No. PCT/US2021/014773, filed on Jan. 22, 2021.
(60) Provisional application No. 62/964,637, filed on Jan. 22, 2020.
(60) Provisional application No. 63/016,141, filed on Apr. 27, 2020.

(51) Int. Cl. C07K 14/725

(52) U.S. Cl. CPC C07K 14/7051; C07K 2319/03; C07K 2319/95

(57) ABSTRACT

The preset disclosure provides chimeric polypeptides comprising a cage polypeptide comprising a degron, wherein the degron is sequestered or caged. Upon activation by a key polypeptide, the degron becomes active. The degron can recruit an ubiquitin ligase and a lysine in the degron or surrounding sequence be ubiquitinated. The ubiquitinated chimeric polypeptide is marked for degradation, together with any biologically active molecule attached to the chimeric polypeptide. The chimeric polypeptides of the present disclosure can be incorporated, for example, to chimeric antigen receptors (CAR). Accordingly, in response to the administration of a key polypeptide or endogenous expression of a key polypeptide mediated, for example, by an inducible promoter, the amount of CAR expressed on the surface of an immune cell can be modulated.

### SEQUENCE LISTING

The instant application contains a Sequence Listing which has been submitted electronically in ASCII format and is hereby incorporated by reference in its entirety. Said ASCII copy, created on Jan. 21, 2021, is named 016-TNP019APCT_SL.txt and is 738,185 bytes in size.

### FIELD

The present disclosure is related to chimeric polypeptides comprising a sequestered degron, which can be used to modulate protein expression in cell and gene therapies. By way of non-limiting example, the chimeric polypeptides comprising a sequestered degron could be used to regulate expression or activity of a CAR, cytokine receptor, costimulatory/coinhibitory receptor, or transcription factor on or in T-cells, and therefore be used in cancer immunotherapy.

### BACKGROUND

Cancer immunotherapy relies on getting T cells—the immune system's primary killers of infected and diseased cells—to attack and kill tumor cells. However, there is an important stumbling block for immunotherapy: T cells' ability to kill can fade, a phenomenon often referred to as exhaustion. Ex vivo-expanded Tumor-Infiltrating Lymphocytes (TILs) therapy, chimeric antigen receptor (CAR) T cell therapy, and T cell receptor-engineered (TCR) T cell therapy are treatments that make use of functionally active T cells isolated from patients and require highly functional T cells to be effective. These T cells are engineered and expanded ex vivo to recognize specific antigens on target cancer cells. T cell therapies have not been effective at curing solid cancers because the T cells lose their ability to proliferate or kill over time. It may be possible to engineer T cells to maintain their ability to proliferate and kill by overcoming the mechanisms that cause T cells to fade. One mechanism that drives loss of T cell function is too much signaling through the CAR/TCR (Lynn, R. C., Weber, E. W., Sotillo, E. et al. Nature 576, 293-300 (2019) doi:10.1038/s41586-019-1805-z). Regulation of CAR or TCR protein stability offers the potential to prevent exhaustion driven by excessive signaling through the CAR/TCR.

The clinical responses of these CAR T cell therapies in hematologic malignancies have been encouraging. Nonetheless, they have demonstrated substantial morbidity and occasionally mortality resulting from toxicity. The most commonly observed CAR T-cell-associated toxicity is cytokine release syndrome (CRS). CRS typically occurs within the first week following CAR T-cell infusion. Peak CAR T-cell levels and serum interleukin-6 (IL-6) levels in patients have strongly correlated with the severity of CRS after CAR T-cell therapy. In some instances, the CRS can be very severe. Regulation of CAR or TCR protein stability offers the potential to tune the immune response to prevent CRS while allowing anti-tumor activity. Therefore, especially for cell and gene therapies, there is also a need to develop off switches capable of down-regulating or ablating unwanted signaling.

### BRIEF SUMMARY

The present disclosure provides a polynucleotide encoding a chimeric polypeptide which comprises a cage polypeptide comprising (i) a structural region comprising an amino acid sequence having at least about 95%, at least about 96%, at least about 97%, at least about 98%, at least about 99%, or about 100% sequence identity to Cage1_N_SR (SEQ ID NO: 142), Cage1_C_SR (SEQ ID NO: 143), Cage2_N_SR (SEQ ID NO: 144), Cage2_C_SR (SEQ ID NO: 145), Cage3_N_SR (SEQ ID NO: 146), Cage3_C_SR (SEQ ID NO: 147), Cage4_N_SR (SEQ ID NO: 148), Cage4_C_SR (SEQ ID NO: 149), Cage5_N_SR (SEQ ID NO: 150), Cage5_C_SR (SEQ ID NO: 151), Cage6_N_SR (SEQ ID NO: 152), Cage6_C_SR (SEQ ID NO: 153), Cage7_N_SR (SEQ ID NO: 154), Cage7_C_SR (SEQ ID NO: 155), Cage8_N_SR (SEQ ID NO: 156), Cage8_C_SR (SEQ ID NO: 157), Cage9_N_SR (SEQ ID NO: 158), Cage9_C_SR (SEQ ID NO: 159), Cage10_N_SR (SEQ ID NO: 160), Cage10_C_SR (SEQ ID NO: 161), Cage 1_N_SR (SEQ ID NO: 162), Cage11_C_SR (SEQ ID NO: 163), Cage12_N_SR (SEQ ID NO: 164), Cage12_C_SR (SEQ ID NO: 165), Cage13_SR (SEQ ID NO: 838), Cage14_SR (SEQ ID NO: 840), Cage15_SR (SEQ ID NO: 842), Cage16_SR (SEQ ID NO: 844), Cage17_SR (SEQ ID NO: 846), Cage18_SR (SEQ ID NO: 848), Cage19_SR (SEQ ID NO: 850), Cage20_SR (SEQ ID NO: 852), Cage21_SR (SEQ ID NO: 854), Cage22_SR (SEQ ID NO: 856), Cage23_SR (SEQ ID NO: 858), Cage24_SR (SEQ ID NO: 860), Cage25_SR (SEQ ID NO: 862), Cage26_SR (SEQ ID NO: 864), or Cage27_SR (SEQ ID NO: 866), and (ii) a latch region comprising a degron peptide consisting of the amino acid sequence as set forth in KTRGVEEVAEGVVLLRRRGN (SEQ ID NO: 1), KTRGVEEVAEGVVLLRRRGNK (DegronA*, SEQ ID NO: 801); KEEVAEGVVLLRRRGNK (DegronA*_Truncation1, SEQ ID NO: 802); KTRGVEEVAEGVVLLRK (DegronA*_Truncation2, SEQ ID NO: 803); EGVVLLRRRGNK (DegronA*_Truncation3, SEQ ID NO: 804); TRGVEEVAEGVVLLRRRGNK (DegronA*_Truncation6, SEQ ID NO: 918); RGVEEVAEGVVLLRRRGNK (DegronA*_Truncation7, SEQ ID NO: 919); GVEEVAEGVVLLRRRGNK (DegronA*_Truncation8, SEQ ID NO: 920); VEEVAEGVVLLRRRGNK (DegronA*_Truncation9, SEQ ID NO: 921); EEVAEGVVLLRRRGNK (DegronA*_Truncation10, SEQ ID NO: 922); EVAEGVVLLRRRGNK (DegronA*_Truncation11, SEQ ID NO: 923); VAEGVVLLRRRGNK (DegronA*_Truncation12, SEQ ID NO: 924); AEGVVLLRRRGNK (DegronA*_Truncation13, SEQ ID NO: 925); GVVLLRRRGNK (DegronA*_Truncation14, SEQ ID NO: 926); VVLLRRRGNK (DegronA*_Truncation15, SEQ ID NO: 927); VLLRRRGNK (DegronA*_Truncation16, SEQ ID NO: 928); LLRRRGNK (DegronA*_Truncation17, SEQ ID NO: 929); LRRRGNK (DegronA*_Truncation18, SEQ ID NO: 930); RRRGNK (DegronA*_Truncation19, SEQ ID NO: 931); KTEVAEGVVLLRRRGNK (DegronA*_Truncation20, SEQ ID NO: 932); KTRGAEGVVLLRRRGNK (DegronA*_Truncation2l, SEQ ID NO: 933); KTRGVEGVVLLRRRGNK (DegronA*_Truncation22, SEQ ID NO: 934); KTRGVEEVVLLRRRGNK (DegronA*_Truncation23, SEQ ID NO: 935); KTRGVEEVAELRRRGNK (DegronA*_Truncation24, SEQ ID NO: 936); KTVLLRRRGNK (DegronA*_Truncation25, SEQ ID NO: 937); KTRGLRRRGNK (DegronA*_Truncation26, SEQ ID NO: 938); CTRGVEEVAEGVVLLRRRGNK (DegronA*_Mutation1, SEQ ID NO: 939); FTRGVEEVAEGVVLLRRRGNK (DegronA*_Mutation2, SEQ ID NO: 940); KIRGVEEVAEGVVLLRRRGNK (DegronA*_Mutation3, SEQ ID NO: 941); KLRGVEEVAEGVVLLRRRGNK (DegronA*_Mutation4, SEQ ID NO: 942); KNRGVEEVAEGVVLLRRRGNK (DegronA*_Mutation5, SEQ ID NO: 943); KRRGVEEVAEGVVLLRRRGNK (DegronA*_Mutation6, SEQ ID NO: 944); KTDGVEEVAEGVVLLRRRGNK (DegronA*_Mutation7, SEQ ID NO: 945); KTFGVEEVAEGVVLLRRRGNK (DegronA*_Mutation8, SEQ ID NO: 946); KTHGVEEVAEGVVLLRRRGNK (DegronA*_Mutation9, SEQ ID NO: 947); KTPGVEEVAEGVVLLRRRGNK (DegronA*_Mutation10, SEQ ID NO: 948); KTRGKEEVAEGVVLLRRRGNK (DegronA*_Mutation11, SEQ ID NO: 949); KTRGMEEVAEGVVLLRRRGNK (DegronA*_Mutation12, SEQ ID NO: 950); KTRGTEEVAEGVVLLRRRGNK (DegronA*_Mutation13, SEQ ID NO: 951); KTRGVAEVAEGVVLLRRRGNK (DegronA*_Mutation14, SEQ ID NO: 952); KTRGVEAVAEGVVLLRRRGNK (DegronA*_Mutation15, SEQ ID NO: 953); KTRGVEDVAEGVVLLRRRGNK (DegronA*_Mutation16, SEQ ID NO: 954); KTRGVEECAEGVVLLRRRGNK (DegronA*_Mutation17, SEQ ID NO: 955); KTRGVEENAEGVVLLRRRGNK (DegronA*_Mutation18, SEQ ID NO: 956); KTRGVEETAEGVVLLRRRGNK (DegronA*_Mutation19, SEQ ID NO: 957); KTRGVEEVACGVVLLRRRGNK (DegronA*_Mutation20, SEQ ID NO: 958); KTRGVEEVAEEVVLLRRRGNK (DegronA*_Mutation21, SEQ ID NO: 959); KTRGVEEVAEGCVLLRRRGNK (DegronA*_Mutation22, SEQ ID NO: 960); KTRGVEEVAEGVGLLRRRGNK (DegronA*_Mutation23, SEQ ID NO: 961); KTRGVEEVAEGVVLLRHRGNK (DegronA*_Mutation24, SEQ ID NO: 962); KTRGVEEVAEGVVLLRQRGNK (DegronA*_Mutation25, SEQ ID NO: 963); KTRGVEEVAEGVVLLRRRGFK (DegronA*_Mutation26, SEQ ID NO: 964); KTRGVEEVAEGVVLLRRRGNH (DegronA*_Mutation27, SEQ ID NO: 965); KTRGVEEVAEGVVLTRRRGNK (DegronA*_Mutation28, SEQ ID NO: 966); KTRGVEEVAEGWVLLRRRGNK (DegronA*_Mutation29, SEQ ID NO: 967); KTRGVEEVAEPVVLLRRRGNK (DegronA*_Mutation30, SEQ ID NO: 968); KTRGVEEVAHGVVLLRRRGNK (DegronA*_Mutation31, SEQ ID NO: 969); KTRGVEEVAPGVVLLRRRGNK (DegronA*_Mutation32, SEQ ID NO: 970); KTRGVEEVAQGVVLLRRRGNK (DegronA*_Mutation33, SEQ ID NO: 971); KTRGVEEVATGVVLLRRRGNK (DegronA*_Mutation34, SEQ ID NO: 972); KTRGVEEVHEGVVLLRRRGNK (DegronA*_Mutation35, SEQ ID NO: 973); KTRGVEEVPEGVVLLRRRGNK (DegronA*_Mutation36, SEQ ID NO: 974); KTRGVEEVREGVVLLRRRGNK (DegronA*_Mutation37, SEQ ID NO: 975); KTRGVEHVAEGVVLLRRRGNK (DegronA*_Mutation38, SEQ ID NO: 976); KTRGVEPVAEGVVLLRRRGNK (DegronA*_Mutation39, SEQ ID NO: 977); KTRGVERVAEGVVLLRRRGNK (DegronA*_Mutation40, SEQ ID NO: 978); KTRGVEVVAEGVVLLRRRGNK (DegronA*_Mutation41, SEQ ID NO: 979); KTRGVQEVAEGVVLLRRRGNK (DegronA*_Mutation42, SEQ ID NO: 980); KTRSVEEVAEGVVLLRRRGNK (DegronA*_Mutation43, SEQ ID NO: 981); or QTRGVEEVAEGVVLLRRRGNK (DegronA*_Mutation44, SEQ ID NO: 982), wherein the latch region is capable of binding to the structural region. In some aspects, a degron useful for the present disclosure is not KTRGVEEV (DegronA*_Truncation4, SEQ ID NO: 805) or KTRGVEEVAEGV (DegronA*_Truncation5, SEQ ID NO: 806). In some aspects, a degron useful for the present disclosure comprises EGVVLLRRRG (SEQ ID NO: 122) or EGVVLLRRRGN (SEQ ID NO: 123). Correspondence between the designations in this Brief Summary and their respective amino acid sequences is provided in the Sequences table provided in the Detailed Description section (thus, e.g., Cage1_N_SR would correspond to SEQ ID NO:142).

In some aspects, the structural region of the cage polypeptide comprises any one of the amino acid sequences as set forth in Cage1_N_SR (SEQ ID NO: 142), Cage1_C_SR (SEQ ID NO: 143), Cage2_N_SR (SEQ ID NO: 144), Cage2_C_SR (SEQ ID NO: 145), Cage3_N_SR (SEQ ID NO: 146), Cage3_C_SR (SEQ ID NO: 147), Cage4_N_SR (SEQ ID NO: 148), Cage4_C_SR (SEQ ID NO: 149), Cage5_N_SR (SEQ ID NO: 150), Cage5_C_SR (SEQ ID NO: 151), Cage6_N_SR (SEQ ID NO: 152), Cage6_C_SR (SEQ ID NO: 153), Cage7_N_SR (SEQ ID NO: 154), Cage7_C_SR (SEQ ID NO: 155), Cage8_N_SR (SEQ ID NO: 156), Cage8_C_SR (SEQ ID NO: 157), Cage9_N_SR (SEQ ID NO: 158), Cage9_C_SR (SEQ ID NO: 159), Cage10_N_SR (SEQ ID NO: 160), Cage10_C_SR (SEQ ID NO: 161), Cage11_N_SR (SEQ ID NO: 162), Cage11_C_SR (SEQ ID NO: 163), Cage12_N_SR (SEQ ID NO: 164), Cage12_C_SR (SEQ ID NO: 165), Cage13_SR (SEQ ID NO: 838), Cage14_SR (SEQ ID NO: 840), Cage15_SR (SEQ ID NO: 842), Cage16_SR (SEQ ID NO: 844), Cage17_SR (SEQ ID NO: 846), Cage18_SR (SEQ ID NO: 848), Cage19_SR (SEQ ID NO: 850), Cage20_SR (SEQ ID NO: 852), Cage21_SR (SEQ ID NO: 854), Cage22_SR (SEQ ID NO: 856), Cage23_SR (SEQ ID NO: 858), Cage24_SR (SEQ ID NO: 860), Cage25_SR (SEQ ID NO: 862), Cage26_SR (SEQ ID NO: 864), or Cage27_SR (SEQ ID NO: 866). In some aspects, the latch region comprises at least at least 10, 15, 20, 21, 22, 23, 24, 25, 26, 27, 28, 29, 30, 31, 32, 33, 34, or 35 consecutive amino acid residues from Cage1_N_Latch (SEQ ID NO: 166), Cage1_C_Latch (SEQ ID NO: 167), Cage2_N_Latch (SEQ ID NO: 168), Cage2_C_Latch (SEQ ID NO: 169), Cage3_N_Latch (SEQ ID NO: 170), Cage3_C_Latch (SEQ ID NO: 171), Cage4_N_Latch (SEQ ID NO: 172), Cage4_C_Latch (SEQ ID NO: 173), Cage5_N_Latch (SEQ ID NO: 174), Cage5_C_Latch (SEQ ID NO: 175), Cage6_N_Latch (SEQ ID NO: 176), Cage6_C_Latch (SEQ ID NO: 177), Cage7_N_Latch (SEQ ID NO: 178), Cage7_C_Latch (SEQ ID NO: 179), Cage8_N_Latch (SEQ ID NO: 180), Cage8_C_Latch (SEQ ID NO: 181), Cage9_N_Latch (SEQ ID NO: 182), Cage9_C_Latch (SEQ ID NO: 183), Cage10_N_Latch (SEQ ID NO: 184), Cage10_C_Latch (SEQ ID NO: 185), Cage11_N_Latch (SEQ ID NO: 186), Cage11_C_Latch (SEQ ID NO: 187), Cage12_N_Latch (SEQ ID NO: 188), Cage12_C_Latch (SEQ ID NO: 189), Cage13_Latch (SEQ ID NO: 839), Cage14_Latch (SEQ ID NO: 841), Cage15_Latch (SEQ ID NO: 843), Cage16_Latch (SEQ ID NO: 845), Cage17_Latch (SEQ ID NO: 847), Cage18_Latch (SEQ ID NO: 849), Cage19_Latch (SEQ ID NO: 851), Cage20_Latch (SEQ ID NO: 853), Cage21_Latch (SEQ ID NO: 855), Cage22_Latch (SEQ ID NO: 857), Cage23_Latch (SEQ ID NO: 859), Cage24_Latch (SEQ ID NO: 861), Cage25_Latch (SEQ ID NO: 863), Cage26_Latch (SEQ ID NO: 865), or Cage27_Latch (SEQ ID NO: 867).

In some aspects, the latch region of the cage polypeptide further comprises one or more amino acids derived from Cage1_N_Latch (SEQ ID NO: 166), Cage1_C_Latch (SEQ ID NO: 167), Cage2_N_Latch (SEQ ID NO: 168), Cage2_C_Latch (SEQ ID NO: 169), Cage3_N_Latch (SEQ ID NO: 170), Cage3_C_Latch (SEQ ID NO: 171), Cage4_N_Latch (SEQ ID NO: 172), Cage4_C_Latch (SEQ ID NO: 173), Cage5_N_Latch (SEQ ID NO: 174), Cage5_C_Latch (SEQ ID NO: 175), Cage6_N_Latch (SEQ ID NO: 176), Cage6_C_Latch (SEQ ID NO: 177), Cage7_N_Latch (SEQ ID NO: 178), Cage7_C_Latch (SEQ ID NO: 179), Cage8_N_Latch (SEQ ID NO: 180), Cage8_C_Latch (SEQ ID NO: 181), Cage9_N_Latch (SEQ ID NO: 182), Cage9_C_Latch (SEQ ID NO: 183), Cage10_N_Latch (SEQ ID NO: 184), Cage10_C_Latch (SEQ ID NO: 185), Cage11_N_Latch (SEQ ID NO: 186), Cage11_C_Latch (SEQ ID NO: 187), Cage12_N_Latch (SEQ ID NO: 188), Cage12_C_Latch (SEQ ID NO: 189), Cage13_Latch (SEQ ID NO: 839), Cage14_Latch (SEQ ID NO: 841), Cage15_Latch (SEQ ID NO: 843), Cage16_Latch (SEQ ID NO: 845), Cage17_Latch (SEQ ID NO: 847), Cage18_Latch (SEQ ID NO: 849), Cage19_Latch (SEQ ID NO: 851), Cage20_Latch (SEQ ID NO: 853), Cage21_Latch (SEQ ID NO: 855), Cage22_Latch (SEQ ID NO: 857), Cage23_Latch (SEQ ID NO: 859), Cage24_Latch (SEQ ID NO: 861), Cage25_Latch (SEQ ID NO: 863), Cage26_Latch (SEQ ID NO: 865), or Cage27_Latch (SEQ ID NO: 867).

In some aspects, the latch region of the cage polypeptide comprises Cage1_N_Latch (SEQ ID NO: 166) or Cage1_C_Latch (SEQ ID NO: 167) except that the sequence comprising amino acids 2 to 21 of Cage1_N_Latch (SEQ ID NO: 166), 6 to 25 of Cage1_N_Latch (SEQ ID NO: 166) 1 to 20 of Cage1_C_Latch (SEQ ID NO: 167), 6 to 25 of Cage1_C Latch (SEQ ID NO: 167), or 9 to 28 of Cage1_C_Latch (SEQ ID NO: 167) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage2_N_Latch (SEQ ID NO: 168) or Cage2_C_Latch (SEQ ID NO: 169) except that the sequence comprising amino acids 35 to 54 of Cage2_N_Latch (SEQ ID NO: 168) or 40 to 59 of Cage2_C_Latch (SEQ ID NO: 169) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage3_N_Latch (SEQ ID NO: 170) or Cage3_C_Latch (SEQ ID NO: 171) except that the sequence comprising amino acids 3 to 22, 10 to 29, 14 to 33, 17 to 36, 21 to 40, 28 to 47, 32 to 51, 35 to 54, or 39 to 58 of Cage3_N_Latch (SEQ ID NO: 170) or 1 to 20, 5 to 24, 8 to 27, 37 to 56, or 41 to 60 of Cage3_C_Latch (SEQ ID NO: 171) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage4_N_Latch (SEQ ID NO: 172) or Cage4_C_Latch (SEQ ID NO: 173) except that the sequence comprising amino acids 6 to 25 or 7 to 26 of Cage4_N_Latch (SEQ ID NO: 172) or amino acids 1 to 19, 5 to 24, 8 to 27, 12 to 31, 15 to 34, 16 to 35, 23 to 42, 26 to 45, 27 to 46, 30 to 49, 34 to 53, 38 to 57, 40 to 59, or 41 to 60 of Cage4_C_Latch (SEQ ID NO: 173) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage5_N_Latch (SEQ ID NO: 174) or Cage5_C_Latch (SEQ ID NO: 175) except that the sequence comprising amino acids 10 to 29 or 35 to 54 of Cage5_N_Latch (SEQ ID NO: 174) or amino acids 3 to 22, 5 to 24, or 12 to 31 of Cage5_C_Latch (SEQ ID NO: 175) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage6_N_Latch (SEQ ID NO: 176) except that the sequence comprising amino acids 6 to 25 of Cage6_N_Latch (SEQ ID NO: 176) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage7_N_Latch (SEQ ID NO: 178) or Cage7_C_Latch (SEQ ID NO: 179) except that the sequence comprising amino acids 3 to 22 of Cage7_N_Latch (SEQ ID NO: 178) or amino acids 30 to 49 of Cage7_C_Latch (SEQ ID NO: 179) is a degron peptide sequence, e.g., a degron consisting of an amino acid sequence as set forth in KTRGVEEVAEGVVLLRRRGN (SEQ ID NO: 1), SEQ ID NOS: 110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage8_C_Latch (SEQ ID NO: 181) except that the sequence comprising amino acids 18 to 37 of Cage8_C_Latch (SEQ ID NO: 181) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage9_N_Latch (SEQ ID NO: 182) or Cage9_C_Latch (SEQ ID NO: 183) except that the sequence comprising amino acids 3 to 22, 6 to 25, or 13 to 32 of Cage9_N Latch (SEQ ID NO: 182) or 41 to 60 of Cage9_C_Latch (SEQ ID NO: 183) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage10_N Latch (SEQ ID NO: 184) or Cage10_C_Latch (SEQ ID NO: 185) except that the sequence comprising amino acids 6 to 25, 13 to 32, or 38 to 57 of Cage10_N_Latch (SEQ ID NO: 184) or amino acids 1 to 20, 4 to 23, 30 to 49, or 33 to 52 of Cage10_C_Latch (SEQ ID NO: 185) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage11_N_Latch (SEQ ID NO: 186) except that the sequence comprising amino acids 13 to 32 of Cage11_N_Latch (SEQ ID NO: 186) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises Cage12_C_Latch (SEQ ID NO: 189) except that the sequence comprising amino acids 23 to 42 of Cage12_C_Latch (SEQ ID NO: 189) is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, a degron useful for the present disclosure comprises EGVVLLRRRG (SEQ ID NO: 122) or EGVVLLRRRGN (SEQ ID NO: 123).

In some aspects, the latch region of the cage polypeptide comprises an amino acid sequence set forth in SEQ ID NO: 839 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 841 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 843 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 845 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 847 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 849 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 851 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 853 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 855 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 857 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 859 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 861 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 863 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 865 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982. In some aspects, the latch region of the cage polypeptide comprises an amino acid sequence set forth in SEQ ID NO: 867 except that the sequence comprises a subsequence that is a degron peptide sequence, e.g., a degron consisting of an amino acid sequence as set forth in KTRGVEEVAEGVVLLRRRGN (SEQ ID NO: 1), SEQ ID NOS:110-141, SEQ ID NOS: 680-804, or SEQ ID NOS: 918-982.

In some aspects, the chimeric polypeptide comprises an amino acid sequence having at least about 95%, at least about 96%, at least about 97%, at least about 98%, at least about 99%, or about 100% sequence identity to Peptide1_121 (SEQ ID NO: 14); Peptide1_125 (SEQ ID NO: 15); Peptide1_128 (SEQ ID NO: 16); Peptide1_2 (SEQ ID NO: 17); or Peptide1_6 (SEQ ID NO: 18). In some aspects, the chimeric polypeptide comprises an amino acid sequence having at least about 95%, at least about 96%, at least about 97%, at least about 98%, at least about 99%, or about 100% sequence identity to Peptide2_154 (SEQ ID NO: 19); Peptide2_35 (SEQ ID NO: 20); Peptide2_35_medium_degron (SEQ ID NO: 818); or Peptide2_35_short_degron (SEQ ID NO: 819). In some aspects, the chimeric polypeptide comprises an amino acid sequence having at least about 95%, at least about 96%, at least about 97%, at least about 98%, at least about 99%, or about 100% sequence identity to Peptide3_10 (SEQ ID NO: 21); Peptide3_123 (SEQ ID NO: 22); Peptide3_127 (SEQ ID NO: 23); Peptide3_130 (SEQ ID NO: 24); Peptide3_14 (SEQ ID NO: 25); Peptide3_159 (SEQ ID NO: 26); Peptide3_163 (SEQ ID NO: 27); Peptide3_17 (SEQ ID NO: 28); Peptide3_21 (SEQ ID NO: 29); Peptide3_28 (SEQ ID NO: 30); Peptide3_3 (SEQ ID NO: 31); Peptide3_32 (SEQ ID NO: 32); Peptide3_35 (SEQ ID NO: 33); Peptide3_39 (SEQ ID NO: 34); Peptide3_147 (SEQ ID NO: 807); Peptide3_147_shortened_9N (SEQ ID NO: 808); Peptide3_147_shortened_9C (SEQ ID NO: 809); Peptide3_147_shortened_9C9N (SEQ ID NO: 810); Peptide3_21_shortened_9N (SEQ ID NO: 811); Peptide3_21_medium_degron (SEQ ID NO: 814); or Peptide3_21_short_degron (SEQ ID NO: 815). In some aspects, the chimeric polypeptide comprises an amino acid sequence having at least about 95%, at least about 96%, at least about 97%, at least about 98%, at least about 99%, or about 100% sequence identity to Peptide4_125 (SEQ ID NO: 35); Peptide4_129 (SEQ ID NO: 36); Peptide4_132 (SEQ ID NO: 37); Peptide4_136 (SEQ ID NO: 38); Peptide4_139 (SEQ ID NO: 39); Peptide4_140 (SEQ ID NO: 40); Peptide4_147 (SEQ ID NO: 41); Peptide4_150 (SEQ ID NO: 42); Peptide4_151 (SEQ ID NO: 43); Peptide4_154 (SEQ ID NO: 44); Peptide4_158 (SEQ ID NO: 45); Peptide4_162 (SEQ ID NO: 46); Peptide4_164 (SEQ ID NO: 47); Peptide4_165 (SEQ ID NO: 48); Peptide4_21 (SEQ ID NO: 49); Peptide4_24 (SEQ ID NO: 50); Peptide4_25 (SEQ ID NO: 51); Peptide4_39 (SEQ ID NO: 52); Peptide4_6 (SEQ ID NO: 53); Peptide4_7 (SEQ ID NO: 54); Peptide4_154_medium_degron (SEQ ID NO: 812); or Peptide4_154_short_degron (SEQ ID NO: 813).

In some aspects, the chimeric polypeptide comprises an amino acid sequence having at least about 95%, at least about 96%, at least about 97%, at least about 98%, at least about 99%, or about 100% sequence identity to Peptide5_190 (SEQ ID NO: 55); Peptide5_192 (SEQ ID NO: 56); Peptide5_10 (SEQ ID NO: 57); Peptide5_199 (SEQ ID NO: 58); or Peptide5_35 (SEQ ID NO: 59). In some aspects, the chimeric polypeptide comprises an amino acid sequence having at least about 95%, at least about 96%, at least about 97%, at least about 98%, at least about 99%, or about 100% sequence identity to Peptide6_6 (SEQ ID NO: 60). In some aspects, the chimeric polypeptide comprises an amino acid sequence having at least about 95%, at least about 96%, at least about 97%, at least about 98%, at least about 99%, or about 100% sequence identity to Peptide7_218 (SEQ ID NO: 61); Peptide7_3 (SEQ ID NO: 62); Peptide7_218_medium_degron (SEQ ID NO: 820); or Peptide7_218_short_degron (SEQ ID NO: 821). In some aspects, the chimeric polypeptide comprises an amino acid sequence having at least about 95%, at least about 96%, at least about 97%, at least about 98%, at least about 99%, or about 100% sequence identity to Peptide8_200 (SEQ ID NO: 63).

In some aspects, the chimeric polypeptide comprises an amino acid sequence having at least about 95%, at least about 96%, at least about 97%, at least about 98%, at least about 99%, or about 100% sequence identity to Peptide9_3 (SEQ ID NO: 64); Peptide9_214 (SEQ ID NO: 65); Peptide9_6 (SEQ ID NO: 66); or Peptide9_13 (SEQ ID NO: 67). In some aspects, the chimeric polypeptide comprises an amino acid sequence having at least about 95%, at least about 96%, at least about 97%, at least about 98%, at least about 99%, or about 100% sequence identity to Peptide10_219 (SEQ ID NO: 68); Peptide10_222 (SEQ ID NO: 69); Peptide10_190 (SEQ ID NO: 70); Peptide10_13 (SEQ ID NO: 71); Peptide10_6 (SEQ ID NO: 72); Peptide10_193 (SEQ ID NO: 73); or Peptide10_38 (SEQ ID NO: 74); Peptide10_38_medium_degron (SEQ ID NO: 816); or Peptide10_38_short_degron (SEQ ID NO: 817). In some aspects, the chimeric polypeptide comprises an amino acid sequence having at least about 95%, at least about 96%, at least about 97%, at least about 98%, at least about 99%, or about 100% sequence identity to Peptide11_13 (SEQ ID NO: 75). In some aspects, the chimeric polypeptide comprises an amino acid sequence having at least about 95%, at least about 96%, at least about 97%, at least about 98%, at least about 99%, or about 100% sequence identity to Peptide12_206 (SEQ ID NO: 76).

In some aspects, the chimeric polypeptide further comprises a signaling domain. In some aspects, the signaling domain comprises a domain derived from CD3zeta, FcR gamma, FcR beta, CD3 gamma, CD3 delta, CD3 epsilon, CD5, CD22, CD79a, CD79b, and CD66d. In some aspects, the chimeric polypeptide further comprises a co-stimulatory domain. In some aspects, the co-stimulatory domain comprises a domain derived from 2B4, HVEM, ICOS, LAG3, DAP10, DAP12, CD27, CD28, 4_1BB (CD137), OX40 (CD134), CD30, CD40, ICOS (CD278), glucocorticoid-induced tumor necrosis factor receptor (GITR), lymphocyte function-associated antigen_1 (LFA_1), CD2, CD7, LIGHT, NKG2C, or B7_H3. In some aspects, the chimeric polypeptide further comprises an antigen-binding domain. In some aspects, the antigen-binding domain comprises an antibody or an antigen-binding fragment thereof that specifically binds to an epitope on a tumor antigen. In some aspects, the antigen binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, or a nanobody. In some aspects, the antigen binding domain specifically binds to CD19, TRAC, TCRβ, BCMA, CLL_1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL_13Ra2, Mesothelin, IL-l lRa, PSCA, PRSS21, VEGFR2, LewisY, CD24, PDGFR-beta, SSEA_4, CD20, Folate receptor alpha, ERBB2 (Her2/neu), MUC1, MUC16, EGFR, NCAM, Prostase, PAP, ELF2M, Ephrin B2, IGF_I receptor, CAIX, LMP2, gplOO, bcr-abl, tyrosinase, EphA2, Fucosyl GM1, sLe, GM3, TGS5, HMWMAA, o_acetyl_GD2, Folate receptor beta, TEM1/CD248, TEM7R, CLDN6, GPRC5D, CXORF61, CD97, CD179a, ALK, Polysialic acid, PLAC1, GloboH, NY_BR_1, UPK2, HAVCR1, ADRB3, PANX3, GPR20, LY6K, OR51E2, TARP, WTI, NY_ESO_1, LAGE-la, MAGE-Al, legumain, HPV E6,E7, MAGE Al, ETV6_AML, sperm protein 17, XAGE1, Tie 2, MAD_CT_1, MAD_CT_2, Fos-related antigen 1, p53, p53 mutant, prostein, survivin and telomerase, PCTA_1/Galectin 8, MelanA/MARTI, Ras mutant, hTERT, sarcoma translocation breakpoints, ML_IAP, ERG (TMPRSS2 ETS fusion gene), NA17, PAX3, Androgen receptor, Cyclin B1, MYCN, RhoC, TRP_2, CYP1B1, BORIS, SART3, PAX5, OY_TES1, LCK, AKAP_4, SSX2, RAGE_1, human telomerase reverse transcriptase, RU1, RU2, intestinal carboxyl esterase, mut hsp70_2, CD79a, CD79b, CD72, LAIR1, FCAR, LILRA2, CD300LF, CLEC12A, BST2, EMR2, LY75, GPC3, FCRL5, IGLL1, or any combinations thereof.

In some aspects, the chimeric polypeptide comprises a chimeric antigen receptor (CAR). In some aspects, the CAR is designed as a standard CAR, a split CAR, an off-switch CAR, an on-switch CAR, a first-generation CAR, a second-generation CAR, a third-generation CAR, or a fourth-generation CAR. In some aspects, the chimeric polypeptide further comprises a transmembrane domain.

In some aspects, the polynucleotide encodes a chimeric antigen receptor (CAR) comprising (i) the chimeric polypeptide encoded by a polynucleotide disclosed herein; (ii) an antigen-binding domain that binds to an epitope on a tumor antigen expressed on a target cell; and (iii) a transmembrane domain. In some aspects, the CAR further comprises a CAR spacer between the antigen-binding domain and the transmembrane domain. In some aspects, the chimeric polypeptide comprises a T cell receptor. In some aspects, the chimeric polypeptide induces an IFNγ and/or IL-2 expression in a cell. In some aspects, the chimeric polypeptide is capable of being degraded through an ubiquitin dependent pathway. In some aspects, the chimeric polypeptide is capable of being degraded when in contact with a key polypeptide.

In some aspects, the polynucleotide is a DNA molecule, an RNA molecule, or any combination thereof.

The present disclosure also provides a polynucleotide set comprising a polynucleotide disclosed above and a second polynucleotide. In some aspects, the second polynucleotide encodes a key polypeptide that is capable of inhibiting binding of the latch region to the structural region of the chimeric polypeptide when the key polypeptide comes in contact with the chimeric polypeptide. In some aspects, the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key1C (SEQ ID NO: 77), Key1N (SEQ ID NO: 78); Key2C (SEQ ID NO: 79); Key2N (SEQ ID NO: 80); Key3C (SEQ ID NO: 81); Key3N (SEQ ID NO: 82); Key4C (SEQ ID NO: 83); Key4N (SEQ ID NO: 84); Key5C (SEQ ID NO: 85); Key5N (SEQ ID NO: 86); Key6N (SEQ ID NO: 87); Key7C (SEQ ID NO: 88); Key7N (SEQ ID NO: 89); Key8C (SEQ ID NO: 90); Key9C (SEQ ID NO: 91); Key9N (SEQ ID NO: 92); Key10C (SEQ ID NO: 93); Key10N (SEQ ID NO: 94); Key11N (SEQ ID NO: 95); Key12C (SEQ ID NO: 96), wherein the key polypeptide is capable of binding to the structural region of the chimeric polypeptide.

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide1_121 (SEQ ID NO: 14); Peptide1_125 (SEQ ID NO: 15); Peptide1_128 (SEQ ID NO: 16); Peptide1_2 (SEQ ID NO: 17); or Peptide1_6 (SEQ ID NO: 18), and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key1C (SEQ ID NO: 77) or Key1N (SEQ ID NO: 78). In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide2_154 (SEQ ID NO: 19); Peptide2_35 (SEQ ID NO: 20); Peptide2_35_medium_degron (SEQ ID NO: 818); or Peptide2_35_short_degron (SEQ ID NO: 819), and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key2C (SEQ ID NO: 79) or Key2N (SEQ ID NO: 80).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide3_10 (SEQ ID NO: 21); Peptide3_123 (SEQ ID NO: 22); Peptide3_127 (SEQ ID NO: 23); Peptide3_130 (SEQ ID NO: 24); Peptide3_14 (SEQ ID NO: 25); Peptide3_159 (SEQ ID NO: 26); Peptide3_163 (SEQ ID NO: 27); Peptide3_17 (SEQ ID NO: 28); Peptide3_21 (SEQ ID NO: 29); Peptide3_28 (SEQ ID NO: 30); Peptide3_3 (SEQ ID NO: 31); Peptide3_32 (SEQ ID NO: 32); Peptide3_35 (SEQ ID NO: 33); Peptide3_39 (SEQ ID NO: 34); Peptide3_147 (SEQ ID NO: 807); Peptide3_147_shortened_9N (SEQ ID NO: 808); Peptide3_147_shortened_9C (SEQ ID NO: 809); Peptide3_147_shortened_9C9N (SEQ ID NO: 810); Peptide3_21_shortened_9N (SEQ ID NO: 811); Peptide3_21_medium_degron (SEQ ID NO: 814); or Peptide3_21_short_degron (SEQ ID NO: 815) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key3N (SEQ ID NO: 82) or Key3C (SEQ ID NO: 81).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide4_125 (SEQ ID NO: 35); Peptide4_129 (SEQ ID NO: 36); Peptide4_132 (SEQ ID NO: 37); Peptide4_136 (SEQ ID NO: 38); Peptide4_139 (SEQ ID NO: 39); Peptide4_140 (SEQ ID NO: 40); Peptide4_147 (SEQ ID NO: 41); Peptide4_150 (SEQ ID NO: 42); Peptide4_151 (SEQ ID NO: 43); Peptide4_154 (SEQ ID NO: 44); Peptide4_158 (SEQ ID NO: 45); Peptide4_162 (SEQ ID NO: 46); Peptide4_164 (SEQ ID NO: 47); Peptide4_165 (SEQ ID NO: 48); Peptide4_21 (SEQ ID NO: 49); Peptide4_24 (SEQ ID NO: 50); Peptide4_25 (SEQ ID NO: 51); Peptide4_39 (SEQ ID NO: 52); Peptide4_6 (SEQ ID NO: 53); Peptide4_7 (SEQ ID NO: 54); Peptide4_154_medium_degron (SEQ ID NO: 812); or Peptide4_154_short_degron (SEQ ID NO: 813), and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key4N (SEQ ID NO: 84) or Key4C (SEQ ID NO: 83).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide5_190 (SEQ ID NO: 55); Peptide5_192 (SEQ ID NO: 56); Peptide5_10 (SEQ ID NO: 57); Peptide5_199 (SEQ ID NO: 58); Peptide5_35 (SEQ ID NO: 59) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key5C (SEQ ID NO: 85) or Key5N (SEQ ID NO: 86).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide6_6 (SEQ ID NO: 60) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key6N (SEQ ID NO: 87).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide7_218 (SEQ ID NO: 61); Peptide7_3 (SEQ ID NO: 62); or Peptide7_218_medium_degron (SEQ ID NO: 820); or Peptide7_218_short_degron (SEQ ID NO: 821), and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key7C (SEQ ID NO: 88) or Key7N (SEQ ID NO: 89).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide8_200 (SEQ ID NO: 63) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key8C (SEQ ID NO: 90).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide9_3 (SEQ ID NO: 64); Peptide9_214 (SEQ ID NO: 65); Peptide9_6 (SEQ ID NO: 66); or Peptide9_13 (SEQ ID NO: 67) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key9C (SEQ ID NO: 91) or Key9N (SEQ ID NO: 92).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide10_219 (SEQ ID NO: 68); Peptide10_222 (SEQ ID NO: 69); Peptide10_190 (SEQ ID NO: 70); Peptide10_13 (SEQ ID NO: 71); or Peptide10_6 (SEQ ID NO: 72); Peptide10_193 (SEQ ID NO: 73); Peptide10_38 (SEQ ID NO: 74); Peptide10_38_medium_degron (SEQ ID NO: 816); or Peptide10_38_short_degron (SEQ ID NO: 817), and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key10C (SEQ ID NO: 93) or Key10N (SEQ ID NO: 94).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide11_13 (SEQ ID NO: 75) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key11N (SEQ ID NO: 95).

In some aspects, the chimeric polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide12_206 (SEQ ID NO: 76) and the key polypeptide comprises an amino acid sequence having at least about 50%, at least about 55%, at least about 60%, at least about 65%, at least about 70%, at least about 75%, at least about 80%, at least about 85%, at least about 90%, at least about 95%, or about 100% sequence identity to the sequence as set forth in Key12C (SEQ ID NO: 96).

The present disclosure also provides a vector comprising a polynucleotide disclosed herein and/or a polynucleotide set disclosed herein operably linked to one or more regulatory elements. In some aspects, the vector is a viral vector, a mammalian vector, or bacterial vector. In some aspects, the vector is a retroviral vector. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector. In some aspects, the vector is a lentivirus. In some aspects, the polynucleotide encoding the chimeric polypeptide and the second polynucleotide encoding the key polypeptide are on the same vector. In some aspects, the polynucleotide encoding the chimeric polypeptide and the second polynucleotide encoding the key polypeptide are on different vectors. In some aspects, the second polynucleotide encoding the key polypeptide is operably linked to an inducible promoter.

The present disclosure also provides a chimeric polypeptide encoded by a polynucleotide disclosed herein or a vector disclosed herein. The disclosure also provides a chimeric polypeptide encoded by a polynucleotide set disclosed herein or a vector disclosed herein.

The present disclosure also provides a chimeric polypeptide comprising:
(i) a structural region comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Cage1_N_SR (SEQ ID NO: 142), Cage1_C_SR (SEQ ID NO: 143), Cage2_N_SR (SEQ ID NO: 144), Cage2_C_SR (SEQ ID NO: 145), Cage3_N_SR (SEQ ID NO: 146), Cage3_C_SR (SEQ ID NO: 147), Cage4_N_SR (SEQ ID NO: 148), Cage4_C_SR (SEQ ID NO: 149), Cage5_N_SR (SEQ ID NO: 150), Cage5_C_SR (SEQ ID NO: 151), Cage6_N_SR (SEQ ID NO: 152), Cage6_C_SR (SEQ ID NO: 153), Cage7_N_SR (SEQ ID NO: 154), Cage7_C_SR (SEQ ID NO: 155), Cage8_N_SR (SEQ ID NO: 156), Cage8_C_SR (SEQ ID NO: 157), Cage9_N_SR (SEQ ID NO: 158), Cage9_C_SR (SEQ ID NO: 159), Cage10_N_SR (SEQ ID NO: 160), Cage10_C_SR (SEQ ID NO: 161), Cage11N_SR (SEQ ID NO: 162), Cage1_C_SR (SEQ ID NO: 163), Cage12_N_SR (SEQ ID NO: 164), Cage12_C_SR (SEQ ID NO: 165), Cage13_SR (SEQ ID NO: 838), Cage14_SR (SEQ ID NO: 840), Cage15_SR (SEQ ID NO: 842), Cage16_SR (SEQ ID NO: 844), Cage17_SR (SEQ ID NO: 846), Cage18_SR (SEQ ID NO: 848), Cage19_SR (SEQ ID NO: 850), Cage20_SR (SEQ ID NO: 852), Cage21_SR (SEQ ID NO: 854), Cage22_SR (SEQ ID NO: 856), Cage23_SR (SEQ ID NO: 858), Cage24_SR (SEQ ID NO: 860), Cage25_SR (SEQ ID NO: 862), Cage26_SR (SEQ ID NO: 864), or Cage27_SR (SEQ ID NO: 866), and
(ii) a latch region comprising (a) at least 20 consecutive amino acid residues from to Cage1_N_Latch (SEQ ID NO: 166), Cage1_C_Latch (SEQ ID NO: 167), Cage2_N_Latch (SEQ ID NO: 168), Cage2_C_Latch (SEQ ID NO: 169), Cage2_N_Latch (SEQ ID NO: 168), Cage2_C_Latch (SEQ ID NO: 169), Cage2_N_Latch (SEQ ID NO: 168), Cage2_C_Latch (SEQ ID NO: 169), Cage5_N_Latch (SEQ ID NO: 174), Cage5_C_Latch (SEQ ID NO: 175), Cage6_N_Latch (SEQ ID NO: 176), Cage6_C_Latch (SEQ ID NO: 177), Cage7_N_Latch (SEQ ID NO: 178), Cage7_C_Latch (SEQ ID NO: 179), Cage8_N_Latch (SEQ ID NO: 180), Cage8_C_Latch (SEQ ID NO: 181), Cage9_N_Latch (SEQ ID NO: 182), Cage9_C_Latch (SEQ ID NO: 183), Cage10_N_Latch (SEQ ID NO: 184), Cage10_C_Latch (SEQ ID NO: 185), Cage11_N_Latch (SEQ ID NO: 186), Cage11_C_Latch (SEQ ID NO: 187), Cage12_N_Latch (SEQ ID NO: 188), Cage12_C_Latch (SEQ ID NO: 189), Cage13_Latch (SEQ ID NO: 839), Cage14_Latch (SEQ ID NO: 841), Cage15_Latch (SEQ ID NO: 843), Cage16_Latch (SEQ ID NO: 845), Cage17_Latch (SEQ ID NO: 847), Cage18 Latch (SEQ ID NO: 849), Cage19_Latch (SEQ ID NO: 851), Cage20_Latch (SEQ ID NO: 853), Cage21_Latch (SEQ ID NO: 855), Cage22_Latch (SEQ ID NO: 857), Cage23_Latch (SEQ ID NO: 859), Cage24_Latch (SEQ ID NO: 861), Cage25_Latch (SEQ ID NO: 863), Cage26_Latch (SEQ ID NO: 865), or Cage27_Latch (SEQ ID NO: 867), respectively, and (b) a degron peptide consisting of an amino acid sequence as set forth in KTRGVEEVAEGVVLLRRRGN (SEQ ID NO: 1), SEQ ID NOS: 110-141, KTRGVEEVAEGVVLLRRRGNK (DegronA*, SEQ ID NO: 801); KEEVAEGVVLLRRRGNK (DegronA*_Truncation1, SEQ ID NO: 802); KTRGVEEVAEGVVLLRK (DegronA*_Truncation2, SEQ ID NO: 803); TRGVEEVAEGVVLLRRRGNK (DegronA*_Truncation6, SEQ ID NO: 918); RGVEEVAEGVVLLRRRGNK (DegronA*_Truncation7, SEQ ID NO: 919); GVEEVAEGVVLLRRRGNK (DegronA*_Truncation8, SEQ ID NO: 920); VEEVAEGVVLLRRRGNK (DegronA*_Truncation9, SEQ ID NO: 921); EEVAEGVVLLRRRGNK (DegronA*_Truncation10, SEQ ID NO: 922); EVAEGVVLLRRRGNK (DegronA*_Truncation11, SEQ ID NO: 923); VAEGVVLLRRRGNK (DegronA*_Truncation12, SEQ ID NO: 924); AEGVVLLRRRGNK (DegronA*_Truncation13, SEQ ID NO: 925); GVVLLRRRGNK (DegronA*_Truncation14, SEQ ID NO: 926); VVLLRRRGNK (DegronA*_Truncation15, SEQ ID NO: 927); VLLRRRGNK (DegronA*_Truncation16, SEQ ID NO: 928); LLRRRGNK (DegronA*_Truncation17, SEQ ID NO: 929); LRRRGNK (DegronA*_Truncation18, SEQ ID NO: 930); RRRGNK (DegronA*_Truncation19, SEQ ID NO: 931); KTEVAEGVVLLRRRGNK (DegronA*_Truncation20, SEQ ID NO: 932); KTRGAEGVVLLRRRGNK (DegronA*_Truncation21, SEQ ID NO: 933); KTRGVEGVVLLRRRGNK (DegronA*_Truncation22, SEQ ID NO: 934); KTRGVEEVVLLRRRGNK (DegronA*_Truncation23, SEQ ID NO: 935); KTRGVEEVAELRRRGNK (DegronA*_Truncation24, SEQ ID NO: 936); KTVLLRRRGNK (DegronA*_Truncation25, SEQ ID NO: 937); KTRGLRRRGNK (DegronA*_Truncation26, SEQ ID NO: 938); CTRGVEEVAEGVVLLRRRGNK (DegronA*_Mutation1, SEQ ID NO: 939); FTRGVEEVAEGVVLLRRRGNK (DegronA*_Mutation2, SEQ ID NO: 940); KIRGVEEVAEGVVLLRRRGNK (DegronA*_Mutation3, SEQ ID NO: 941); KLRGVEEVAEGVVLLRRRGNK (DegronA*_Mutation4, SEQ ID NO: 942); KNRGVEEVAEGVVLLRRRGNK (DegronA*_Mutation5, SEQ ID NO: 943); KRRGVEEVAEGVVLLRRRGNK (DegronA*_Mutation6, SEQ ID NO: 944); KTDGVEEVAEGVVLLRRRGNK (DegronA*_Mutation7, SEQ ID NO: 945); KTFGVEEVAEGVVLLRRRGNK (DegronA*_Mutation8, SEQ ID NO: 946); KTHGVEEVAEGVVLLRRRGNK (DegronA*_Mutation9, SEQ ID NO: 947); KTPGVEEVAEGVVLLRRRGNK (DegronA*_Mutation10, SEQ ID NO: 948); KTRGKEEVAEGVVLLRRRGNK (DegronA*_Mutation11, SEQ ID NO: 949); KTRGMEEVAEGVVLLRRRGNK (DegronA*_Mutation12, SEQ ID NO: 950); KTRGTEEVAEGVVLLRRRGNK (DegronA*_Mutation13, SEQ ID NO: 951); KTRGVAEVAEGVVLLRRRGNK (DegronA*_Mutation14, SEQ ID NO: 952); KTRGVEAVAEGVVLLRRRGNK (DegronA*_Mutation15, SEQ ID NO: 953); KTRGVEDVAEGVVLLRRRGNK (DegronA*_Mutation16, SEQ ID NO: 954); KTRGVEECAEGVVLLRRRGNK (DegronA*_Mutation17, SEQ ID NO: 955); KTRGVEENAEGVVLLRRRGNK (DegronA*_Mutation18, SEQ ID NO: 956); KTRGVEETAEGVVLLRRRGNK (DegronA*_Mutation19, SEQ ID NO: 957); KTRGVEEVACGVVLLRRRGNK (DegronA*_Mutation20, SEQ ID NO: 958); KTRGVEEVAEEVVLLRRRGNK (DegronA*_Mutation21, SEQ ID NO: 959); KTRGVEEVAEGCVLLRRRGNK (DegronA*_Mutation22, SEQ ID NO: 960); KTRGVEEVAEGVGLLRRRGNK (DegronA*_Mutation23, SEQ ID NO: 961); KTRGVEEVAEGVVLLRHRGNK (DegronA*_Mutation24, SEQ ID NO: 962); KTRGVEEVAEGVVLLRQRGNK (DegronA*_Mutation25, SEQ ID NO: 963); KTRGVEEVAEGVVLLRRRGFK (DegronA*_Mutation26, SEQ ID NO: 964); KTRGVEEVAEGVVLLRRRGNH (DegronA*_Mutation27, SEQ ID NO: 965); KTRGVEEVAEGVVLTRRRGNK (DegronA*_Mutation28, SEQ ID NO: 966); KTRGVEEVAEGWVLLRRRGNK (DegronA*_Mutation29, SEQ ID NO: 967); KTRGVEEVAEPVVLLRRRGNK (DegronA*_Mutation30, SEQ ID NO: 968); KTRGVEEVAHGVVLLRRRGNK (DegronA*_Mutation31, SEQ ID NO: 969); KTRGVEEVAPGVVLLRRRGNK (DegronA*_Mutation32, SEQ ID NO: 970); KTRGVEEVAQGVVLLRRRGNK (DegronA*_Mutation33, SEQ ID NO: 971); KTRGVEEVATGVVLLRRRGNK (DegronA*_Mutation34, SEQ ID NO: 972); KTRGVEEVHEGVVLLRRRGNK (DegronA*_Mutation35, SEQ ID NO: 973); KTRGVEEVPEGVVLLRRRGNK (DegronA*_Mutation36, SEQ ID NO: 974); KTRGVEEVREGVVLLRRRGNK (DegronA*_Mutation37, SEQ ID NO: 975); KTRGVEHVAEGVVLLRRRGNK (DegronA*_Mutation38, SEQ ID NO: 976); KTRGVEPVAEGVVLLRRRGNK (DegronA*_Mutation39, SEQ ID NO: 977); KTRGVERVAEGVVLLRRRGNK (DegronA*_Mutation40, SEQ ID NO: 978); KTRGVEVVAEGVVLLRRRGNK (DegronA*_Mutation41, SEQ ID NO: 979); KTRGVQEVAEGVVLLRRRGNK (DegronA*_Mutation42, SEQ ID NO: 980); KTRSVEEVAEGVVLLRRRGNK (DegronA*_Mutation43, SEQ ID NO: 981); or QTRGVEEVAEGVVLLRRRGNK (DegronA*_Mutation44, SEQ ID NO: 982), EGVVLLRRRGNK (DegronA*_Truncation3, SEQ ID NO: 804); wherein the degron peptide is linked to the latch region, wherein the latch region is capable of binding to the structural region. In some aspects, a degron useful for the present disclosure is not KTRGVEEV (DegronA*_Truncation4, SEQ ID NO: 805); or KTRGVEEVAEGV (DegronA*_Truncation5, SEQ ID NO: 806). In some aspects, a degron useful for the present disclosure comprises EGVVLLRRRG (SEQ ID NO: 122) or EGVVLLRRRGN (SEQ ID NO: 123).

In some aspects, the structural region of the cage polypeptide comprises any one of the amino acid sequences as set forth in Cage1_N_SR (SEQ ID NO: 142), Cage1_C_SR (SEQ ID NO: 143), Cage2_N_SR (SEQ ID NO: 144), Cage2_C_SR (SEQ ID NO: 145), Cage3_N_SR (SEQ ID NO: 146), Cage3_C_SR (SEQ ID NO: 147), Cage4_N_SR (SEQ ID NO: 148), Cage4_C_SR (SEQ ID NO: 149), Cage5_N_SR (SEQ ID NO: 150), Cage5_C_SR (SEQ ID NO: 151), Cage6_N_SR (SEQ ID NO: 152), Cage6_C_SR (SEQ ID NO: 153), Cage7_N_SR (SEQ ID NO: 154), Cage7_C_SR (SEQ ID NO: 155), Cage8_N_SR (SEQ ID NO: 156), Cage8_C_SR (SEQ ID NO: 157), Cage9_N_SR (SEQ ID NO: 158), Cage9_C_SR (SEQ ID NO: 159), Cage10_N_SR (SEQ ID NO: 160), Cage10_C_SR (SEQ ID NO: 161), Cage11_N_SR (SEQ ID NO: 162), Cage11_C_SR (SEQ ID NO: 163), Cage12_N_SR (SEQ ID NO: 164), Cage12_C_SR (SEQ ID NO: 165) Cage13_SR (SEQ ID NO: 838), Cage14_SR (SEQ ID NO: 840), Cage15_SR (SEQ ID NO: 842), Cage16_SR (SEQ ID NO: 844), Cage17_SR (SEQ ID NO: 846), Cage18_SR (SEQ ID NO: 848), Cage19_SR (SEQ ID NO: 850), Cage20_SR (SEQ ID NO: 852), Cage21_SR (SEQ ID NO: 854), Cage22_SR (SEQ ID NO: 856), Cage23_SR (SEQ ID NO: 858), Cage24_SR (SEQ ID NO: 860), Cage25_SR (SEQ ID NO: 862), Cage26_SR (SEQ ID NO: 864), or Cage27_SR (SEQ ID NO: 866). In some aspects, the latch region comprises at least 21, 22, 23, 24, 25, 26, 27, 28, 29, 30, 31, 32, 33, 34, or 35 consecutive amino acid residues from Cage1_N_Latch (SEQ ID NO: 166), Cage1_C_Latch (SEQ ID NO: 167), Cage2_N_Latch (SEQ ID NO: 168), Cage2_C_Latch (SEQ ID NO: 169), Cage3_N_Latch (SEQ ID NO: 170), Cage3_C_Latch (SEQ ID NO: 171), Cage4_N_Latch (SEQ ID NO: 172), Cage4_C_Latch (SEQ ID NO: 173), Cage5_N_Latch (SEQ ID NO: 174), Cage5_C_Latch (SEQ ID NO: 175), Cage6_N_Latch (SEQ ID NO: 176), Cage6_C_Latch (SEQ ID NO: 177), Cage7_N_Latch (SEQ ID NO: 178), Cage7_C_Latch (SEQ ID NO: 179), Cage8_N_Latch (SEQ ID NO: 180), Cage8_C_Latch (SEQ ID NO: 181), Cage9_N_Latch (SEQ ID NO: 182), Cage9_C_Latch (SEQ ID NO: 183), Cage10_N_Latch (SEQ ID NO: 184), Cage10_C_Latch (SEQ ID NO: 185), Cage11_N_Latch (SEQ ID NO: 186), Cage11_C_Latch (SEQ ID NO: 187), Cage12_N_Latch (SEQ ID NO: 188), Cage12_C_Latch (SEQ ID NO: 189), Cage13_Latch (SEQ ID NO: 839), Cage14_Latch (SEQ ID NO: 841), Cage15_Latch (SEQ ID NO: 843), Cage16_Latch (SEQ ID NO: 845), Cage17_Latch (SEQ ID NO: 847), Cage18_Latch (SEQ ID NO: 849), Cage19_Latch (SEQ ID NO: 851), Cage20_Latch (SEQ ID NO: 853), Cage21_Latch (SEQ ID NO: 855), Cage22_Latch (SEQ ID NO: 857), Cage23_Latch (SEQ ID NO: 859), Cage24_Latch (SEQ ID NO: 861), Cage25_Latch (SEQ ID NO: 863), Cage26_Latch (SEQ ID NO: 865), or Cage27_Latch (SEQ ID NO: 867).

In some aspects, the latch region of the cage polypeptide further comprises one or more amino acids derived from Cage1_N_Latch (SEQ ID NO: 166), Cage1_C_Latch (SEQ ID NO: 167), Cage2_N_Latch (SEQ ID NO: 168), Cage2_C_Latch (SEQ ID NO: 169), Cage3_N_Latch (SEQ ID NO: 170), Cage3_C_Latch (SEQ ID NO: 171), Cage4_N_Latch (SEQ ID NO: 172), Cage4_C_Latch (SEQ ID NO: 173), Cage5_N_Latch (SEQ ID NO: 174), Cage5_C_Latch (SEQ ID NO: 175), Cage6_N_Latch (SEQ ID NO: 176), Cage6_C_Latch (SEQ ID NO: 177), Cage7_N_Latch (SEQ ID NO: 178), Cage7_C_Latch (SEQ ID NO: 179), Cage8_N_Latch (SEQ ID NO: 180), Cage8_C_Latch (SEQ ID NO: 181), Cage9_N_Latch (SEQ ID NO: 182), Cage9_C_Latch (SEQ ID NO: 183), Cage10_N_Latch (SEQ ID NO: 184), Cage10_C_Latch (SEQ ID NO: 185), Cage11_N_Latch (SEQ ID NO: 186), Cage11_C_Latch (SEQ ID NO: 187), Cage12_N_Latch (SEQ ID NO: 188), Cage12_C_Latch (SEQ ID NO: 189), Cage13_Latch (SEQ ID NO: 839), Cage14_Latch (SEQ ID NO: 841), Cage15_Latch (SEQ ID NO: 843), Cage16_Latch (SEQ ID NO: 845), Cage17_Latch (SEQ ID NO: 847), Cage18_Latch (SEQ ID NO: 849), Cage19_Latch (SEQ ID NO: 851), Cage20_Latch (SEQ ID NO: 853), Cage21_Latch (SEQ ID NO: 855), Cage22_Latch (SEQ ID NO: 857), Cage23_Latch (SEQ ID NO: 859), Cage24_Latch (SEQ ID NO: 861), Cage25_Latch (SEQ ID NO: 863), Cage26_Latch (SEQ ID NO: 865), or Cage27_Latch (SEQ ID NO: 867).

The present disclosure also provides a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide1_121 (SEQ ID NO: 14); Peptide1_125 (SEQ ID NO: 15); Peptide1_128 (SEQ ID NO: 16); Peptide1_2 (SEQ ID NO: 17); or Peptide1_6 (SEQ ID NO: 18). Also provided is a chimeric polypeptide comprising an amino acid sequence having at least about 95%, at least about 96%, at least about 97%, at least about 98%, at least about 99%, or about 100% sequence identity to Peptide2_154 (SEQ ID NO: 19); Peptide2_35 (SEQ ID NO: 20); or Peptide2_35_medium_degron (SEQ ID NO: 818); or Peptide2_35_short_degron (SEQ ID NO: 819). Also provided is a chimeric polypeptide comprising an amino acid sequence having at least about 95%, at least about 96%, at least about 97%, at least about 98%, at least about 99%, or about 100% sequence identity to Peptide3_10 (SEQ ID NO: 21); Peptide3_123 (SEQ ID NO: 22); Peptide3_127 (SEQ ID NO: 23); Peptide3_130 (SEQ ID NO: 24); Peptide3_14 (SEQ ID NO: 25); Peptide3_159 (SEQ ID NO: 26); Peptide3_163 (SEQ ID NO: 27); Peptide3_17 (SEQ ID NO: 28); Peptide3_21 (SEQ ID NO: 29); Peptide3_28 (SEQ ID NO: 30); Peptide3_3 (SEQ ID NO: 31); Peptide3_32 (SEQ ID NO: 32); Peptide3_35 (SEQ ID NO: 33); Peptide3_39 (SEQ ID NO: 34); Peptide3_147 (SEQ ID NO: 807); Peptide3_147_shortened_9N (SEQ ID NO: 808); Peptide3_147_shortened_9C (SEQ ID NO: 809); Peptide3_147_shortened_9C9N (SEQ ID NO: 810); Peptide3_21_shortened_9N (SEQ ID NO: 811); Peptide3_21_medium_degron (SEQ ID NO: 814); or Peptide3_21_short_degron (SEQ ID NO: 815).

The disclosure also provides a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide4_125 (SEQ ID NO: 35); Peptide4_129 (SEQ ID NO: 36); Peptide4_132 (SEQ ID NO: 37); Peptide4_136 (SEQ ID NO: 38); Peptide4_139 (SEQ ID NO: 39); Peptide4_140 (SEQ ID NO: 40); Peptide4_147 (SEQ ID NO: 41); Peptide4_150 (SEQ ID NO: 42); Peptide4_151 (SEQ ID NO: 43); Peptide4_154 (SEQ ID NO: 44); Peptide4_158 (SEQ ID NO: 45); Peptide4_162 (SEQ ID NO: 46); Peptide4_164 (SEQ ID NO: 47); Peptide4_165 (SEQ ID NO: 48); Peptide4_21 (SEQ ID NO: 49); Peptide4_24 (SEQ ID NO: 50); Peptide4_25 (SEQ ID NO: 51); Peptide4_39 (SEQ ID NO: 52); Peptide4_6 (SEQ ID NO: 53); Peptide4_7 (SEQ ID NO: 54); Peptide4_154_medium_degron (SEQ ID NO: 812); or Peptide4_154_short_degron (SEQ ID NO: 813). Also provided is a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide5_190 (SEQ ID NO: 55); Peptide5_192 (SEQ ID NO: 56); Peptide5_10 (SEQ ID NO: 57); Peptide5_199 (SEQ ID NO: 58); Peptide5_35 (SEQ ID NO: 59). Also provided is a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide6_6 (SEQ ID NO: 60).

The disclosure also provides a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide7_218 (SEQ ID NO: 61); Peptide7_3 (SEQ ID NO: 62); Peptide7_218_medium_degron (SEQ ID NO: 820); or Peptide7_218_short_degron (SEQ ID NO: 821). Also provided is a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide8_200 (SEQ ID NO: 63). Also provided is a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide9_3 (SEQ ID NO: 64); Peptide9_214 (SEQ ID NO: 65); Peptide9_6 (SEQ ID NO: 66); or Peptide9_13 (SEQ ID NO: 67). Also provided is a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide10_219 (SEQ ID NO: 68); Peptide10_222 (SEQ ID NO: 69); Peptide10_190 (SEQ ID NO: 70); Peptide10_13 (SEQ ID NO: 71); or Peptide10_6 (SEQ ID NO: 72); Peptide10_193 (SEQ ID NO: 73); Peptide10_38 (SEQ ID NO: 74); Peptide10_38_medium_degron (SEQ ID NO: 816); or Peptide10_38_short_degron (SEQ ID NO: 817). Also provided is a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide11_13 (SEQ ID NO: 75). The present disclosure also provides a chimeric polypeptide comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Peptide12_206 (SEQ ID NO: 76).

In some aspects, the chimeric polypeptide disclosed herein is a chimeric antigen receptor. The present disclosure also provides a cell comprising a polynucleotide disclosed herein, a polynucleotide set disclosed herein, a vector disclosed herein, or a chimeric polypeptide disclosed herein. The disclosure also provides a cell genetically modified to express a CAR, comprising a polynucleotide disclosed herein, a polynucleotide set disclosed herein, a vector disclosed herein, or a chimeric polypeptide disclosed herein. In some aspects, the cell is a T cell, a natural killer (NK) cell, a natural killer T (NKT) cell, or an ILC cell.

The disclosure also provides a composition comprising a polynucleotide disclosed herein, a polynucleotide set disclosed herein, a vector disclosed herein, a chimeric polypeptide disclosed herein, or a cell disclosed herein. The disclosure provides a composition for treating a subject in need of a CAR therapy.

Also provided is a kit comprising a polynucleotide disclosed herein, a polynucleotide set disclosed herein, a vector disclosed herein, a chimeric polypeptide disclosed herein, or a cell disclosed herein. The disclosure also provides a method of making an engineered cell comprising transfecting a polynucleotide disclosed herein, a polynucleotide set disclosed herein, or a vector disclosed herein in a cell. In some aspects, the chimeric polypeptide is expressed in the cell. In some aspects, the method further comprises administering the cell to a subject in need thereof. In some aspects, the method further comprises administering a key polypeptide or an inducer that can express a key polypeptide to the subject.

The present disclosure also provides a method of controlling a T cell-mediated immune response in a subject in need thereof comprising administering an effective amount of a cell of the present disclosure. Also provided is a method of stimulating a T cell-mediated immune response to a target cell population or tissue in a subject, comprising administering an effective amount of a cell of the present disclosure. Also provided is a method of providing an anti-tumor immunity in a subject in need thereof, the method comprising administering an effective amount of a cell of the present disclosure to the subject. Also provided is a method of treating cancer in a subject in need thereof comprising administering an effective amount of a cell of the present disclosure to the subject. In some aspects, the cell is a T cell. In some aspects, the cell is an autologous T cell. In some aspects, the method further comprises administering a key polypeptide. In some aspects, the method further comprises administering an inducer of a key polypeptide.

The present disclosure also provides the use of a polynucleotide of the present disclosure, a polynucleotide set of the present disclosure, a vector of the present disclosure, a chimeric polypeptide of the present disclosure, a cell of the present disclosure, a composition of the present disclosure, or a kit of the present disclosure for the manufacture of a medicament for treating cancer in a subject in need thereof.

### BRIEF DESCRIPTION OF THE DRAWINGS/FIGURES

FIG. 1 is a schematic representation of a cell-intrinsic feedback control system to mitigate exhaustion, e.g., via degron-induced reduction of CAR surface expression as disclosed in the present application.

FIG. 2 is a schematic representation of ubiquitin-dependent and ubiquitin-independent membrane protein regulation. Stability of most membrane proteins is determined by regulated trafficking of proteins to endocytic vesicles, followed by fusion with lysosomal bodies and protein degradation. Mono or poly-ubiquitination is one driver of this process. Recruitment of trafficking proteins can also lead to membrane protein internalization and degradation. Degradation would result in a reduction of membrane protein surface expression.

FIG. 3 shows the sequence of Degron A, an exemplary 20-amino acid long degron sequence used in chimeric polypeptides of the present disclosure. A lysine amino acid that may undergo ubiquitination is underlined.

FIG. 4 is a schematic representation of two cage polypeptides. The 3plus1 cage polypeptide is a four-helix bundle comprising three structural region alpha helices and one latch region alpha helix. The 2plus1 cage polypeptide is a three alpha helix bundle comprising two structural region alpha helices and one latch region alpha helix.

FIG. 5 shows a 3plus1 cage polypeptide in which the degron of SEQ ID NO:1 (Degron A) has been inserted in the N-terminal portion of the C-terminal latch region.

FIG. 6 is a schematic representation of the mechanism of action of a chimeric polypeptide of the present disclosure, showing a degron (D) motif integrated in the latch region (L). The latch region is initially bound to the structural region, sequestering the degron. Upon binding of a key polypeptide (K), which displaces the latch region, the degron becomes activated and triggers protein degradation, e.g., via recruitment of ubiquitin ligases and subsequent ubiquitination of cage amino acids.

FIG. 7 shows screening of peptide motifs for destabilization of membrane protein expression. Lentiviral expression constructs were based on a ROR1 CAR linked via p2a cleavable peptide to a truncated EGFR transduction marker (EGFRt). Regulation motifs were screened by inserting a peptide motif between the 4-1BB and CD3 zeta signaling domains. Jurkat and SupT1 human T cell lines were transduced with lentiviral constructs. Expression of CAR and transduction marker was determined by binding of recombinant ROR1-Fc fusion protein and anti-EGFR antibodies, respectively. Mean fluorescence intensity of ROR1-Fc binding on live EGFRt+ cells is shown for each motif, with the control ROR1 CAR and the Degron A sequence containing CAR (ROR1 CAR-Degron A Motif) labeled. As a control for background fluorescence, the mean fluorescence intensity of ROR1-Fc binding to untransduced, EGFRt cells is shown (No Tx).

FIG. 8 shows that insertion of the Degron A sequence between the 4-1BB and CD3 zeta signaling domains of a ROR1 CAR significantly reduces surface expression of the CAR. Primary human T cells were untransduced (left panel), transduced with the parental ROR1 CAR linked via p2a cleavable peptide to a truncated EGFR transduction marker, or the Degron A-containing ROR1 CAR (ROR1 CAR-Degron A Motif) linked via p2a cleavable peptide to a truncated EGFR transduction marker. Expression of CAR and transduction marker was determined by binding of recombinant ROR1-Fc fusion protein and anti-EGFR antibodies, respectively.

FIG. 9 shows quantification of the mean fluorescence intensity of bound ROR1-Fc fusion protein from FIG. 8 in untransduced total live T cells (no transduction), EGFRt+ cells transduced with the parental ROR1 CAR, and EGFRt+ cells transduced with the Degron A-containing ROR1 CAR (ROR1 CAR-Degron A Motif). Percent downregulation was calculated by comparing MFI in the Degron A-containing ROR1 CAR to the MFI of the parental ROR1 CAR after subtraction of the background fluorescence intensity from the no transduction condition.

FIG. 10 shows testing conditions for functional assessment of CAR regulation by the Degron A motif Transduced primary CAR T cells (either ROR1 CAR or ROR1 CAR+Degron A Motif) were mixed at defined ratios with NUCLIGHT™ Red expressing Jeko1 target cells, or matched NUCLIGHT™ Red Jeko1 cells lacking ROR1 protein expression (Jeko1-ROR1KO) as a control. After co-culture, samples were either tested at 24 hours for surface marker expression (using flow cytometry) and cytokine secretion, or co-cultured for 96 hours on an IncuCyte machine to assess T cell killing of NUCLIGHT™ red target cells.

FIG. 11 shows reduced CD69 and PD1 activation marker expression on Degron A ROR1 CAR-T compared to the parental ROR1 CAR-T exposed to Jeko1 target cells. Surface expression of PD1 and CD69 on primary T cells after 1:5 co-culture with Jeko1 cells was performed as described in FIG. 10. CD69 and PD1 expression on transduced T cells co-cultured with Jeko1 target cells (EGFRt+), untransduced T cells co-cultured with Jeko1 target cells (EGFRt−), and transduced T cells co-cultured with Jeko1 cells lacking the ROR1 target protein (ROR1KO Jeko1) for the parental ROR1 CAR and the Degron A-containing ROR1 CAR are shown.

FIG. 12 shows reduced cytolytic activity in Degron A ROR1 CAR-T (ROR1-CAR+Degron A Motif) compared to the parental ROR1 CAR-T. Killing of co-cultured Jeko1 cells over 96 hours was performed as described in FIG. 10 using T cells from two independent normal donors. Shown are total NUCLIGHT™ Red cells counted per well in target cell alone, or target cells co-cultured with CAR-T cells at the indicated ratios.

FIG. 13 shows reduced cytokine secretion in Degron A ROR1 CAR-T (labeled “Degron A”) compared to the parental ROR1 CAR-T (labeled “ROR1-CAR”) after exposure to ROR1+ target cells. Co-culture of CAR-T and Jeko1 or Jeko1-ROR1KO cells was performed as described in FIG. 10. Culture supernatant was taken at 24 hours and frozen at −80° C. until analysis. IL-2 (gray) and IFN-gamma (black) secretion in 1:5 diluted supernatant of indicated culture conditions was determined using the Meso Scale Discovery kit according to manufacturer's instructions.

FIG. 14 shows successful caging and expression of LOCKR (latching orthogonal cage-key proteins) containing the Degron A motif fused to the C-terminal end of ROR1 CARs in primary T cells. Top shows the design of lentiviral expression constructs, consisting of the MND promoter, truncated CD19 (tCD19) as a transduction control, a p2a skip sequence, the full ROR1 CAR, and a C-terminal LOCKR containing the Degron A motif in the latch. Dead cells were excluded by staining with the EFLUOR™ 780 fixable viability dye, and expression of CAR and transduction marker was determined by binding of recombinant ROR1-Fc fusion protein and anti-CD19 antibodies, respectively. Shown are representative flow plots for one 3plus1 (ROR1 CAR-Degron A in 3+1 LockR) and one 2plus1 (ROR1 CAR-Degron A in 3+1 LockR) LOCKR cage fused to the CAR.

FIG. 15 shows surface expression of multiple independent LOCKR designs in the SupT1 human T cell line. SupT1 cells were transduced with lentiviral constructs designed as described for FIG. 14, consisting of the MND promoter, truncated CD19 (tCD19) as a transduction control, a p2a skip sequence, the full ROR1 CAR construct, and a C-terminal LOCKR containing the Degron A motif (SEQ ID NO: 1) in the latch region. After transduction, dead cells were excluded by staining with the EFLUOR™ 780 fixable viability dye, and expression of CAR and transduction marker was determined by binding of recombinant ROR1-Fc fusion protein and anti-EGFR antibodies, respectively. Mean fluorescence intensity of ROR1 binding on live tCD19+ cells is shown for each construct, including the parental ROR1 CAR as well as ROR1 CARs fused to 6 distinct Degron A-containing 2plus1 LOCKR cages or 3 distinct Degron A-containing 3plus1 LOCKR cages. As a control for background fluorescence, the mean fluorescence intensity of ROR1-Fc binding to untransduced, tCD19-cells is shown (untransduced).

FIG. 16 shows activation of SupT1 cells transduced with ROR1 CAR (black symbols) and ROR1 CAR-LOCKR (gray symbols) by ROR1-expressing Jeko1 target cells. SupT1 cells were transduced with the parental ROR1 CAR (*) as well as ROR1 CAR fused to 6 distinct Degron A-containing 2plus1 LOCKR cages (**) or 3 distinct Degron A-containing 3plus1 LOCKRs (***). Transduced SupT1 cells were co-cultured+/−Jeko1 cells at a 1:1 ratio of SupT1 to Jeko1 for 18 hours. To analyze surface phenotype, dead cells were excluded by staining with the EFLUOR™ 780 fixable viability dye, and expression of CAR, transduction marker, and the activation marker CD69 was determined by binding of recombinant ROR1-Fc fusion protein, anti-EGFR antibody, and anti-CD69 antibodies, respectively. Mean fluorescence intensity of ROR1-Fc binding and CD69 expression on live tCD19+ cells is shown for each construct without (Triangles) or with (Squares) Jeko1 co-culture.

FIG. 17 shows surface expression of ROR1 CAR with C-terminal fusion to multiple independent LOCKR cage designs in primary human T cells. T cells were transduced with lentiviral constructs designed as described for FIG. 14, consisting of the MND promoter, truncated CD19 (tCD19) as a transduction control, a p2a skip sequence, the full ROR1 CAR construct, and a C-terminal LOCKR cage containing the Degron A motif in the latch region. After transduction, dead cells were excluded by staining with the EFLUOR™ 780 fixable viability dye, and expression of CAR and transduction marker was determined by binding of recombinant ROR1-Fc fusion protein and anti-EGFR antibodies, respectively. Mean fluorescence intensity of ROR1-Fc binding on live tCD19+ cells is shown for each construct, including the parental ROR1-CAR as well as ROR1 CAR fused to 6 distinct Degron A-containing 2plus1 LOCKR cages or 2 distinct Degron A-containing 3plus1 LOCKR cages, labeled by SEQ ID NO. As a control for background fluorescence, the mean fluorescence intensity of ROR1-Fc binding to untransduced, tCD19-cells is shown (untransduced).

FIG. 18 shows activation of primary T cells transduced with parental ROR1 CAR (black bar) and ROR1 CAR-LOCKR (gray bars) by ROR1 expressing Jeko1 target cells. T cells were transduced with parental ROR1 CAR (*) as well as ROR1 CAR fused to 6 distinct Degron A-containing 2plus1 LOCKR cages (**) or 2 distinct Degron A-containing 3plus1 LOCKR cages (***). Transduced T cells were co-cultured+/−Jeko1 cells at a 1:1 ratio of T cells to Jeko1 for 18 hours. To analyze surface phenotype, dead cells were excluded by staining with the EFLUOR™ 780 fixable viability dye, and expression of CAR, transduction marker, and the activation marker CD69 was determined by binding of recombinant ROR1-Fc fusion protein, anti-EGFR antibody, and anti-CD69 antibodies, respectively. Mean fluorescence intensity of ROR1 binding and CD69 expression on live T cells without (no Tx, left) or with (+Jeko1 Target, right) is shown.

FIG. 19 shows activation of primary T cells transduced with parental ROR1 CAR and ROR1 CAR-LOCKR by ROR1 expressing Jeko1 target cells. T cells were transduced with parental ROR1 CAR (*) as well as ROR1 CAR fused to 6 distinct Degron A-containing 2plus1 LOCKR cages (**) or 2 distinct Degron A-containing 3plus1 LOCKR cages (***). Transduced T cells were co-cultured+/−Jeko1 cells at a 1:1 ratio of T cells to Jeko1 for 18 hours. To analyze surface phenotype, dead cells were excluded by staining with the EFLUOR™ 780 fixable viability dye, and expression of CAR, transduction marker, and the activation/exhaustion marker PD1 was determined by binding of recombinant ROR1 fusion protein, anti-EGFR antibody, and anti-PD1 antibodies, respectively. Mean fluorescence intensity of ROR1-Fc binding and PD1 expression on live T cells without (no Tx, left) or with (+Jeko1 Target, right) is shown.

FIG. 20 shows ineffective membrane degradation by C-terminal fusion of the mouse ornithine decarboxylase (cODC) enzyme to the truncated epidermal growth factor receptor (EGFRt). Shown are representative histogram plots of EGFRt surface expression on untransduced SupT1 cells (Dashed line), SupT1 cells transduced with ROR1 CAR p2a-linked to the truncated EGFR membrane protein, (solid line, gated on ROR1 CAR+ cells), or SupT1 cells transduced with ROR1 CAR p2a-linked to a truncated EGFR membrane protein with a flexible glycine-serine linker followed by cODC (CAR-p2a-EGFRt-G4S_cODC; filled gray histogram, gated on ROR1 CAR+ cells) (full degron sequence: SGGGGSGGGGSLPMSCAQESGMDRHPAACASARINV; SEQ ID NO: 822).

FIGS. 21B and 21B show the effect of position within the construct on Degron A activity. FIG. 21A is a schematic view of tested CAR constructs with no Degron A (top), Degron A between the 41BB and CD3 zeta signaling domains (middle), and Degron A after the CD3 zeta signaling domain (bottom). Indicated is the number of amino acids separating the start of the Degron A sequence from the end of the transmembrane domain. FIG. 21B shows the surface expression of ROR1 CAR in test constructs. Dead cells were excluded by staining with the EFLUOR™ 780 fixable viability dye, and expression of CAR and transduction marker was determined by binding of recombinant ROR1-Fc fusion protein and anti-EGFR antibodies, respectively. Data shown is gated on live, EGFR+ cells.

FIG. 22 shows the effect of truncations of the Degron A sequence (extended Degron A sequence in an additional K amino acid, i.e., DegronA*) on activity. SupT1 cells were transduced with lentiviral constructs encoding CAR with no Degron A (CAR) or constructs with the indicated amino acid sequence inserted between the 41BB and CD3 zeta signaling domains. Dead cells were excluded by staining with the EFLUOR™ 780 fixable viability dye, and expression of CAR and transduction marker was determined by binding of recombinant ROR1-Fc fusion protein and anti-EGFR antibodies, respectively. Data shown is gated on live, EGFR+ cells, with the exception of untransduced samples, which are gated on total live cells. The truncation tested were Truncation 1 (KEEVAEGVVLLRRRGNK; DegronA*_Truncation1; SEQ ID NO: 802), Truncation 2 (KTRGVEEVAEGVVLLRK; DegronA*_Truncation2; SEQ ID NO: 803), or Truncation 3 (EGVVLLRRRGNK; DegronA*_Truncation3; SEQ ID NO: 804), Truncation 4 (KTRGVEEV; DegronA*_Truncation4 SEQ ID NO: 805) and Truncation 5 (KTRGVEEVAEGV; DegronA*_Truncation5; SEQ ID NO: 806).

FIG. 23 shows that C-terminal fusion of LOCKR designs containing the Degron A motif does not prevent cytolytic activity driven by the CAR construct in primary human T cells. Primary human T cells were transduced with the ROR1-specific CAR construct (“ROR1-CAR”) or ROR1-specific CAR constructs linked at the C terminus to 10 independent LOCKR designs containing the Degron A motif, labeled by SEQ ID NO. T cells were cultured with Jeko1 target cells at a 1:5 ratio of CAR-T:Jeko1 for 72 hrs. Expression of CAR on the surface of T cells and CAR specific killing of Jeko1 cells was determined by flow cytometry.

FIG. 24 shows that CAR constructs containing C-terminal fusion of LOCKR designs containing the Degron A motif are able to secrete IFNg (black bars) and IL-2 (white bars) in response to target antigen cells. Primary human T cells were transduced with the ROR1-specific CAR construct (“ROR1-CAR”) or ROR1-specific CAR constructs linked at the C terminus to 10 independent LOCKR designs containing the Degron A motif, labeled by SEQ ID NO. T cells were cultured with Jeko1 target cells at a 1:5 ratio of CAR-T:Jeko1 cells. After 24 hours of co-culture, supernatant samples were taken for measurement of IFNg and IL-2.

FIGS. 25A and 25B demonstrate inducible degradation of a CAR construct linked to a C-terminal LOCKR (DegronA_2plus1_5419medium_thread-21, SEQ ID NO: 814) containing the Degron A motif in the presence of its corresponding Key protein (SEQ ID NO: 82). SupT1 cells were transduced with a single lentivirus containing a CAR construct. Indicated conditions were transduced with a second lentivirus encoding a RQR8 synthetic membrane protein linked to a matching key. CAR expression was assessed by ROR1-Fc binding in untransduced (gated on total live cells), CAR single transduced cells (Gated on live, tCD19 CAR transduction marker+), and CAR-Key double transduced cells (Gated on live, tCD19 CAR transduction marker+, CD20 key transduction marker+). FIG. 25A shows histogram of ROR1-fc binding. FIG. 25B shows geometric MFI for each population.

FIG. 26 Gating strategy for sorting degron library. Cells were gated based on FSC-A and SSC-A to identify the lymphocyte population (R1). Cells from R1 were gated on FSC-A and FSC-H to identify singlets (R2). Cells from R2 were gated on 7-AAD negative live cells (R3), and live cells were then sorted into four EGFR+ bins with varying levels of CAR expression as measured by ROR1-Fc-AF647 staining: CAR low-low/EGFR+ (R4), CAR med-low/EGFR+ (R5), CAR med-high/EGFR+ (R6), and CAR high-high/EGFR+ (R7). The reference unselected population was sorted to include all EGFR+ cells in bins R4-7. The last 10,000 events collected are shown.

FIG. 27 Comprehensive degron truncation identifies critical residues within an LRRRGN motif (SEQ ID NO: 113). A library of degron mutants appended to the C-terminus of CD3z was transduced into primary T cells and sorted into four bins based on CAR expression. The four bins (high-high (hh), medium-high (mh), medium-low (ml), and low-low (11) are shown as columns. Enrichment ratios (ER) for degron truncation mutants (rows) are summarized in a heatmap, with darker colors indicating a higher enrichment ratio (higher frequency in a given bin compared to the unselected control). The WT degron sequence is indicated with an asterisk. Sequences containing truncations that overlap the LRRRGN motif (SEQ ID NO: 113) show enrichment in the high-high and medium-high CAR expression bins, with ER profiles that are distinct from the WT degron sequence and a number of truncations involving residues outside of the LRRRGN motif (SEQ ID NO: 113), which show enrichment in the medium-low and low-low CAR expression bins. Functional Degron A truncations identified in this library are summarized in SEQ ID NO: 918-938. FIG. 27 discloses SEQ ID NOS 985, 140, 929, 918, 1, 931, 681, 937, 930, 935, 801, 932-933, 938, 934, 936, 920-923, 926, 925, 919, 928, 927, 924, 804, 986, 688, 683, 803, 694, 693, 691, 689-690, 987, 687, 686, 685, 988-989, 682, 692, 990 and 684, respectively in order of appearance.

FIG. 28 Comprehensive degron truncation and mutagenesis identifies allowable mutations. A library of degron mutants appended to the C-terminus of CD3z was transduced into primary T cells and sorted into four bins based on CAR expression. Mutants that showed enrichment ratio profiles similar to WT were selected using more stringent filtering parameters (slope>0.1, intercept<1, and relative ERs in ml and ll bins>0.8) and the enrichment ratio of each sequence in each bin is summarized in a heatmap. Functional Degron A mutations identified in this library are summarized in SEQ ID NO: 939-982. FIG. 28 discloses SEQ ID NOS 939-982 and 801, respectively in order of appearance.

FIG. 29 shows a sequence logo representing degron mutants that retain degradation activity comparable to the original Degron A sequence. A library of degron mutants appended to the C-terminus of CD3z was transduced into primary T cells and sorted into four bins based on CAR expression. Mutants that showed enrichment ratio profiles similar to the original Degron A sequence were selected using stringent filtering parameters (slope>0.1, intercept<1, and relative ERs in ml and ll bins>0.8) and the enrichment ratio of each sequence in the ml bin was used to generate a sequence logo summarizing the amino acids at each position that do not appear to diminish degron function. The consensus sequence for these variants is SEQ ID NO: 983.

FIG. 30 shows a sequence logo representing degron mutants that retain degradation activity. A library of degron mutants appended to the C-terminus of CD3z was transduced into primary T cells and sorted into four bins based on CAR expression. Mutants that showed enrichment ratio profiles were selected and used to generate a sequence logo summarizing the amino acids at each position that do not appear to diminish degron function. The consensus sequence for these variants is SEQ ID NO: 984.

### DETAILED DESCRIPTION

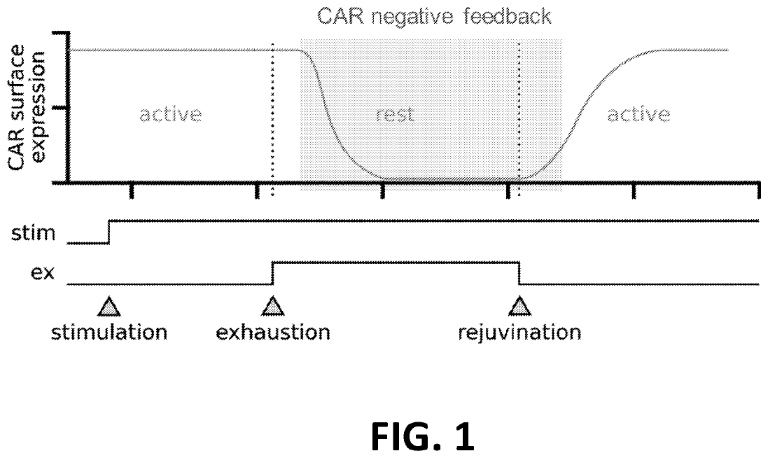

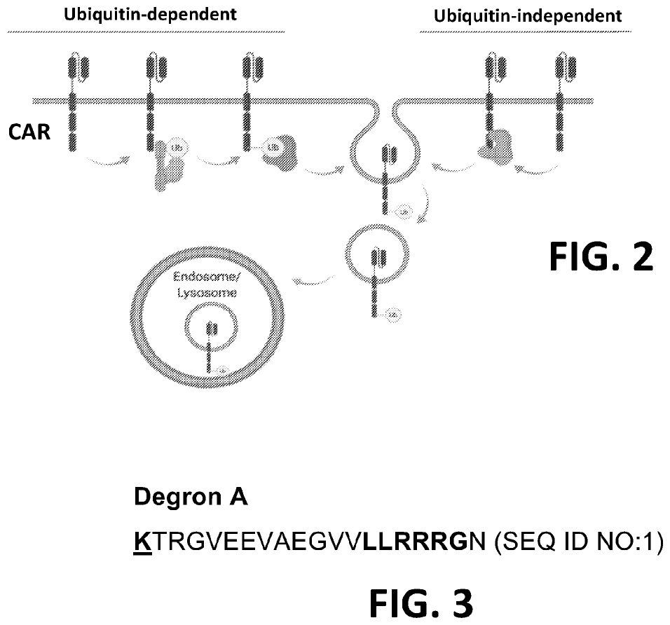

The present disclosure is directed to chimeric polypeptides that can be used to control CAR expression, e.g., to mitigate CAR T-cell exhaustion by reducing CAR surface expression. See FIG. 1. Reduction of CAR surface expression could also serve to match CAR expression to tumor burden, minimizing immune activation mediated toxicity under high tumor burden, while maximizing effective recognition of low-expressing escape mutations or small amounts of residual disease. Reduction of CAR surface expression can be accomplished, for example, by increasing CAR degradation, e.g., via ubiquitin-dependent membrane protein internalization and degradation. See FIG. 2.

The chimeric polypeptides disclosed herein comprise a cage polypeptide that includes a sequestered degron, which can be activated with a key polypeptide. The cage polypeptide and the key polypeptide can be two separate polypeptide chains.

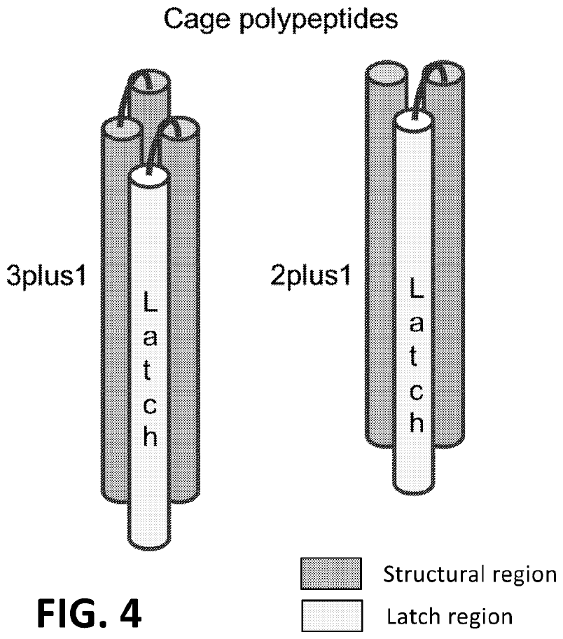

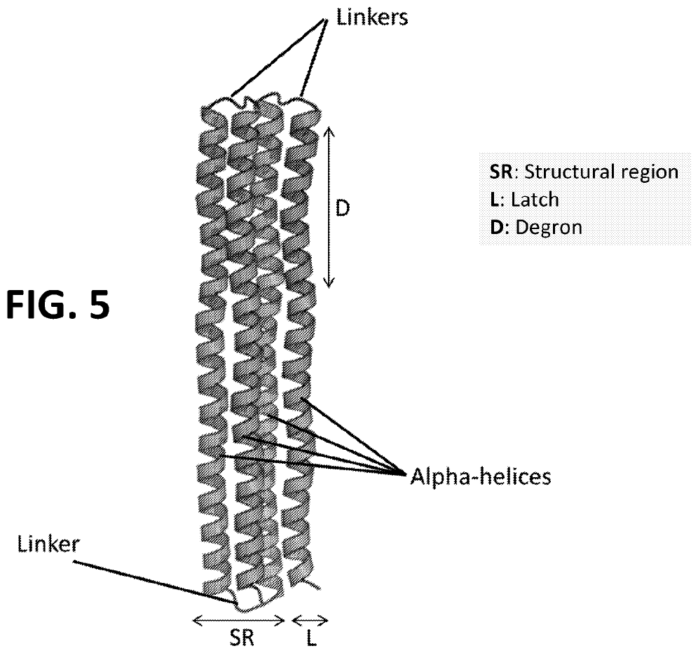

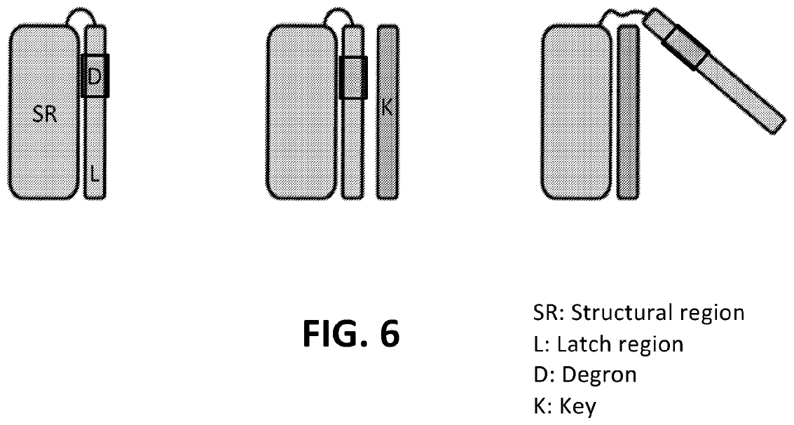

The cage polypeptide is a helix bundle comprising a structural region and a latch region (see FIG. 4). The latch region of the cage polypeptide comprises the “sequestered” (i.e., caged) degron (e.g., linked to the C-terminus of the latch). See FIG. 5. The key polypeptide displaces the latch polypeptide through competitive intermolecular binding that induces a conformational change, exposing the degron and activating the system. In turn, the exposed degron can be ubiquitinated by an ubiquitin ligase, leading to the degradation of the chimeric polypeptide, including any bioactive molecule fused to the chimeric polypeptide, e.g., a chimeric antigen receptor (CAR) or a T cell receptor (TCR) fused or conjugated to the cage polypeptide. See FIG. 6.

Accordingly, in some aspects, the chimeric polypeptides disclosed herein can be incorporated into CARs, TCRs, or other engineered receptors and thereby, modulate the expression of CARs, TCRs, or other engineered receptors on immune cells. In some aspects, by modulating the expression of CARs, TCRs, or other engineered receptors on immune cells, it is possible to prevent, mitigate, modulate, or reverse immune cell exhaustion in immune therapy. Such effect can be accomplished, for example, via the administration of the key polypeptide (e.g., in a format capable of being transported through cell membranes) or via endogenous expression of the key polypeptide in response to an inducing agent.

Cytosolic proteins are predominantly regulated through proteasomal degradation, including direct recruitment to the proteasome by degradation motifs such as found in mouse ornithine decarboxylase (Bhattacharyya et al. 2014. Regulated protein turnover: Snapshots of the proteasome in action. Nat Rev Mol Cell Biol 15:122-133; Takeuchi et al. 2008 Structural elements of the ubiquitin-independent proteasome degron of ornithine decarboxylase. Biochem J. 410(2):401-7). In contrast, membrane proteins are predominantly degraded through endo-lysosomal mechanisms or via autophagy (Luzio et al. 2007. Lysosomes: Fusion and function. Nat Rev Mol Cell Biol 8:622-632).

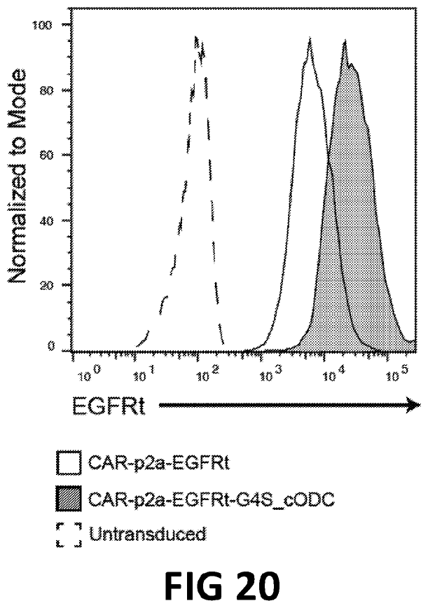

The present disclosure shows that when a Degron A peptide or a fragment thereof is linked to a Cage polypeptide of the present disclosure and is activated, the Degron A peptide or a fragment thereof is capable of degrading membrane proteins. However, a C-terminal degron of mouse ornithine decarboxylase (cODC) is not capable of degrading membrane proteins (see FIG. 20). Accordingly, compared to motifs such as cODC, the degron peptides disclosed herein (e.g., Degron A) allow for one or more improved properties when integrated into a chimeric polypeptide of the present disclosure. For example, in some aspects, integrating the Degron A peptides disclosed herein at the C-terminal end of a CAR or TCR results in greater downregulation of CAR or TCR expression. In certain aspects, downregulation of the expression of a CAR or TCR comprising a Degron A peptide (or variants thereof) is greater than about 0.5-fold, greater than about 1-fold, greater than about 2-fold, greater than about 3-fold, greater than about 4-fold, greater than about 5-fold, greater than about 10-fold, greater than about 20-fold, greater than about 25-fold, greater than about 30-fold, greater than about 35-fold, greater than about 40-fold, greater than about 45-fold, greater than about 50-fold, greater than about 75-fold, or greater than about 100-fold or more, compared to the expression of a corresponding CAR or TCR comprising a cODC.

As described herein, the ability to decrease CAR or TCR expression can improve one or more aspects of an immune response. For example, the greater downregulation of CAR or TCR on immune cells (e.g., T cells) can reduce the exhaustion of the immune cells. Accordingly, in certain aspects, immune cells (e.g., T cells) expressing CARs or TCRs comprising the Degron A peptide of the present disclosure are less exhausted compared to reference immune cells expressing CARs or TCRs that do not comprise the Degron A peptide (e.g., comprises cODC). In some aspects, exhaustion of the immune cells is reduced by at least about 5%, at least about 10%, at least about 20%, at least about 30%, at least about 40%, at least about 50%, at least about 60%, at least about 70%, at least about 80%, at least about 90%, or about 100% compared to the reference immune cells.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein (e.g., a CAR comprising a chimeric polypeptide disclosed herein) include intravenous, intraperitoneal, intramuscular, subcutaneous, spinal or other parenteral routes of administration, for example by injection or infusion.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricular, intravitreal, epidural, and intrasternal injection and infusion, as well as in vivo electroporation.

Alternatively, a therapeutic agent described herein (e.g., a CAR comprising a chimeric polypeptide disclosed herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell).

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

It is to be understood that in the disclosed sequences T and U are interchangeable depending on whether the sequence is a DNA or an RNA. For example, gRNA spacer sequences are presented as DNAs (A/T/C/G) in the present disclosure, whereas the gRNA chimeric frames are presented as RNAs (A/U/C/G).

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid,” as used herein, is intended to include DNA molecules and RNA molecules. A nucleic acid molecule can be single-stranded or double-stranded and can be cDNA.

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including mRNAs and gRNAs, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors can direct the expression of genes to which they are operatively linked. Such vectors are referred to herein as “recombinant expression vectors” (or simply, “expression vectors”).

In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the terms “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

As used herein, the term “anti-tumor immune response” refers to an immune response against a tumor antigen.

An increased ability to stimulate an immune response or the immune system can result from an enhanced agonist activity of T cell costimulatory receptors and/or an enhanced antagonist activity of inhibitory receptors. An increased ability to stimulate an immune response or the immune system can be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. In some aspects, the ability to stimulate an immune response or the immune system activity can be enhanced, e.g., by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. In some aspects, the ability to stimulate an immune response or the immune system activity can be enhanced, e.g., at least about 1.2-fold, at least about 1.4-fold, at least about 1.6-fold, at least about 1.8 fold, at least about 2 fold, at least about 3 fold, at least about 4 fold, at least about 5 fold, at least about 6 fold, at least about 7 fold, at least about 8 fold, at least about 9 fold, at least about 10 fold, or more.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein. As used herein, the phrase “subject in need thereof” includes subjects, such as mammalian subjects, that would benefit, e.g., from administration of a composition comprising a chimeric polypeptide of the present disclosure such as a CAR or a TCR, e.g., to control tumor growth.

The term “therapeutically effective amount” or “therapeutically effective dosage” refers to an amount of an agent (e.g., a polynucleotide encoding a CAR or TCR comprising a chimeric polypeptide disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., a CAR or TCR comprising a chimeric polypeptide disclosed herein or a cell comprising such CAR or TCR) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., a CAR or TCR comprising a chimeric polypeptide of the present disclosure, or a cell comprising such CAR or TCR), which is clinically proven to effect a significant decrease in cancer or slowing of progression (regression) of cancer, such as an advanced solid tumor. The ability of a therapeutic agent of the present disclosure (e.g., a CAR or TCR comprising a chimeric polypeptide disclosed herein, or a cell comprising such CAR or TCR) to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeably with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the anti-cancer agent (e.g., a CAR or TCR comprising a chimeric polypeptide disclosed herein, or a cell comprising such CAR or TCR) promotes cancer regression to the point of eliminating the cancer.

“Promoting cancer regression” means that administering an effective amount of a composition disclosed herein (e.g., a CAR or TCR comprising a chimeric polypeptide disclosed herein, or a cell comprising such CAR or TCR), alone or in combination with an anti-neoplastic agent (e.g., a small molecule chemotherapeutic agent), results in a reduction in tumor growth or size, necrosis of the tumor, a decrease in severity of at least one disease symptom, an increase in frequency and duration of disease symptom-free periods, or a prevention of impairment or disability due to the disease affliction.

The terms “effective” and “effectiveness” with regard to a treatment include both pharmacological effectiveness and physiological safety. Pharmacological effectiveness refers to the ability of a composition disclosed herein (e.g., a CAR or TCR comprising a chimeric polypeptide disclosed herein, or a cell comprising such CAR or TCR) to promote cancer regression in the patient. Physiological safety refers to the level of toxicity, or other adverse physiological effects at the cellular, organ, and/or organism level (adverse effects) resulting from administration of a composition disclosed herein (e.g., a CAR or TCR comprising a chimeric polypeptide disclosed herein, or a cell comprising such CAR or TCR).

As used herein, the term “immune checkpoint inhibitor” refers to molecules that totally or partially reduce, inhibit, interfere with, or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or can be derived from antibodies.

As used herein, the terms “elevated concentrations” or “elevated levels” and grammatical variants thereof refer to above-normal levels of a substance compared to appropriate controls (e.g., healthy tissue or cells).

The terms “chimeric antigen receptor” and “CAR,” as used herein, refer to a recombinant fusion protein that has an antigen-specific extracellular domain coupled to an intracellular domain that directs the cell to perform a specialized function upon binding of an antigen to the extracellular domain. In some aspects, a chimeric antigen receptor disclosed herein comprises a chimeric polypeptide of the present disclosure.

The terms “artificial T cell receptor,” “chimeric T-cell receptor,” and “chimeric immunoreceptor” can each be used interchangeably herein with the term “chimeric antigen receptor.” Chimeric antigen receptors are distinguished from other antigen binding agents by their ability to both bind MHC-independent antigen and transduce activation signals via their intracellular domain.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (K_{D}) between about 0.1 μM to about 10 μM, for example, about 0.1 μM to about 1 μM or about 0.1 μM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains such as cAb VHH (camelid antibody variable domains) and humanized versions thereof, lgNAR VH (shark antibody variable domains) and humanized versions thereof, sdAb VH (single domain antibody variable domains), and “camelized” antibody variable domains are also suitable for use in a CAR of the present disclosure. In some aspects, T cell receptor (TCR) based recognition domains, such as single chain TCR (scTv, i.e., single chain two-domain TCR containing V.alpha.V.beta.) are also suitable for use in a TCR of the present disclosure.

A chimeric antigen receptor disclosed herein (e.g., comprising a chimeric polypeptide of the present disclosure) can also include an intracellular domain that provides an intracellular signal to the cell (e.g., a cell expressing the CAR) upon antigen binding to the antigen-specific extracellular domain. In some aspects, the intracellular signaling domain of a CAR is responsible for activation of at least one of the effector functions of the T cell in which the CAR is expressed.

The term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, 829, Fc RIII, Fc RI, and combinations of signaling molecules, such as CD3.zeta. and CD28, CD27, 4-1BB, DAP-10, OX40, and combinations thereof, as well as other similar molecules and fragments. Intracellular signaling portions of other members of the families of activating proteins can be used, such as FcγRIII and FcεRI. While usually the entire intracellular domain will be employed, in many cases it will not be necessary to use the entire intracellular polypeptide. To the extent that a truncated portion of the intracellular signaling domain can find use, such truncated portion can be used in place of the intact chain as long as it still transduces the effector function signal. The term intracellular domain is thus meant to include any truncated portion of the intracellular domain sufficient to transduce the effector function signal. Typically, the antigen-specific extracellular domain is linked to the intracellular domain of the chimeric antigen receptor by a transmembrane domain. A “transmembrane domain” traverses the cell membrane, anchors the CAR to the T cell surface, and connects the extracellular domain to the intracellular signaling domain, thus impacting expression of the CAR on the T cell surface. Chimeric antigen receptors can also further comprise one or more costimulatory domains and/or one or more spacers. A costimulatory domain is derived from the intracellular signaling domains of costimulatory proteins that enhance cytokine production, proliferation, cytotoxicity, and/or persistence in vivo.

A “peptide hinge” or “spacer” connects the antigen-specific extracellular domain to the transmembrane domain. The transmembrane domain is fused to the costimulatory domain, optionally a costimulatory domain is fused to a second costimulatory domain, and the costimulatory domain is fused to a signaling domain, not limited to CD3ζ. For example, inclusion of a spacer domain between the antigen-specific extracellular domain and the transmembrane domain, and between multiple scFvs in the case of tandem CAR, can affect flexibility of the antigen-binding domain(s) and thereby CAR function. Suitable transmembrane domains, costimulatory domains, and spacers are known in the art.

As used herein, the terms “ug” and “uM” are used interchangeably with “μg” and “μM,” respectively.

“Coding sequence” or “encoding nucleic acid” as used herein means the nucleic acids (RNA or DNA molecule) that comprise a nucleotide sequence which encodes a protein, e.g., CAR or TCR comprising a chimeric polypeptide disclosed herein, or a key polypeptide. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein refers to Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 75% identical, at least about 80% identical, at least about 85% identical, at least about 90% identical, or at least about 95% identical to one another. In some aspects, two or more sequences are said to be “highly conserved” if they are about 70% identical, about 75% identical, about 80% identical, about 85% identical, about 90% identical, about 95% identical, about 98% identical, or about 99% identical to one another.

In some aspects, two or more sequences are said to be “conserved” if they are at least about 30% identical, at least about 35% identical, at least about 40% identical, at least about 45% identical, at least about 50% identical, at least about 55%, at least about 60% identical, at least about 65% identical, at least about 70% identical, at least about 75% identical, at least about 80% identical, at least about 85% identical, at least about 90% identical, or at least about 95% identical to one another.

In some aspects, two or more sequences are said to be “conserved” if they are about 30% identical, about 35% identical, about 40% identical, about 45% identical, about 50% identical, about 55% identical, about 60% identical, about 65% identical, about 70% identical, about 75% identical, about 80% identical, about 85% identical, about 90% identical, about 95% identical, about 98% identical, or about 99% identical to one another. Conservation of sequence may apply to the entire length of a polynucleotide or polypeptide or may apply to a portion, region or feature thereof.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

Non-conservative amino acid substitutions include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

Other amino acid substitutions can also be used. For example, for the amino acid alanine, a substitution can be taken from any one of D-alanine, glycine, beta-alanine, L-cysteine and D-cysteine. For lysine, a replacement can be any one of D-lysine, arginine, D-arginine, homo-arginine, methionine, D-methionine, ornithine, or D-ornithine. Generally, substitutions in functionally important regions that can be expected to induce changes in the properties of isolated polypeptides are those in which (i) a polar residue, e.g., serine or threonine, is substituted for (or by) a hydrophobic residue, e.g., leucine, isoleucine, phenylalanine, or alanine; (ii) a cysteine residue is substituted for (or by) any other residue; (iii) a residue having an electropositive side chain, e.g., lysine, arginine or histidine, is substituted for (or by) a residue having an electronegative side chain, e.g., glutamic acid or aspartic acid; or (iv) a residue having a bulky side chain, e.g., phenylalanine, is substituted for (or by) one not having such a side chain, e.g., glycine. The likelihood that one of the foregoing non-conservative substitutions can alter functional properties of the protein is also correlated to the position of the substitution with respect to functionally important regions of the protein: some non-conservative substitutions can accordingly have little or no effect on biological properties.

As used herein, the term “identity” refers to the overall monomer conservation between polymeric molecules, e.g., between polypeptide molecules or polynucleotide molecules (e.g., DNA molecules and/or RNA molecules). The term “identical” without any additional qualifiers, e.g., protein A is identical to protein B, implies the sequences are 100% identical (100% sequence identity). Describing two sequences as, e.g., “70% identical,” is equivalent to describing them as having, e.g., “70% sequence identity.”

Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions are then compared.

The terms “percent identity,” “percent sequence identity,” and “percentage identity” are used interchangeably.

When a position in the first sequence is occupied by the same amino acid as the corresponding position in the second sequence, then the molecules are identical at that position. The percent identity between the two sequences is a function of the number of identical positions shared by the sequences, considering the number of gaps, and the length of each gap, which needs to be introduced for optimal alignment of the two sequences. The comparison of sequences and determination of percent identity between two sequences can be accomplished using a mathematical algorithm. Sequence alignments can be conducted using methods known in the art such as BLAST, MAFFT, Clustal (ClustalW, Clustal X or Clustal Omega), MUSCLE, Needle, Stretcher, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The terms “covalently linked,” “fused,” and grammatical variants thereof are used interchangeably and refer to a first moiety, e.g., a first amino acid sequence or nucleotide sequence, covalently or non-covalently joined to a second moiety, e.g., a second amino acid sequence or nucleotide sequence, respectively. The first moiety can be directly joined or juxtaposed to the second moiety or alternatively an intervening moiety can covalently join the first moiety to the second moiety. The term “linked” means not only a fusion of a first moiety to a second moiety at the C-terminus or the N-terminus, but also includes insertion of the whole first moiety (or the second moiety) into any two points, e.g., amino acids, in the second moiety (or the first moiety, respectively). In some aspects, the first moiety is linked to a second moiety by a peptide bond or a linker. The first moiety can be linked to a second moiety by a phosphodiester bond or a linker. The linker can be a peptide or a polypeptide (for polypeptide chains) or a nucleotide or a nucleotide chain (for nucleotide chains) or any chemical moiety (for polypeptide or polynucleotide chains or any chemical molecules).

The term “domain,” as used herein, refers to a portion of a polypeptide that retains one or more particular functions. The functional polypeptide domains can be synthetic or naturally occurring and can comprise a full protein or a domain of a protein that possesses a specific function.

As used herein, the terms “preferential binding”, “preferentially binds” or any grammatically similar terms indicate that a higher number of key polypeptide binds to the structural region of the cage polypeptide than the latch region to the structural region at the equal concentration of the latch region and the key polypeptide in a solution, e.g., in vivo, and/or the key polypeptide binds to the structural region of the cage polypeptide with a higher binding affinity than the latch region when both the latch region and the key polypeptide are present at the equal concentration in a solution, e.g., in vivo.

Various aspects described herein are described in further detail in the following subsections.

### II. Chimeric Polypeptides

The present disclosure provides chimeric polypeptide comprising a caged degron, wherein the degron is sequestered or caged in the chimeric polypeptide (e.g., a CAR or TCR). As used herein, the term “chimeric polypeptide” refers to a molecular system comprising a cage polypeptide that contains a sequestered degron, wherein the degron can be activated via an interaction between the cage polypeptide and a key polypeptide. The chimeric polypeptide, in some aspects, does not contain a key polypeptide, but instead, the key polypeptide can be added when necessary. In some aspects, the chimeric polypeptide further comprises a key polypeptide as a chimeric protein complex formed by the cage polypeptide and the key polypeptide. The cage polypeptide is a helix bundle comprising the sequestered or caged degron in a region of the cage polypeptide denoted as the latch region (e.g., integrated in the latch region), which can be displaced from the cage by a key polypeptide through competitive intermolecular binding. After the displacement of the latch region by the key polypeptide, the degron is exposed and accessible, e.g., to ubiquitin ligases.

The exposed degron can, for example, be ubiquitinated by a ubiquitin ligase, leading to the degradation of the degron and any bioactive molecules fused or conjugated to the chimeric polypeptide. Any functional polypeptide of interest (e.g., a CAR or a TCR) can be expressed as a fusion protein with a chimeric polypeptide disclosed herein (e.g., a chimeric polypeptide of SEQ ID NO: 14 to 76) such that the functional polypeptide of interest can be conditionally degraded in a titratable manner via endogenous expression (e.g., inducible expression) of the key polypeptide or administration of the key polypeptide.

Activation of a chimeric polypeptide integrated in a chimeric antigen receptor (CAR) or a T cell receptor (TCR) by its key polypeptide can lead to internalization and degradation of the CAR or TCR, and subsequent decrease of the amount of CAR or TCR expressed in the cell surface. Accordingly, the chimeric polypeptides disclosed herein can be used to modulate the level of active CAR or TCR present in the cell membrane. The modulation of CAR or TCR activity levels can in turn be used to control the number of engineered immune cells in vivo and/or to prevent, mitigate, or reverse immune cell exhaustion in immune therapy.

In some aspects, a chimeric polypeptide disclosed herein comprises a cage polypeptide comprising a structural region disclosed herein and a latch region that is capable of binding to the structural region, wherein the latch region further comprises a degron peptide comprising RRRG (SEQ ID NO:110).

In some aspects, the degron peptide replaces the same number of amino acids in the latch region. For example, to generate a chimeric polypeptide comprising a degron peptide 20 amino acids long (e.g., KTRGVEEVAEGVVLLRRRGN, SEQ ID NO:1), a latch region (e.g., the Cage1_N_Latch of SEQ ID NO:142) is engineered so that 20 consecutive amino acids, e.g., amino acids 2 to 21 of the Cage1_N_Latch of SEQ ID NO:142 are substituted (i.e., replaced) with the 20 amino acids of the degron peptide sequence. Therefore, the resulting latch region comprises some portions of the sequence identical to the parent latch sequence and some portions consisting of the degron peptide. In the example, amino acids 1 and 22 to 60 of the latch region are identical to the corresponding amino acids of the Cage1_N_Latch of SEQ ID NO:142, and amino acids 2 to 21 of the latch region comprise a degron peptide sequence, e.g., the 20 amino acids of the degron of SEQ ID NO:1 (Degron A). In some aspects, the latch region can be further engineered to remove one or more amino acids at the C terminus or N terminus.

As another example, to generate a chimeric polypeptide comprising a degron peptide comprising RRRG (SEQ ID NO: 110), a latch region (e.g., Cage1_N_Latch of SEQ ID NO:142) is engineered so that 4 consecutive amino acids (or any number of amino acids between 5 to 20 for other degron peptides of SEQ ID NOS:110 to 141, 680-804, or 918-9824 in the latch region are substituted (i.e., replaced) with a degron peptide sequence of the same length.

Thus, in some aspects, the present disclosure provides chimeric polypeptides in which a subsequence of a latch region disclosed herein is replaced with a subsequence from SEQ ID NO: 1 having the same length. Accordingly, in some aspects, 4, 5, 6, 7, 8, 9, 10, 11, 12, 13, 14, 15, 16, 17, 18, 19, or 20 consecutive amino acids from a latch region sequence of SEQ ID NOS:166-189, i.e., a latch region from a cage of SEQ ID NOS:2-13, can be replaced with a Degron A subsequence of identical length, selected from SEQ ID NOs: 1, 110-141, or 680-804. In some aspects, a degron useful for the present disclosure comprises EGVVLLRRRG (SEQ ID NO: 122) or EGVVLLRRRGN (SEQ ID NO: 123). In some aspects, 4, 5, 6, 7, 8, 9, 10, 11, 12, 13, 14, 15, 16, 17, 18, 19, or 20 consecutive amino acids from a latch region sequence of SEQ ID NOS:839, 841, 843, 845, 847, 849, 851, 853, 855, 857, 859, 861, 863, 865, or 867, i.e., a latch region from a cage of SEQ ID NOS:823-837, can be replaced with a Degron A subsequence of identical length, selected from SEQ ID NOs: 1, 110-141, or 680-804. In some aspects, a degron useful for the present disclosure comprises EGVVLLRRRG (SEQ ID NO: 122) or EGVVLLRRRGN (SEQ ID NO: 123).

In some aspects, a chimeric polypeptide of the present disclosure comprises a cage polypeptide comprising:
(i) a structural region comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a cage polypeptide structural region as set forth in SEQ ID NOS:142-165, 838, 840, 842, 844, 846, 848, 850, 852, 854, 856, 858, 860, 862, 864, or 866; and
(ii) a latch region comprising a degron peptide comprising any one of the peptide sequences set forth in SEQ ID NOs: 1, 110-141, 680-804, or 918-9824, e.g., SEQ ID NO: 122 or 123, wherein the latch region is capable of binding to the structural region.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 142, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 143, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 144, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 145, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 146, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 147, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 148, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 149, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 150, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 151, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 152, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 153, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 154, and the latch region is capable of binding to the structural region and comprises a degron peptide comprising any one of the peptide sequences set forth in SEQ ID NOs:1, 110-141, 680-804, or 918-9824.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 155, and the latch region is capable of binding to the structural region and comprises a degron peptide comprising any one of the peptide sequences set forth in SEQ ID NOs:1, 110-141, or 680-804.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 156, and the latch region is capable of binding to the structural region and comprises a degron peptide comprising any one of the peptide sequences set forth in SEQ ID NOs:1, 110-141, 680-804, or 918-982804.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 157, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 158, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 159, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 160, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 161, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 162, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 163, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 164, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 165, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 838, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 840, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 842, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 844, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 846, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 848, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 850, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 852, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 854, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 856, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 858, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 860, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 862, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 864, and the latch region is capable of binding to the structural region and comprises a degron peptide comprising any one of the peptide sequences set forth in SEQ ID NOs:1, 110-141, 680-804, or 918-982.

In some aspects, a chimeric polypeptide of the present disclosure comprises a cage polypeptide comprising a structural region and a latch region, wherein the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to a structural region of SEQ ID NO: 866, and the latch region is capable of binding to the structural region and comprises a degron peptide comprising any one of the peptide sequences set forth in SEQ ID NOs:1, 110-141, 680-804, or 918-982.

In some aspects, the latch region can form hydrogen bond networks and hydrophobic interactions with the structural region. In some aspects, the latch region comprises an amino acid sequence having at least about 40%, at least about 50%, at least about 60%, at least about 70%, at least about 80%, at least about 90%, at least about 95%, at least about 99%, or about 100% sequence identity to at least 50 consecutive amino acids within the structural region, wherein the latch region binds to the structural region.

In some aspects, the latch region comprises (a) at least about 10, at least about 15, at least about 20, at least about 25, at least about 30, or at least about 35 consecutive amino acid residues from a cage polypeptide latch region set forth in SEQ ID NOS:166-189, 839, 841, 843, 845, 847, 849, 851, 853, 855, 857, 859, 861, 863, 865, or 867; and (b) a degron peptide described herein, e.g., a degron peptide consisting of an amino acid sequence set forth in SEQ ID NOS: 1, 110-141, 680-804, or 918-982. In some aspects, the degron peptide useful for the chimeric polypeptide consists of the amino acid sequence as set forth in KTRGVEEVAEGVVLLRRRGN (SEQ ID NO: 1) or a fragment thereof (e.g., a 4 to 19 amino acid long fragment such as those set forth in SEQ ID NOs: 110-141 680-804, or 918-982).

In some aspects, the chimeric polypeptide comprises:
(i) a structural region comprising an amino acid sequence having at least about 50%, at least about 55%, at least about 60%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to a cage polypeptide structural region set forth in SEQ ID NO:142-165, 838, 840, 842, 844, 846, 848, 850, 852, 854, 856, 858, 860, 862, 864, or 866; and,
(ii) a latch region that is capable of binding to the structural region and comprises a degron peptide which consists or consists essentially of the amino acid sequence as set forth in KTRGVEEVAEGVVLLRRRGN (SEQ ID NO: 1) or a fragment thereof (e.g., a 4 to 19 amino acid long fragment such as those set forth in SEQ ID NOs: 110-141 680-804, or 918-982). In some aspects, the structural region of the cage polypeptide is capable of preferentially binding to the corresponding key polypeptide when exposed to the key polypeptide.

In some aspects, a given amino acid in a chimeric polypeptide disclosed herein can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that the desired activity is retained. Amino acids can be grouped according to similarities in the properties of their side chains: (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some aspects, the chimeric polypeptide of the present disclosure can include additional amino acid residues at the N-terminus, C-terminus, internal to the polypeptide, or a combination thereof. Such amino acid residues can be, e.g., detectable moieties such as fluorescent proteins, antibody epitope tags, etc. Additional moieties that can be included in a chimeric polypeptide described herein are provided elsewhere in the present disclosure.

VI.a. Cage Polypeptide

In some aspects, a cage polypeptide of a chimeric polypeptide disclosed herein comprises a structural region and a latch region, wherein the structural region comprises 2 or 3 alpha helices and the latch region is capable of binding to the structural region. Latch regions can be present near either terminus of a cage polypeptide of a chimeric polypeptide. In some aspects, the latch region can comprise a part or all of a single alpha helix at the N-terminal or C-terminal portion of the cage polypeptide. In some aspects, the latch region can comprise a part or all of a first, second or third alpha helix in the cage polypeptide. In further aspects, the latch region can comprise all or part of two or more different alpha helices in the cage polypeptide; for example, a C-terminal part of one alpha helix and an N-terminal portion of the next alpha helix, or all of two consecutive alpha helices.

In some aspects, the cage polypeptide of the present disclosure comprises at least three or at least four alpha helices, wherein the N-terminal alpha helix or the C-terminal alpha helix is a latch region, and the other alpha helices are a structural region. In some aspects, the cage polypeptide of the disclosure comprises at least three alpha helices, wherein the at least three of the alpha helices can form hydrogen bond networks and hydrophobic interactions. In some aspects, the cage polypeptide of the disclosure comprises at least four alpha helices, wherein each of the alpha helices is the same, i.e., the same sequence. In some aspects, the sequence of the latch region is similar to one or more of the alpha helices in the structural region but is not identical. In some aspects, the sequence of the latch region has at least one amino acid, at least two amino acids, at least three amino acids, at least four amino acids, at least five amino acids, at least six amino acids, at least seven amino acids, at least eight amino acids, at least nine amino acids, at least ten amino acids, at least 15 amino acids, at least 20 amino acids, at least 25 amino acids, at least 30 amino acids, at least 35 amino acids, or at least 40 amino acids, or at least 45 amino acids different from the sequence of the other alpha helices in the cage polypeptide. The difference in sequence can include amino acid mutation, deletion, or insertion. In some aspects, the sequence of the latch region is at least one amino acid, at least two amino acids, at least three amino acids, at least four amino acids, at least five amino acids, at least six amino acids, at least seven amino acids, at least eight amino acids, at least nine amino acids, at least ten amino acids, at least 15 amino acids, at least 20 amino acids, at least 25 amino acids, at least 30 amino acids, at least 35 amino acids, or at least 40 amino acids, or at least 45 amino acids shorter than the sequence of the other alpha helices in the cage polypeptide.

In some aspects, the cage polypeptide comprises three alpha helices, wherein two alpha helices form a structural region and one alpha helix at the N or C terminus forms a latch region. In some aspects, the cage polypeptide comprises four alpha helices, wherein three alpha helices form a structural region and one alpha helix at the N or C terminus forms a latch region.

In some aspects, the latch region of a cage polypeptide of the present disclosure is placed at the C-terminal helix so as to position the degron for maximum burial of the functional residues that need to be sequestered to maintain the degron in an inactive state while simultaneously burying hydrophobic residues and promoting solvent exposure/compensatory hydrogen bonds of polar residues. In some aspects, the alpha helices of the structural region and the latch region can interact with each other via a combination of hydrophobic contacts and hydrogen bond networks formed between helical interfaces.

In some aspects, the structural region can comprise three alpha helices and the interaction with the latch region results in a four-helix bundle cage polypeptide (“3plus1” design). In still further aspects, the structural region can comprise two alpha helices and the interaction with the latch region results in a three-helix bundle cage polypeptide (“2plus1” design). In some aspects, the alpha helices of the structural region and the latch region can interact with each other via a combination of hydrophobic contacts and hydrogen bond networks formed between helical interfaces.

Various modifications can be employed to modulate the affinities of latch regions, structural regions, and key polypeptides, including but not limited to modifications of the overall length of the region or polypeptide, or a portion (such as an alpha helix) of the region, introduction of destabilizing mutations, etc. Thus, in some aspects, the dynamic range of activation by key polypeptides can be tuned by truncating the latch region length to be shorter than the alpha-helices in the structural region, simultaneously weakening the cage polypeptide-latch region interaction and opening an exposed region on the cage polypeptide that the key polypeptide can bind to. Similarly, the dynamic range of activation by key polypeptides can also be tuned in a similar manner by designing mutations into the latch region that weaken the cage polypeptide-latch region interaction, therefore facilitating the displacement of the latch region by a key polypeptide, and as a result facilitating the activation of the degron.

In some aspects, a cage polypeptide comprises 4 alpha helices (e.g., 3 alpha helices that make up the structural region and 1 alpha helix that make up the latch region; “3plus1” design). In still further aspects, a cage polypeptide comprises 3 alpha helices (e.g., 2 alpha helices that make up the structural region and 1 alpha helix that make up the latch region; “2plus1” design).

Design of the helical bundle cage polypeptides of the disclosure can be carried out by any suitable means. In certain aspects, a BUNDLEGRIDSAMPLER™ in the ROSETTA™ program can be used to generate backbone geometry based on the Crick expression for a coiled-coil and allows efficient, parallel sampling of a regular grid of coiled-coil expression parameter values, which correspond to a continuum of peptide backbone conformations. This can be supplemented by design for hydrogen bond networks using any suitable means, including but not limited to, as described in Boyken et. al., (Science 352:680-687 (2016)), followed by ROSETTA™ side chain design. In further aspects, best scoring designs, based on total score, number of unsatisfied hydrogen bonds, and lack of voids in the core of the protein can be selected for helical bundle cage polypeptide design.

Each alpha helix of a cage polypeptide disclosed herein can be of any suitable length and/or amino acid composition as appropriate for an intended use (e.g., to effectively sequester a degron of the present disclosure, e.g., a degron comprising, consisting of, or consisting essentially of the amino acid sequence set forth in any one of SEQ ID NOS:1, 110-141, 680-804, or 918-982). In some aspects, a degron useful for the present disclosure comprises EGVVLLRRRG (SEQ ID NO: 122) or EGVVLLRRRGN (SEQ ID NO: 123). In some aspects, each helix is independently 38 to 58 amino acids in length. In some aspects, one or more helices in the cage polypeptide comprises 38 to 45 amino acids, e.g., 38, 39, 40, 41, 42, 43, 44, or 45. In some aspects, one or more helices in the cage polypeptide comprises 45 to 52 amino acids, e.g., 45, 46, 47, 48, 49, 50, 51, or 52. In some aspects, one or more helices in the cage polypeptide comprises 52 to 58 amino acids, 52, 53, 54, 55, 56, 57, or 58.

As described above, the cage polypeptide of a chimeric polypeptide disclosed herein can be used to sequester a degron (e.g., a degron comprising, consisting of, or consisting essentially of the amino acid sequence set forth in any one of SEQ ID NOS:1, 110-141, 680-804, or 918-982) in the cage polypeptide, holding the degron in an inactive (“off”) state, until combined with an activation component (the “key” polypeptide). Due to the binding of the key polypeptide to the cage polypeptide, the degron can be exposed and now be active (“on”).

In some aspects, the alpha helices of a cage polypeptide are joined by one or more linkers. The amino acid linkers connecting each alpha helix can be of any suitable length and/or amino acid composition as appropriate for an intended use (e.g., to effectively sequester a degron of the present disclosure, e.g., a degron comprising, consisting of, or consisting essentially of the amino acid sequence set forth in any one of SEQ ID NOS:1, 110-141, 680-804, or 918-982). In some aspects, each amino acid linker is independently between 2 and 10 amino acids in length. In some aspects, the cage polypeptide comprises one linker, two linkers, three linkers, four linkers, five linkers, or six linkers. In some aspects, one or more linkers in the cage polypeptide is 2, 3, 4, 5, 6, 7, 8, 9, or 10 amino acids in length.

In some aspects, the linkers can be structured or flexible (e.g., poly-GS). In certain aspects, these linkers can encode further functional sequences, including but not limited to protease cleavage sites or one half of a split intein system. As described herein, in some aspects, linkers can further comprise one or more functional polypeptide domains—in such aspects, the linkers can be of any size suitable to include the one or more functional polypeptide domains, while maintaining the ability of the structural region and the latch region to interact.

Suitable linkers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers (including, for example, (GS)n, (GSGGS)n (SEQ ID NO:101) and (GGGS)n (SEQ ID NO:102), where n is an integer of at least one), glycinealanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine and is much less restricted than residues with longer side chains (see Scheraga, Rev. Computational Chem. 11173-142 (1992)). Exemplary linkers can comprise amino acid sequences including, but not limited to, GGSG (SEQ ID NO:103), GGSGG (SEQ ID NO:104), GSGSG (SEQ ID NO:105), GSGGG (SEQ ID NO:106), GGGSG (SEQ ID NO:107), GSSSG (SEQ ID NO:108), and the like.

While the above linkers are described in the context of joining two or more alpha helices of a cage polypeptide, it will be apparent to those skilled in the art that such linkers can be used to link any two or more polypeptides present in a chimeric polypeptide of the present disclosure.

In some aspects, a cage polypeptide of a chimeric polypeptide disclosed herein comprises any one of the structural regions comprising the amino acid sequences set forth in SEQ ID NO:142 to 165, 838, 840, 842, 844, 846, 848, 850, 852, 854, 856, 858, 860, 862, 864, or 866.

In some aspects, the cage polypeptide of the present disclosure comprises a structural region and a latch region, wherein the structural region of the cage polypeptide comprises any one of the amino acid sequences as set forth in SEQ ID NOS:142-165, 838, 840, 842, 844, 846, 848, 850, 852, 854, 856, 858, 860, 862, 864, or 866; and the latch region comprises at least 20, 21, 22, 23, 24, 25, 26, 27, 28, 29, 30, 31, 32, 33, 34, or 35 consecutive amino acid residues from a corresponding latch region as set forth in SEQ ID NOS: 166-189, 839, 841, 843, 845, 847, 849, 851, 853, 855, 857, 859, 861, 863, 865, or 867, respectively.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 142 and the latch region comprises at least 20, 21, 22, 23, 24, 25, 26, 27, 28, 29, 30, 31, 32, 33, 34, or 35 consecutive amino acid residues from SEQ ID NO: 166. In some aspects, the latch region of the cage polypeptide further comprises one or more amino acids derived from SEQ ID NO:166. In some aspects, the latch region of the cage polypeptide comprises amino acids 22 to 60 of SEQ ID NO:166 and a degron peptide sequence described herein, e.g., a sequence selected from SEQ ID NO: 1, 110-141, 680-804, or 918-982. In some aspects, the latch region of the cage polypeptide comprises amino acids 26 to 60 of SEQ ID NO:166 and a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:166 except that amino acids 2 to 21 of SEQ ID NO:166 or 6 to 25 of SEQ ID NO:166 are a degron peptide sequence described herein. In some aspects, the latch region of the cage polypeptide comprises the amino acid sequence of SEQ ID NO:166 except that amino acids 2 to 21 of SEQ ID NO:166 or 6 to 25 of SEQ ID NO:166 are a degron peptide sequence described herein, e.g., a sequence selected from SEQ ID NO: 1, 110-141, 680-804, or 918-982. In some aspects, a degron useful for the present disclosure comprises EGVVLLRRRG (SEQ ID NO: 122) or EGVVLLRRRGN (SEQ ID NO: 123).

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:143 and the latch region comprises at least 20, 21, 22, 23, 24, 25, 26, 27, 28, 29, 30, 31, 32, 33, 34, or 35 consecutive amino acid residues SEQ ID NO:167. In some aspects, the latch region of the cage polypeptide further comprises one or more amino acids derived from SEQ ID NO:167. In some aspects, the latch region of the cage polypeptide comprises amino acids 21 to 60 of SEQ ID NO:167 and a degron peptide sequence described herein, e.g., a sequence selected from SEQ ID NO: 1, 110-141, 680-804, or 918-982. In some aspects, the latch region of the cage polypeptide comprises amino acids 26 to 60 of SEQ ID NO:167 and a degron peptide sequence described herein, e.g., a sequence selected from SEQ ID NO: 1, 110-141, 680-804, or 918-982. In some aspects, the latch region of the cage polypeptide comprises amino acids 29 to 60 of SEQ ID NO:167 and a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:167 except that amino acids 1 to 20 of SEQ ID NO:167 or 6 to 25 of SEQ ID NO:167 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:167 except that amino acids 9 to 28 of SEQ ID NO:167 are a degron peptide sequence described herein, e.g., a sequence selected from SEQ ID NO: 1, 110-141, 680-804, or 918-982. In some aspects, a degron useful for the present disclosure comprises EGVVLLRRRG (SEQ ID NO: 122) or EGVVLLRRRGN (SEQ ID NO: 123).

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:144 and the latch region comprises at least 20, 21, 22, 23, 24, 25, 26, 27, 28, 29, 30, 31, 32, 33, 34, or 35 consecutive amino acid residues from SEQ ID NO:168. In some aspects, the latch region of the cage polypeptide further comprises one or more amino acids derived from SEQ ID NO:168. In some aspects, the latch region of the cage polypeptide comprises amino acids 1 to 34 of SEQ ID NO:168, a degron peptide sequence described herein, e.g., a sequence selected from SEQ ID NO: 1, 110-141, 680-804, or 918-982, and optionally one or more from amino acids 55 to 60 of SEQ ID NO: 168. In some aspects, the latch region of the cage polypeptide comprises the amino acid sequence of SEQ ID NO:168 except that amino acids 35 to 54 of SEQ ID NO:168 are a degron peptide sequence described herein, e.g., a sequence selected from SEQ ID NO: 1, 110-141, 680-804, or 918-982. In some aspects, a degron useful for the present disclosure comprises EGVVLLRRRG (SEQ ID NO: 122) or EGVVLLRRRGN (SEQ ID NO: 123).

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:145 and the latch region comprises at least 20, 21, 22, 23, 24, 25, 26, 27, 28, 29, 30, 31, 32, 33, 34, or 35 consecutive amino acid residues from SEQ ID NO:169. In some aspects, the latch region of the cage polypeptide further comprises one or more amino acids derived from SEQ ID NO:169. In some aspects, the latch region of the cage polypeptide comprises amino acids 1 to 39 of SEQ ID NO:169, and a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:169 except that amino acids 40 to 59 of SEQ ID NO:169 are a degron peptide sequence described herein, e.g., a sequence selected from SEQ ID NO: 1, 110-141, 680-804, or 918-982. In some aspects, a degron useful for the present disclosure comprises EGVVLLRRRG (SEQ ID NO: 122) or EGVVLLRRRGN (SEQ ID NO: 123).

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:146 structural region and the latch region comprises at least 20, 21, 22, 23, 24, 25, 26, 27, 28, 29, 30, 31, 32, 33, 34, or 35 consecutive amino acid residues from SEQ ID NO:170. In some aspects, the latch region of the cage polypeptide further comprises one or more amino acids derived from SEQ ID NO:170. In some aspects, the latch region of the cage polypeptide comprises amino acids 23 to 60 of SEQ ID NO:170 and a degron peptide sequence described herein, e.g., a sequence selected from SEQ ID NO: 1, 110-141, 680-804, or 918-982. In some aspects, the latch region of the cage polypeptide comprises amino acids 30 to 60 of SEQ ID NO:170 and a degron peptide described herein, e.g., a sequence selected from SEQ ID NO: 1, 110-141, 680-804, or 918-982. In some aspects, the latch region of the cage polypeptide comprises amino acids 34 to 60 of SEQ ID NO:170, a degron peptide sequence described herein, and optionally one or more of amino acids 1 to 13 of SEQ ID NO:170. In some aspects, the latch region of the cage polypeptide comprises amino acids 37 to 60 of SEQ ID NO:170, a degron peptide sequence described herein, and one or more of amino acids 1 to 16 of SEQ ID NO:170. In some aspects, the latch region of the cage polypeptide comprises amino acids 1 to 27 of SEQ ID NO:170, a degron peptide sequence described herein, and one or more of amino acids 48 to 60 of SEQ ID NO:170. In some aspects, the latch region of the cage polypeptide comprises amino acids 41 to 60 of SEQ ID NO:170, a degron peptide sequence described herein, and optionally one or more of amino acids 1 to 20 of SEQ ID NO:170. In some aspects, the latch region of the cage polypeptide comprises amino acids 1 to 31 of SEQ ID NO:170, a degron peptide sequence described herein, e.g., a sequence selected from SEQ ID NO: 1, 110-141, 680-804, or 918-982, and one or more of amino acids 52 to 60 of SEQ ID NO:170. In some aspects, the latch region of the cage polypeptide comprises amino acids 1 to 34 of SEQ ID NO:170, a degron peptide sequence described herein, e.g., a sequence selected from SEQ ID NO: 1, 110-141, 680-804, or 918-982, and one or more of amino acids 55 to 60 of SEQ ID NO: 170. In some aspects, the latch region of the cage polypeptide comprises amino acids 1 to 38 of SEQ ID NO:170, a degron peptide sequence described herein, e.g., a sequence selected from SEQ ID NO: 1, 110-141, 680-804, or 918-982, and one or more of amino acids 59 and 60 of SEQ ID NO:170. In some aspects, the latch region of the cage polypeptide comprises the amino acid sequence of SEQ ID NO:170 except that amino acids 3 to 22 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 10 to 29 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 14 to 33 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 17 to 36 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 21 to 40 of SEQ ID NO:170 are a degron peptide sequence described herein. In some aspects, the latch region of the cage polypeptide comprises the amino acid sequence of SEQ ID NO:170 except that amino acids 28 to 47 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 32 to 51 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 35 to 54 of SEQ ID NO:170 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:170 except that amino acids 39 to 58 of SEQ ID NO:170 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:147 region, and the latch region comprises at least 20, 21, 22, 23, 24, 25, 26, 27, 28, 29, 30, 31, 32, 33, 34, or 35 consecutive amino acid residues from SEQ ID NO:171. In some aspects, the latch region of the cage polypeptide further comprises one or more amino acids derived from SEQ ID NO:171. In some aspects, the latch region of the cage polypeptide comprises amino acids 21 to 60 of SEQ ID NO:171 and a degron peptide sequence described herein, e.g., a sequence selected from SEQ ID NO: 1, 110-141, 680-804, or 918-982. In some aspects, the latch region of the cage polypeptide comprises amino 25 to 60 of SEQ ID NO:171, a degron peptide sequence described herein, e.g., a sequence selected from SEQ ID NO: 1, 110-141, 680-804, or 918-982, and optionally one or more amino acids of 1 to 4 of SEQ ID NO:171. In some aspects, the latch region of the cage polypeptide comprises amino 28 to 60 of SEQ ID NO:171, a degron peptide sequence described herein, e.g., a sequence selected from SEQ ID NO: 1, 110-141, 680-804, or 918-982, and optionally one or more amino acids of 1 to 7 of SEQ ID NO:171. In some aspects, the latch region of the cage polypeptide comprises amino 1 to 36 of SEQ ID NO:171, a degron peptide sequence described herein, e.g., a sequence selected from SEQ ID NO: 1, 110-141, 680-804, or 918-982, and optionally one or more amino acids of 57 to 60 of SEQ ID NO:171. In some aspects, the latch region of the cage polypeptide comprises amino acids 1 to 40 of SEQ ID NO:171 and a degron peptide sequence described herein, e.g., a sequence selected from SEQ ID NO: 1, 110-141, 680-804, or 918-982.

In some aspects, the latch region of the cage polypeptide comprises the amino acid sequence of SEQ ID NO:171 except that amino acids 1 to 20 of SEQ ID NO:171 are a degron peptide sequence described herein. In some aspects, the latch region of the cage polypeptide comprises the amino acid sequence of SEQ ID NO:171 except that amino acids 5 to 24 of SEQ ID NO:171 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:171 except that amino acids 8 to 27 of SEQ ID NO:171 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:171 except that amino acids 37 to 56 of SEQ ID NO:171 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:171 except that amino acids 41 to 60 of SEQ ID NO:171 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:148, and the latch region comprises at least 20, 21, 22, 23, 24, 25, 26, 27, 28, 29, 30, 31, 32, 33, 34, or 35 consecutive amino acid residues from SEQ ID NO:172. In some aspects, the latch region of the cage polypeptide further comprises one or more amino acids derived from SEQ ID NO:172. In some aspects, the latch region of the cage polypeptide comprises amino 26 to 60 of SEQ ID NO:172, a degron peptide sequence described herein, e.g., a sequence selected from SEQ ID NO: 1, 110-141, 680-804, or 918-982, and optionally one or more amino acids of 1 to 5 of SEQ ID NO: 172. In some aspects, the latch region of the cage polypeptide comprises amino 27 to 60 of SEQ ID NO: 172, a degron peptide sequence described herein, e.g., a sequence selected from SEQ ID NO: 1, 110-141, 680-804, or 918-982, and optionally one or more amino acids of 1 to 6 of SEQ ID NO:172. In some aspects, the latch region of the cage polypeptide comprises the amino acid sequence of SEQ ID NO:172 except that amino acids 6 to 25 of SEQ ID NO:172 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:172 except that amino acids 7 to 26 of SEQ ID NO:172 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:149, and the latch region comprises at least 20, 21, 22, 23, 24, 25, 26, 27, 28, 29, 30, 31, 32, 33, 34, or 35 consecutive amino acid residues from SEQ ID NO:173. In some aspects, the latch region of the cage polypeptide further comprises one or more amino acids derived from SEQ ID NO:173. In some aspects, the latch region of the cage polypeptide comprises amino 20 to 60 of SEQ ID NO: 173 and a degron peptide sequence described herein, e.g., a sequence selected from SEQ ID NO: 1, 110-141, 680-804, or 918-982. In some aspects, the latch region of the cage polypeptide comprises amino 25 to 60 of SEQ ID NO:173, a degron peptide sequence described herein, e.g., a sequence selected from SEQ ID NO: 1, 110-141, 680-804, or 918-982, and optionally one or more amino acids of 1 to 4 of SEQ ID NO:173. In some aspects, the latch region of the cage polypeptide comprises amino 28 to 60 of SEQ ID NO:173, a degron peptide sequence described herein, e.g., a sequence selected from SEQ ID NO: 1, 110-141, 680-804, or 918-982, and optionally one or more amino acids of 1 to 7 of SEQ ID NO:173. In some aspects, the latch region of the cage polypeptide comprises amino 32 to 60 of SEQ ID NO: 173, a degron peptide sequence described herein, e.g., a sequence selected from SEQ ID NO: 1, 110-141, 680-804, or 918-982, and optionally one or more amino acids of 1 to 14 of SEQ ID NO:173. In some aspects, the latch region of the cage polypeptide comprises amino 36 to 60 of SEQ ID NO:173, a degron peptide sequence described herein, e.g., a sequence selected from SEQ ID NO: 1, 110-141, 680-804, or 918-982, and optionally one or more amino acids of 1 to 15 of SEQ ID NO:173. In some aspects, the latch region of the cage polypeptide comprises amino 1 to 22 of SEQ ID NO:173, a degron peptide sequence described herein, e.g., a sequence selected from SEQ ID NO: 1, 110-141, 680-804, or 918-982, and optionally one or more amino acids of 43 to 60 of SEQ ID NO:173. In some aspects, the latch region of the cage polypeptide comprises amino 1 to 25 of SEQ ID NO:173, a degron peptide sequence described herein, e.g., a sequence selected from SEQ ID NO: 1, 110-141, 680-804, or 918-982, and optionally one or more amino acids of 46 to 60 of SEQ ID NO:173. In some aspects, the latch region of the cage polypeptide comprises amino 1 to 26 of SEQ ID NO:173, a degron peptide sequence described herein, e.g., a sequence selected from SEQ ID NO: 1, 110-141, 680-804, or 918-982, and optionally one or more amino acids of 47 to 60 of SEQ ID NO:173. In some aspects, the latch region of the cage polypeptide comprises amino 1 to 29 of SEQ ID NO:173, a degron peptide sequence described herein, e.g., a sequence selected from SEQ ID NO: 1, 110-141, 680-804, or 918-982, and optionally one or more amino acids of 50 to 60 of SEQ ID NO:173. In some aspects, the latch region of the cage polypeptide comprises amino 1 to 33 of SEQ ID NO:173, a degron peptide sequence described herein, e.g., a sequence selected from SEQ ID NO: 1, 110-141, 680-804, or 918-982, and optionally one or more amino acids of 54 to 60 of SEQ ID NO:173. In some aspects, the latch region of the cage polypeptide comprises amino 1 to 37 of SEQ ID NO:173, a degron peptide sequence described herein, e.g., a sequence selected from SEQ ID NO: 1, 110-141, 680-804, or 918-982, and optionally one or more amino acids of 58 to 60 of SEQ ID NO:173. In some aspects, the latch region of the cage polypeptide comprises amino 1 to 39 of SEQ ID NO:173, a degron peptide sequence described herein, e.g., a sequence selected from SEQ ID NO: 1, 110-141, 680-804, or 918-982, and optionally amino acid 60 of SEQ ID NO:173. In some aspects, the latch region of the cage polypeptide comprises amino 1 to 40 of SEQ ID NO:173 and a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 150 and the latch region comprises at least 20, 21, 22, 23, 24, 25, 26, 27, 28, 29, 30, 31, 32, 33, 34, or 35 consecutive amino acid residues from SEQ ID NO:174. In some aspects, the latch region of the cage polypeptide further comprises one or more amino acids derived from SEQ ID NO:174. In some aspects, the latch region of the cage polypeptide comprises amino 30 to 60 of SEQ ID NO:174, a degron peptide sequence described herein, and optionally one or more amino acids of 1 to 9 of SEQ ID NO:174. In some aspects, the latch region of the cage polypeptide comprises amino 1 to 34 of SEQ ID NO:174, a degron peptide sequence described herein, e.g., a sequence selected from SEQ ID NO: 1, 110-141, 680-804, or 918-982, and optionally one or more amino acids of 55 to 60 of SEQ ID NO:174. In some aspects, the latch region of the cage polypeptide comprises the amino acid sequence of SEQ ID NO:174 except that amino acids 10 to 29 of SEQ ID NO:174 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:174 except that amino acids 35 to 54 of SEQ ID NO:174 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 151 and the latch region comprises at least 20, 21, 22, 23, 24, 25, 26, 27, 28, 29, 30, 31, 32, 33, 34, or 35 consecutive amino acid residues from SEQ ID NO:175. In some aspects, the latch region of the cage polypeptide further comprises one or more amino acids derived from SEQ ID NO:175. In some aspects, the latch region of the cage polypeptide comprises amino 23 to 60 of SEQ ID NO:175, a degron peptide sequence described herein, e.g., a sequence selected from SEQ ID NO: 1, 110-141, 680-804, or 918-982, and optionally one or more amino acids of 1 to 2 of SEQ ID NO:175. In some aspects, the latch region of the cage polypeptide comprises amino 25 to 60 of SEQ ID NO:175, a degron peptide sequence described herein, e.g., a sequence selected from SEQ ID NO: 1, 110-141, 680-804, or 918-982, and optionally one or more amino acids of 1 to 4 of SEQ ID NO:175. In some aspects, the latch region of the cage polypeptide comprises amino 32 to 60 of SEQ ID NO:175, a degron peptide sequence described herein, e.g., a sequence selected from SEQ ID NO: 1, 110-141, 680-804, or 918-982, and optionally one or more amino acids of 1 to 11 of SEQ ID NO:175. In some aspects, the latch region of the cage polypeptide comprises the amino acid sequence of SEQ ID NO:175 except that amino acids 3 to 22 of SEQ ID NO:175 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:175 except that amino acids 5 to 24 of SEQ ID NO:175 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:175 except that amino acids 12 to 31 of SEQ ID NO:175 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 152 structural region and the latch region comprises at least 20, 21, 22, 23, 24, 25, 26, 27, 28, 29, 30, 31, 32, 33, 34, or 35 consecutive amino acid residues from SEQ ID NO: 176. In some aspects, the latch region of the cage polypeptide further comprises one or more amino acids derived from SEQ ID NO:176. In some aspects, the latch region of the cage polypeptide comprises amino 26 to 60 of SEQ ID NO:176, a degron peptide sequence described herein, e.g., a sequence selected from SEQ ID NO: 1, 110-141, 680-804, or 918-982, and optionally one or more amino acids of 1 to 5 of SEQ ID NO:176. The polynucleotide of claim 3, wherein the latch region of the cage polypeptide comprises SEQ ID NO:176 except that amino acids 6 to 25 of SEQ ID NO:176 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:153 and the latch region comprises at least 20, 21, 22, 23, 24, 25, 26, 27, 28, 29, 30, 31, 32, 33, 34, or 35 consecutive amino acid residues from SEQ ID NO:177. In some aspects, the latch region of the cage polypeptide further comprises one or more amino acids derived from SEQ ID NO:177.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:154 and the latch region comprises at least 20, 21, 22, 23, 24, 25, 26, 27, 28, 29, 30, 31, 32, 33, 34, or 35 consecutive amino acid residues from SEQ ID NO:178. In some aspects, the latch region of the cage polypeptide further comprises one or more amino acids derived from SEQ ID NO:178. In some aspects, the latch region of the cage polypeptide comprises amino 23 to 60 of SEQ ID NO:178, a degron peptide sequence described herein, e.g., a sequence selected from SEQ ID NO: 1, 110-141, 680-804, or 918-982, and optionally one or more amino acids of 1 to 2 of SEQ ID NO:178. In some aspects, the latch region of the cage polypeptide comprises the amino acid sequence of SEQ ID NO:178 except that amino acids 3 to 22 of SEQ ID NO:178 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:155 and the latch region comprises at least 20, 21, 22, 23, 24, 25, 26, 27, 28, 29, 30, 31, 32, 33, 34, or 35 consecutive amino acid residues from SEQ ID NO:179. In some aspects, the latch region of the cage polypeptide further comprises one or more amino acids derived from SEQ ID NO:179. In some aspects, the latch region of the cage polypeptide comprises amino 1 to 29 of SEQ ID NO:179, a degron peptide sequence described herein, e.g., a sequence selected from SEQ ID NO: 1, 110-141, 680-804, or 918-982, and optionally one or more amino acids of 50 to 60 of SEQ ID NO:179. In some aspects, the latch region of the cage polypeptide comprises the amino acid sequence of SEQ ID NO:179 except that amino acids 30 to 49 of SEQ ID NO:179 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:156 and the latch region comprises at least 20, 21, 22, 23, 24, 25, 26, 27, 28, 29, 30, 31, 32, 33, 34, or 35 consecutive amino acid residues from SEQ ID NO:180. In some aspects, the latch region of the cage polypeptide further comprises one or more amino acids derived from SEQ ID NO:180.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:157 and the latch region comprises at least 20, 21, 22, 23, 24, 25, 26, 27, 28, 29, 30, 31, 32, 33, 34, or 35 consecutive amino acid residues from SEQ ID NO:181. In some aspects, the latch region of the cage polypeptide further comprises one or more amino acids derived from SEQ ID NO:181. In some aspects, the latch region of the cage polypeptide comprises amino 38 to 60 of SEQ ID NO:181, a degron peptide sequence described herein, e.g., a sequence selected from SEQ ID NO: 1, 110-141, 680-804, or 918-982, and optionally one or more amino acids of 1 to 17 of SEQ ID NO:181. In some aspects, the latch region of the cage polypeptide comprises the amino acid sequence of SEQ ID NO:181 except that amino acids 18 to 37 of SEQ ID NO:181 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:158 and the latch region comprises at least 20, 21, 22, 23, 24, 25, 26, 27, 28, 29, 30, 31, 32, 33, 34, or 35 consecutive amino acid residues from SEQ ID NO:182. In some aspects, the latch region of the cage polypeptide further comprises one or more amino acids derived from SEQ ID NO:182. In some aspects, the latch region of the cage polypeptide comprises amino 23 to 60 of SEQ ID NO:182, a degron peptide sequence described herein, e.g., a sequence selected from SEQ ID NO: 1, 110-141, 680-804, or 918-982, and optionally one or more amino acids of 1 to 2 of SEQ ID NO: 182. In some aspects, the latch region of the cage polypeptide comprises amino 26 to 60 of SEQ ID NO:182, a degron peptide sequence described herein, e.g., a sequence selected from SEQ ID NO: 1, 110-141, 680-804, or 918-982, and optionally one or more amino acids of 1 to 5 of SEQ ID NO:182. In some aspects, the latch region of the cage polypeptide comprises amino 33 to 60 of SEQ ID NO:182, a degron peptide sequence described herein, e.g., a sequence selected from SEQ ID NO: 1, 110-141, 680-804, or 918-982, and optionally one or more amino acids of 1 to 12 of SEQ ID NO:182. In some aspects, the latch region of the cage polypeptide comprises the amino acid sequence of SEQ ID NO:182 except that amino acids 3 to 22 of SEQ ID NO:182 is a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:182 except that amino acids 6 to 25 of SEQ ID NO:182 are a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:182 except that amino acids 13 to 32 of SEQ ID NO:182 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:159 and the latch region comprises at least 20, 21, 22, 23, 24, 25, 26, 27, 28, 29, 30, 31, 32, 33, 34, or 35 consecutive amino acid residues from SEQ ID NO:183. In some aspects, the latch region of the cage polypeptide further comprises one or more amino acids derived from SEQ ID NO:183. In some aspects, the latch region of the cage polypeptide comprises amino 1 to 40 of SEQ ID NO:183 and a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:183 except that amino acids 41 to 60 of SEQ ID NO:183 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:160 and the latch region comprises at least 20, 21, 22, 23, 24, 25, 26, 27, 28, 29, 30, 31, 32, 33, 34, or 35 consecutive amino acid residues SEQ ID NO:184. In some aspects, the latch region of the cage polypeptide further comprises one or more amino acids derived from SEQ ID NO:184. In some aspects, the latch region of the cage polypeptide comprises amino 26 to 60 of SEQ ID NO:184, a degron peptide sequence described herein, e.g., a sequence selected from SEQ ID NO: 1, 110-141, 680-804, or 918-982, and optionally one or more amino acids of 1 to 5 of SEQ ID NO:184. In some aspects, the latch region of the cage polypeptide comprises the amino acid sequence of SEQ ID NO:184 except that amino acids 6 to 25 of SEQ ID NO:184 are a degron peptide sequence described herein, e.g., a sequence selected from SEQ ID NO: 1, 110-141, 680-804, or 918-982. In some aspects, the latch region of the cage polypeptide comprises amino 33 to 60 of SEQ ID NO:184, a degron peptide sequence described herein, and optionally one or more amino acids of 1 to 12 of SEQ ID NO:184. In some aspects, the latch region of the cage polypeptide comprises the amino acid sequence of SEQ ID NO:184 except that amino acids 13 to 32 of SEQ ID NO:184 are a degron peptide sequence described herein, e.g., a sequence selected from SEQ ID NO: 1, 110-141, 680-804, or 918-982. In some aspects, the latch region of the cage polypeptide comprises amino 1 to 37 of SEQ ID NO:184, a degron peptide sequence described herein, e.g., a sequence selected from SEQ ID NO: 1, 110-141, 680-804, or 918-982, and optionally one or more amino acids of 58 to 60 of SEQ ID NO:184. In some aspects, the latch region of the cage polypeptide comprises the amino acid sequence of SEQ ID NO:184 except that amino acids 38 to 57 of SEQ ID NO:184 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:161 and the latch region comprises at least 20, 21, 22, 23, 24, 25, 26, 27, 28, 29, 30, 31, 32, 33, 34, or 35 consecutive amino acid residues from SEQ ID NO:185. In some aspects, the latch region of the cage polypeptide further comprises one or more amino acids derived from SEQ ID NO:185. In some aspects, the latch region of the cage polypeptide comprises amino 21 to 60 of SEQ ID NO:185 and a degron peptide sequence described herein, e.g., a sequence selected from SEQ ID NO: 1, 110-141, 680-804, or 918-982. In some aspects, the latch region of the cage polypeptide comprises the amino acid sequence of SEQ ID NO:185 except that amino acids 1 to 20 of SEQ ID NO:185 are a degron peptide sequence described herein, e.g., a sequence selected from SEQ ID NO: 1, 110-141, 680-804, or 918-982. In some aspects, the latch region of the cage polypeptide comprises amino 24 to 60 of SEQ ID NO:185, a degron peptide sequence described herein, e.g., a sequence selected from SEQ ID NO: 1, 110-141, 680-804, or 918-982, and optionally one or more amino acids of 1 to 3 of SEQ ID NO:185. In some aspects, the latch region of the cage polypeptide comprises the amino acid sequence of SEQ ID NO:185 except that amino acids 4 to 23 of SEQ ID NO:185 are a degron peptide sequence described herein, e.g., a sequence selected from SEQ ID NO: 1, 110-141, 680-804, or 918-982. In some aspects, the latch region of the cage polypeptide comprises amino 1 to 29 of SEQ ID NO:185, a degron peptide sequence described herein, and optionally one or more amino acids of 50 to 60 of SEQ ID NO:185. In some aspects, the latch region of the cage polypeptide comprises the amino acid sequence of SEQ ID NO:185 except that amino acids 30 to 49 of SEQ ID NO:185 are a degron peptide sequence described herein, e.g., a sequence selected from SEQ ID NO: 1, 110-141, 680-804, or 918-982. In some aspects, the latch region of the cage polypeptide comprises amino 1 to 32 of SEQ ID NO:185, a degron peptide sequence described herein, e.g., a sequence selected from SEQ ID NO: 1, 110-141, 680-804, or 918-982, and optionally one or more amino acids of 53 to 60 of SEQ ID NO:185. In some aspects, the latch region of the cage polypeptide comprises the amino acid sequence of SEQ ID NO:185 except that amino acids 33 to 52 of SEQ ID NO:185 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:162 and the latch region comprises at least 20, 21, 22, 23, 24, 25, 26, 27, 28, 29, 30, 31, 32, 33, 34, or 35 consecutive amino acid residues from SEQ ID NO:186. In some aspects, the latch region of the cage polypeptide further comprises one or more amino acids derived from SEQ ID NO:186. In some aspects, the latch region of the cage polypeptide comprises amino 33 to 60 of SEQ ID NO:186, a degron peptide sequence described herein, e.g., a sequence selected from SEQ ID NO: 1, 110-141, 680-804, or 918-982, and optionally one or more of amino acids 1 to 12 of SEQ ID NO:186. In some aspects, the latch region of the cage polypeptide comprises the amino acid sequence of SEQ ID NO:186 except that amino acids 13 to 32 of SEQ ID NO:186 are a degron peptide sequence described herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:163 and the latch region comprises at least 20, 21, 22, 23, 24, 25, 26, 27, 28, 29, 30, 31, 32, 33, 34, or 35 consecutive amino acid residues from SEQ ID NO:187. In some aspects, the latch region of the cage polypeptide further comprises one or more amino acids derived from SEQ ID NO:187.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:164 and the latch region comprises at least 20, 21, 22, 23, 24, 25, 26, 27, 28, 29, 30, 31, 32, 33, 34, or 35 consecutive amino acid residues from SEQ ID NO:188. In some aspects, the latch region of the cage polypeptide further comprises one or more amino acids derived from SEQ ID NO:188.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO:165 and the latch region comprises at least 20, 21, 22, 23, 24, 25, 26, 27, 28, 29, 30, 31, 32, 33, 34, or 35 consecutive amino acid residues from SEQ ID NO:189. In some aspects, the latch region of the cage polypeptide further comprises one or more amino acids derived from SEQ ID NO:189. In some aspects, the latch region of the cage polypeptide comprises amino 1 to 22 of SEQ ID NO:189, a degron peptide sequence described herein, e.g., a sequence selected from SEQ ID NO: 1, 110-141, 680-804, or 918-982, and optionally one or more of amino acids 43 to 60 of SEQ ID NO:189. In some aspects, the latch region of the cage polypeptide comprises SEQ ID NO:189 except amino acids 23 to 42 of SEQ ID NO:189 are a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 838 and the latch region comprises at least 20, 21, 22, 23, 24, 25, 26, 27, 28, 29, 30, 31, 32, 33, 34, or 35 consecutive amino acid residues from SEQ ID NO: 839. In some aspects, the latch region of the cage polypeptide further comprises one or more amino acids derived from SEQ ID NO: 839. In some aspects, the latch region of the cage polypeptide comprises SEQ ID NO: 839 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 840 and the latch region comprises at least 20, 21, 22, 23, 24, 25, 26, 27, 28, 29, 30, 31, 32, 33, 34, or 35 consecutive amino acid residues from SEQ ID NO: 841. In some aspects, the latch region of the cage polypeptide further comprises one or more amino acids derived from SEQ ID NO: 841. In some aspects, the latch region of the cage polypeptide comprises SEQ ID NO: 841 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 842 and the latch region comprises at least 20, 21, 22, 23, 24, 25, 26, 27, 28, 29, 30, 31, 32, 33, 34, or 35 consecutive amino acid residues from SEQ ID NO: 843. In some aspects, the latch region of the cage polypeptide further comprises one or more amino acids derived from SEQ ID NO: 843. In some aspects, the latch region of the cage polypeptide comprises SEQ ID NO: 843 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 844 and the latch region comprises at least 20, 21, 22, 23, 24, 25, 26, 27, 28, 29, 30, 31, 32, 33, 34, or 35 consecutive amino acid residues from SEQ ID NO: 845. In some aspects, the latch region of the cage polypeptide further comprises one or more amino acids derived from SEQ ID NO: 845. In some aspects, the latch region of the cage polypeptide comprises SEQ ID NO: 845 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 846 and the latch region comprises at least 20, 21, 22, 23, 24, 25, 26, 27, 28, 29, 30, 31, 32, 33, 34, or 35 consecutive amino acid residues from SEQ ID NO: 847. In some aspects, the latch region of the cage polypeptide further comprises one or more amino acids derived from SEQ ID NO: 847. In some aspects, the latch region of the cage polypeptide comprises SEQ ID NO: 847 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 848 and the latch region comprises at least 20, 21, 22, 23, 24, 25, 26, 27, 28, 29, 30, 31, 32, 33, 34, or 35 consecutive amino acid residues from SEQ ID NO: 849. In some aspects, the latch region of the cage polypeptide further comprises one or more amino acids derived from SEQ ID NO: 849. In some aspects, the latch region of the cage polypeptide comprises SEQ ID NO: 849 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 850 and the latch region comprises at least 20, 21, 22, 23, 24, 25, 26, 27, 28, 29, 30, 31, 32, 33, 34, or 35 consecutive amino acid residues from SEQ ID NO: 851. In some aspects, the latch region of the cage polypeptide further comprises one or more amino acids derived from SEQ ID NO: 851. In some aspects, the latch region of the cage polypeptide comprises SEQ ID NO: 851 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 852 and the latch region comprises at least 20, 21, 22, 23, 24, 25, 26, 27, 28, 29, 30, 31, 32, 33, 34, or 35 consecutive amino acid residues from SEQ ID NO: 853. In some aspects, the latch region of the cage polypeptide further comprises one or more amino acids derived from SEQ ID NO: 853. In some aspects, the latch region of the cage polypeptide comprises SEQ ID NO: 853 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 854 and the latch region comprises at least 20, 21, 22, 23, 24, 25, 26, 27, 28, 29, 30, 31, 32, 33, 34, or 35 consecutive amino acid residues from SEQ ID NO: 855. In some aspects, the latch region of the cage polypeptide further comprises one or more amino acids derived from SEQ ID NO: 855. In some aspects, the latch region of the cage polypeptide comprises SEQ ID NO: 855 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 856 and the latch region comprises at least 20, 21, 22, 23, 24, 25, 26, 27, 28, 29, 30, 31, 32, 33, 34, or 35 consecutive amino acid residues from SEQ ID NO: 857. In some aspects, the latch region of the cage polypeptide further comprises one or more amino acids derived from SEQ ID NO: 857. In some aspects, the latch region of the cage polypeptide comprises SEQ ID NO: 857 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 858 and the latch region comprises at least 20, 21, 22, 23, 24, 25, 26, 27, 28, 29, 30, 31, 32, 33, 34, or 35 consecutive amino acid residues from SEQ ID NO: 859. In some aspects, the latch region of the cage polypeptide further comprises one or more amino acids derived from SEQ ID NO: 859. In some aspects, the latch region of the cage polypeptide comprises SEQ ID NO: 859 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 860 and the latch region comprises at least 20, 21, 22, 23, 24, 25, 26, 27, 28, 29, 30, 31, 32, 33, 34, or 35 consecutive amino acid residues from SEQ ID NO: 861. In some aspects, the latch region of the cage polypeptide further comprises one or more amino acids derived from SEQ ID NO: 861. In some aspects, the latch region of the cage polypeptide comprises SEQ ID NO: 861 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 862 and the latch region comprises at least 20, 21, 22, 23, 24, 25, 26, 27, 28, 29, 30, 31, 32, 33, 34, or 35 consecutive amino acid residues from SEQ ID NO: 863. In some aspects, the latch region of the cage polypeptide further comprises one or more amino acids derived from SEQ ID NO: 863. In some aspects, the latch region of the cage polypeptide comprises SEQ ID NO: 863 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 864 and the latch region comprises at least 20, 21, 22, 23, 24, 25, 26, 27, 28, 29, 30, 31, 32, 33, 34, or 35 consecutive amino acid residues from SEQ ID NO: 865. In some aspects, the latch region of the cage polypeptide further comprises one or more amino acids derived from SEQ ID NO: 865. In some aspects, the latch region of the cage polypeptide comprises SEQ ID NO: 865 except for a subsequence corresponding to a degron peptide sequence disclosed herein, e.g., a sequence selected from SEQ ID NO: 1, 110-141, 680-804, or 918-982.

In some aspects, the cage polypeptide of the present disclosure comprises a structural region and a latch region, wherein the structural region of the cage polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 866 and the latch region comprises at least 20, 21, 22, 23, 24, 25, 26, 27, 28, 29, 30, 31, 32, 33, 34, or 35 consecutive amino acid residues from SEQ ID NO: 867. In some aspects, the latch region of the cage polypeptide further comprises one or more amino acids derived from SEQ ID NO: 867. In some aspects, the latch region of the cage polypeptide comprises SEQ ID NO: 867 except for a subsequence corresponding to a degron peptide sequence disclosed herein, e.g., a sequence selected from SEQ ID NO: 1, 110-141, 680-804, or 918-982.

VI.b. Degron Peptide

As described herein, a chimeric polypeptide of the present disclosure comprises a degron peptide.

As used herein, a “degron” is a peptide capable of targeting the cage polypeptide and any functional polypeptide domain fused to the cage polypeptide for degradation. In some aspects, the degron is a ubiquitin-dependent degron, i.e., a polypeptide recognized by an ubiquitin ligase which enzymatically attaches a ubiquitin protein to the degron, which marks the degron and any polypeptide (e.g., a CAR or TCR) attached to the degron for degradation/targeting to the proteasome. When a degron peptide disclosed herein is integrated in a chimeric polypeptide of the present disclosure, the terms “degron peptide” and “degron peptide sequence” are used interchangeably.

In some aspects, the chimeric polypeptide comprises a degron peptide within the amino acid sequence of the cage polypeptide. In some aspects, the degron peptide is within the latch region of the cage polypeptide. In some aspects, the latch region comprises more than one copy of the degron peptide. In some aspects, a chimeric polypeptide disclosed herein can comprise two or more degron peptides. In some aspects, the chimeric polypeptide disclosed comprises more than one copy of the same degron. In some aspects, the chimeric polypeptide disclosed herein comprises different degrons.

In some aspects, the degron peptide can be any one of the peptides set forth in SEQ ID NOS:1, 110-141 680-804, or 918-982. In some aspects, a degron useful for the present disclosure comprises EGVVLLRRRG (SEQ ID NO: 122) or EGVVLLRRRGN (SEQ ID NO: 123).

When a key polypeptide is expressed or administered and activates the cage polypeptide by interacting with the structural region, the degron targets the cage polypeptide, and any functional polypeptide domains and/or additional bioactive domain fused to the cage polypeptide, for degradation. In this way, a functional polypeptide domain of interest (e.g., a CAR or TCR) fused to a cage polypeptide having a degron (e.g., a degron of SEQ ID NO:1, or any degrons of SEQ ID NOS: 110-141 680-804, or 918-982) can be conditionally degraded in a titratable manner via expression of the key.

In some aspects, the degron of the present disclosure comprises, consists or consists essentially of a polypeptide of sequence KTRGVEEVAEGVVLLRRRGN (SEQ ID NO:1), wherein the N-terminal lysine is the ubiquitinated amino acid residue and the sequence from Arg16 to Gly20 is the substrate region recognized by the ubiquitin ligase. See Melvin et al. (2013) PloS ONE 8(10):e78082 and Bonger et al. (2011) Nature Chemical Biology 7: 531-537, both of which are herein incorporated by reference in their entireties. In some aspects, the degron of the present disclosure comprises, consists, or consists essentially of a polypeptide of sequence KTRGVEEVAEGVVLLRRRGNK (DegronA*, SEQ ID NO: 801); KEEVAEGVVLLRRRGNK (DegronA*_Truncation1, SEQ ID NO: 802); KTRGVEEVAEGVVLLRK (DegronA*_Truncation2, SEQ ID NO: 803); EGVVLLRRRGNK (DegronA*_Truncation3, SEQ ID NO: 804) TRGVEEVAEGVVLLRRRGNK (DegronA*_Truncation6, SEQ ID NO: 918); RGVEEVAEGVVLLRRRGNK (DegronA*_Truncation7, SEQ ID NO: 919); GVEEVAEGVVLLRRRGNK (DegronA*_Truncation8, SEQ ID NO: 920); VEEVAEGVVLLRRRGNK (DegronA*_Truncation9, SEQ ID NO: 921); EEVAEGVVLLRRRGNK (DegronA*_Truncation10, SEQ ID NO: 922); EVAEGVVLLRRRGNK (DegronA*_Truncation11, SEQ ID NO: 923); VAEGVVLLRRRGNK (DegronA*_Truncation12, SEQ ID NO: 924); AEGVVLLRRRGNK (DegronA*_Truncation13, SEQ ID NO: 925); GVVLLRRRGNK (DegronA*_Truncation14, SEQ ID NO: 926); VVLLRRRGNK (DegronA*_Truncation15, SEQ ID NO: 927); VLLRRRGNK (DegronA*_Truncation16, SEQ ID NO: 928); LLRRRGNK (DegronA*_Truncation17, SEQ ID NO: 929); LRRRGNK (DegronA*_Truncation18, SEQ ID NO: 930); RRRGNK (DegronA*_Truncation19, SEQ ID NO: 931); KTEVAEGVVLLRRRGNK (DegronA*_Truncation20, SEQ ID NO: 932); KTRGAEGVVLLRRRGNK (DegronA*_Truncation21, SEQ ID NO: 933); KTRGVEGVVLLRRRGNK (DegronA*_Truncation22, SEQ ID NO: 934); KTRGVEEVVLLRRRGNK (DegronA*_Truncation23, SEQ ID NO: 935); KTRGVEEVAELRRRGNK (DegronA*_Truncation24, SEQ ID NO: 936); KTVLLRRRGNK (DegronA*_Truncation25, SEQ ID NO: 937); KTRGLRRRGNK (DegronA*_Truncation26, SEQ ID NO: 938); CTRGVEEVAEGVVLLRRRGNK (DegronA*_Mutation1, SEQ ID NO: 939); FTRGVEEVAEGVVLLRRRGNK (DegronA*_Mutation2, SEQ ID NO: 940); KIRGVEEVAEGVVLLRRRGNK (DegronA*_Mutation3, SEQ ID NO: 941); KLRGVEEVAEGVVLLRRRGNK (DegronA*_Mutation4, SEQ ID NO: 942); KNRGVEEVAEGVVLLRRRGNK (DegronA*_Mutation5, SEQ ID NO: 943); KRRGVEEVAEGVVLLRRRGNK (DegronA*_Mutation6, SEQ ID NO: 944); KTDGVEEVAEGVVLLRRRGNK (DegronA*_Mutation7, SEQ ID NO: 945); KTFGVEEVAEGVVLLRRRGNK (DegronA*_Mutation8, SEQ ID NO: 946); KTHGVEEVAEGVVLLRRRGNK (DegronA*_Mutation9, SEQ ID NO: 947); KTPGVEEVAEGVVLLRRRGNK (DegronA*_Mutation10, SEQ ID NO: 948); KTRGKEEVAEGVVLLRRRGNK (DegronA*_Mutation11, SEQ ID NO: 949); KTRGMEEVAEGVVLLRRRGNK (DegronA*_Mutation12, SEQ ID NO: 950); KTRGTEEVAEGVVLLRRRGNK (DegronA*_Mutation13, SEQ ID NO: 951); KTRGVAEVAEGVVLLRRRGNK (DegronA*_Mutation14, SEQ ID NO: 952); KTRGVEAVAEGVVLLRRRGNK (DegronA*_Mutation15, SEQ ID NO: 953); KTRGVEDVAEGVVLLRRRGNK (DegronA*_Mutation16, SEQ ID NO: 954); KTRGVEECAEGVVLLRRRGNK (DegronA*_Mutation17, SEQ ID NO: 955); KTRGVEENAEGVVLLRRRGNK (DegronA*_Mutation18, SEQ ID NO: 956); KTRGVEETAEGVVLLRRRGNK (DegronA*_Mutation19, SEQ ID NO: 957); KTRGVEEVACGVVLLRRRGNK (DegronA*_Mutation20, SEQ ID NO: 958); KTRGVEEVAEEVVLLRRRGNK (DegronA*_Mutation21, SEQ ID NO: 959); KTRGVEEVAEGCVLLRRRGNK (DegronA*_Mutation22, SEQ ID NO: 960); KTRGVEEVAEGVGLLRRRGNK (DegronA*_Mutation23, SEQ ID NO: 961); KTRGVEEVAEGVVLLRHRGNK (DegronA*_Mutation24, SEQ ID NO: 962); KTRGVEEVAEGVVLLRQRGNK (DegronA*_Mutation25, SEQ ID NO: 963); KTRGVEEVAEGVVLLRRRGFK (DegronA*_Mutation26, SEQ ID NO: 964); KTRGVEEVAEGVVLLRRRGNH (DegronA*_Mutation27, SEQ ID NO: 965); KTRGVEEVAEGVVLTRRRGNK (DegronA*_Mutation28, SEQ ID NO: 966); KTRGVEEVAEGWVLLRRRGNK (DegronA*_Mutation29, SEQ ID NO: 967); KTRGVEEVAEPVVLLRRRGNK (DegronA*_Mutation30, SEQ ID NO: 968); KTRGVEEVAHGVVLLRRRGNK (DegronA*_Mutation31, SEQ ID NO: 969); KTRGVEEVAPGVVLLRRRGNK (DegronA*_Mutation32, SEQ ID NO: 970); KTRGVEEVAQGVVLLRRRGNK (DegronA*_Mutation33, SEQ ID NO: 971); KTRGVEEVATGVVLLRRRGNK (DegronA*_Mutation34, SEQ ID NO: 972); KTRGVEEVHEGVVLLRRRGNK (DegronA*_Mutation35, SEQ ID NO: 973); KTRGVEEVPEGVVLLRRRGNK (DegronA*_Mutation36, SEQ ID NO: 974); KTRGVEEVREGVVLLRRRGNK (DegronA*_Mutation37, SEQ ID NO: 975); KTRGVEHVAEGVVLLRRRGNK (DegronA*_Mutation38, SEQ ID NO: 976); KTRGVEPVAEGVVLLRRRGNK (DegronA*_Mutation39, SEQ ID NO: 977); KTRGVERVAEGVVLLRRRGNK (DegronA*_Mutation40, SEQ ID NO: 978); KTRGVEVVAEGVVLLRRRGNK (DegronA*_Mutation41, SEQ ID NO: 979); KTRGVQEVAEGVVLLRRRGNK (DegronA*_Mutation42, SEQ ID NO: 980); KTRSVEEVAEGVVLLRRRGNK (DegronA*_Mutation43, SEQ ID NO: 981); or QTRGVEEVAEGVVLLRRRGNK (DegronA*_Mutation44, SEQ ID NO: 982). In some aspects, the degron of the present disclosure is not KTRGVEEV (DegronA*_Truncation4, SEQ ID NO: 805); or KTRGVEEVAEGV (DegronA*_Truncation5, SEQ ID NO: 806). In some aspects, a degron useful for the present disclosure comprises EGVVLLRRRG (SEQ ID NO: 122) or EGVVLLRRRGN (SEQ ID NO: 123).

In some aspects, the degron peptide useful for the chimeric polypeptide of the present disclosure comprises, consists, or consists essentially of an amino acid sequence that is at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to SEQ ID NO:1.

In some aspects, the degron peptide useful for the chimeric polypeptide of the present disclosure comprises, consists, or consists essentially of an amino acid sequence that is at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identical to a SEQ ID NO:1 fragment of SEQ ID NO: 110-141 680-804, or 918-982.

In some aspects, the degron peptide useful for the chimeric polypeptide of the present disclosure comprises, consists, or consists essentially of an amino acid sequence that is identical to SEQ ID NO:1 to a SEQ ID NO:1 fragment of SEQ ID NO: 110-141 or 680-804 except for 1, 2, 3, 4, 5, 6, 7, 8, 9, or 10 amino acid substitutions.

In some aspects, substituted degron can recruit an ubiquitin ligase and being ubiquitinated. For example, a degron of the present disclosure can recruit a E3 ubiquitin ligase, i.e., a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein (e.g., a lysine present in the degron peptide sequence or in its vicinity in the latch region) by an isopeptide bond. A chimeric polypeptide disclosed herein can interact with a corresponding key polypeptide. Chimeric polypeptides of the present disclosure comprising a degron of SEQ ID NO:1, 110-141, or 801-804 (e.g., chimeric polypeptides of SEQ ID NOS: 14 to 76) can interact with a corresponding key of SEQ ID NOS: 77 to 96, as indicated in TABLE 2, below.

In some aspects, the degron is present within about 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0 amino acids from either the N-terminus or the C-terminus of the latch region.

In some aspects, the cage polypeptide comprises a single degron peptide within the latch region. In other aspects, the cage polypeptide can comprise more than one degron peptides within the latch region. In some aspects, the cage polypeptide can comprise more than one latch region, e.g., a C-terminal helix latch region and an N-terminal helix latch region. In some aspects, the degron may replace (substitute) one or more amino acids in the latch region or may be added to the latch region without removal of any amino acid residues from the latch region.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 14, wherein the chimeric polypeptide is capable of preferentially binding to the key polypeptide consisting of SEQ ID NO: 77, and upon binding to the key polypeptide, is capable of being degraded by a ubiquitin dependent pathway. In certain aspects, the chimeric polypeptide comprises the amino acid sequence set forth in SEQ ID NO: 14.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 14, wherein the chimeric polypeptide is capable of preferentially binding to the key polypeptide consisting of SEQ ID NO: 77, and upon binding to the key polypeptide, is capable of being degraded by a ubiquitin dependent pathway. In certain aspects, the chimeric polypeptide consists or consists essentially of the amino acid sequence set forth in SEQ ID NO: 14.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 15, wherein the chimeric polypeptide is capable of preferentially binding to the key polypeptide consisting of SEQ ID NO: 77, and upon binding to the key polypeptide, is capable of being degraded by a ubiquitin dependent pathway. In certain aspects, the chimeric polypeptide comprises the amino acid sequence set forth in SEQ ID NO: 15.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 15, wherein the chimeric polypeptide is capable of preferentially binding to the key polypeptide consisting of SEQ ID NO: 77, and upon binding to the key polypeptide, is capable of being degraded by a ubiquitin dependent pathway. In certain aspects, the chimeric polypeptide consists or consists essentially of the amino acid sequence set forth in SEQ ID NO: 15.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 16, wherein the chimeric polypeptide is capable of preferentially binding to the key polypeptide consisting of SEQ ID NO: 77, and upon binding to the key polypeptide, is capable of being degraded by a ubiquitin dependent pathway. In certain aspects, the chimeric polypeptide comprises the amino acid sequence set forth in SEQ ID NO: 16.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 16, wherein the chimeric polypeptide is capable of preferentially binding to the key polypeptide consisting of SEQ ID NO: 77, and upon binding to the key polypeptide, is capable of being degraded by a ubiquitin dependent pathway. In certain aspects, the chimeric polypeptide consists or consists essentially of the amino acid sequence set forth in SEQ ID NO: 16.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 17, wherein the chimeric polypeptide is capable of preferentially binding to the key polypeptide consisting of SEQ ID NO:78, and upon binding to the key polypeptide, is capable of being degraded by a ubiquitin dependent pathway. In certain aspects, the chimeric polypeptide comprises the amino acid sequence set forth in SEQ ID NO: 17.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 17, wherein the chimeric polypeptide is capable of preferentially binding to the key polypeptide consisting of SEQ ID NO: 78, and upon binding to the key polypeptide, is capable of being degraded by a ubiquitin dependent pathway. In certain aspects, the chimeric polypeptide consists or consists essentially of the amino acid sequence set forth in SEQ ID NO: 17.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 18, wherein the chimeric polypeptide is capable of preferentially binding to the key polypeptide consisting of SEQ ID NO: 78, and upon binding to the key polypeptide, is capable of being degraded by a ubiquitin dependent pathway. In certain aspects, the chimeric polypeptide comprises the amino acid sequence set forth in SEQ ID NO: 18.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 18, wherein the chimeric polypeptide is capable of preferentially binding to the key polypeptide consisting of SEQ ID NO: 78, and upon binding to the key polypeptide, is capable of being degraded by a ubiquitin dependent pathway. In certain aspects, the chimeric polypeptide consists or consists essentially of the amino acid sequence set forth in SEQ ID NO: 18.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 19, wherein the chimeric polypeptide is capable of preferentially binding to the key polypeptide consisting of SEQ ID NO: 79, and upon binding to the key polypeptide, is capable of being degraded by a ubiquitin dependent pathway. In certain aspects, the chimeric polypeptide comprises the amino acid sequence set forth in SEQ ID NO: 19.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 19, wherein the chimeric polypeptide is capable of preferentially binding to the key polypeptide consisting of SEQ ID NO: 79, and upon binding to the key polypeptide, is capable of being degraded by a ubiquitin dependent pathway. In certain aspects, the chimeric polypeptide consists or consists essentially of the amino acid sequence set forth in SEQ ID NO: 19.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 20, wherein the chimeric polypeptide is capable of preferentially binding to the key polypeptide consisting of SEQ ID NO: 80, and upon binding to the key polypeptide, is capable of being degraded by a ubiquitin dependent pathway. In certain aspects, the chimeric polypeptide comprises the amino acid sequence set forth in SEQ ID NO: 20.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 20, wherein the chimeric polypeptide is capable of preferentially binding to the key polypeptide consisting of SEQ ID NO: 80, and upon binding to the key polypeptide, is capable of being degraded by a ubiquitin dependent pathway. In certain aspects, the chimeric polypeptide consists or consists essentially of the amino acid sequence set forth in SEQ ID NO: 20.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 21, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 21.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 21, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 21.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 22, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 22.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 22, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 22.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 23, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 23.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 23, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 23.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 24, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 24.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 24, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 24.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 25, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 25.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 25, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 25.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 26, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 26.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 26, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 26.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 27, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 27.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 27, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 27.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 28, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 28.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 28, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 28.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 29, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 29.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 29, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 29.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 30, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 30.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 30, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 30.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 31, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 31.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 31, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 31.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 32, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 32.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 32, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 32.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 33, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 33.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 33, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 33.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 34, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 34.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 34, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 34.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 35, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 35.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 35, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 35.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 36, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 36.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 36, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 36.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 37, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 37.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 37, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 37.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 38, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 38.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 38, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 38.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 39, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 39.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 39, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 39.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 40, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 40.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 40, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 40.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 41, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 41.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 41, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 41.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 42, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 42.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 42, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 42.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 43, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 43.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 43, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 43.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 44, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 44.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 44, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 44.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 45, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 45.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 45, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 45.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 46, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 46.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 46, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 46.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 47, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 47.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 47, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 47.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 48, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 48.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 48, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 48.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 49, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide comprises the amino acid sequence set forth in SEQ ID NO: 49.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 49, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide consists or consists essentially of the amino acid sequence set forth in SEQ ID NO: 49.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 50, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide comprises the amino acid sequence set forth in SEQ ID NO: 50.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 50, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide consists or consists essentially of the amino acid sequence set forth in SEQ ID NO: 50.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 51, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide comprises the amino acid sequence set forth in SEQ ID NO: 51.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 51, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide consists or consists essentially of the amino acid sequence set forth in SEQ ID NO: 51.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 52, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide comprises the amino acid sequence set forth in SEQ ID NO: 52.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 52, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide consists or consists essentially of the amino acid sequence set forth in SEQ ID NO: 52.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 53, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide comprises the amino acid sequence set forth in SEQ ID NO: 53.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 53, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide consists or consists essentially of the amino acid sequence set forth in SEQ ID NO: 53.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 54, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide comprises the amino acid sequence set forth in SEQ ID NO: 54.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 54, wherein the chimeric polypeptide is capable of preferentially binding to the key polypeptide consisting of SEQ ID NO: 84, and upon binding to the key polypeptide, is capable of being degraded by a ubiquitin dependent pathway. In certain aspects, the chimeric polypeptide consists or consists essentially of the amino acid sequence set forth in SEQ ID NO: 54.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 55, wherein the chimeric polypeptide is capable of preferentially binding to the key polypeptide consisting of SEQ ID NO: 85, and upon binding to the key polypeptide, is capable of being degraded by a ubiquitin dependent pathway. In certain aspects, the chimeric polypeptide comprises the amino acid sequence set forth in SEQ ID NO: 55.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 55, wherein the chimeric polypeptide is capable of preferentially binding to the key polypeptide consisting of SEQ ID NO: 85, and upon binding to the key polypeptide, is capable of being degraded by a ubiquitin dependent pathway. In certain aspects, the chimeric polypeptide consists or consists essentially of the amino acid sequence set forth in SEQ ID NO: 55.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 56, wherein the chimeric polypeptide is capable of preferentially binding to the key polypeptide consisting of SEQ ID NO: 85, and upon binding to the key polypeptide, is capable of being degraded by a ubiquitin dependent pathway. In certain aspects, the chimeric polypeptide comprises the amino acid sequence set forth in SEQ ID NO: 56.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 56, wherein the chimeric polypeptide is capable of preferentially binding to the key polypeptide consisting of SEQ ID NO: 85, and upon binding to the key polypeptide, is capable of being degraded by a ubiquitin dependent pathway. In certain aspects, the chimeric polypeptide consists or consists essentially of the amino acid sequence set forth in SEQ ID NO: 56.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 57, wherein the chimeric polypeptide is capable of preferentially binding to the key polypeptide consisting of SEQ ID NO: 86, and upon binding to the key polypeptide, is capable of being degraded by a ubiquitin dependent pathway. In certain aspects, the chimeric polypeptide comprises the amino acid sequence set forth in SEQ ID NO: 57.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 57, wherein the chimeric polypeptide is capable of preferentially binding to the key polypeptide consisting of SEQ ID NO: 86, and upon binding to the key polypeptide, is capable of being degraded by a ubiquitin dependent pathway. In certain aspects, the chimeric polypeptide consists or consists essentially of the amino acid sequence set forth in SEQ ID NO: 57.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 58, wherein the chimeric polypeptide is capable of preferentially binding to the key polypeptide consisting of SEQ ID NO: 85, and upon binding to the key polypeptide, is capable of being degraded by a ubiquitin dependent pathway. In certain aspects, the chimeric polypeptide comprises the amino acid sequence set forth in SEQ ID NO: 58.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 58, wherein the chimeric polypeptide is capable of preferentially binding to the key polypeptide consisting of SEQ ID NO: 85, and upon binding to the key polypeptide, is capable of being degraded by a ubiquitin dependent pathway. In certain aspects, the chimeric polypeptide consists or consists essentially of the amino acid sequence set forth in SEQ ID NO: 58.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 59, wherein the chimeric polypeptide is capable of preferentially binding to the key polypeptide consisting of SEQ ID NO: 86, and upon binding to the key polypeptide, is capable of being degraded by a ubiquitin dependent pathway. In certain aspects, the chimeric polypeptide comprises the amino acid sequence set forth in SEQ ID NO: 59.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 59, wherein the chimeric polypeptide is capable of preferentially binding to the key polypeptide consisting of SEQ ID NO: 86, and upon binding to the key polypeptide, is capable of being degraded by a ubiquitin dependent pathway. In certain aspects, the chimeric polypeptide consists or consists essentially of the amino acid sequence set forth in SEQ ID NO: 59.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 60, wherein the chimeric polypeptide is capable of preferentially binding to the key polypeptide consisting of SEQ ID NO: 87, and upon binding to the key polypeptide, is capable of being degraded by a ubiquitin dependent pathway. In certain aspects, the chimeric polypeptide comprises the amino acid sequence set forth in SEQ ID NO: 60.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 60, wherein the chimeric polypeptide is capable of preferentially binding to the key polypeptide consisting of SEQ ID NO: 87, and upon binding to the key polypeptide, is capable of being degraded by a ubiquitin dependent pathway. In certain aspects, the chimeric polypeptide consists or consists essentially of the amino acid sequence set forth in SEQ ID NO: 60.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 61, wherein the chimeric polypeptide is capable of preferentially binding to the key polypeptide consisting of SEQ ID NO: 88, and upon binding to the key polypeptide, is capable of being degraded by a ubiquitin dependent pathway. In certain aspects, the chimeric polypeptide comprises the amino acid sequence set forth in SEQ ID NO: 61.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 61, wherein the chimeric polypeptide is capable of preferentially binding to the key polypeptide consisting of SEQ ID NO: 88, and upon binding to the key polypeptide, is capable of being degraded by a ubiquitin dependent pathway. In certain aspects, the chimeric polypeptide consists or consists essentially of the amino acid sequence set forth in SEQ ID NO: 61.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 62, wherein the chimeric polypeptide is capable of preferentially binding to the key polypeptide consisting of SEQ ID NO: 89, and upon binding to the key polypeptide, is capable of being degraded by a ubiquitin dependent pathway. In certain aspects, the chimeric polypeptide comprises the amino acid sequence set forth in SEQ ID NO: 62.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 62, wherein the chimeric polypeptide is capable of preferentially binding to the key polypeptide consisting of SEQ ID NO: 89, and upon binding to the key polypeptide, is capable of being degraded by a ubiquitin dependent pathway. In certain aspects, the chimeric polypeptide consists or consists essentially of the amino acid sequence set forth in SEQ ID NO: 62.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 63, wherein the chimeric polypeptide is capable of preferentially binding to the key polypeptide consisting of SEQ ID NO: 90, and upon binding to the key polypeptide, is capable of being degraded by a ubiquitin dependent pathway. In certain aspects, the chimeric polypeptide comprises the amino acid sequence set forth in SEQ ID NO: 63.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 63, wherein the chimeric polypeptide is capable of preferentially binding to the key polypeptide consisting of SEQ ID NO: 90, and upon binding to the key polypeptide, is capable of being degraded by a ubiquitin dependent pathway. In certain aspects, the chimeric polypeptide consists or consists essentially of the amino acid sequence set forth in SEQ ID NO: 63.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 64, wherein the chimeric polypeptide is capable of preferentially binding to the key polypeptide consisting of SEQ ID NO: 92, and upon binding to the key polypeptide, is capable of being degraded by a ubiquitin dependent pathway. In certain aspects, the chimeric polypeptide comprises the amino acid sequence set forth in SEQ ID NO: 64.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 64, wherein the chimeric polypeptide is capable of preferentially binding to the key polypeptide consisting of SEQ ID NO: 92, and upon binding to the key polypeptide, is capable of being degraded by a ubiquitin dependent pathway. In certain aspects, the chimeric polypeptide consists or consists essentially of the amino acid sequence set forth in SEQ ID NO: 64.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 65, wherein the chimeric polypeptide is capable of preferentially binding to the key polypeptide consisting of SEQ ID NO: 91, and upon binding to the key polypeptide, is capable of being degraded by a ubiquitin dependent pathway. In certain aspects, the chimeric polypeptide comprises the amino acid sequence set forth in SEQ ID NO: 65.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 65, wherein the chimeric polypeptide is capable of preferentially binding to the key polypeptide consisting of SEQ ID NO: 91, and upon binding to the key polypeptide, is capable of being degraded by a ubiquitin dependent pathway. In certain aspects, the chimeric polypeptide consists or consists essentially of the amino acid sequence set forth in SEQ ID NO: 65.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 66, wherein the chimeric polypeptide is capable of preferentially binding to the key polypeptide consisting of SEQ ID NO: 92, and upon binding to the key polypeptide, is capable of being degraded by a ubiquitin dependent pathway. In certain aspects, the chimeric polypeptide comprises the amino acid sequence set forth in SEQ ID NO: 66.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 66, wherein the chimeric polypeptide is capable of preferentially binding to the key polypeptide consisting of SEQ ID NO: 92, and upon binding to the key polypeptide, is capable of being degraded by a ubiquitin dependent pathway. In certain aspects, the chimeric polypeptide consists or consists essentially of the amino acid sequence set forth in SEQ ID NO: 66.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 67, wherein the chimeric polypeptide is capable of preferentially binding to the key polypeptide consisting of SEQ ID NO: 92, and upon binding to the key polypeptide, is capable of being degraded by a ubiquitin dependent pathway. In certain aspects, the chimeric polypeptide comprises the amino acid sequence set forth in SEQ ID NO: 67.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 67, wherein the chimeric polypeptide is capable of preferentially binding to the key polypeptide consisting of SEQ ID NO: 92, and upon binding to the key polypeptide, is capable of being degraded by a ubiquitin dependent pathway. In certain aspects, the chimeric polypeptide consists or consists essentially of the amino acid sequence set forth in SEQ ID NO: 67.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 68, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide comprises the amino acid sequence set forth in SEQ ID NO: 68.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 68, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide consists or consists essentially of the amino acid sequence set forth in SEQ ID NO: 68.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 69, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide comprises the amino acid sequence set forth in SEQ ID NO: 69.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 69, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide consists or consists essentially of the amino acid sequence set forth in SEQ ID NO: 69.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 70, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide comprises the amino acid sequence set forth in SEQ ID NO: 70.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 70, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide consists or consists essentially of the amino acid sequence set forth in SEQ ID NO: 70.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 71, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide comprises the amino acid sequence set forth in SEQ ID NO: 71.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 71, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide consists or consists essentially of the amino acid sequence set forth in SEQ ID NO: 71.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 72, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide comprises the amino acid sequence set forth in SEQ ID NO: 72.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 72, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide consists or consists essentially of the amino acid sequence set forth in SEQ ID NO: 72.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 73, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide comprises the amino acid sequence set forth in SEQ ID NO: 73.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 73, wherein the chimeric polypeptide is capable of preferentially binding to the key polypeptide consisting of SEQ ID NO: 93, and upon binding to the key polypeptide, is capable of being degraded by a ubiquitin dependent pathway. In certain aspects, the chimeric polypeptide consists or consists essentially of the amino acid sequence set forth in SEQ ID NO: 73.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 74, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide comprises the amino acid sequence set forth in SEQ ID NO: 74.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 74, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide consists or consists essentially of the amino acid sequence set forth in SEQ ID NO: 74.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 75, wherein the chimeric polypeptide is capable of preferentially binding to the key polypeptide consisting of SEQ ID NO: 95, and upon binding to the key polypeptide, is capable of being degraded by a ubiquitin dependent pathway. In certain aspects, the chimeric polypeptide comprises the amino acid sequence set forth in SEQ ID NO: 75.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 75, wherein the chimeric polypeptide is capable of preferentially binding to the key polypeptide consisting of SEQ ID NO: 95, and upon binding to the key polypeptide, is capable of being degraded by a ubiquitin dependent pathway. In certain aspects, the chimeric polypeptide consists or consists essentially of the amino acid sequence set forth in SEQ ID NO: 75.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 76, wherein the chimeric polypeptide is capable of preferentially binding to the key polypeptide consisting of SEQ ID NO: 96, and upon binding to the key polypeptide, is capable of being degraded by a ubiquitin dependent pathway. In certain aspects, the chimeric polypeptide comprises the amino acid sequence set forth in SEQ ID NO: 76.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 76, wherein the chimeric polypeptide is capable of preferentially binding to the key polypeptide consisting of SEQ ID NO: 96, and upon binding to the key polypeptide, is capable of being degraded by a ubiquitin dependent pathway. In certain aspects, the chimeric polypeptide consists or consists essentially of the amino acid sequence set forth in SEQ ID NO: 76.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 807, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 807.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 807, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 807.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 808, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 808.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 808, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 808.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 809, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 809.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 809, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 809.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 810, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide comprises the amino acid sequence set forth in SEQ ID NO: 810.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 810, wherein the chimeric polypeptide is capable of preferentially binding to the key polypeptide consisting of SEQ ID NO: 81, and upon binding to the key polypeptide, is capable of being degraded by a ubiquitin dependent pathway. In certain aspects, the chimeric polypeptide consists or consists essentially of the amino acid sequence set forth in SEQ ID NO: 810.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 811, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 811.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 811, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 811.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 812, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 812.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 812, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 812.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 813, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide comprises the amino acid sequence set forth in SEQ ID NO: 813.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 813, wherein the chimeric polypeptide is capable of preferentially binding to the key polypeptide consisting of SEQ ID NO: 83, and upon binding to the key polypeptide, is capable of being degraded by a ubiquitin dependent pathway. In certain aspects, the chimeric polypeptide consists or consists essentially of the amino acid sequence set forth in SEQ ID NO: 813.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 814, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 814.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 814, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 814.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 815, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide comprises the amino acid sequence set forth in SEQ ID NO: 815.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 815, wherein the chimeric polypeptide is capable of preferentially binding to the key polypeptide consisting of SEQ ID NO: 82, and upon binding to the key polypeptide, is capable of being degraded by a ubiquitin dependent pathway. In certain aspects, the chimeric polypeptide consists or consists essentially of the amino acid sequence set forth in SEQ ID NO: 815.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 816, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide comprises the amino acid sequence set forth in SEQ ID NO: 816.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 816, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide consists or consists essentially of the amino acid sequence set forth in SEQ ID NO: 816.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 817, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide comprises the amino acid sequence set forth in SEQ ID NO: 817.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 817, wherein the chimeric polypeptide is capable of preferentially binding to the key polypeptide consisting of SEQ ID NO: 94, and upon binding to the key polypeptide, is capable of being degraded by a ubiquitin dependent pathway. In certain aspects, the chimeric polypeptide consists or consists essentially of the amino acid sequence set forth in SEQ ID NO: 817.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 818, wherein the chimeric polypeptide is capable of preferentially binding to the key polypeptide consisting of SEQ ID NO: 80, and upon binding to the key polypeptide, is capable of being degraded by a ubiquitin dependent pathway. In certain aspects, the chimeric polypeptide comprises the amino acid sequence set forth in SEQ ID NO: 818.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 818, wherein the chimeric polypeptide is capable of preferentially binding to the key polypeptide consisting of SEQ ID NO: 80, and upon binding to the key polypeptide, is capable of being degraded by a ubiquitin dependent pathway. In certain aspects, the chimeric polypeptide consists or consists essentially of the amino acid sequence set forth in SEQ ID NO: 818.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 819, wherein the chimeric polypeptide is capable of preferentially binding to the key polypeptide consisting of SEQ ID NO: 80, and upon binding to the key polypeptide, is capable of being degraded by a ubiquitin dependent pathway. In certain aspects, the chimeric polypeptide comprises the amino acid sequence set forth in SEQ ID NO: 819.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 819, wherein the chimeric polypeptide is capable of preferentially binding to the key polypeptide consisting of SEQ ID NO: 80, and upon binding to the key polypeptide, is capable of being degraded by a ubiquitin dependent pathway. In certain aspects, the chimeric polypeptide consists or consists essentially of the amino acid sequence set forth in SEQ ID NO: 819.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 820, wherein the chimeric polypeptide is capable of preferentially binding to the key polypeptide consisting of SEQ ID NO: 88, and upon binding to the key polypeptide, is capable of being degraded by a ubiquitin dependent pathway. In some aspects, the chimeric polypeptide comprises the amino acid sequence set forth in SEQ ID NO: 820.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 820, wherein the chimeric polypeptide is capable of preferentially binding to the key polypeptide consisting of SEQ ID NO: 88, and upon binding to the key polypeptide, is capable of being degraded by a ubiquitin dependent pathway. In certain aspects, the chimeric polypeptide consists or consists essentially of the amino acid sequence set forth in SEQ ID NO: 820.

In some aspects, a chimeric polypeptide of the present disclosure comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 821, wherein the chimeric polypeptide is capable of preferentially binding to the key polypeptide consisting of SEQ ID NO: 88, and upon binding to the key polypeptide, is capable of being degraded by a ubiquitin dependent pathway. In certain aspects, the chimeric polypeptide comprises the amino acid sequence set forth in SEQ ID NO: 821.

In some aspects, a chimeric polypeptide of the present disclosure consists or consist essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 821, wherein the chimeric polypeptide is capable of preferentially binding to the key polypeptide consisting of SEQ ID NO: 88, and upon binding to the key polypeptide, is capable of being degraded by a ubiquitin dependent pathway. In certain aspects, the chimeric polypeptide consists or consists essentially of the amino acid sequence set forth in SEQ ID NO: 821.

In some aspects, the present disclosure comprises a chimeric polypeptide which comprises a cage polypeptide comprising:
(i) a structural region comprising an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO:191) and/or helix 3 (SEQ ID NO:192) of Cage1 (SEQ ID NO:2), helix 1 (SEQ ID NO: 190) and/or helix 2 (SEQ ID NO: 191) of Cage1 (SEQ ID NO:2), helix 2 (SEQ ID NO: 194) and/or helix 3 (SEQ ID NO: 195) of Cage2 (SEQ ID NO:3), helix 1 (SEQ ID NO: 193) and/or helix 2 (SEQ ID NO: 194) of Cage2 (SEQ ID NO:3), helix 2 (SEQ ID NO: 197) and/or helix 3 (SEQ ID NO: 198) of Cage3 (SEQ ID NO:4), helix 1 (SEQ ID NO: 196) and/or helix 2 (SEQ ID NO: 197) of Cage3 (SEQ ID NO:4), helix 2 (SEQ ID NO: 200) and/or helix 3 (SEQ ID NO: 201) of Cage4 (SEQ ID NO:5), helix 1 (SEQ ID NO: 199) and/or helix 2 (SEQ ID NO: 200) of Cage4 (SEQ ID NO:5), helix 2 (SEQ ID NO: 203), helix 3 (SEQ ID NO: 204), and/or helix 4 (SEQ ID NO: 205) of Cage5 (SEQ ID NO:6), helix 1 (SEQ ID NO: 202), helix 2 (SEQ ID NO: 203), and/or helix 3 (SEQ ID NO: 204) of Cage5 (SEQ ID NO:6), helix 2 (SEQ ID NO: 207), helix 3 (SEQ ID NO: 208), and/or helix 4 (SEQ ID NO: 209) of Cage6 (SEQ ID NO:7), helix 1 (SEQ ID NO: 206), helix 2 (SEQ ID NO: 207), and/or helix 3 (SEQ ID NO: 208) of Cage6 (SEQ ID NO:7), helix 2 (SEQ ID NO: 211), helix 3 (SEQ ID NO: 212), and/or helix 4 (SEQ ID NO: 213) of Cage7 (SEQ ID NO: 8), helix 1 (SEQ ID NO: 210), helix 2 (SEQ ID NO: 211), and/or helix 3 (SEQ ID NO: 212) of Cage7 (SEQ ID NO: 8), helix 2 (SEQ ID NO: 215), helix 3 (SEQ ID NO: 216), and/or helix 4 (SEQ ID NO: 217) of Cage8 (SEQ ID NO:9), helix 1 (SEQ ID NO: 214), helix 2 (SEQ ID NO: 215), and/or helix 3 (SEQ ID NO: 216) of Cage8 (SEQ ID NO:9), helix 2 (SEQ ID NO: 219), helix 3 (SEQ ID NO: 220), and/or helix 4 (SEQ ID NO: 221) of Cage9 (SEQ ID NO:10), helix 1 (SEQ ID NO: 218), helix 2 (SEQ ID NO: 219), and/or helix 3 (SEQ ID NO: 220) of Cage9 (SEQ ID NO:10), helix 2 (SEQ ID NO: 223), helix 3 (SEQ ID NO: 224), and/or helix 4 (SEQ ID NO: 225) of Cage10 (SEQ ID NO:11), helix 1 (SEQ ID NO: 222), helix 2 (SEQ ID NO: 223), and/or helix 3 (SEQ ID NO: 224) of Cage10 (SEQ ID NO:11), helix 2 (SEQ ID NO: 227), helix 3 (SEQ ID NO: 228), and/or helix 4 (SEQ ID NO: 229) of Cage11 (SEQ ID NO:12), helix 1 (SEQ ID NO: 226), helix 2 (SEQ ID NO: 227), and/or helix 3 (SEQ ID NO: 228) of Cage11 (SEQ ID NO:12), helix 2 (SEQ ID NO: 231), helix 3 (SEQ ID NO: 232), and/or helix 4 (SEQ ID NO: 233) of Cage12 (SEQ ID NO:13), helix 1 (SEQ ID NO: 230), helix 2 (SEQ ID NO: 231), and/or helix 3 (SEQ ID NO: 232) of Cage12 (SEQ ID NO:13), helix 1 (SEQ ID NO: 868), helix 2 (SEQ ID NO: 869), and/or helix 3 (SEQ ID NO: 870) of Cage13 (SEQ ID NO: 823), helix 1 (SEQ ID NO: 871), and/or helix 2 (SEQ ID NO: 872) of Cage14 (SEQ ID NO: 824), helix 1 (SEQ ID NO: 873) and/or helix 2 (SEQ ID NO: 874) of Cage15 (SEQ ID NO: 825), helix 1 (SEQ ID NO: 875) and/or helix 2 (SEQ ID NO: 876) of Cage16 (SEQ ID NO: 826), helix 1 (SEQ ID NO: 877) and/or helix 2 (SEQ ID NO: 878) of Cage17 (SEQ ID NO: 827), helix 1 (SEQ ID NO: 879) and/or helix 2 (SEQ ID NO: 880) of Cage18 (SEQ ID NO: 828), helix 1 (SEQ ID NO: 881) and/or helix 2 (SEQ ID NO: 882) of Cage19 (SEQ ID NO: 829), helix 1 (SEQ ID NO: 883) and/or helix 2 (SEQ ID NO: 884) of Cage20 (SEQ ID NO: 830), helix 1 (SEQ ID NO: 885) and/or helix 2 (SEQ ID NO: 886) of Cage21 (SEQ ID NO: 831), helix 1 (SEQ ID NO: 887), helix 2 (SEQ ID NO: 888), and/or helix 3 (SEQ ID NO: 889) of Cage22 (SEQ ID NO: 832), helix 1 (SEQ ID NO: 890), helix 2 (SEQ ID NO: 891), and/or helix 3 (SEQ ID NO: 892) of Cage23 (SEQ ID NO: 833), helix 1 (SEQ ID NO: 893) and/or helix 2 (SEQ ID NO: 894) of Cage24 (SEQ ID NO: 834), helix 1 (SEQ ID NO: 895) and/or helix 2 (SEQ ID NO: 896) of Cage25 (SEQ ID NO: 835), helix 1 (SEQ ID NO: 897), helix 2 (SEQ ID NO: 898), and/or helix 3 (SEQ ID NO: 899) of Cage26 (SEQ ID NO: 836), helix 1 (SEQ ID NO:900), helix 2 (SEQ ID NO:901), and/or helix 3 (SEQ ID NO:902) of Cage27 (SEQ ID NO: 837); and,
(ii) a latch region comprising a degron peptide consisting of the amino acid sequence as set forth in SEQ ID NOS: 1, 110-141, or SEQ ID NOS: 680-804, or SEQ ID NOS: 918-982, wherein the latch region is capable of binding to the structural region.

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 191) and/or helix 3 (SEQ ID NO: 192) of Cage1 (SEQ ID NO:2). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 190) and/or helix 2 (SEQ ID NO: 191) of Cage1 (SEQ ID NO:2). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 194) and/or helix 3 (SEQ ID NO: 195) of Cage2 (SEQ ID NO:3). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 193) and/or helix 2 (SEQ ID NO: 194) of Cage2 (SEQ ID NO:3). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 197) and/or helix 3 (SEQ ID NO: 198) of Cage3 (SEQ ID NO:4). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 196) and/or helix 2 (SEQ ID NO: 197) of Cage3 (SEQ ID NO:4). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 200) and/or helix 3 (SEQ ID NO: 201) of Cage4 (SEQ ID NO:5). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 199) and/or helix 2 (SEQ ID NO: 200) of Cage4 (SEQ ID NO:5). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 203), helix 3 (SEQ ID NO: 204), and/or helix 4 (SEQ ID NO: 205) of Cage5 (SEQ ID NO:6). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 202), helix 2 (SEQ ID NO: 203), and/or helix 3 (SEQ ID NO: 204) of Cage5 (SEQ ID NO:6). In some aspects, the structural region comprises an amino acid sequence having at least about 50%, at least about 55%, at least about 60%, a least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 207), helix 3 (SEQ ID NO: 208), and/or helix 4 (SEQ ID NO: 209) of Cage6 (SEQ ID NO:7). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 206), helix 2 (SEQ ID NO: 207), and/or helix 3 (SEQ ID NO: 208) of Cage6 (SEQ ID NO:7). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 211), helix 3 (SEQ ID NO: 212), and/or helix 4 (SEQ ID NO: 213) of Cage7 (SEQ ID NO: 8). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 210), helix 2 (SEQ ID NO: 211), and/or helix 3 (SEQ ID NO: 212) of Cage7 (SEQ ID NO: 8). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 215), helix 3 (SEQ ID NO: 216), and/or helix 4 (SEQ ID NO: 217) of Cage8 (SEQ ID NO:9). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 214), helix 2 (SEQ ID NO: 215), and/or helix 3 (SEQ ID NO: 216) of Cage8 (SEQ ID NO:9). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 219), helix 3 (SEQ ID NO: 220), and/or helix 4 (SEQ ID NO: 221) of Cage9 (SEQ ID NO:10). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 218), helix 2 (SEQ ID NO: 219), and/or helix 3 (SEQ ID NO: 220) of Cage9 (SEQ ID NO:10). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 223), helix 3 (SEQ ID NO: 224), and/or helix 4 (SEQ ID NO: 225) of Cage10 (SEQ ID NO:11). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 222), helix 2 (SEQ ID NO: 223), and/or helix 3 (SEQ ID NO: 224) of Cage10 (SEQ ID NO:11). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 227), helix 3 (SEQ ID NO: 228), and/or helix 4 (SEQ ID NO: 229) of Cage11 (SEQ ID NO:12). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 226), helix 2 (SEQ ID NO: 227), and/or helix 3 (SEQ ID NO: 228) of Cage11 (SEQ ID NO:12). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 2 (SEQ ID NO: 231), helix 3 (SEQ ID NO: 232), and/or helix 4 (SEQ ID NO: 233) of Cage12 (SEQ ID NO:13). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 230), helix 2 (SEQ ID NO: 231), and/or helix 3 (SEQ ID NO: 232) of Cage12 (SEQ ID NO:13).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 868), helix 2 (SEQ ID NO: 869), and/or helix 3 (SEQ ID NO: 870) of Cage13 (SEQ ID NO: 823). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 868), helix 2 (SEQ ID NO: 869), and/or helix 3 (SEQ ID NO: 870) of Cage13 (SEQ ID NO: 823).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 871), and/or helix 2 (SEQ ID NO: 872) of Cage14 (SEQ ID NO: 824). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 871), and/or helix 2 (SEQ ID NO: 872) of Cage14 (SEQ ID NO: 824).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 873) and/or helix 2 (SEQ ID NO: 874) of Cage15 (SEQ ID NO: 825). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 873) and/or helix 2 (SEQ ID NO: 874) of Cage15 (SEQ ID NO: 825).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 875) and/or helix 2 (SEQ ID NO: 876) of Cage16 (SEQ ID NO: 826). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 875) and/or helix 2 (SEQ ID NO: 876) of Cage16 (SEQ ID NO: 826).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 877) and/or helix 2 (SEQ ID NO: 878) of Cage17 (SEQ ID NO: 827). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 877) and/or helix 2 (SEQ ID NO: 878) of Cage17 (SEQ ID NO: 827).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 879) and/or helix 2 (SEQ ID NO: 880) of Cage18 (SEQ ID NO: 828). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 879) and/or helix 2 (SEQ ID NO: 880) of Cage18 (SEQ ID NO: 828).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 881) and/or helix 2 (SEQ ID NO: 882) of Cage19 (SEQ ID NO: 829). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 881) and/or helix 2 (SEQ ID NO: 882) of Cage19 (SEQ ID NO: 829).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 883) and/or helix 2 (SEQ ID NO: 884) of Cage20 (SEQ ID NO: 830). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 883) and/or helix 2 (SEQ ID NO: 884) of Cage20 (SEQ ID NO: 830).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 885) and/or helix 2 (SEQ ID NO: 886) of Cage21 (SEQ ID NO: 831). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 885) and/or helix 2 (SEQ ID NO: 886) of Cage21 (SEQ ID NO: 831).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 887), helix 2 (SEQ ID NO: 888), and/or helix 3 (SEQ ID NO: 889) of Cage22 (SEQ ID NO: 832). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 887), helix 2 (SEQ ID NO: 888), and/or helix 3 (SEQ ID NO: 889) of Cage22 (SEQ ID NO: 832).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 890), helix 2 (SEQ ID NO: 891), and/or helix 3 (SEQ ID NO: 892) of Cage23 (SEQ ID NO: 833). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 890), helix 2 (SEQ ID NO: 891), and/or helix 3 (SEQ ID NO: 892) of Cage23 (SEQ ID NO: 833).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 893) and/or helix 2 (SEQ ID NO: 894) of Cage24 (SEQ ID NO: 834). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 893) and/or helix 2 (SEQ ID NO: 894) of Cage24 (SEQ ID NO: 834).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 895) and/or helix 2 (SEQ ID NO: 896) of Cage25 (SEQ ID NO: 835). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 895) and/or helix 2 (SEQ ID NO: 896) of Cage25 (SEQ ID NO: 835).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 897), helix 2 (SEQ ID NO: 898), and/or helix 3 (SEQ ID NO: 899) of Cage26 (SEQ ID NO: 836). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 897), helix 2 (SEQ ID NO: 898), and/or helix 3 (SEQ ID NO: 899) of Cage26 (SEQ ID NO: 836).

In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO:900), helix 2 (SEQ ID NO:901), and/or helix 3 (SEQ ID NO:902) of Cage27 (SEQ ID NO: 837). In some aspects, the structural region comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO:900), helix 2 (SEQ ID NO:901), and/or helix 3 (SEQ ID NO:902) of Cage27 (SEQ ID NO: 837).

In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 191) and/or at least about 10, at least about 15, at least about 20, at least about 25, at least about 30, at least about 35, at least about 40, at least about 45, or at least about 50 consecutive amino acids of helix 3 (SEQ ID NO: 192) of Cage1 (SEQ ID NO:2). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 190) and/or at least about 10, at least about 15, at least about 20, at least about 25, at least about 30, at least about 35, at least about 40, at least about 45, or at least about 50 consecutive amino acids of helix 2 (SEQ ID NO: 191) of Cage1 (SEQ ID NO:2). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 194) and/or at least about 10, at least about 15, at least about 20, at least about 25, at least about 30, at least about 35, at least about 40, at least about 45, or at least about 50 consecutive amino acids of helix 3 (SEQ ID NO: 195) of Cage2 (SEQ ID NO:3). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 193) and/or at least about 10, at least about 15, at least about 20, at least about 25, at least about 30, at least about 35, at least about 40, at least about 45, or at least about 50 consecutive amino acids of helix 2 (SEQ ID NO: 194) of Cage2 (SEQ ID NO:3). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 197) and/or at least about 10, at least about 15, at least about 20, at least about 25, at least about 30, at least about 35, at least about 40, at least about 45, or at least about 50 consecutive amino acids of helix 3 (SEQ ID NO: 198) of Cage3 (SEQ ID NO:4). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 196) and/or at least about 10, at least about 15, at least about 20, at least about 25, at least about 30, at least about 35, at least about 40, at least about 45, or at least about 50 consecutive amino acids of helix 2 (SEQ ID NO: 197) of Cage3 (SEQ ID NO:4). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 200) and/or at least about 10, at least about 15, at least about 20, at least about 25, at least about 30, at least about 35, at least about 40, at least about 45, or at least about 50 consecutive amino acids of helix 3 (SEQ ID NO: 201) of Cage4 (SEQ ID NO:5). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 199) and/or at least about 10, at least about 15, at least about 20, at least about 25, at least about 30, at least about 35, at least about 40, at least about 45, or at least about 50 consecutive amino acids of helix 2 (SEQ ID NO: 200) of Cage4 (SEQ ID NO:5). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 203), at least about 10, at least about 15, at least about 20, at least about 25, at least about 30, at least about 35, at least about 40, at least about 45, or at least about 50 consecutive amino acids of helix 3 (SEQ ID NO: 204), and/or at least about 10, at least about 15, at least about 20, at least about 25, at least about 30, at least about 35, at least about 40, at least about 45, or at least about 50 consecutive amino acids of helix 4 (SEQ ID NO: 205) of Cage5 (SEQ ID NO:6). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 202), at least about 10, at least about 15, at least about 20, at least about 25, at least about 30, at least about 35, at least about 40, at least about 45, or at least about 50 consecutive amino acids of helix 2 (SEQ ID NO: 203), and/or at least about 10, at least about 15, at least about 20, at least about 25, at least about 30, at least about 35, at least about 40, at least about 45, or at least about 50 consecutive amino acids of helix 3 (SEQ ID NO: 204) of Cage5 (SEQ ID NO:6). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 207), at least about 10, at least about 15, at least about 20, at least about 25, at least about 30, at least about 35, at least about 40, at least about 45, or at least about 50 consecutive amino acids of helix 3 (SEQ ID NO: 208), and/or at least about 10, at least about 15, at least about 20, at least about 25, at least about 30, at least about 35, at least about 40, at least about 45, or at least about 50 consecutive amino acids of helix 4 (SEQ ID NO: 209) of Cage6 (SEQ ID NO:7). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 206), at least about 10, at least about 15, at least about 20, at least about 25, at least about 30, at least about 35, at least about 40, at least about 45, or at least about 50 consecutive amino acids of helix 2 (SEQ ID NO: 207), and/or at least about 10, at least about 15, at least about 20, at least about 25, at least about 30, at least about 35, at least about 40, at least about 45, or at least about 50 consecutive amino acids of helix 3 (SEQ ID NO: 208) of Cage6 (SEQ ID NO:7). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 211), at least about 10, at least about 15, at least about 20, at least about 25, at least about 30, at least about 35, at least about 40, at least about 45, or at least about 50 consecutive amino acids of helix 3 (SEQ ID NO: 212), and/or at least about 10, at least about 15, at least about 20, at least about 25, at least about 30, at least about 35, at least about 40, at least about 45, or at least about 50 consecutive amino acids of helix 4 (SEQ ID NO: 213) of Cage7 (SEQ ID NO: 8). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 210), at least about 10, at least about 15, at least about 20, at least about 25, at least about 30, at least about 35, at least about 40, at least about 45, or at least about 50 consecutive amino acids of helix 2 (SEQ ID NO: 211), and/or at least about 10, at least about 15, at least about 20, at least about 25, at least about 30, at least about 35, at least about 40, at least about 45, or at least about 50 consecutive amino acids of helix 3 (SEQ ID NO: 212) of Cage7 (SEQ ID NO: 8). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 215), at least about 10, at least about 15, at least about 20, at least about 25, at least about 30, at least about 35, at least about 40, at least about 45, or at least about 50 consecutive amino acids of helix 3 (SEQ ID NO: 216), and/or at least about 10, at least about 15, at least about 20, at least about 25, at least about 30, at least about 35, at least about 40, at least about 45, or at least about 50 consecutive amino acids of helix 4 (SEQ ID NO: 217) of Cage8 (SEQ ID NO:9). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 214), at least about 10, at least about 15, at least about 20, at least about 25, at least about 30, at least about 35, at least about 40, at least about 45, or at least about 50 consecutive amino acids of helix 2 (SEQ ID NO: 215), and/or at least about 10, at least about 15, at least about 20, at least about 25, at least about 30, at least about 35, at least about 40, at least about 45, or at least about 50 consecutive amino acids of helix 3 (SEQ ID NO: 216) of Cage8 (SEQ ID NO:9). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 219), at least about 10, at least about 15, at least about 20, at least about 25, at least about 30, at least about 35, at least about 40, at least about 45, or at least about 50 consecutive amino acids of helix 3 (SEQ ID NO: 220), and/or at least about 10, at least about 15, at least about 20, at least about 25, at least about 30, at least about 35, at least about 40, at least about 45, or at least about 50 consecutive amino acids of helix 4 (SEQ ID NO: 221) of Cage9 (SEQ ID NO:10). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 218), at least about 10, at least about 15, at least about 20, at least about 25, at least about 30, at least about 35, at least about 40, at least about 45, or at least about 50 consecutive amino acids of helix 2 (SEQ ID NO: 219), and/or at least about 10, at least about 15, at least about 20, at least about 25, at least about 30, at least about 35, at least about 40, at least about 45, or at least about 50 consecutive amino acids of helix 3 (SEQ ID NO: 220) of Cage9 (SEQ ID NO:10). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 223), at least about 10, at least about 15, at least about 20, at least about 25, at least about 30, at least about 35, at least about 40, at least about 45, or at least about 50 consecutive amino acids of helix 3 (SEQ ID NO: 224), and/or at least about 10, at least about 15, at least about 20, at least about 25, at least about 30, at least about 35, at least about 40, at least about 45, or at least about 50 consecutive amino acids of helix 4 (SEQ ID NO: 225) of Cage10 (SEQ ID NO:11). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 222), at least about 10, at least about 15, at least about 20, at least about 25, at least about 30, at least about 35, at least about 40, at least about 45, or at least about 50 consecutive amino acids of helix 2 (SEQ ID NO: 223), and/or at least about 10, at least about 15, at least about 20, at least about 25, at least about 30, at least about 35, at least about 40, at least about 45, or at least about 50 consecutive amino acids of helix 3 (SEQ ID NO: 224) of Cage10 (SEQ ID NO:11). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 227), at least about 10, at least about 15, at least about 20, at least about 25, at least about 30, at least about 35, at least about 40, at least about 45, or at least about 50 consecutive amino acids of helix 3 (SEQ ID NO: 228), and/or at least about 10, at least about 15, at least about 20, at least about 25, at least about 30, at least about 35, at least about 40, at least about 45, or at least about 50 consecutive amino acids of helix 4 (SEQ ID NO: 229) of Cage11 (SEQ ID NO:12). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 226), at least about 10, at least about 15, at least about 20, at least about 25, at least about 30, at least about 35, at least about 40, at least about 45, or at least about 50 consecutive amino acids of helix 2 (SEQ ID NO: 227), and/or at least about 10, at least about 15, at least about 20, at least about 25, at least about 30, at least about 35, at least about 40, at least about 45, or at least about 50 consecutive amino acids of helix 3 (SEQ ID NO: 228) of Cage11 (SEQ ID NO:12). In some aspects, the structural region comprises at least about 10, at least about 15, at least about 20, at least about 25, at least about 30, at least about 35, at least about 40, at least about 45, or at least about 50 consecutive amino acids of helix 2 (SEQ ID NO: 231), at least about 10, at least about 15, at least about 20, at least about 25, at least about 30, at least about 35, at least about 40, at least about 45, or at least about 50 consecutive amino acids of helix 3 (SEQ ID NO: 232), and/or at least about 10, at least about 15, at least about 20, at least about 25, at least about 30, at least about 35, at least about 40, at least about 45, or at least about 50 consecutive amino acids of helix 4 (SEQ ID NO: 233) of Cage12 (SEQ ID NO:13). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 230), at least about 10, at least about 15, at least about 20, at least about 25, at least about 30, at least about 35, at least about 40, at least about 45, or at least about 50 consecutive amino acids of helix 2 (SEQ ID NO: 231), and/or at least about 10, at least about 15, at least about 20, at least about 25, at least about 30, at least about 35, at least about 40, at least about 45, or at least about 50 consecutive amino acids of helix 3 (SEQ ID NO: 232) of Cage12 (SEQ ID NO:13).

In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 868), at least about 10, at least about 15, at least about 20, at least about 25, at least about 30, at least about 35, at least about 40, at least about 45, or at least about 50 consecutive amino acids of helix 2 (SEQ ID NO: 869), and/or at least about 10, at least about 15, at least about 20, at least about 25, at least about 30, at least about 35, at least about 40, at least about 45, or at least about 50 consecutive amino acids of helix 3 (SEQ ID NO: 870) of Cage13 (SEQ ID NO: 823). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 871) and/or at least about 10, at least about 15, at least about 20, at least about 25, at least about 30, at least about 35, at least about 40, at least about 45, or at least about 50 consecutive amino acids of helix 2 (SEQ ID NO: 872) of Cage14 (SEQ ID NO: 824). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 873) and/or at least about 10, at least about 15, at least about 20, at least about 25, at least about 30, at least about 35, at least about 40, at least about 45, or at least about 50 consecutive amino acids of helix 2 (SEQ ID NO: 874) of Cage15 (SEQ ID NO: 825). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 875) and/or at least about 10, at least about 15, at least about 20, at least about 25, at least about 30, at least about 35, at least about 40, at least about 45, or at least about 50 consecutive amino acids of helix 2 (SEQ ID NO: 876) of Cage16 (SEQ ID NO: 826). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 877) and/or at least about 10, at least about 15, at least about 20, at least about 25, at least about 30, at least about 35, at least about 40, at least about 45, or at least about 50 consecutive amino acids of helix 2 (SEQ ID NO: 878) of Cage17 (SEQ ID NO: 827). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 879) and/or at least about 10, at least about 15, at least about 20, at least about 25, at least about 30, at least about 35, at least about 40, at least about 45, or at least about 50 consecutive amino acids of helix 2 (SEQ ID NO: 880) of Cage18 (SEQ ID NO: 828). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 881) and/or at least about 10, at least about 15, at least about 20, at least about 25, at least about 30, at least about 35, at least about 40, at least about 45, or at least about 50 consecutive amino acids of helix 2 (SEQ ID NO: 882) of Cage19 (SEQ ID NO: 829). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 883) and/or at least about 10, at least about 15, at least about 20, at least about 25, at least about 30, at least about 35, at least about 40, at least about 45, or at least about 50 consecutive amino acids of helix 2 (SEQ ID NO: 884) of Cage20 (SEQ ID NO: 830). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 885) and/or at least about 10, at least about 15, at least about 20, at least about 25, at least about 30, at least about 35, at least about 40, at least about 45, or at least about 50 consecutive amino acids of helix 2 (SEQ ID NO: 886) of Cage21 (SEQ ID NO: 831). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 887), at least about 10, at least about 15, at least about 20, at least about 25, at least about 30, at least about 35, at least about 40, at least about 45, or at least about 50 consecutive amino acids of helix 2 (SEQ ID NO: 888), and/or at least about 10, at least about 15, at least about 20, at least about 25, at least about 30, at least about 35, at least about 40, at least about 45, or at least about 50 consecutive amino acids of helix 3 (SEQ ID NO: 889) of Cage22 (SEQ ID NO: 832). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 890), at least about 10, at least about 15, at least about 20, at least about 25, at least about 30, at least about 35, at least about 40, at least about 45, or at least about 50 consecutive amino acids of helix 2 (SEQ ID NO: 891), and/or at least about 10, at least about 15, at least about 20, at least about 25, at least about 30, at least about 35, at least about 40, at least about 45, or at least about 50 consecutive amino acids of helix 3 (SEQ ID NO: 892) of Cage23 (SEQ ID NO: 833). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 893) and/or at least about 10, at least about 15, at least about 20, at least about 25, at least about 30, at least about 35, at least about 40, at least about 45, or at least about 50 consecutive amino acids of helix 2 (SEQ ID NO: 894) of Cage24 (SEQ ID NO: 834). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 895) and/or at least about 10, at least about 15, at least about 20, at least about 25, at least about 30, at least about 35, at least about 40, at least about 45, or at least about 50 consecutive amino acids of helix 2 (SEQ ID NO: 896) of Cage25 (SEQ ID NO: 835). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO: 897), at least about 10, at least about 15, at least about 20, at least about 25, at least about 30, at least about 35, at least about 40, at least about 45, or at least about 50 consecutive amino acids of helix 2 (SEQ ID NO: 898), and/or at least about 10, at least about 15, at least about 20, at least about 25, at least about 30, at least about 35, at least about 40, at least about 45, or at least about 50 consecutive amino acids of helix 3 (SEQ ID NO: 899) of Cage26 (SEQ ID NO: 836). In some aspects, the structural region comprises at least about 10, at least about 15, at least about 20, at least about 25, at least about 30, at least about 35, at least about 40, at least about 45, or at least about 50 consecutive amino acids of helix 1 (SEQ ID NO:900), at least about 10, at least about 15, at least about 20, at least about 25, at least about 30, at least about 35, at least about 40, at least about 45, or at least about 50 consecutive amino acids of helix 2 (SEQ ID NO:901), and/or at least about 10, at least about 15, at least about 20, at least about 25, at least about 30, at least about 35, at least about 40, at least about 45, or at least about 50 consecutive amino acids of helix 3 (SEQ ID NO:902) of Cage27 (SEQ ID NO: 837).

In some aspects, the latch region comprises at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to helix 1 (SEQ ID NO: 190) of Cage1 (SEQ ID NO:2), helix 3 (SEQ ID NO: 192) of Cage1 (SEQ ID NO:2), helix 1 (SEQ ID NO: 193) of Cage2 (SEQ ID NO:3), helix 3 (SEQ ID NO: 195) of Cage2 (SEQ ID NO:3), helix 1 (SEQ ID NO: 196) of Cage3 (SEQ ID NO:4), helix 3 (SEQ ID NO: 198) of Cage3 (SEQ ID NO:4), helix 1 (SEQ ID NO: 199) of Cage4 (SEQ ID NO:5), helix 3 (SEQ ID NO: 201) of Cage4 (SEQ ID NO:5), helix 1 (SEQ ID NO: 202) of Cage5 (SEQ ID NO:6), helix 4 (SEQ ID NO: 205) of Cage5 (SEQ ID NO:6), helix 1 (SEQ ID NO: 206) of Cage6 (SEQ ID NO:7), helix 4 (SEQ ID NO: 209) of Cage6 (SEQ ID NO:7), helix 1 (SEQ ID NO: 210) of Cage7 (SEQ ID NO: 8), helix 4 (SEQ ID NO: 213) of Cage7 (SEQ ID NO: 8), helix 1 (SEQ ID NO: 214) of Cage8 (SEQ ID NO:9), helix 4 (SEQ ID NO: 217) of Cage8 (SEQ ID NO:9), helix 1 (SEQ ID NO: 218) of Cage9 (SEQ ID NO:10), helix 4 (SEQ ID NO: 221) of Cage9 (SEQ ID NO:10), helix 1 (SEQ ID NO: 222) of Cage10 (SEQ ID NO: 11), helix 4 (SEQ ID NO: 225) of Cage10 (SEQ ID NO: 11), helix 1 (SEQ ID NO: 226) of Cage11 (SEQ ID NO:12), helix 4 (SEQ ID NO: 229) of Cage11 (SEQ ID NO:12), helix 1 (SEQ ID NO: 230) of Cage12 (SEQ ID NO:13), or helix 4 (SEQ ID NO: 233) of Cage12 (SEQ ID NO: 13).

In some aspects, the latch region comprises at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to Cage 13 Latch helix (SEQ ID NO: 903), Cage 14 Latch helix (SEQ ID NO: 904), Cage 15 Latch helix (SEQ ID NO: 905), Cage 16 Latch helix (SEQ ID NO: 906), Cage 17 Latch helix (SEQ ID NO: 907), Cage 18 Latch helix (SEQ ID NO: 908), Cage 19 Latch helix (SEQ ID NO: 909), Cage 20 Latch helix (SEQ ID NO: 910), Cage 21 Latch helix (SEQ ID NO: 911), Cage 22 Latch helix (SEQ ID NO: 912), Cage 23 Latch helix (SEQ ID NO: 913), Cage 24 Latch helix (SEQ ID NO: 914), Cage 25 Latch helix (SEQ ID NO: 915), Cage 26 Latch helix (SEQ ID NO: 916), or Cage 27 Latch helix (SEQ ID NO: 917).

In some aspects, the latch region that corresponds to each structural region comprises at least 3, at least 4, at least about 5, at least about 10, at least about 15, at least about 20, at least about 25, at least about 30, at least about 35, at least about 40, at least about 45, or at least about 50 consecutive amino acids of helix 1 (SEQ ID NO: 190) of Cage1 (SEQ ID NO:2), helix 3 (SEQ ID NO: 192) of Cage1 (SEQ ID NO:2), helix 1 (SEQ ID NO: 193) of Cage2 (SEQ ID NO:3), helix 3 (SEQ ID NO: 195) of Cage2 (SEQ ID NO:3), helix 1 (SEQ ID NO: 196) of Cage3 (SEQ ID NO:4), helix 3 (SEQ ID NO: 198) of Cage3 (SEQ ID NO:4), helix 1 (SEQ ID NO: 199) of Cage4 (SEQ ID NO:5), helix 3 (SEQ ID NO: 201) of Cage4 (SEQ ID NO:5), helix 1 (SEQ ID NO: 202) of Cage5 (SEQ ID NO:6), helix 4 (SEQ ID NO: 205) of Cage5 (SEQ ID NO:6), helix 1 (SEQ ID NO: 206) of Cage6 (SEQ ID NO:7), helix 4 (SEQ ID NO: 209) of Cage6 (SEQ ID NO:7), helix 1 (SEQ ID NO: 210) of Cage7 (SEQ ID NO: 8), helix 4 (SEQ ID NO: 213) of Cage7 (SEQ ID NO: 8), helix 1 (SEQ ID NO: 214) of Cage8 (SEQ ID NO:9), helix 4 (SEQ ID NO: 217) of Cage8 (SEQ ID NO:9), helix 1 (SEQ ID NO: 218) of Cage9 (SEQ ID NO:10), helix 4 (SEQ ID NO: 221) of Cage9 (SEQ ID NO:10), helix 1 (SEQ ID NO: 222) of Cage10 (SEQ ID NO:11), helix 4 (SEQ ID NO: 225) of Cage10 (SEQ ID NO:11), helix 1 (SEQ ID NO: 226) of Cage11 (SEQ ID NO:12), helix 4 (SEQ ID NO: 229) of Cage11 (SEQ ID NO:12), helix 1 (SEQ ID NO: 230) of Cage12 (SEQ ID NO:13), or helix 4 (SEQ ID NO: 233) of Cage12 (SEQ ID NO:13).

In some aspects, the latch region that corresponds to each structural region comprises at least 3, at least 4, at least about 5, at least about 10, at least about 15, at least about 20, at least about 25, at least about 30, at least about 35, at least about 40, at least about 45, or at least about 50 consecutive amino acids of Cage 13 Latch helix (SEQ ID NO: 903), Cage 14 Latch helix (SEQ ID NO: 904), Cage 15 Latch helix (SEQ ID NO: 905), Cage 16 Latch helix (SEQ ID NO: 906), Cage 17 Latch helix (SEQ ID NO: 907), Cage 18 Latch helix (SEQ ID NO: 908), Cage 19 Latch helix (SEQ ID NO: 909), Cage 20 Latch helix (SEQ ID NO: 910), Cage 21 Latch helix (SEQ ID NO: 911), Cage 22 Latch helix (SEQ ID NO: 912), Cage 23 Latch helix (SEQ ID NO: 913), Cage 24 Latch helix (SEQ ID NO: 914), Cage 25 Latch helix (SEQ ID NO: 915), Cage 26 Latch helix (SEQ ID NO: 916), or Cage 27 Latch helix (SEQ ID NO: 917).

VI.c. Key Polypeptide

As described herein, a key polypeptide plays an essential role in the activation of the caged degron in a chimeric polypeptide of the present disclosure. In some aspects, the dynamic range of activation by key polypeptides can be tuned by truncating the latch region length to be shorter than the alpha-helices in the structural region, simultaneously weakening the cage polypeptide-latch region interaction and opening an exposed region on the cage polypeptide that the key polypeptide can bind to. Therefore, in some aspects, a key polypeptide is longer (e.g., at least 2, 3, 4, 5, 6, 7, 8, 9, 10 amino acids) in length than the latch region of the cage polypeptide. Similarly, the dynamic range of activation by key polypeptides can also be tuned in a similar manner by designing mutations into the latch region that weaken the cage polypeptide-latch region interaction, and thereby, facilitating the displacement of the latch region by a key polypeptide and activation of the degron. Therefore, in some aspects, a key polypeptide and the latch region of the cage polypeptide are not identical. In some aspects, the key polypeptide and the latch region of the cage polypeptide are at least about 50%, at least about 55%, at least about 60%, at least about 65%, at least about 70%, at least about 75%, at least about 80%, at least about 85%, at least about 90%, or at least about 95% identical to each other. In some aspects, the key polypeptide and the latch region of the cage polypeptide are identical except 2, 3, 4, 5, 6, 7, 8, 9, 10, 11, or 12 amino acids. In some aspects, the key polypeptide and the latch region of the cage polypeptides comprise similar amino acid sequences, e.g., at least about 75% sequence identity, except that the latch region binds to the structural region of the cage polypeptide with a lower affinity, e.g., less hydrophobic interaction, than the key polypeptide does. In other words, the key polypeptide can bind to the structural region with a higher affinity that the latch region does, e.g., higher hydrophobic interaction.

In some aspects, the K_{D} of the interaction between the latch region and the structural region of the cage is higher than the K_{D} of the interaction between the key polypeptide and the structural region of the cage. In some aspects, the K_{D} of the interaction between the key polypeptide and the structural region of the cage is at least about 0.1, at least about 0.2, at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, or at least about 2 orders of magnitude lower than the K_{D} of the interaction between the latch region and the structural region of the cage.

TABLE 1
Polypeptide Keys

| SEQ ID NO | Description |
|---|---|
| 77 | Key1C |
| 78 | Key1N |
| 79 | Key2C |
| 80 | Key2N |
| 81 | Key3C |
| 82 | Key3N |
| 83 | Key4C |
| 84 | Key4N |
| 85 | Key5C |
| 86 | Key5N |
| 87 | Key6N |
| 88 | Key7C |
| 89 | Key7N |
| 90 | Key8C |
| 91 | Key9C |
| 92 | Key9N |
| 93 | Key10C |
| 94 | Key10N |
| 95 | Key11N |
| 96 | Key12C |

In some aspects, a key polypeptide that can be used with a chimeric polypeptide of the present disclosure comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NOS: 77-93, wherein the wherein the key polypeptide is capable of binding to the structural region of the cage polypeptide of a corresponding chimeric polypeptide disclosed herein.

In some aspects, a key polypeptide that can be used with a chimeric polypeptide of the present disclosure consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NOS: 77-93, wherein the wherein the key polypeptide is capable of binding to the structural region of the cage polypeptide of a corresponding chimeric polypeptide disclosed herein.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 77, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 14, 15 or 16. In certain aspects, the key polypeptide comprises the amino acid sequence set forth in SEQ ID NO: 77, and the chimeric polypeptide comprises the amino acid sequence set forth in SEQ ID NO: 14, 15 or 16.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 77, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 14, 15 or 16. In certain aspects, the key polypeptide consists or consists essentially of the amino acid sequence set forth in SEQ ID NO: 77, and the chimeric polypeptide consists or consists essentially of the amino acid sequence set forth in SEQ ID NO: 14, 15 or 16.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 78, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 17 or 18. In certain aspects, the key polypeptide comprises the amino acid sequence set forth in SEQ ID NO: 78, and the chimeric polypeptide comprises the amino acid sequence set forth in SEQ ID NO: 17 or 18.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 78, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 17 or 18. In certain aspects, the key polypeptide consists or consists essentially of the amino acid sequence set forth in SEQ ID NO: 78, and the chimeric polypeptide consists or consists essentially of the amino acid sequence set forth in SEQ ID NO: 17 or 18.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 79, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 19. In certain aspects, the key polypeptide comprises the amino acid sequence set forth in SEQ ID NO: 79, and the chimeric polypeptide comprises the amino acid sequence set forth in SEQ ID NO: 19.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 79, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 19. In certain aspects, the key polypeptide consists or consists essentially of the amino acid sequence set forth in SEQ ID NO: 79, and the chimeric polypeptide consists or consists essentially of the amino acid sequence set forth in SEQ ID NO: 19.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 80, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 20, 818, or 819. In certain aspects, the key polypeptide comprises the amino acid sequence set forth in SEQ ID NO: 80, and the chimeric polypeptide comprises the amino acid sequence set forth in SEQ ID NO: 20, 818, or 819.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 80, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%. at least about 85%, at least about 90%, at least about 95%, at least about 96%, at least about 97%, at least about 98%, at least about 99%, or about 100% sequence identity to the sequence set forth in SEQ ID NO: 20, 818, or 819. In certain aspects, the key polypeptide consists or consists essentially of the amino acid sequence set forth in SEQ ID NO: 80, and the chimeric polypeptide consists or consists essentially of the amino acid sequence set forth in SEQ ID NO: 20, 818, or 819.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 81, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 22, 23, 24, 26, 27, or 807-810. In certain aspects, the key polypeptide comprises the amino acid sequence set forth in SEQ ID NO: 81, and the chimeric polypeptide comprises the amino acid sequence set forth in SEQ ID NO: 22, 23, 24, 26, 27, or 807-810.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 81, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 22, 23, 24, 26, 27, or 807-810. In certain aspects, the key polypeptide consists or consists essentially of the amino acid sequence set forth in SEQ ID NO: 81, and the chimeric polypeptide consists or consists essentially of the amino acid sequence set forth in SEQ ID NO: 22, 23, 24, 26, 27, or 807-810.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 82, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 21, 25, 28, 29, 30, 31, 32, 33, 34, 811, 814 or 815. In certain aspects, the key polypeptide comprises the amino acid sequence set forth in SEQ ID NO: 82, and the chimeric polypeptide comprises the amino acid sequence set forth in SEQ ID NO: 21, 25, 28, 29, 30, 31, 32, 33, 34, 811, 814 or 815.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 82, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 21, 25, 28, 29, 30, 31, 32, 33 34, 811, 814 or 815. In certain aspects, the key polypeptide consists or consists essentially of the amino acid sequence set forth in SEQ ID NO: 82, and the chimeric polypeptide consists or consists essentially of the amino acid sequence set forth in SEQ ID NO: 21, 25, 28, 29, 30, 31, 32, 33, 34, 811, 814 or 815.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 83, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 35, 36, 37, 38, 39, 40, 41, 42, 43, 44, 45, 46, 47, 48, 812 or 813. In certain aspects, the key polypeptide comprises the amino acid sequence set forth in SEQ ID NO: 83, and the chimeric polypeptide comprises the amino acid sequence set forth in SEQ ID NO: 35, 36, 37, 38, 39, 40, 41, 42, 43, 44, 45, 46, 47, 48, 812 or 813.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 83, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 35, 36, 37, 38, 39, 40, 41, 42, 43, 44, 45, 46, 47, 48, 812 or 813. In certain aspects, the key polypeptide consists or consists essentially of the amino acid sequence set forth in SEQ ID NO: 83, and the chimeric polypeptide consists or consists essentially of the amino acid sequence set forth in SEQ ID NO: 35, 36, 37, 38, 39, 40, 41, 42, 43, 44, 45, 46, 47, 48, 812 or 813.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 84, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 49, 50, 51, 52, 53 or 54. In certain aspects, the key polypeptide comprises the amino acid sequence set forth in SEQ ID NO: 84, and the chimeric polypeptide comprises the amino acid sequence set forth in SEQ ID NO: 49, 50, 51, 52, 53 or 54.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 84, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 49, 50, 51, 52, 53 or 54. In certain aspects, the key polypeptide consists or consists essentially of the amino acid sequence set forth in SEQ ID NO: 84, and the chimeric polypeptide consists or consists essentially of the amino acid sequence set forth in SEQ ID NO: 49, 50, 51, 52, 53 or 54.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 85, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 55, 56 or 58. In certain aspects, the key polypeptide comprises the amino acid sequence set forth in SEQ ID NO: 85, and the chimeric polypeptide comprises the amino acid sequence set forth in SEQ ID NO: 55, 56 or 58.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 85, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 55, 56 or 58. In certain aspects, the key polypeptide consists or consists essentially of the amino acid sequence set forth in SEQ ID NO: 85, and the chimeric polypeptide consists or consists essentially of the amino acid sequence set forth in SEQ ID NO: 55, 56 or 58.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 86, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 57 or 59. In certain aspects, the key polypeptide comprises the amino acid sequence set forth in SEQ ID NO: 86, and the chimeric polypeptide comprises the amino acid sequence set forth in SEQ ID NO: 57 or 59.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 86, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 57 or 59. In certain aspects, the key polypeptide consists or consists essentially of the amino acid sequence set forth in SEQ ID NO: 86, and the chimeric polypeptide consists or consists essentially of the amino acid sequence set forth in SEQ ID NO: 57 or 59.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 87, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 60. In certain aspects, the key polypeptide comprises the amino acid sequence set forth in SEQ ID NO: 87, and the chimeric polypeptide comprises the amino acid sequence set forth in SEQ ID NO: 60.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 87, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 60. In certain aspects, the key polypeptide consists or consists essentially of the amino acid sequence set forth in SEQ ID NO: 87, and the chimeric polypeptide consists or consists essentially of the amino acid sequence set forth in SEQ ID NO: 60.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 88, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 61, 820, or 821. In certain aspects, the key polypeptide comprises the amino acid sequence set forth in SEQ ID NO: 88, and the chimeric polypeptide comprises the amino acid sequence set forth in SEQ ID NO: 61, 820, or 821.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 88, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 61, 820, or 821. In certain aspects, the key polypeptide consists or consists essentially of the amino acid sequence set forth in SEQ ID NO: 88, and the chimeric polypeptide consists or consists essentially of the amino acid sequence set forth in SEQ ID NO: 61, 820, or 821.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 89, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 62. In certain aspects, the key polypeptide comprises the amino acid sequence set forth in SEQ ID NO: 89, and the chimeric polypeptide comprises the amino acid sequence set forth in SEQ ID NO: 62.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 89, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 62. In certain aspects, the key polypeptide consists or consists essentially of the amino acid sequence set forth in SEQ ID NO: 89, and the chimeric polypeptide consists or consists essentially of the amino acid sequence set forth in SEQ ID NO: 62.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 90, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 63. In certain aspects, the key polypeptide comprises the amino acid sequence set forth in SEQ ID NO: 90, and the chimeric polypeptide comprises the amino acid sequence set forth in SEQ ID NO: 63.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 90, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 63. In certain aspects, the key polypeptide consists or consists essentially of the amino acid sequence set forth in SEQ ID NO: 90, and the chimeric polypeptide consists or consists essentially of the amino acid sequence set forth in SEQ ID NO: 63.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 91, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 65. In certain aspects, the key polypeptide comprises the amino acid sequence set forth in SEQ ID NO: 91, and the chimeric polypeptide comprises the amino acid sequence set forth in SEQ ID NO: 65.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 91, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 65. In certain aspects, the key polypeptide consists or consists essentially of the amino acid sequence set forth in SEQ ID NO: 91, and the chimeric polypeptide consists or consists essentially of the amino acid sequence set forth in SEQ ID NO: 65.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 92, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 64, 66 or 67. In certain aspects, the key polypeptide comprises the amino acid sequence set forth in SEQ ID NO: 92, and the chimeric polypeptide comprises the amino acid sequence set forth in SEQ ID NO: 64, 66 or 67.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 92, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 64, 66 or 67. In certain aspects, the key polypeptide consists or consists essentially of the amino acid sequence set forth in SEQ ID NO: 92, and the chimeric polypeptide consists or consists essentially of the amino acid sequence set forth in SEQ ID NO: 64, 66 or 67.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 93, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 68, 69, 70, or 73. In certain aspects, the key polypeptide comprises the amino acid sequence set forth in SEQ ID NO: 93, and the chimeric polypeptide comprises the amino acid sequence set forth in SEQ ID NO: 68, 69, 70, or 73.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 93, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 68, 69, 70, or 73. In certain aspects, the key polypeptide consists or consists essentially of the amino acid sequence set forth in SEQ ID NO: 93, and the chimeric polypeptide consists or consists essentially of the amino acid sequence set forth in SEQ ID NO: 68, 69, 70, or 73.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 94, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 71, 72, 74, 816 or 817. In certain aspects, the key polypeptide comprises the amino acid sequence set forth in SEQ ID NO: 94, and the chimeric polypeptide comprises the amino acid sequence set forth in SEQ ID NO: 71, 72, 74, 816 or 817.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 94, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 71, 72, 74, 816 or 817. In certain aspects, the key polypeptide consists or consists essentially of the amino acid sequence set forth in SEQ ID NO: 94, and the chimeric polypeptide consists or consists essentially of the amino acid sequence set forth in SEQ ID NO: 71, 72, 74, 816 or 817.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 95, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 75. In certain aspects, the key polypeptide comprises the amino acid sequence set forth in SEQ ID NO: 95, and the chimeric polypeptide comprises the amino acid sequence set forth in SEQ ID NO: 75.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 95, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 75. In certain aspects, the key polypeptide consists or consists essentially of the amino acid sequence set forth in SEQ ID NO: 95, and the chimeric polypeptide consists or consists essentially of the amino acid sequence set forth in SEQ ID NO: 75.

In some aspects, (i) a key polypeptide comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 96, and (ii) the chimeric polypeptide to which the key polypeptide can bind comprises an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 76. In certain aspects, the key polypeptide comprises the amino acid sequence set forth in SEQ ID NO: 96, and the chimeric polypeptide comprises the amino acid sequence set forth in SEQ ID NO: 76.

In some aspects, (i) a key polypeptide consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 96, and (ii) the chimeric polypeptide to which the key polypeptide can bind consists or consists essentially of an amino acid sequence having at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% sequence identity to the sequence set forth in SEQ ID NO: 76. In certain aspects, the key polypeptide consists or consists essentially of the amino acid sequence set forth in SEQ ID NO: 96, and the chimeric polypeptide consists or consists essentially of the amino acid sequence set forth in SEQ ID NO: 76.

TABLE 2
Chimeric polypeptides, corresponding keys, and
latch helices where degrons are inserted.

| | | | Helix (C-term or |
|---|---|---|---|
| Polypeptide | | Key | N-term) where |
| SEQ ID NO | Key | SEQ ID NO | Degron inserted |
| 14 | Key1C | 77 | C-helix |
| 15 | Key1C | 77 | C-helix |
| 16 | Key1C | 77 | C-helix |
| 17 | Key1N | 78 | N-helix |
| 18 | Key1N | 78 | N-helix |
| 19 | Key2C | 79 | C-helix |
| 20 | Key2N | 80 | N-helix |
| 21 | Key3N | 82 | N-helix |
| 22 | Key3C | 81 | C-helix |
| 23 | Key3C | 81 | C-helix |
| 24 | Key3C | 81 | C-helix |
| 25 | Key3N | 82 | N-helix |
| 26 | Key3C | 81 | C-helix |
| 27 | Key3C | 81 | C-helix |
| 28 | Key3N | 82 | N-helix |
| 29 | Key3N | 82 | N-helix |
| 30 | Key3N | 82 | N-helix |
| 31 | Key3N | 82 | N-helix |
| 32 | Key3N | 82 | N-helix |
| 33 | Key3N | 82 | N-helix |
| 34 | Key3N | 82 | N-helix |
| 35 | Key4C | 83 | C-helix |
| 36 | Key4C | 83 | C-helix |
| 37 | Key4C | 83 | C-helix |
| 38 | Key4C | 83 | C-helix |
| 39 | Key4C | 83 | C-helix |
| 40 | Key4C | 83 | C-helix |
| 41 | Key4C | 83 | C-helix |
| 42 | Key4C | 83 | C-helix |
| 43 | Key4C | 83 | C-helix |
| 44 | Key4C | 83 | C-helix |
| 45 | Key4C | 83 | C-helix |
| 46 | Key4C | 83 | C-helix |
| 47 | Key4C | 83 | C-helix |
| 48 | Key4C | 83 | C-helix |
| 49 | Key4N | 84 | N-helix |
| 50 | Key4N | 84 | N-helix |
| 51 | Key4N | 84 | N-helix |
| 52 | Key4N | 84 | N-helix |
| 53 | Key4N | 84 | N-helix |
| 54 | Key4N | 84 | N-helix |
| 55 | Key5C | 85 | C-helix |
| 56 | Key5C | 85 | C-helix |
| 57 | Key5N | 86 | N-helix |
| 58 | Key5C | 85 | C-helix |
| 59 | Key5N | 86 | N-helix |
| 60 | Key6N | 87 | N-helix |
| 61 | Key7C | 88 | C-helix |
| 62 | Key7N | 89 | N-helix |
| 63 | Key8C | 90 | C-helix |
| 64 | Key9N | 92 | N-helix |
| 65 | Key9C | 91 | C-helix |
| 66 | Key9N | 92 | N-helix |
| 67 | Key9N | 92 | N-helix |
| 68 | Key10C | 93 | C-helix |
| 69 | Key10C | 93 | C-helix |
| 70 | Key10C | 93 | C-helix |
| 71 | Key10N | 94 | N-helix |
| 72 | Key10N | 94 | N-helix |
| 73 | Key10C | 93 | C-helix |
| 74 | Key10N | 94 | N-helix |
| 75 | Key11N | 95 | N-helix |
| 76 | Key12C | 96 | C-helix |
| 807 | Key3C | 81 | C-helix |
| 808 | Key3C | 81 | C-helix |
| 809 | Key3C | 81 | C-helix |
| 810 | Key3C | 81 | C-helix |
| 811 | Key3N | 82 | N-helix |
| 812 | Key4C | 83 | C-helix |
| 813 | Key4C | 83 | C-helix |
| 814 | Key3N | 82 | N-helix |
| 815 | Key3N | 82 | N-helix |
| 816 | Key10N | 94 | N-helix |
| 817 | Key10N | 94 | N-helix |
| 818 | Key2N | 80 | N-helix |
| 819 | Key2N | 80 | N-helix |
| 820 | Key7C | 88 | C-helix |
| 821 | Key7C | 88 | C-helix |

C-helix is the helix comprising the C-terminal amino acid of the helix bundle; N-helix is the helix comprising the N-terminal amino acid of the helix.

VI.d. Other Moieties

In some aspects, a chimeric polypeptide of the present disclosure can be used in combination with one or more additional moieties. For instance, as described herein, a chimeric polypeptide disclosed herein can be used in combination with a CAR or a TCR, where the chimeric polypeptide is incorporated in the intracellular portion of the CAR or TCR. In certain aspects, the chimeric polypeptide of the present disclosure is attached (e.g., fused) to the C-terminus of the CAR or TCR.

In some aspects, the chimeric polypeptide is inserted between two functional domains in the intracellular portion of the CAR or TCR. The transmembrane domain of a CAR is fused to the costimulatory domain, optionally a costimulatory domain is fused to a second costimulatory domain, and the costimulatory domain is fused to a signaling domain, not limited to CD3ζ. Accordingly, in some aspects, the chimeric polypeptide is inserted between the transmembrane domain and a costimulatory domain (e.g., a first costimulatory domain). In some aspects, the chimeric polypeptide is inserted between a first costimulatory domain and a second costimulatory domain. In some aspects, the chimeric polypeptide is inserted between a costimulatory domain and a signaling domain.

In some aspects, a chimeric polypeptide disclosed herein further comprises a signaling domain. In some aspects, the signaling domain comprises a domain derived from CD3zeta, FcR gamma, FcR beta, CD3 gamma, CD3 delta, CD3 epsilon, CD5, CD22, CD79a, CD79b, CD66d, or combinations thereof.

In some aspects, a chimeric polypeptide disclosed herein further comprises a co-stimulatory domain. In some aspects, the co-stimulatory domain comprises a domain derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, B7-H3, or combinations thereof.

In some aspects, a chimeric polypeptide disclosed herein further comprises an antigen-binding domain. In some aspects, the antigen-binding domain comprises an antibody or an antigen-binding fragment thereof that specifically binds to an epitope on a tumor antigen. In some aspects, the antigen binding domain is an IgNAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, or combinations thereof. Non-limiting examples of antigens that the antigen-binding domain can bind to includes CD19, TRAC, TCRβ, BCMA, CLL-1, CS1, CD38, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, and any combinations thereof.

In some aspects, a chimeric polypeptide disclosed herein further comprises transmembrane domain.

In some aspects, a chimeric polypeptide disclosed herein further comprises a chimeric antigen receptor (CAR), e.g., a standard CAR, a split CAR, an off-CAR, an on-CAR, a first-generation CAR, a second-generation CAR, a third-generation CAR, or a fourth-generation CAR.

In some aspects, a chimeric polypeptide disclosed herein comprises a CAR comprising:

(i) a chimeric polypeptide disclosed herein;

(ii) an antigen-binding domain that binds to an epitope on a tumor antigen expressed on a target cell; and,

(iii) a transmembrane domain.

In some aspects, the CAR further comprises a CAR spacer between the antigen binding domain and the transmembrane domain. In some aspects, the chimeric polypeptide is distally linked to the cytoplasmic region of the CAR. In some aspects, the chimeric polypeptide is interposed between two domains in the cytoplasmic region of the CAR. In some aspects, the chimeric polypeptide is attached to the CAR via a linker or spacer. In some aspects, the CAR can comprise more than one chimeric polypeptide, which can be activated by the same key polypeptide or by different key polypeptides.

In some aspects, a chimeric polypeptide disclosed herein further comprises a T cell receptor (TCR).

In some aspects, a chimeric polypeptide disclosed herein, e.g., a CAR or a TCR, can induce IFNγ and/or IL-2 expression in a cell (e.g., a T cell).

In some aspects, the CAR that can be linked to the chimeric polypeptide of the present disclosure comprises a standard CAR, a split CAR, an off-switch CAR, an on-switch CAR, a first-generation CAR, a second-generation CAR, a third-generation CAR, or a fourth-generation CAR.

In some aspects, a polypeptide of the present disclosure (e.g., a polypeptide comprising a chimeric polypeptide disclosed herein) is a CAR as set forth in SEQ ID NO: 97. In other aspects, a polypeptide of the present disclosure (e.g., a polypeptide comprising a chimeric polypeptide disclosed herein) is a CAR as set forth in SEQ ID NO: 99.

In some aspects, the present disclosure also includes a chimeric protein complex comprising the cage polypeptide of the present disclosure and the key polypeptide of the present disclosure, wherein the cage and key polypeptides are bound to each other. Once the key polypeptide is bound to the cage polypeptide, the degron peptide linked to the latch region can activate a ubiquitin dependent degradation pathway and the complex can be degraded by a ubiquitin system.

### III. Polynucleotides

In some aspects, the present disclosure provides a polynucleotide encoding one or more components (e.g., cage or key) of a chimeric polypeptide of the present disclosure (e.g., comprising a degron integrated into the latch region).

In some aspects, a polynucleotide disclosed herein encodes a chimeric polypeptide which comprises:
(i) a cage polypeptide comprising an amino acid sequence having at least about 50%, at least about 55%, at least about 60%, at least about 65%, at least about 70%, at least about 75%, at least about 80 at least about 85%, at least about 90%, at least about 95%, at least about 96%, at least about 97%, at least about 98%, at least about 99%, or about 100% sequence identity to the amino acid sequence set forth in SEQ ID NO:142-165, 838, 840, 842, 844, 846, 848, 850, 852, 854, 856, 858, 860, 862, 864, or 866; and,
(ii) a degron peptide comprising the amino acid sequence as set forth in SEQ ID NO: 1 or a fragment thereof (e.g., a 4 to 19 amino acid long fragment such as those set forth in SEQ ID NOs: 110-141 680-804, or 918-982), wherein the cage polypeptide comprises a structural region and a latch region, and wherein the latch region is capable of binding to a structural region.

In some aspects, a polynucleotide disclosed herein encodes a chimeric polypeptide which comprises:
(i) a cage polypeptide comprising an amino acid sequence having at least about 50%, at least about 55%, at least about 60%, at least about 65%, at least about 70%, at least about 75%, at least about 80 at least about 85%, at least about 90%, at least about 95%, at least about 96%, at least about 97%, at least about 98%, at least about 99%, or about 100% sequence identity to the amino acid sequence set forth in SEQ ID NO:142-165, 838, 840, 842, 844, 846, 848, 850, 852, 854, 856, 858, 860, 862, 864, or 866; and,
(ii) a degron peptide consisting or consisting essentially of the amino acid sequence as set forth in SEQ ID NO: 1 or a fragment thereof (e.g., a 4 to 19 amino acid long fragment such as those set forth in SEQ ID NOs: 110-141 680-804, or 918-982), wherein the cage polypeptide comprises a structural region and a latch region, and wherein the latch region is capable of binding to a structural region.

As disclosed herein, in some aspects, a polynucleotide disclosed herein encodes a chimeric polypeptide comprising a sequestered degron peptide, wherein the degron peptide comprises, consists, or consists essentially of the amino acid sequence of SEQ ID NO:1 or a fragment thereof (e.g., a 4 to 19 amino acid long fragment such as those set forth in SEQ ID NOs: 110-141 680-804, or 918-982). In certain aspects, a polynucleotide disclosed herein encodes a chimeric polypeptide comprising a sequestered degron peptide, wherein the degron peptide comprises, consists, or consists essentially of an amino acid sequence that is at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to SEQ ID NO:1 or a fragment thereof (e.g., a 4 to 19 amino acid long fragment such as those set forth in SEQ ID NOs: 110-141 680-804, or 918-982). In some aspects, the mutant degron (i.e., a degron with less than 100% sequence identity to SEQ ID NO:1 or a fragment thereof, e.g., a 4 to 19 amino acid long fragment such as those set forth in SEQ ID NOs: 110-141 680-804, or 918-982) can be targeted by an ubiquitin ligase.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 14. In certain aspects, a polynucleotide disclosed herein encodes a chimeric polypeptide which comprises the amino acid sequence set forth in SEQ ID NO:14.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 14. In certain aspects, a polynucleotide disclosed herein encodes a chimeric polypeptide which consists or consists essentially of the amino acid sequence set forth in SEQ ID NO:14.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 15. In certain aspects, a polynucleotide disclosed herein encodes a chimeric polypeptide which comprises the amino acid sequence set forth in SEQ ID NO:15.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 15. In certain aspects, a polynucleotide disclosed herein encodes a chimeric polypeptide which consists or consists essentially of the amino acid sequence set forth in SEQ ID NO:15.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 16. In certain aspects, a polynucleotide disclosed herein encodes a chimeric polypeptide which comprises the amino acid sequence set forth in SEQ ID NO:16.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 16. In certain aspects, a polynucleotide disclosed herein encodes a chimeric polypeptide which consists or consists essentially of the amino acid sequence set forth in SEQ ID NO:16.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 17. In certain aspects, a polynucleotide disclosed herein encodes a chimeric polypeptide which comprises the amino acid sequence set forth in SEQ ID NO:17.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 17. In certain aspects, a polynucleotide disclosed herein encodes a chimeric polypeptide which consists or consists essentially of the amino acid sequence set forth in SEQ ID NO:17.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 18. In certain aspects, a polynucleotide disclosed herein encodes a chimeric polypeptide which comprises the amino acid sequence set forth in SEQ ID NO:18.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 18. In certain aspects, a polynucleotide disclosed herein encodes a chimeric polypeptide which consists or consists essentially of the amino acid sequence set forth in SEQ ID NO:18.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 19. In certain aspects, a polynucleotide disclosed herein encodes a chimeric polypeptide which comprises the amino acid sequence set forth in SEQ ID NO:19.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 19. In certain aspects, a polynucleotide disclosed herein encodes a chimeric polypeptide which consists or consists essentially of the amino acid sequence set forth in SEQ ID NO:19.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 20. In certain aspects, a polynucleotide disclosed herein encodes a chimeric polypeptide which comprises the amino acid sequence set forth in SEQ ID NO:20.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 20. In certain aspects, a polynucleotide disclosed herein encodes a chimeric polypeptide which consists or consists essentially of the amino acid sequence set forth in SEQ ID NO:20.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 818. In certain aspects, a polynucleotide disclosed herein encodes a chimeric polypeptide which comprises the amino acid sequence set forth in SEQ ID NO: 818.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 818. In certain aspects, a polynucleotide disclosed herein encodes a chimeric polypeptide which consists or consists essentially of the amino acid sequence set forth in SEQ ID NO: 818.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 819. In certain aspects, a polynucleotide disclosed herein encodes a chimeric polypeptide which comprises the amino acid sequence set forth in SEQ ID NO: 819.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 819. In certain aspects, a polynucleotide disclosed herein encodes a chimeric polypeptide which consists or consists essentially of the amino acid sequence set forth in SEQ ID NO: 819.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 21. In certain aspects, a polynucleotide disclosed herein encodes a chimeric polypeptide which comprises the amino acid sequence set forth in SEQ ID NO:21.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 21. In certain aspects, a polynucleotide disclosed herein encodes a chimeric polypeptide which consists or consists essentially of the amino acid sequence set forth in SEQ ID NO:21.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 22. In certain aspects, a polynucleotide disclosed herein encodes a chimeric polypeptide which comprises the amino acid sequence set forth in SEQ ID NO:22.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 22. In certain aspects, a polynucleotide disclosed herein encodes a chimeric polypeptide which consists or consists essentially of the amino acid sequence set forth in SEQ ID NO:22.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 23. In certain aspects, a polynucleotide disclosed herein encodes a chimeric polypeptide which comprises the amino acid sequence set forth in SEQ ID NO:23.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 23. In certain aspects, a polynucleotide disclosed herein encodes a chimeric polypeptide which consists or consists essentially of the amino acid sequence set forth in SEQ ID NO:23.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 24. In certain aspects, a polynucleotide disclosed herein encodes a chimeric polypeptide which comprises the amino acid sequence set forth in SEQ ID NO:24.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 24. In certain aspects, a polynucleotide disclosed herein encodes a chimeric polypeptide which consists or consists essentially of the amino acid sequence set forth in SEQ ID NO:24.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 25. In certain aspects, a polynucleotide disclosed herein encodes a chimeric polypeptide which comprises the amino acid sequence set forth in SEQ ID NO:25.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 25. In certain aspects, a polynucleotide disclosed herein encodes a chimeric polypeptide which consists or consists essentially of the amino acid sequence set forth in SEQ ID NO:25.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 26. In certain aspects, a polynucleotide disclosed herein encodes a chimeric polypeptide which comprises the amino acid sequence set forth in SEQ ID NO:26.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 26. In certain aspects, a polynucleotide disclosed herein encodes a chimeric polypeptide which consists or consists essentially of the amino acid sequence set forth in SEQ ID NO:26.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 27. In certain aspects, a polynucleotide disclosed herein encodes a chimeric polypeptide which comprises the amino acid sequence set forth in SEQ ID NO:27.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 27. In certain aspects, a polynucleotide disclosed herein encodes a chimeric polypeptide which consists or consists essentially of the amino acid sequence set forth in SEQ ID NO:27.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 28. In certain aspects, a polynucleotide disclosed herein encodes a chimeric polypeptide which comprises the amino acid sequence set forth in SEQ ID NO:28.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 28. In certain aspects, a polynucleotide disclosed herein encodes a chimeric polypeptide which consists or consists essentially of the amino acid sequence set forth in SEQ ID NO:28.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 29. In certain aspects, a polynucleotide disclosed herein encodes a chimeric polypeptide which comprises the amino acid sequence set forth in SEQ ID NO:29.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 29. In certain aspects, a polynucleotide disclosed herein encodes a chimeric polypeptide which consists or consists essentially of the amino acid sequence set forth in SEQ ID NO:29.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 30. In certain aspects, a polynucleotide disclosed herein encodes a chimeric polypeptide which comprises the amino acid sequence set forth in SEQ ID NO:30.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 30. In certain aspects, a polynucleotide disclosed herein encodes a chimeric polypeptide which consists or consists essentially of the amino acid sequence set forth in SEQ ID NO:30.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 31. In certain aspects, a polynucleotide disclosed herein encodes a chimeric polypeptide which comprises the amino acid sequence set forth in SEQ ID NO:31.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 31. In certain aspects, a polynucleotide disclosed herein encodes a chimeric polypeptide which consists or consists essentially of the amino acid sequence set forth in SEQ ID NO:31.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 32. In certain aspects, a polynucleotide disclosed herein encodes a chimeric polypeptide which comprises the amino acid sequence set forth in SEQ ID NO:32.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 32. In certain aspects, a polynucleotide disclosed herein encodes a chimeric polypeptide which consists or consists essentially of the amino acid sequence set forth in SEQ ID NO:32.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 33. In certain aspects, a polynucleotide disclosed herein encodes a chimeric polypeptide which comprises the amino acid sequence set forth in SEQ ID NO:33.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 33. In certain aspects, a polynucleotide disclosed herein encodes a chimeric polypeptide which consists or consists essentially of the amino acid sequence set forth in SEQ ID NO:33.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 34. In certain aspects, a polynucleotide disclosed herein encodes a chimeric polypeptide which comprises the amino acid sequence set forth in SEQ ID NO:34.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 34. In certain aspects, a polynucleotide disclosed herein encodes a chimeric polypeptide which consists or consists essentially of the amino acid sequence set forth in SEQ ID NO:34.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 807. In certain aspects, a polynucleotide disclosed herein encodes a chimeric polypeptide which comprises the amino acid sequence set forth in SEQ ID NO: 807.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 807. In certain aspects, a polynucleotide disclosed herein encodes a chimeric polypeptide which consists or consists essentially of the amino acid sequence set forth in SEQ ID NO: 807.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 808. In certain aspects, a polynucleotide disclosed herein encodes a chimeric polypeptide which comprises the amino acid sequence set forth in SEQ ID NO: 808.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 808. In certain aspects, a polynucleotide disclosed herein encodes a chimeric polypeptide which consists or consists essentially of the amino acid sequence set forth in SEQ ID NO: 808.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 809. In certain aspects, a polynucleotide disclosed herein encodes a chimeric polypeptide which comprises the amino acid sequence set forth in SEQ ID NO: 809.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 809. In certain aspects, a polynucleotide disclosed herein encodes a chimeric polypeptide which consists or consists essentially of the amino acid sequence set forth in SEQ ID NO: 809.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 810. In certain aspects, a polynucleotide disclosed herein encodes a chimeric polypeptide which comprises the amino acid sequence set forth in SEQ ID NO: 810.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 810. In certain aspects, a polynucleotide disclosed herein encodes a chimeric polypeptide which consists or consists essentially of the amino acid sequence set forth in SEQ ID NO: 810.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 814. In certain aspects, a polynucleotide disclosed herein encodes a chimeric polypeptide which comprises the amino acid sequence set forth in SEQ ID NO: 814.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 814. In certain aspects, a polynucleotide disclosed herein encodes a chimeric polypeptide which consists or consists essentially of the amino acid sequence set forth in SEQ ID NO: 814.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 815. In certain aspects, a polynucleotide disclosed herein encodes a chimeric polypeptide which comprises the amino acid sequence set forth in SEQ ID NO: 815.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 815. In certain aspects, a polynucleotide disclosed herein encodes a chimeric polypeptide which consists or consists essentially of the amino acid sequence set forth in SEQ ID NO: 815.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 35. In certain aspects, a polynucleotide disclosed herein encodes a chimeric polypeptide which comprises the amino acid sequence set forth in SEQ ID NO:35.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 35. In certain aspects, a polynucleotide disclosed herein encodes a chimeric polypeptide which consists or consists essentially of the amino acid sequence set forth in SEQ ID NO:35.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 36. In certain aspects, a polynucleotide disclosed herein encodes a chimeric polypeptide which comprises the amino acid sequence set forth in SEQ ID NO:36.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 36. In certain aspects, a polynucleotide disclosed herein encodes a chimeric polypeptide which consists or consists essentially of the amino acid sequence set forth in SEQ ID NO:36.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 37. In certain aspects, a polynucleotide disclosed herein encodes a chimeric polypeptide which comprises the amino acid sequence set forth in SEQ ID NO:37.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 37. In certain aspects, a polynucleotide disclosed herein encodes a chimeric polypeptide which consists or consists essentially of the amino acid sequence set forth in SEQ ID NO:37.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 38. In certain aspects, a polynucleotide disclosed herein encodes a chimeric polypeptide which comprises the amino acid sequence set forth in SEQ ID NO:38.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 38. In certain aspects, a polynucleotide disclosed herein encodes a chimeric polypeptide which consists or consists essentially of the amino acid sequence set forth in SEQ ID NO:38.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 39. In certain aspects, a polynucleotide disclosed herein encodes a chimeric polypeptide which comprises the amino acid sequence set forth in SEQ ID NO:39.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 39. In certain aspects, a polynucleotide disclosed herein encodes a chimeric polypeptide which consists or consists essentially of the amino acid sequence set forth in SEQ ID NO:39.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 40. In certain aspects, a polynucleotide disclosed herein encodes a chimeric polypeptide which comprises the amino acid sequence set forth in SEQ ID NO:40.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 40. In certain aspects, a polynucleotide disclosed herein encodes a chimeric polypeptide which consists or consists essentially of the amino acid sequence set forth in SEQ ID NO:40.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 41. In certain aspects, a polynucleotide disclosed herein encodes a chimeric polypeptide which comprises the amino acid sequence set forth in SEQ ID NO:41.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 41. In certain aspects, a polynucleotide disclosed herein encodes a chimeric polypeptide which consists or consists essentially of the amino acid sequence set forth in SEQ ID NO:41.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 42. In certain aspects, a polynucleotide disclosed herein encodes a chimeric polypeptide which comprises the amino acid sequence set forth in SEQ ID NO:42.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 42. In certain aspects, a polynucleotide disclosed herein encodes a chimeric polypeptide which consists or consists essentially of the amino acid sequence set forth in SEQ ID NO:42.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 43. In certain aspects, a polynucleotide disclosed herein encodes a chimeric polypeptide which comprises the amino acid sequence set forth in SEQ ID NO:43.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 43. In certain aspects, a polynucleotide disclosed herein encodes a chimeric polypeptide which consists or consists essentially of the amino acid sequence set forth in SEQ ID NO:43.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 44. In certain aspects, a polynucleotide disclosed herein encodes a chimeric polypeptide which comprises the amino acid sequence set forth in SEQ ID NO:44.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 44. In certain aspects, a polynucleotide disclosed herein encodes a chimeric polypeptide which consists or consists essentially of the amino acid sequence set forth in SEQ ID NO:44.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 45. In certain aspects, a polynucleotide disclosed herein encodes a chimeric polypeptide which comprises the amino acid sequence set forth in SEQ ID NO:45.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 45. In certain aspects, a polynucleotide disclosed herein encodes a chimeric polypeptide which consists or consists essentially of the amino acid sequence set forth in SEQ ID NO:45.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 46. In certain aspects, a polynucleotide disclosed herein encodes a chimeric polypeptide which comprises the amino acid sequence set forth in SEQ ID NO:46.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 46. In certain aspects, a polynucleotide disclosed herein encodes a chimeric polypeptide which consists or consists essentially of the amino acid sequence set forth in SEQ ID NO:46.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 47. In certain aspects, a polynucleotide disclosed herein encodes a chimeric polypeptide which comprises the amino acid sequence set forth in SEQ ID NO:47.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 47. In certain aspects, a polynucleotide disclosed herein encodes a chimeric polypeptide which consists or consists essentially of the amino acid sequence set forth in SEQ ID NO:47.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 48. In certain aspects, a polynucleotide disclosed herein encodes a chimeric polypeptide which comprises the amino acid sequence set forth in SEQ ID NO:48.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 48. In certain aspects, a polynucleotide disclosed herein encodes a chimeric polypeptide which consists or consists essentially of the amino acid sequence set forth in SEQ ID NO:48.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 49. In certain aspects, a polynucleotide disclosed herein encodes a chimeric polypeptide which comprises the amino acid sequence set forth in SEQ ID NO:49.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 49. In certain aspects, a polynucleotide disclosed herein encodes a chimeric polypeptide which consists or consists essentially of the amino acid sequence set forth in SEQ ID NO:49.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 50. In certain aspects, a polynucleotide disclosed herein encodes a chimeric polypeptide which comprises the amino acid sequence set forth in SEQ ID NO:50.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 50. In certain aspects, a polynucleotide disclosed herein encodes a chimeric polypeptide which consists or consists essentially of the amino acid sequence set forth in SEQ ID NO:50.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 51. In certain aspects, a polynucleotide disclosed herein encodes a chimeric polypeptide which comprises the amino acid sequence set forth in SEQ ID NO:51.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 51. In certain aspects, a polynucleotide disclosed herein encodes a chimeric polypeptide which consists or consists essentially of the amino acid sequence set forth in SEQ ID NO:51.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 52. In certain aspects, a polynucleotide disclosed herein encodes a chimeric polypeptide which comprises the amino acid sequence set forth in SEQ ID NO:52.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 52. In certain aspects, a polynucleotide disclosed herein encodes a chimeric polypeptide which consists or consists essentially of the amino acid sequence set forth in SEQ ID NO:52.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 53. In certain aspects, a polynucleotide disclosed herein encodes a chimeric polypeptide which comprises the amino acid sequence set forth in SEQ ID NO:53.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 53. In certain aspects, a polynucleotide disclosed herein encodes a chimeric polypeptide which consists or consists essentially of the amino acid sequence set forth in SEQ ID NO:53.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 54. In certain aspects, a polynucleotide disclosed herein encodes a chimeric polypeptide which comprises the amino acid sequence set forth in SEQ ID NO:54.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 54. In certain aspects, a polynucleotide disclosed herein encodes a chimeric polypeptide which consists or consists essentially of the amino acid sequence set forth in SEQ ID NO:54.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 812. In certain aspects, a polynucleotide disclosed herein encodes a chimeric polypeptide which comprises the amino acid sequence set forth in SEQ ID NO: 812.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 812. In certain aspects, a polynucleotide disclosed herein encodes a chimeric polypeptide which consists or consists essentially of the amino acid sequence set forth in SEQ ID NO: 812.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 813. In certain aspects, a polynucleotide disclosed herein encodes a chimeric polypeptide which comprises the amino acid sequence set forth in SEQ ID NO: 813.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 813. In certain aspects, a polynucleotide disclosed herein encodes a chimeric polypeptide which consists or consists essentially of the amino acid sequence set forth in SEQ ID NO: 813.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 55. In certain aspects, a polynucleotide disclosed herein encodes a chimeric polypeptide which comprises the amino acid sequence set forth in SEQ ID NO:55.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 55. In certain aspects, a polynucleotide disclosed herein encodes a chimeric polypeptide which consists or consists essentially of the amino acid sequence set forth in SEQ ID NO:55.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 56. In certain aspects, a polynucleotide disclosed herein encodes a chimeric polypeptide which comprises the amino acid sequence set forth in SEQ ID NO:56.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 56. In certain aspects, a polynucleotide disclosed herein encodes a chimeric polypeptide which consists or consists essentially of the amino acid sequence set forth in SEQ ID NO:56.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 57. In certain aspects, a polynucleotide disclosed herein encodes a chimeric polypeptide which comprises the amino acid sequence set forth in SEQ ID NO:57.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 57. In certain aspects, a polynucleotide disclosed herein encodes a chimeric polypeptide which consists or consists essentially of the amino acid sequence set forth in SEQ ID NO:57.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 58. In certain aspects, a polynucleotide disclosed herein encodes a chimeric polypeptide which comprises the amino acid sequence set forth in SEQ ID NO:58.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 58. In certain aspects, a polynucleotide disclosed herein encodes a chimeric polypeptide which consists or consists essentially of the amino acid sequence set forth in SEQ ID NO:58.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 59. In certain aspects, a polynucleotide disclosed herein encodes a chimeric polypeptide which comprises the amino acid sequence set forth in SEQ ID NO:59.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 59. In certain aspects, a polynucleotide disclosed herein encodes a chimeric polypeptide which consists or consists essentially of the amino acid sequence set forth in SEQ ID NO:59.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 60. In certain aspects, a polynucleotide disclosed herein encodes a chimeric polypeptide which comprises the amino acid sequence set forth in SEQ ID NO:60.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 60. In certain aspects, a polynucleotide disclosed herein encodes a chimeric polypeptide which consists or consists essentially of the amino acid sequence set forth in SEQ ID NO:60.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 61. In certain aspects, a polynucleotide disclosed herein encodes a chimeric polypeptide which comprises the amino acid sequence set forth in SEQ ID NO:61.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 61. In certain aspects, a polynucleotide disclosed herein encodes a chimeric polypeptide which consists or consists essentially of the amino acid sequence set forth in SEQ ID NO:61.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 62. In certain aspects, a polynucleotide disclosed herein encodes a chimeric polypeptide which comprises the amino acid sequence set forth in SEQ ID NO:62.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 62. In certain aspects, a polynucleotide disclosed herein encodes a chimeric polypeptide which consists or consists essentially of the amino acid sequence set forth in SEQ ID NO:62.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 820. In certain aspects, a polynucleotide disclosed herein encodes a chimeric polypeptide which comprises the amino acid sequence set forth in SEQ ID NO: 820.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 820. In certain aspects, a polynucleotide disclosed herein encodes a chimeric polypeptide which consists or consists essentially of the amino acid sequence set forth in SEQ ID NO: 820.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 821. In certain aspects, a polynucleotide disclosed herein encodes a chimeric polypeptide which comprises the amino acid sequence set forth in SEQ ID NO: 821.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 821. In certain aspects, a polynucleotide disclosed herein encodes a chimeric polypeptide which consists or consists essentially of the amino acid sequence set forth in SEQ ID NO: 821.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 63. In certain aspects, a polynucleotide disclosed herein encodes a chimeric polypeptide which comprises the amino acid sequence set forth in SEQ ID NO:63.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 63. In certain aspects, a polynucleotide disclosed herein encodes a chimeric polypeptide which consists or consists essentially of the amino acid sequence set forth in SEQ ID NO:63.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 64. In certain aspects, a polynucleotide disclosed herein encodes a chimeric polypeptide which comprises the amino acid sequence set forth in SEQ ID NO:64.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 64. In certain aspects, a polynucleotide disclosed herein encodes a chimeric polypeptide which consists or consists essentially of the amino acid sequence set forth in SEQ ID NO:64.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 65. In certain aspects, a polynucleotide disclosed herein encodes a chimeric polypeptide which comprises the amino acid sequence set forth in SEQ ID NO:65.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 65. In certain aspects, a polynucleotide disclosed herein encodes a chimeric polypeptide which consists or consists essentially of the amino acid sequence set forth in SEQ ID NO:65.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 66. In certain aspects, a polynucleotide disclosed herein encodes a chimeric polypeptide which comprises the amino acid sequence set forth in SEQ ID NO:66.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 66. In certain aspects, a polynucleotide disclosed herein encodes a chimeric polypeptide which consists or consists essentially of the amino acid sequence set forth in SEQ ID NO:66.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 67. In certain aspects, a polynucleotide disclosed herein encodes a chimeric polypeptide which comprises the amino acid sequence set forth in SEQ ID NO:67.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 67. In certain aspects, a polynucleotide disclosed herein encodes a chimeric polypeptide which consists or consists essentially of the amino acid sequence set forth in SEQ ID NO:67.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 68. In certain aspects, a polynucleotide disclosed herein encodes a chimeric polypeptide which comprises the amino acid sequence set forth in SEQ ID NO:68.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 68. In certain aspects, a polynucleotide disclosed herein encodes a chimeric polypeptide which consists or consists essentially of the amino acid sequence set forth in SEQ ID NO:68.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 69. In certain aspects, a polynucleotide disclosed herein encodes a chimeric polypeptide which comprises the amino acid sequence set forth in SEQ ID NO:69.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 69. In certain aspects, a polynucleotide disclosed herein encodes a chimeric polypeptide which consists or consists essentially of the amino acid sequence set forth in SEQ ID NO:69.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 70. In certain aspects, a polynucleotide disclosed herein encodes a chimeric polypeptide which comprises the amino acid sequence set forth in SEQ ID NO:70.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 70. In certain aspects, a polynucleotide disclosed herein encodes a chimeric polypeptide which consists or consists essentially of the amino acid sequence set forth in SEQ ID NO:70.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 71. In certain aspects, a polynucleotide disclosed herein encodes a chimeric polypeptide which comprises the amino acid sequence set forth in SEQ ID NO:71.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 71. In certain aspects, a polynucleotide disclosed herein encodes a chimeric polypeptide which consists or consists essentially of the amino acid sequence set forth in SEQ ID NO:71.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 72. In certain aspects, a polynucleotide disclosed herein encodes a chimeric polypeptide which comprises the amino acid sequence set forth in SEQ ID NO:72.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 72. In certain aspects, a polynucleotide disclosed herein encodes a chimeric polypeptide which consists or consists essentially of the amino acid sequence set forth in SEQ ID NO:72.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 73. In certain aspects, a polynucleotide disclosed herein encodes a chimeric polypeptide which comprises the amino acid sequence set forth in SEQ ID NO:73.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 73. In certain aspects, a polynucleotide disclosed herein encodes a chimeric polypeptide which consists or consists essentially of the amino acid sequence set forth in SEQ ID NO:73.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 74. In certain aspects, a polynucleotide disclosed herein encodes a chimeric polypeptide which comprises the amino acid sequence set forth in SEQ ID NO:74.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 74. In certain aspects, a polynucleotide disclosed herein encodes a chimeric polypeptide which consists or consists essentially of the amino acid sequence set forth in SEQ ID NO:74.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 816. In certain aspects, a polynucleotide disclosed herein encodes a chimeric polypeptide which comprises the amino acid sequence set forth in SEQ ID NO: 816.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 816. In certain aspects, a polynucleotide disclosed herein encodes a chimeric polypeptide which consists or consists essentially of the amino acid sequence set forth in SEQ ID NO: 816.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 817. In certain aspects, a polynucleotide disclosed herein encodes a chimeric polypeptide which comprises the amino acid sequence set forth in SEQ ID NO: 817.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 817. In certain aspects, a polynucleotide disclosed herein encodes a chimeric polypeptide which consists or consists essentially of the amino acid sequence set forth in SEQ ID NO: 817.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 75. In certain aspects, a polynucleotide disclosed herein encodes a chimeric polypeptide which comprises the amino acid sequence set forth in SEQ ID NO:75.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 75. In certain aspects, a polynucleotide disclosed herein encodes a chimeric polypeptide which consists or consists essentially of the amino acid sequence set forth in SEQ ID NO:75.

In some aspects, a polynucleotide disclosed herein encodes a chimeric polypeptide which comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 76. In certain aspects, a polynucleotide disclosed herein encodes a chimeric polypeptide which comprises the amino acid sequence set forth in SEQ ID NO:76.

In some aspects, a polynucleotide disclosed herein encodes a chimeric polypeptide which consists or consists essentially of an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 76. In certain aspects, a polynucleotide disclosed herein encodes a chimeric polypeptide which consists or consists essentially of the amino acid sequence set forth in SEQ ID NO:76.

In some aspects, a polynucleotide encoding a chimeric polypeptide disclosed herein further comprises a nucleic acid sequence encoding a signaling domain. In some aspects, the signaling domain comprises a domain derived from CD3zeta, FcR gamma, FcR beta, CD3 gamma, CD3 delta, CD3 epsilon, CD5, CD22, CD79a, CD79b, CD66d, or combinations thereof.

In some aspects, a polynucleotide encoding a chimeric polypeptide disclosed herein further comprises a nucleic acid sequence encoding a co-stimulatory domain. In some aspects, the co-stimulatory domain comprises a domain derived from 2B4, HVEM, ICOS, LAG3, DAP10, DAP12, CD27, CD28, 4-1BB (CD137), OX40 (CD134), CD30, CD40, ICOS (CD278), glucocorticoid-induced tumor necrosis factor receptor (GITR), lymphocyte function-associated antigen-1 (LFA-1), CD2, CD7, LIGHT, NKG2C, B7-H3, or combinations thereof.

In some aspects, a polynucleotide encoding a chimeric polypeptide disclosed herein further comprises a nucleic acid sequence encoding an antigen binding domain. In some aspects, the antigen-binding domain comprises an antibody or an antigen binding fragment thereof that specifically binds to an epitope on a tumor antigen. In some aspects, the antigen binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, a nanobody, or combinations thereof. Non-limiting examples of antigens that the antigen binding domain can bind specifically to include CD19, TRAC, TCRβ, BCMA, CLL-1, CS1, CD38, CD19, TSHR, CD123, CD22, CD30, CD70, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WTI, NY-ESO-1, LAGE-la, MAGE-Al, legumain, HPV E6,E7, MAGE Al, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, and any combinations thereof.

In some aspects, a polynucleotide encoding a chimeric polypeptide disclosed herein further comprises a nucleic acid sequence encoding a transmembrane domain.

In some aspects, a polynucleotide encoding a chimeric polypeptide disclosed herein further comprises a nucleic acid sequence encoding a chimeric antigen receptor (CAR), e.g., a standard CAR, a split CAR, an off-switch CAR, an on-switch CAR, a first-generation CAR, a second-generation CAR, a third-generation CAR, or a fourth-generation CAR.

In some aspects, a polynucleotide encoding a chimeric polypeptide disclosed herein encodes a CAR comprising:
(i) a chimeric polypeptide disclosed herein;
(ii) an antigen-binding domain that binds to an epitope on a tumor antigen expressed on a target cell; and,
(iii) a transmembrane domain.

In some aspects, a polynucleotide encoding a chimeric polypeptide disclosed herein further comprises a nucleic acid sequence encoding a T cell receptor (TCR).

In some aspects, a polynucleotide encoding a chimeric polypeptide disclosed herein, e.g., a CAR or a TCR comprising a chimeric polypeptide of the present disclosure, can induce IFNγ and/or IL-2 expression in a cell (e.g., a T cell).

In some aspects, a polynucleotide comprising a nucleic acid sequence encoding a chimeric polypeptide disclosed herein can further comprise a nucleic acid sequence encoding a key polypeptide, e.g., an inducible key polypeptide.

In some aspects, a polynucleotide comprising a nucleic acid sequence encoding a chimeric polypeptide disclosed herein can further comprise a nucleic acid sequence encoding a key polypeptide, e.g., an inducible key polypeptide. In some aspects, the nucleic acid sequence encoding the chimeric polypeptide and the nucleic acid sequence encoding the key polypeptide can be on the same sequence. In some aspects, the nucleic acid sequence encoding the chimeric polypeptide and the nucleic acid sequence encoding the key polypeptide can be on different sequences.

As described herein, the key polypeptide is capable of inhibiting binding of the latch region to the structural region of the chimeric polypeptide when the key polypeptide comes in contact with the chimeric polypeptide. In other words, the presence of a key polypeptide that preferentially binds to the structural region of the cage displaces the latch, and therefore the degron sequence contained in the latch becomes physiologically active.

In some aspects, the present disclosure also provides a polynucleotide set comprising a first polynucleotide and a second polynucleotide, wherein the first polynucleotide comprises a nucleic acid sequence encoding a chimeric polypeptide (e.g., those described above), and the second polynucleotide comprises a nucleic acid sequence encoding a key polypeptide.

In some aspects, the second polynucleotide of a polynucleotide set disclosed herein encodes a key polypeptide, which comprises, consists, or consists essentially of an amino acid sequence having at least about 50%, at least about 55%, at least about 60%, at least about 65% least about 70%, at least about 75%, at least about 80%, at least about 85%, at least about 90%, at least about 95%, at least about 96%, at least about 97%, at least about 98%, at least about 99%, or about 100% sequence identity to the sequence as set forth in SEQ ID NOS: 77 to 96, wherein the key polypeptide is capable of binding to the structural region of the chimeric polypeptide.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:14, SEQ ID NO:15, or, SEQ ID NO:16; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 77.

In certain aspects, (i) the chimeric polypeptide comprises the amino acid sequence set forth in SEQ ID NO:14, SEQ ID NO:15 or SEQ ID NO: 16, and (ii) the key polypeptide comprises the amino acid sequence set forth in SEQ ID NO: 77.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:17 or SEQ ID NO:18; and
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 78.

In certain aspects, (i) the chimeric polypeptide comprises the amino acid sequence set forth in SEQ ID NO:17 or SEQ ID NO: 18, and (ii) the key polypeptide comprises the amino acid sequence set forth in SEQ ID NO: 78.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 19; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 79.

In certain aspects, (i) the chimeric polypeptide comprises the amino acid sequence set forth in SEQ ID NO: 19, and (ii) the key polypeptide comprises the amino acid sequence set forth in SEQ ID NO: 79.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 20, 818, or 819; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 80.

In certain aspects, (i) the chimeric polypeptide comprises the amino acid sequence set forth in SEQ ID NO: 20, 818, or 819, and (ii) the key polypeptide comprises the amino acid sequence set forth in SEQ ID NO: 80.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any one of the amino acid sequences set forth in SEQ ID NOS: 22, 23, 24, 26, 27, 807, 808, 809, or 810; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the sequence as set forth in SEQ ID NO: 81.

In certain aspects, (i) the chimeric polypeptide comprises an amino acid sequence set forth in SEQ ID NO: 22, 23, 24, 26, 27, 807, 808, 809, or 810, and (ii) the key polypeptide comprises the amino acid sequence set forth in SEQ ID NO: 81.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any one of the amino acid sequences set forth in SEQ ID NOS: 21, 25, 28-34, 811, 814, or 815; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 82.

In certain aspects, (i) the chimeric polypeptide comprises the amino acid sequence set forth in any one of SEQ ID NOS: 21, 25, 28-34, 811, 814, or 815, and (ii) the key polypeptide comprises the amino acid sequence set forth in SEQ ID NO: 82.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any one of the amino acid sequences set forth in SEQ ID NO:35-48, 812 or 813; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 83.

In certain aspects, (i) the chimeric polypeptide comprises an amino acid sequence set forth in any one of SEQ ID NOS: 35-48, 812 or 813, and (ii) the key polypeptide comprises the amino acid sequence set forth in SEQ ID NO: 83.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any one of the amino acid sequences set forth in SEQ ID NOS:49-54; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 84.

In certain aspects, (i) the chimeric polypeptide comprises any one of the amino acid sequences set forth in SEQ ID NOS:49, 50, 51, 52, 53, or 54, and (ii) the key polypeptide comprises the amino acid sequence set forth in SEQ ID NO: 84.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any of the amino acid sequences set forth in SEQ ID NOS:55, 56, or 58; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 85.

In certain aspects, (i) the chimeric polypeptide comprises the amino acid sequence set forth in any one of SEQ ID NO:55, 56, or 58, and (ii) the key polypeptide comprises the amino acid sequence set forth in SEQ ID NO: 85.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:57 or SEQ ID NO:59; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 86.

In certain aspects, (i) the chimeric polypeptide comprises the amino acid sequence set forth in SEQ ID NO:57 or SEQ ID NO:59, and (ii) the key polypeptide comprises the amino acid sequence set forth in SEQ ID NO: 86.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acids sequence set forth in SEQ ID NO:60; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 87.

In certain aspects, (i) the chimeric polypeptide comprises the amino acid sequence set forth in SEQ ID NO:60, and (ii) the key polypeptide comprises the amino acid sequence set forth in SEQ ID NO: 87.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:61, 820 or 821; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acids sequence set forth in SEQ ID NO: 88.

In certain aspects, (i) the chimeric polypeptide comprises the amino acid sequence set forth in SEQ ID NO:61, 820 or 821, and (ii) the key polypeptide comprises the amino acid sequence set forth in SEQ ID NO: 88.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:62; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 89.

In certain aspects, (i) the chimeric polypeptide comprises the amino acid sequence set forth in SEQ ID NO:62, and (ii) the key polypeptide comprises the amino acid sequence set forth in SEQ ID NO: 89.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:63; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 90.

In certain aspects, (i) the chimeric polypeptide comprises the amino acid sequence set forth in SEQ ID NO:63, and (ii) the key polypeptide comprises the amino acid set forth in SEQ ID NO: 90.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO:65; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 91.

In certain aspects, (i) the chimeric polypeptide comprises the amino acid sequence set forth in SEQ ID NO:65, and (ii) the key polypeptide comprises the amino acid set forth in SEQ ID NO: 91.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any of the amino acid sequences set forth in SEQ ID NO:64, 66, or 67; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 92.

In certain aspects, (i) the chimeric polypeptide comprises any of the amino acid sequences set forth in SEQ ID NOS:64, 66, or 67, and (ii) the key polypeptide comprises the amino acid set forth in SEQ ID NO: 92.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any one of the amino acid sequences set forth in SEQ ID NOS: 68, 69, 70, or 73; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 93.

In certain aspects, (i) the chimeric polypeptide comprises any one of the amino acid sequences set forth in SEQ ID NOS:68, 69, 70, or 73, and (ii) the key polypeptide comprises the amino acid sequence set forth in SEQ ID NO: 93.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to any one of the amino acid sequences set forth in SEQ ID NOS:71, 72, 74, 816, or 817; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 94.

In certain aspects, (i) the chimeric polypeptide comprises any one of the amino acid sequences set forth in SEQ ID NOS:71, 72, 74, 816, or 817, and (ii) the key polypeptide comprises the amino acid set forth in SEQ ID NO: 94.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 75; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to the amino acid sequence set forth in SEQ ID NO: 95.

In certain aspects, (i) the chimeric polypeptide comprises the amino acid sequence set forth in SEQ ID NO: 75, and (ii) the key polypeptide comprises the amino acid set forth in SEQ ID NO: 95.

In some aspects, the present disclosure provides
(a) a polynucleotide that comprises a nucleic acid sequence encoding a chimeric polypeptide disclosed herein and further comprises a nucleic acid sequence encoding a key polypeptide, e.g., an inducible key polypeptide; or,
(b) a polynucleotide set, wherein a first polynucleotide comprises a nucleic acid sequence encoding a chimeric polypeptide disclosed herein, and a second polynucleotide comprises a nucleic acid sequence encoding a key polypeptide,
   wherein
(i) the chimeric polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 76; and,
(ii) the key polypeptide comprises an amino acid sequence having at least about 50%, at least about 55%, at least about 60%, at least about 65%, at least about 70%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% sequence identity to the amino acid sequence set forth in SEQ ID NO: 96.

In certain aspects, (i) the chimeric polypeptide comprises the amino acid sequence set forth in SEQ ID NO: 76, and (ii) the key polypeptide comprises the amino acid set forth in SEQ ID NO: 96.

### IV. Vectors

In some aspects, the present disclosure provides a vector comprising an isolated nucleic acid molecule that encodes a chimeric polypeptide disclosed herein, an isolated nucleic acid molecule that encodes a key polypeptide (or a plurality of key polypeptides), or a combination thereof. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In certain aspects, the nucleic acid is a cDNA molecule. Nucleic acids described herein can be obtained using standard molecular biology techniques known in the art.

In some aspects, the disclosure provides a vector comprising (a) a polynucleotide encoding a chimeric polypeptide disclosed herein, and (b) a polynucleotide encoding one or more key polypeptides capable of binding to the structural region of the cage polypeptide of the chimeric polypeptide.

In some aspects, the present disclosure provides a vector comprising
(a) one or more polynucleotides encoding a chimeric polypeptide disclosed herein, operatively linked to a first promoter; and
(b) one or more polynucleotides encoding one or more key polypeptides capable of binding to the structural region of the cage polypeptide of the chimeric polypeptide, wherein the one or more polynucleotides encoding the one or more key polypeptides are operatively linked to a second promoter.

In some aspects, multiple open reading frames encoding a key polypeptide are operatively linked to a single promoter (e.g., an inducible promoter). In some aspects, each polynucleotide encoding a key polypeptide is operatively linked to a different promoter, i.e., the expression of each key polypeptide is independently controlled by its own promoter (e.g., an inducible promoter). When multiple inducible promoters are present, they can the induced by the same inducer molecule or a different inducer.

In some aspects, a nucleic acid molecule encoding a chimeric polypeptide disclosed herein, a nucleic acid molecule encoding a key polypeptide, or a combination thereof can be operably linked to one or more regulatory elements. Regulatory elements can include, e.g., promoters/enhancers (such as exhaustion-responsive promoters, activation-responsive promoters, cytokine-responsive promoters, calcium-responsive promoters, and the like), localization sequences (such as membrane-localization sequences, nuclear localization sequences, nuclear exclusion sequences, proteasomal targeting sequences, and the like), post-translational modification sequences (such as ubiquitination, phosphorylation, dephosphorylation, and the like).

Suitable vectors for the disclosure include expression vectors, viral vectors, and plasmid vectors. In some aspects, the vector is a viral vector, a mammalian vector, or a bacterial vector. In some aspects, the vector is a viral vector. As used herein, the terms “vector” and “expression vector” refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

One can readily employ any vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, the vector is a retroviral vector. As used herein, viral vectors include, but are not limited to, selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector. In some specific aspects, the vector is a lentivirus. Examples of lentiviral vectors are disclosed in International Application Publication Nos. WO9931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, Calif.). Other plasmids are well-known to those of ordinary skill in the art. Additionally, plasmids can be custom designed using standard molecular biology techniques to remove and/or add specific fragments of DNA.

In some aspects, the vector comprises a first polynucleotide encoding a chimeric polypeptide of the present disclosure and a second polynucleotide encoding a key polypeptide (or a plurality of key polypeptides). Thus, in some aspects, the polynucleotide encoding the chimeric polypeptide and the polynucleotide encoding the key polypeptide are on the same vector. In other aspects, the polynucleotide encoding the chimeric polypeptide and the second polynucleotide encoding the key polypeptide (or a plurality of key polypeptides) are on different vectors.

A polynucleotide encoding a key polypeptide can be operably linked to a promoter, e.g., an inducible promoter. Thus, the expression of the key polypeptide can be triggered or modulated by the administration of a ligand that, upon activation of the inducible promoter, results in the expression of the key polypeptide. Inducing expression of the key polypeptide can allow tunable control of the chimeric polypeptide by modulated the population of cage polypeptides that become activated. Tunable control of the cage polypeptide activation therefore allows tunable control of the endocytotic processing of a membrane protein, e.g., a CAR or TCR, comprising the chimeric polypeptide. In turn, tunable control of endocytotic processing and protein degradation allows tunable control of surface expression levels of the membrane protein, e.g., a CAR or TCR. Accordingly, the tunable control of the surface expression levels of the membrane protein, e.g., a CAR or TCR, would allow modulating, preventing, or reverting immune cell exhaustion.

In some aspects, the polynucleotides disclosed herein are integrated into the genome of a host cell (e.g., a nucleic acid encoding a CAR or TCR comprising a chimeric polypeptide of the present disclosure can be integrated into the genome of an immune cell, e.g., a T-cell).

In some specific aspects, a polynucleotide of the present disclosure (e.g., a polynucleotide encoding a chimeric polypeptide disclosed herein) is a polynucleotide encoding a CAR as set forth, e.g., in SEQ ID NO: 98. In other specific aspects, a polynucleotide of the present disclosure (e.g., a polynucleotide encoding a chimeric polypeptide disclosed herein) is a polynucleotide encoding a CAR as set forth in SEQ ID NO: 100.

In some aspects, the polynucleotides disclosed herein are DNA (e.g., a DNA molecule or a combination thereof), RNA (e.g., an RNA molecule or a combination thereof), or any combination thereof. The polynucleotides disclosed herein comprise nucleic acid sequences comprising single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure (e.g., a chimeric polypeptide of the present disclosure or a key polypeptide of the present disclosure). The disclosure further provides expression vectors comprising the polynucleotides of the disclosure (e.g., a polynucleotide encoding a chimeric polypeptide of the present disclosure or a polynucleotide encoding a key polypeptide of the present disclosure) operatively linked to a promoter, e.g., an inducible promoter. The disclosure further provides cells comprising the expression vectors comprising polynucleotides of the present disclosure operatively linked to a promoter.

The vectors disclosed herein can comprises a nucleic acid coding region (e.g., a polynucleotide encoding a chimeric polypeptide disclosed herein, a polynucleotide encoding a CAR or TCR comprising a chimeric polypeptide disclosed herein, a polynucleotide encoding a key polypeptide, or any combination thereof) operatively linked to any control sequences capable of effecting expression of the gene product (e.g., a chimeric polypeptide disclosed herein, a protein comprising a chimeric polypeptide disclosed herein, a CAR or TCR comprising a chimeric polypeptide disclosed herein, a key polypeptide, or a combination thereof). “Control sequences” operably linked to the nucleic acid sequences of the disclosure (e.g., a polynucleotide encoding a chimeric polypeptide disclosed herein, a polynucleotide encoding a CAR or TCR comprising a chimeric polypeptide disclosed herein, a polynucleotide encoding a key polypeptide, or any combination thereof) are nucleic acid sequences capable of effecting the expression of the nucleic acid molecules. The control sequences need not be contiguous with the nucleic acid sequences of the disclosure, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various aspects, the expression vector may comprise a plasmid, viral-based vector, or any other suitable expression vector as discussed above.

### V. Nucleic Acid Modifications

The polynucleotides of the present disclosure (e.g., a polynucleotide encoding a chimeric polypeptide disclosed herein or a polypeptide key) can comprise nucleic acids with one or more modifications. Accordingly, in some aspects, a polynucleotide sequence disclosed herein can comprise at least one nucleotide analogue. In some aspects, at least one nucleotide analogue introduced by using in vitro translation (IVT) or chemical synthesis is selected from the group consisting of a 2′-O-methoxyethyl-RNA (2′-MOE-RNA) monomer, a 2′-fluoro-DNA monomer, a 2′-O-alkyl-RNA monomer, a 2′-amino-DNA monomer, a locked nucleic acid (LNA) monomer, a cEt monomer, a cMOE monomer, a 5′-Me-LNA monomer, a 2′-(3-hydroxy)propyl-RNA monomer, an arabino nucleic acid (ANA) monomer, a 2′-fluoro-ANA monomer, an anhydrohexitol nucleic acid (HNA) monomer, an intercalating nucleic acid (INA) monomer, and a combination of two or more of said nucleotide analogues. In some aspects, the modified nucleic acid molecule comprises at least one backbone modification, for example, a phosphorothioate internucleoside linkage.

In some aspects, nucleic acids disclosed herein can be chemically modified at terminal locations, for example by introducing M (2′-O-methyl), MS (2′-O-methyl 3′ phosphorothioate), or MSP (2′-O-methy 3′thioPACE, phosphonoacetate) modifications, or combinations thereof at positions 1, 2, 3 respect to the 5′ and/or 3′ termini.

In some aspects, the nucleic acids provided herein are synthetic, e.g., modified DNA molecules encoding RNA (e.g., an mRNA encoding a CAR or a TCR) and/or modified RNA encoding polypeptides (e.g., encoding a key polypeptide), where the synthetic, modified DNA or RNA molecules comprise one or more modifications.

The modified (e.g., synthetic) polynucleotides disclosed herein, e.g., a polynucleotide encoding a chimeric polypeptide of the present disclosure, a polynucleotide encoding a key polypeptide, or a polynucleotide or set of polynucleotides encoding a CAR or a TCR, include modifications to prevent rapid degradation by endo- and exo-nucleases.

Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

Specific examples of modified (e.g., synthetic) polynucleotides disclosed herein, e.g., a polynucleotide encoding a chimeric polypeptide of the present disclosure, a polynucleotide encoding a key polypeptide, or a polynucleotide or set of polynucleotides encoding a CAR or a TCR, useful with the methods described herein include, are not limited to, modified nucleic acids containing modified or non-natural internucleoside linkages.

Modified (e.g., synthetic) polynucleotides disclosed herein, e.g., a polynucleotide encoding a chimeric polypeptide of the present disclosure, a polynucleotide encoding a key polypeptide, or a polynucleotide or set of polynucleotides encoding a CAR or a TCR, having modified internucleoside linkages include, among others, those that do not have a phosphorus atom in the internucleoside linkage.

In other aspect, a modified (e.g., synthetic) polynucleotide disclosed herein, e.g., a polynucleotide encoding a chimeric polypeptide of the present disclosure, a polynucleotide encoding a key polypeptide, or a polynucleotide or set of polynucleotides encoding a CAR or a TCR, has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, T-5′ linked analogs of these, and those) having inverted polarity wherein the adjacent pairs of nucleoside units are linked 3′-5′ to 5′-3′ or T-5′ to 5′-T. Various salts, mixed salts and free acid forms are also included.

As non-limiting examples, modified (e.g., synthetic) polynucleotides disclosed herein, e.g., a polynucleotide encoding a chimeric polypeptide of the present disclosure, a polynucleotide encoding a key polypeptide, or a polynucleotide or set of polynucleotides encoding a CAR or a TCR, can include at least one modified nucleoside including a 2′-O-methyl modified nucleoside, a nucleoside comprising a 5′ phosphorothioate group, a 2′-amino-modified nucleoside, 2′-alkyl-modified nucleoside, morpholino nucleoside, a phosphoramidate or a non-natural base comprising nucleoside, or any combination thereof.

Standard methods can be applied to synthesize an isolated polynucleotide sequence encoding an isolated polypeptide of interest. For example, a single DNA or RNA oligomer containing a codon-optimized nucleotide sequence coding for the particular isolated polypeptide can be synthesized. In other aspects, several small oligonucleotides coding for portions of the desired polypeptide can be synthesized and then ligated. In some aspects, the individual oligonucleotides typically contain 5′ or 3′ overhangs for complementary assembly.

A polynucleotide disclosed herein (e.g., a polynucleotide encoding a chimeric polypeptide disclosed herein, a polynucleotide encoding a key polypeptide, or a polynucleotide or set of polynucleotides encoding a CAR or a TCR disclosed herein) can be chemically synthesized using chemical synthesis methods and potential nucleobase substitutions known in the art. See, for example, International Publication Nos. WO2014093924, WO2013052523; WO2013039857, WO2012135805, and WO2013151671; U.S. Publ. No. US20130115272; or U.S. Pat. Nos. 8,999,380 and 8,710,200, all of which are herein incorporated by reference in their entireties.

### VI. Cell and Methods of Making Engineered Cells

The present disclosure provides methods of generating or preparing cells expressing a chimeric polypeptide disclosed herein. The present disclosure provides methods of making an engineered cell, e.g., an engineered immune cell (e.g., an engineered T cell), comprising transfecting (i) a polynucleotide encoding a chimeric polypeptide disclosed herein, (ii) a polynucleotide encoding a chimeric polypeptide disclosed herein and a corresponding key polypeptide, (iii) a polynucleotide set comprising a first polynucleotide encoding a chimeric polypeptide disclosed herein and a second polynucleotide encoding a corresponding key polypeptide, (iv) a polynucleotide encoding a CAR or a TCR comprising a chimeric polypeptide disclosed herein, or (v) a vector or set of vectors comprising (i), (ii), (iii) or (iv), in a cell, e.g., an immune cell such as a T cell.

The expression of a chimeric polypeptide of the present disclosure as part of a CAR or a TCR can modulate the levels of CAR or TCR (e.g., level of CAR or TCR expressed on the surface of a cell such as a T cell) in response to activation of the chimeric polypeptide by a key polypeptide. In some aspects, the chimeric polypeptide (e.g., as part of a CAR or a TCR) is expressed in the cell following transfection. In some aspects, the transfected cell can be administered to a subject in need thereof. In some aspect, the administration of the transfected cell to the subject can be followed by the administration of a key polypeptide (or a plurality of key polypeptides), or the administration of an inducer capable of inducing the endogenous expression of a key polypeptide (or a plurality of key polypeptides).

The present disclosure also provides cells prepared as disclosed above. Accordingly, the present disclosure provides cells comprising a polynucleotide disclosed herein, a vector disclosed herein, or a chimeric polypeptide disclosed herein.

The present disclosure also provides a cell genetically modified to express a CAR or a TCR, wherein the cell comprises a polynucleotide disclosed herein, a vector disclosed herein, or a chimeric polypeptide disclosed herein. In some aspects, the cell is a T cell, a NK cell (natural killer cell), a NKT cell (natural killer T cell), or an ILC cell (innate lymphoid cell).

In some aspects, the disclosure provides a cell comprising
(a) a polynucleotide encoding a chimeric polypeptide disclosed herein, and
(b) a polynucleotide encoding one or more key polypeptides capable of binding to the structural region of the cage polypeptide of the chimeric polypeptide.

In some aspects, the present disclosure provides cells (e.g., recombinant host cells or immune cells) comprising (a) one or more polynucleotides encoding a chimeric polypeptide disclosed herein, operatively linked to a first promoter; and

(b) one or more polynucleotides encoding one or more key polypeptides capable of binding to the structural region of the cage polypeptide of the chimeric polypeptide of the disclosure, wherein the one or more polynucleotides encoding the one or more key polypeptides are operatively linked to a second promoter.

In some aspects, the present disclosure provides cells (e.g., recombinant host cells or immune cells) that comprise the expression vectors disclosed herein, wherein cells host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated-transfection.

A method of producing a chimeric polypeptide disclosed herein using cells disclosed herein is an additional part of the disclosure. In one aspect, the method comprises the steps of (a) culturing a cell (e.g., a host cell) under conditions conducive to the expression of the polypeptide (e.g., a CAR or TCR comprising a chimeric polypeptide of the present disclosure, a key polypeptide, or a combination of key polypeptides), and (b) optionally, recovering the expressed polypeptide or polypeptides. The expressed polypeptide or polypeptides can be recovered from the cell free extract or recovered from the culture medium. In another aspect, the method comprises chemically synthesizing the polypeptides, e.g., using solid phase peptide synthesis.

### VI. Methods of Reducing Exhaustion/Dysfunction

The present disclosure also provides methods of reducing, ameliorating, or inhibiting exhaustion or dysfunction of a cell (e.g., an immune cell) comprising modifying the cell to express a CAR or a TCR comprising a chimeric polypeptide of the present disclosure that can modulate the levels of CAR or TCR in response to activation of the chimeric polypeptide by a key polypeptide.

One of the various ways that tumor cells can evade a host immune response is by causing tumor-specific immune cells, e.g., T cells, to become exhausted. As used herein, the term “exhaustion,” or more specifically, “T cell exhaustion,” refers to the loss of T cell function, which can occur as a result of an infection or a disease (e.g., cancer). T cell exhaustion can be used interchangeably with “T cell dysfunction” or “T cell anergy” in the present disclosure. In some aspects, T cell exhaustion is associated with increased expression of various immune checkpoint inhibitory molecules (e.g., PD-1, TIM-3, and LAG-3), apoptosis, and reduced effector function (e.g., cytokine production and expression of cytotoxic molecules, such as perforin and granzymes). Accordingly, the terms “reduce T cell exhaustion,” “ameliorate T cell exhaustion,” “inhibit T cell exhaustion,” and the like, refer to a condition of restored functionality of T cells characterized by one or more of the following: (i) decreased expression of one or more immune checkpoint inhibitory molecules (e.g., PD-1, TIM-3, and LAG-3), (ii) increased memory formation and/or maintenance of memory markers (e.g., CD45RO, CD62L, and/or CCR7), (iii) prevention of apoptosis, (iv) increased cytokine production (e.g., IL-2, IFN-γ, and/or TNF-α), (v) enhanced killing capacity, (vi) increased recognition of tumor targets with low surface antigen, (vii) enhanced proliferation in response to antigen, and (viii) any combination thereof.

In some aspects, modifying a CAR or a TCR by incorporating a chimeric polypeptide disclosed herein results in a cell, e.g., T cell, with increased resistance or tolerance to exhaustion. Accordingly, in certain aspects, the present disclosure relates to methods of reducing exhaustion in an immune cell, e.g., T cell (e.g., tumor-specific T cell) by modifying a CAR or TCR by incorporating a chimeric polypeptide disclosed herein. In certain aspects, reducing immune cell, e.g., T cell, exhaustion comprises reversing the dysfunction that has already occurred in the immune cell, e.g., T cell, (i.e., making exhausted T cells become less exhausted). In further aspects, reducing immune cell, e.g., T cell, exhaustion comprises preventing a newly activated immune cell, e.g., T cell, from becoming exhausted.

In some aspects, reducing immune cell, e.g., T cell, exhaustion comprises both reversing and preventing exhaustion in an immune cell, e.g., a T cell.

The exhaustion state of an immune cell, e.g., a T cell, can be determined by various methods known in the art. In some aspects, the exhaustion state of an immune cell, e.g., a T cell, can be measured by evaluating the resistance of the immune cell, e.g., a T cell, to apoptosis. In certain aspects, increased resistance to apoptosis can promote the long-term persistence or survival of the T cell.

In some aspects, the exhaustion state of an immune cell, e.g., a T cell, can be measured by evaluating the resistance of the immune cell, e.g., a T cell, to immune checkpoint molecules. Examples of immune checkpoint molecules are known in the art and include, but are not limited to, PD-1, TIM-3, LAG-3, BTLA, SIGLEC7, CD200R, TIGIT, VISTA, and any combination thereof.

In some aspects, the exhaustion state of an immune cell, e.g., a T cell, can be measured by evaluating the ability of the immune cell, e.g., a T cell, to produce cytokines upon stimulation, e.g., T-cell receptor (TCR) stimulation. Non-limiting examples of cytokines include IFN-γ, IL-2, TNF-α, GM-CSF, IL-6, IL-10, IL-4, IL-5, IL-8, IL-9, IL-13, IL-17, IL-22, CCL2, CCL3, and any combination thereof.

In some aspects, the exhaustion state of an immune cell, e.g., a T cell, can be measured by evaluating the ability of the immune cell, e.g., a T cell, to kill tumor cells after repeated tumor challenge. In certain aspects, killing tumor cells comprises preventing the outgrowth of tumor cells.

### VII. Pharmaceutical Compositions

The present disclosure also provides a composition comprising a polynucleotide encoding a chimeric polypeptide of the present disclosure, a polynucleotide encoding a CAR or TCR comprising a chimeric polypeptide of the present disclosure, a vector comprising a polynucleotide encoding a chimeric polypeptide of the present disclosure, a vector comprising a polynucleotide encoding a CAR or TCR comprising a chimeric polypeptide of the present disclosure, a polynucleotide encoding a key polypeptide, a vector comprising a polynucleotide encoding a key polypeptide, or a cell expressing a polypeptide comprising a chimeric polypeptide disclosed herein (e.g., a cell expressing a CAR or TCR comprising a chimeric polypeptide of the present disclosure). In some aspects, the composition is used for treating a subject in need of a CAR therapy.

In some aspects, the composition is a pharmaceutical composition. Accordingly, the present disclosure provides, e.g., pharmaceutical compositions comprising (i) a cell which has been modified to express a chimeric polypeptide disclosed herein, (ii) a key disclosed herein, or (iii) a combination thereof, and a pharmaceutically acceptable carrier, excipient, or stabilizer. As described herein, such pharmaceutical compositions can be used to prevent and/or treat a cancer. In some aspects, an immune cell of the present disclosure (i.e., a cell expressing a CAR or TCR comprising a chimeric polypeptide disclosed herein), present in a pharmaceutical composition disclosed herein is a T cell (e.g., a CAR or TCR-expressing T cell) or an NK cells (e.g., a CAR or TCR-expressing NK cell).

As used herein, the term “pharmaceutical composition” refers to one or more of the compounds described herein, such as, e.g., a CAR of the present disclosure (e.g., a polynucleotide encoding the CAR, a vector comprising a polynucleotide encoding the CAR, or a CAR polypeptide) or a cell expressing a CAR of the present disclosure, mixed or intermingled with, or suspended in one or more other chemical components, such as pharmaceutically acceptable carriers and excipients. One purpose of a pharmaceutical composition is to facilitate administration of preparations of, e.g., cell expressing a CAR of the present disclosure to a subject.

The terms “excipient” and “carrier” are used interchangeably and refer to an inert substance added to a pharmaceutical composition to further facilitate administration of a compound, e.g., a CAR of the present disclosure.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricularly, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles, and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutical compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, each of which is herein incorporated by reference in its entirety.

The compositions to be used for in vivo administration can be sterile. This is readily accomplished by filtration through, e.g., sterile filtration membranes.

The present disclosure also provides a cell composition comprising a nucleic acid encoding a chimeric polypeptide disclosed herein. In some aspects, the present disclosure provides a cell composition comprising a means for reducing, ameliorating, or inhibiting exhaustion and/or dysfunction in a population of immune cells, e.g., immune cells expressing a CAR or a TCR. In some aspects, the means comprises modifying a nucleic acid encoding a CAR or a TCR by inserting a sequence encoding a chimeric polypeptide disclosed herein in the CAR or TCR encoding sequence, wherein the nucleic acid expresses a CAR or TCR comprising the chimeric polypeptide. In some aspects, the means comprises modifying a CAR or a TCR as described above in the population of immune cells.

### IX. Methods of Treatment

Provided herein are methods for treating a cancer, e.g., a tumor, in a subject in need thereof, comprising administering to the subject an effective amount of a cell composition of the disclosure, e.g., a cell expressing a chimeric polypeptide disclosed herein. In some aspects, the method for treating cancer further comprises administering a key polypeptide. In some aspects, the method for treating cancer further comprises administering an inducer of a key polypeptide (e.g., a compound or physicochemical change than induces endogenous expression of the key polypeptide).

The incorporation of a chimeric polypeptide disclosed herein into a CAR or TCR allows the modulation of the function of the CAR or TCR. The cage portion of the chimeric polypeptide sequesters a degron until a key is provided (e.g., an inducible key endogenously expressed in the cell or a polypeptide key administered to the cell). The key activates the cage polypeptide, releasing the degron, and the degron targets the cage and any functional peptide fused to it (i.e., the CAR or TCR) for degradation. Accordingly, the present disclosure provides a method of controlling a T-cell mediated immune response in a subject in need thereof comprising administering an effective amount of a cell comprising a chimeric disclosed herein (e.g., as part of a CAR or TCR) to the subject.

The present disclosure also provides a method of stimulating a T cell-mediated immune response to a target cell population or tissue in a subject, comprising administering an effective amount of a cell composition of the disclosure, e.g., a cell expressing a chimeric polypeptide disclosed herein to the subject. In some aspects, the method of stimulating a T cell-mediated immune response further comprises administering a key polypeptide. In some aspects, the method of stimulating a T cell-mediated immune response further comprises administering an inducer of a key polypeptide (e.g., a compound or physicochemical change than induces endogenous expression of the key polypeptide).

The present disclosure also provides a method of providing an anti-tumor immunity in a subject in need thereof, the method comprising administering a cell composition of the disclosure, e.g., a cell expressing a chimeric polypeptide disclosed herein, to the subject. In some aspects, the method of providing an anti-tumor immunity further comprises administering a key polypeptide. In some aspects, the method of providing an anti-tumor immunity further comprises administering an inducer of a key polypeptide (e.g., a compound or physicochemical change than induces endogenous expression of the key polypeptide).

In some aspects, the cell administered in the cell composition of the disclosure (e.g., a cell expressing CAR comprising a chimeric polypeptide disclosed herein) is a T cell. In some aspects, the cell is an autologous T cell.

In some aspects, administering the cell composition of the disclosure (e.g., an immune cell such as a T cell expressing a CAR comprising a chimeric polypeptide disclosed herein) reduces a tumor volume in the subject compared to a reference tumor volume. In some aspects, the reference tumor volume is the tumor volume in the subject prior to the administration of the modified cell. In further aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In certain aspects, administering the cell composition of the disclosure (e.g., an immune cell such as a T cell expressing a CAR comprising a chimeric polypeptide disclosed herein) can reduce the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration of the cell composition of the disclosure. In further aspects, the reference tumor weight is the tumor weight in a corresponding subject that did not receive the administration.

In some aspects, administering the cell composition of the disclosure (e.g., an immune cell such as a T cell expressing a CAR comprising a chimeric polypeptide disclosed herein) to a subject, e.g., suffering from a tumor, can increase the number and/or percentage of TILs (e.g., CD4⁺ or CD8⁺) in a tumor and/or TME of the subject. In certain aspects, the number and/or percentage of TTLs in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the cell composition of the present disclosure or the same subject prior to the administration of the cell composition of the present disclosure).

In addition to the above, administering the cell composition of the disclosure (e.g., an immune cell such as a T cell expressing a CAR comprising a chimeric polypeptide disclosed herein) can have other effects which are conducive for the treatment of a tumor.

As described herein, the cell composition of the disclosure (e.g., a cell expressing a CAR or TCR comprising a chimeric polypeptide of the present disclosure) can be used to treat variety of cancer types, e.g., a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

In some aspects, the cell composition of the disclosure (e.g., an immune cell such as a T cell expressing a CAR comprising a chimeric polypeptide disclosed herein) can be used in combination with other therapeutic agents (e.g., anti-cancer agents and/or immunomodulating agents). Accordingly, in certain aspects, a method of treating a tumor disclosed herein comprises administering the cell composition of the disclosure in combination with one or more additional therapeutic agents.

In some aspects, the cell composition of the disclosure (e.g., an immune cell such as a T cell expressing a CAR comprising a chimeric polypeptide disclosed herein) can be used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway).

Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. A comprehensive and non-limiting list of combination treatment is disclosed in detail elsewhere in this application.

In some aspects, the cell composition of the disclosure (e.g., an immune cell such as a T cell expressing a CAR comprising a chimeric polypeptide disclosed herein) is administered to the subject prior to or after the administration of the additional therapeutic agent. In other aspects, the cell composition of the disclosure (e.g., an immune cell such as a T cell expressing a CAR comprising a chimeric polypeptide disclosed herein) is administered to the subject concurrently with the additional therapeutic agent. In certain aspects, the cell composition of the disclosure (e.g., an immune cell such as a T cell expressing a CAR comprising a chimeric polypeptide disclosed herein) and the additional therapeutic agent can be administered concurrently as a single composition in a pharmaceutically acceptable carrier. In other aspects, the cell composition of the disclosure (e.g., an immune cell such as a T cell expressing a CAR comprising a chimeric polypeptide disclosed herein) and the additional therapeutic agent are administered concurrently as separate compositions.

In some aspects, a subject that can be treated with the composition and methods of the present disclosure is a nonhuman animal such as a rat or a mouse. In some aspects, the subject that can be treated is a human.

In some aspects, an immune cell disclosed herein (e.g., a cell expressing a CAR or TCR comprising a chimeric polypeptide of the present disclosure) can be used in combination with other therapeutic agents (e.g., anti-cancer agents and/or immunomodulating agents). Accordingly, in certain aspects, a method of treating a tumor disclosed herein comprises administering an immune cell of the present disclosure (e.g., a cell expressing a CAR or TCR comprising a chimeric polypeptide of the present disclosure) in combination with one or more additional therapeutic agents to a subject. Such agents can include, for example, chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof.

In some aspects, an immune cell disclosed herein (e.g., a cell expressing a CAR or TCR comprising a chimeric polypeptide of the present disclosure) can be used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, an immune cell of the present disclosure (e.g., a cell expressing a CAR or TCR comprising a chimeric polypeptide of the present disclosure) can be used in combination with one or more anti-cancer agents, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that systemically increases the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno-repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab (OPDIVO®), pembrolizumab (KEYTRUDA®; MK-3475), pidilizumab (CT-011), PDR001, MEDI0680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRIQ®; RG7446; MPDL3280A; RO5541267), durvalumab (MEDI4736, IMFINZI®), BMS-936559, avelumab (BAVENCIO®), LY3300054, CX-072 (Proclaim-CX-072), FAZ053, KN035, MDX-1105, and combinations thereof); and anti-CTLA-4 antibody (e.g., ipilimumab (YERVOY®), tremelimumab (ticilimumab; CP-675,206), AGEN-1884, ATOR-1015, and combinations thereof).

In some aspects, an anti-cancer agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g., anti-LAG-3 antibody), 4-1BB (CD137) agonist (e.g., anti-CD137 antibody), GITR agonist (e.g., anti-GITR antibody), TIM3 agonist (e.g., anti-TIM3 antibody), or combinations thereof.

In some aspects, an immune cell disclosed herein (e.g., a cell expressing a CAR or TCR comprising a chimeric polypeptide of the present disclosure) can be administered to the subject prior to or after the administration of the additional therapeutic agent. In other aspects, the immune cell is administered to the subject concurrently with the additional therapeutic agent. In certain aspects, the immune cell (e.g., a T cell) and the additional therapeutic agent can be administered concurrently as a single composition in a pharmaceutically acceptable carrier. In other aspects, the immune cell (e.g., a T cell) and the additional therapeutic agent are administered concurrently as separate compositions. In some aspects, the additional therapeutic agent and the immune cell are administered sequentially.

The present disclosure also provides the use of any polypeptides, polynucleotides, vectors, cells, compositions, or kits disclosed herein for the manufacture of a medicament for treating cancer in a subject in need thereof.

### X. Kits

The present disclosure also provides kits for practicing any of the methods of the present disclosure. The present disclosure provides kits comprising any of the polypeptides (e.g., chimeric polypeptides of the present disclosure), polynucleotides, vectors, or cells disclosed herein, or any combinations thereof.

In some aspects, the kit comprises (a) a polynucleotide encoding a chimeric polypeptide disclosed herein, and (b) a polynucleotide encoding one or more key polypeptides capable of binding to the structural region of the cage polypeptide of a chimeric polypeptide of the present disclosure.

In some aspects, the kit comprises
(a) one or more expression vectors comprising one or more polynucleotides encoding a chimeric polypeptide disclosed herein, operatively linked to a promoter; and,
(b) one or more expression vectors comprising one or more polynucleotides encoding one or more key polypeptides capable of binding to the structural region of the cage polypeptide of a chimeric polypeptide disclosed herein, wherein the one or more polynucleotides encoding the one or more key polypeptides are operatively linked to at least one promoter (e.g., an inducible promoter).

In some aspects, the disclosure provides a kit comprising (i) a polynucleotide (e.g., a vector) comprising a nucleic acid encoding a chimeric polypeptide of the present disclosure, a key polypeptide, or a combination thereof, (ii) a chimeric antigen receptor (CAR) or a T cell receptor (TCR), or (iii) a combination thereof, and optionally instructions for treating a tumor according to any of the methods disclosed herein.

Also provided is a kit comprising (i) a polynucleotide (e.g., a vector) comprising a nucleic acid encoding a chimeric polypeptide of the present disclosure, a key polypeptide, or a combination thereof, (ii) a vector comprising a chimeric antigen receptor (CAR) or a T cell receptor (TCR), or (iii) a combination thereof, and optionally instructions for preparing a cell composition according to the methods disclosed herein.

In some aspects, the present disclosure provides kits comprising the compositions disclosed herein, for example, (i) a cell, e.g., an immune cell, that expresses a CAR or TCR comprising a chimeric polypeptide of the present disclosure, (ii) a polynucleotide comprising a nucleic acid encoding a chimeric polypeptide of the present disclosure, (iii) a polynucleotide comprising a nucleic acid encoding a key polypeptide disclosed herein, (iv) a polypeptide comprising a key polypeptide disclosed herein (e.g., a key polypeptide conjugated or fused to a membrane permeating peptide), (v) a key polypeptide disclosed herein, (vi) a single vector comprising a nucleic acid encoding at least one chimeric polypeptide of the present disclosure and at least one key polypeptide, (vii) a single vector comprising a nucleic acid encoding at least one chimeric polypeptide of the present disclosure, (viii) a single vector comprising a nucleic acid encoding at least one key polypeptide, (ix) a set of vectors wherein a vector comprises a nucleic acid encoding at least one chimeric polypeptide of the present disclosure and another vector comprises a nucleic acid encoding at least one key polypeptide, (x) a single vector comprising a nucleic acid encoding at least one chimeric polypeptide of the present disclosure and at least one key polypeptide, (xi) a vector or set of vectors encoding a CAR or a TCR disclosed herein, or (xi) a combination thereof. In some aspects, the kits further comprise instructions for their use.

The present disclosure provides kits for the treatment of cancer comprising an immune cell disclosed herein, wherein the cell expresses a CAR or TCR comprising a chimeric polypeptide disclosed herein. The present disclosure also provides kits for the treatment of cancer comprising an immune cell disclosed herein, wherein the cell expresses a CAR or TCR comprising a chimeric polypeptide disclosed herein, and at least one polypeptide key.

TABLE 3
Sequences. For amino acid sequences with degron, the Degron A sequence is boxed.

| | | |
|---|---|---|
| SEQ ID | | |
| NO | Description | Sequence |

Full degron

| | | |
|---|---|---|
| 1 | Degron A | KTRGVEEVAEGVVLLRRRGN |

Cages

| | | |
|---|---|---|
| 2 | Cage1 | SVEELLRKLEEVLRKIREENERSLKELRDRAREIVKRNRETNRELEEVIKELEKRLSGA |
| | | DKEKVEELVRRIRRIVERVVEEDRRTVEEIEKIAREVVKRDRDSADRVRRTVEDVLRKA |
| | | TGSEDIVRKIERIVETIEREVRESVKKVEEIARDIRRKVDESVKNVEKLLRDVDKKARD |
| | | RKK |
| 3 | Cage2 | SESDDVIRKLRELLEELRTHVEKSIRDLRKILEDSTRHAKRSIEELERLLEEVRKKPGD |
| | | EEVRKTVEEISRRVAENVKRLEDLYRRMEEEVKKNLDRLRKRVEDIIREVEEARKKGVD |
| | | EEKLKDLIRKLRDILRRAAEAHKKLIDDARESLERAKREHEKLIDRLKKILEELER |
| 4 | Cage3 | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 5 | Cage4 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 6 | Cage5 | SEAEDLEELIKELAELLKDVIRKLEKINRRLVKILEDIIRRLKEISKEAEEELRKGTVE |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | IERILREVGKDKDEAERRRRELKDKLDRLREEHEEVKRRLEEELTRLRETHKKIEKELR |
| | | EALKRVRDRST |
| 7 | Cage6 | SEAEDLEELIKELAELLKDVIRKLEKINRRLVKILEDIIRRLKEISKEAEEELRKGTVE |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | IERILREVGKDKDEAERRRRELKDKLDRLREEHEEVKRRLEEELTRLRETHKKIEKELR |
| | | EALKRVRDRST |
| 8 | Cage7 | DEDRIIEEIARLLEELLRELLELIKKLIETNRRLNEEHERAVRELARLLEELLDRLVKK |
| | | GISDEKLKRIRERLKRALDDLERLHREINKRLEDLVRELEKLVREILKELKDALEELRR |
| | | ASARAGGEEVLRRLEEIVKKLLDLVRRILERLKEIHKDNVRLLRELNERLTRIVEDLVR |
| | | LIREILREAGVDEKIAEEIERELEELRRMIKRLHEDLERKLKESEDELREIEARLEEKI |
| | | RRLEKLERKRR |
| 9 | Cage 8 | SKKELLEEVVRRAIELLKRHLEKLKRILEEIVRLLEEHLEKVERVLEAILSLLDDLLRR |
| | | GGDERAIRTLEDVKRRLREILERLADENAKAIKRLADLLDKLEKRNKEAIERLEEILEE |
| | | LKRVRRDEELLRVLETLLKIIEDILRENTKVLEDLLRLVEEILEANLRVVEELLRLARE |
| | | ILTEIVGDEDKLKEIEDELRRLLEELRRLDKAIKDRLRELKKDLDEANRRIKETLKKLL |
| | | REVEK |
| 10 | Cage 9 | SKEEKLKDDVRAVLEDLDRVLKELEKLSEDNLRELKRVLDRITDLHRRILDELRKGIGS |
| | | EELLRRVEKVLKDNLDLLRKLVEEHKESSERDLKRVEDLVREIKEVLRKLLELEDRGTD |
| | | IRKIEEEIERLLRKIRKAVEESKDLNRRNSERIEEVARRSEELARRLLKEIRE |
| | | RGDSKAAEDILRVLEKLVKVSREAIKLILELSEHHVRVSTRIARLLLDVARKLAEVIKE |
| | | AER |
| 11 | Cage10 | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAE |
| | | IVEELIRIIEDLLRKGNLDEDVKRALEELVSRLRKLLEDVKKASEDIVREVERIVRELA |
| | | KRSDEILKKLEDIVEKLRE |
| 12 | Cage11 | DAEEVVKRLADVLRENDETIRKVVEDLVRIAEENDRLVVWLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | RIIEDLLRKGNLDEDVKRALEELVSRLRKLLEDVKKASEDIVREVERIVRELAKRSDEI |
| | | LKKLEDIVEKLRE |
| 13 | Cage12 | DEEETLRRLLERKVELAKEYLDVSKEVIDRTTKLLDEYLKTSKRIVDATVELLERGDLG |
| | | PDELIKRLAEELERSLRELEEEIKRLKRELEESLKKLKEIIDRLAEEAEKLLAVLKRGE |
| | | GSEEEALRALASLVRELIEVLRENDERLRDVLRRLIEALRKNNEILERVLRKLVRAAEE |
| | | RGRDESSREALEEARRRLEELLRELNEITKDLEAKLEKLLRDLNELTKALEEELKRLLD |
| | | ELKKRTD |

Cages with degron

| | | |
|---|---|---|
| 14 | Peptide1_121 | SVEELLRKLEEVLRKIREENERSLKELRDRAREIVKRNRETNRELEEVIKELEKRLSGA |
| | | DKEKVEELVRRIRRIVERVVEEDRRTVEEIEKIAREVVKRDRDSADRVRRTVEDVLRKA |
| | | 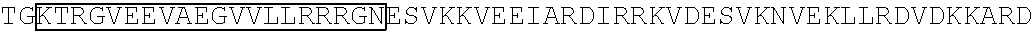 |
| | | RKK |
| 15 | Peptide1_125 | SVEELLRKLEEVLRKIREENERSLKELRDRAREIVKRNRETNRELEEVIKELEKRLSGA |
| | | DKEKVEELVRRIRRIVERVVEEDRRTVEEIEKIAREVVKRDRDSADRVRRTVEDVLRKA |
| | | 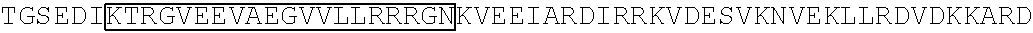 |
| | | RKK |
| 16 | Peptide1_128 | SVEELLRKLEEVLRKIREENERSLKELRDRAREIVKRNRETNRELEEVIKELEKRLSGA |
| | | DKEKVEELVRRIRRIVERVVEEDRRTVEEIEKIAREVVKRDRDSADRVRRTVEDVLRKA |
| | | 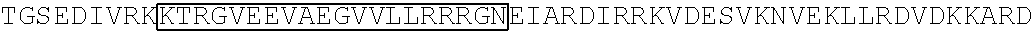 |
| | | RKK |
| 17 | Peptide1_2 | 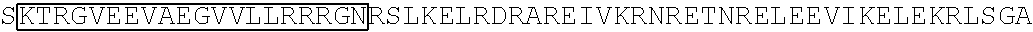 |
| | | DKEKVEELVRRIRRIVERVVEEDRRTVEEIEKIAREVVKRDRDSADRVRRTVEDVLRKA |
| | | TGSEDIVRKIERIVETIEREVRESVKKVEEIARDIRRKVDESVKNVEKLLRDVDKKARD |
| | | RKK |
| 18 | Peptide1_6 | 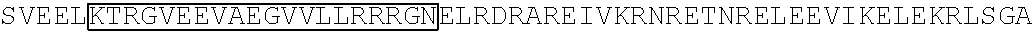 |
| | | DKEKVEELVRRIRRIVERVVEEDRRTVEEIEKIAREVVKRDRDSADRVRRTVEDVLRKA |
| | | TGSEDIVRKIERIVETIEREVRESVKKVEEIARDIRRKVDESVKNVEKLLRDVDKKARD |
| | | RKK |
| 19 | Peptide2_154 | SESDDVIRKLRELLEELRTHVEKSIRDLRKILEDSTRHAKRSIEELERLLEEVRKKPGD |
| | | EEVRKTVEEISRRVAENVKRLEDLYRRMEEEVKKNLDRLRKRVEDIIREVEEARKKGVD |
| | | 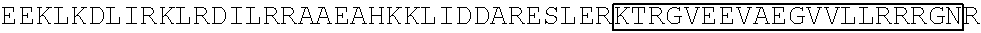 |
| 20 | Peptide2_35 | 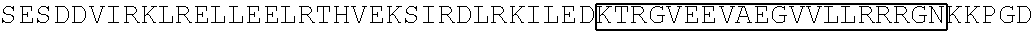 |
| | | EEVRKTVEEISRRVAENVKRLEDLYRRMEEEVKKNLDRLRKRVEDIIREVEEARKKGVD |
| | | EEKLKDLIRKLRDILRRAAEAHKKLIDDARESLERAKREHEKLIDRLKKILEELER |
| 21 | Peptide3_10 | 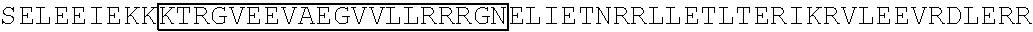 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 22 | Peptide3_123 | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | 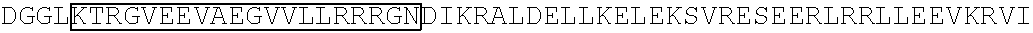 |
| | | EEVAT |
| 23 | Peptide3_127 | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | 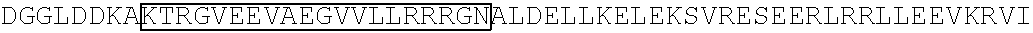 |
| | | EEVAT |
| 24 | Peptide3_130 | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | 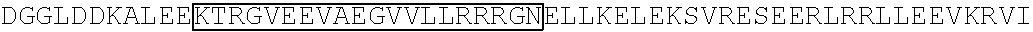 |
| | | EEVAT |
| 25 | Peptide3_14 | 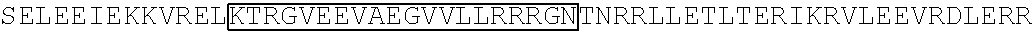 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 26 | Peptide3_159 | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | 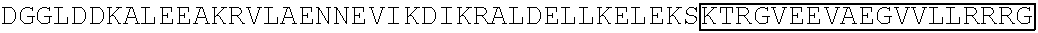 |
| | | 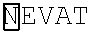 |
| 27 | Peptide3_163 | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | 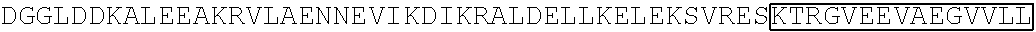 |
| | | 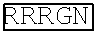 |
| 28 | Peptide3_17 | 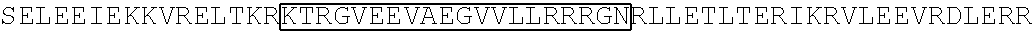 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 29 | Peptide3_21 | 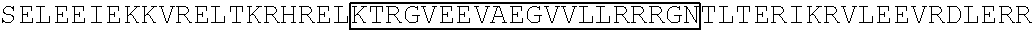 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 30 | Peptide3_28 | 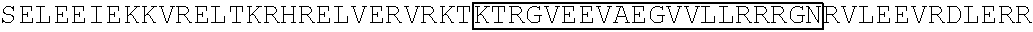 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 31 | Peptide3_3 | 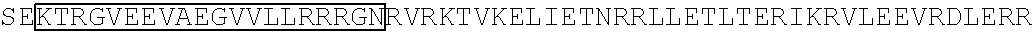 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 32 | Peptide3_32 | 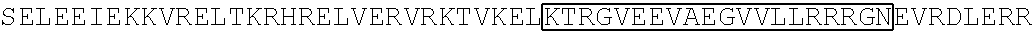 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 33 | Peptide3_35 | 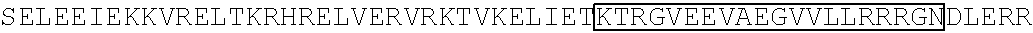 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 34 | Peptide3_39 | 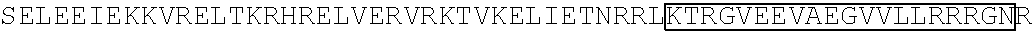 |
| | | GEPKEDLKARLEDIIRRLAEDIERVKRETDKLLEELARIIEEVEKEMRETNKELDKEIK |
| | | DGGLDDKALEEAKRVLAENNEVIKDIKRALDELLKELEKSVRESEERLRRLLEEVKRVI |
| | | EEVAT |
| 35 | Peptide4_125 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 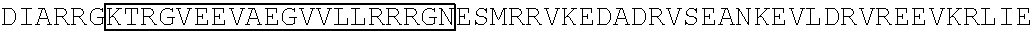 |
| | | EVRETLR |
| 36 | Peptide4_129 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 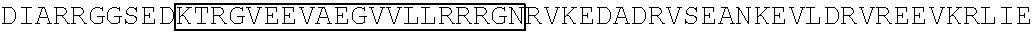 |
| | | EVRETLR |
| 37 | Peptide4_132 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 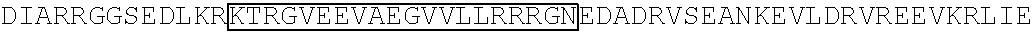 |
| | | EVRETLR |
| 38 | Peptide4_136 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 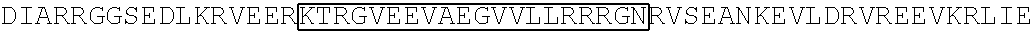 |
| | | EVRETLR |
| 39 | Peptide4_139 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 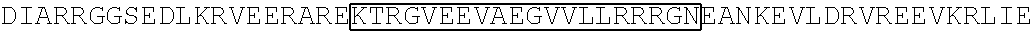 |
| | | EVRETLR |
| 40 | Peptide4_140 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 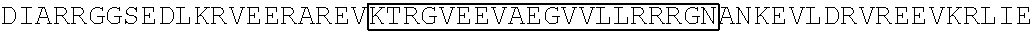 |
| | | EVRETLR |
| 41 | Peptide4_147 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 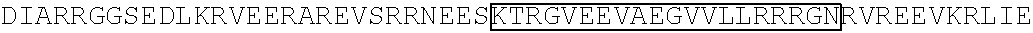 |
| | | EVRETLR |
| 42 | Peptide4_150 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 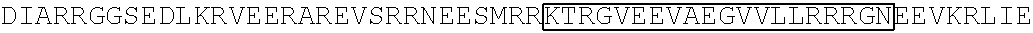 |
| | | EVRETLR |
| 43 | Peptide4_151 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 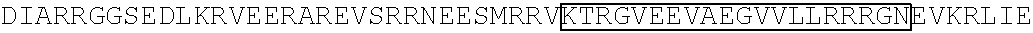 |
| | | EVRETLR |
| 44 | Peptide4_154 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 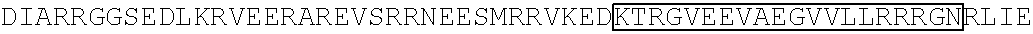 |
| | | EVRETLR |
| 45 | Peptide4_158 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 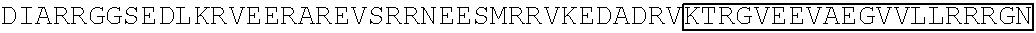 |
| | | EVRETLR |
| 46 | Peptide4_162 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 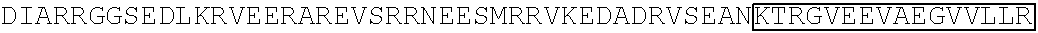 |
| | | 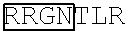 |
| 47 | Peptide4_164 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 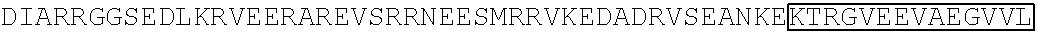 |
| | | 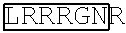 |
| 48 | Peptide4_165 | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | 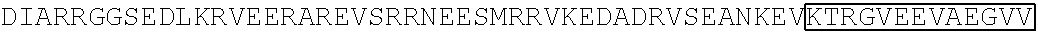 |
| | | 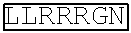 |
| 49 | Peptide4_21 | 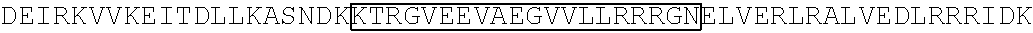 |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 50 | Peptide4_24 | 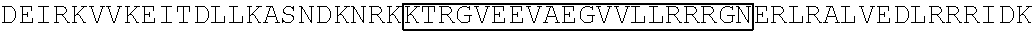 |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 51 | Peptide4_25 | 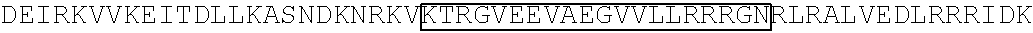 |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 52 | Peptide4_39 | 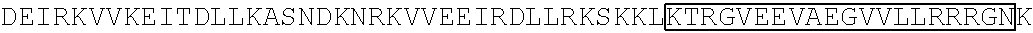 |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 53 | Peptide4_6 | 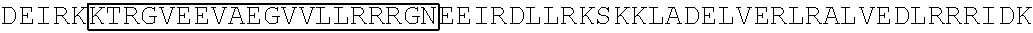 |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 54 | Peptide4_7 | 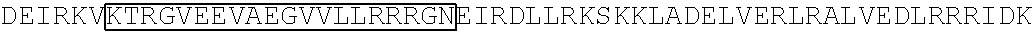 |
| | | SGDKETAEDIVRRIIEELKRILKEIEDLARRINREIERLVEEVERDNRDVNRAIEELLK |
| | | DIARRGGSEDLKRVEERAREVSRRNEESMRRVKEDADRVSEANKEVLDRVREEVKRLIE |
| | | EVRETLR |
| 55 | Peptide5_190 | SEAEDLEELIKELAELLKDVIRKLEKINRRLVKILEDIIRRLKEISKEAEEELRKGTVE |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | 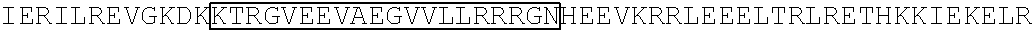 |
| | | EALKRVRDRST |
| 56 | Peptide5_192 | SEAEDLEELIKELAELLKDVIRKLEKINRRLVKILEDIIRRLKEISKEAEEELRKGTVE |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | 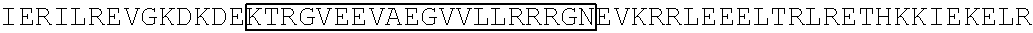 |
| | | EALKRVRDRST |
| 57 | Peptide5_10 | 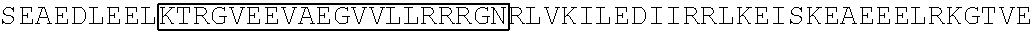 |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | IERILREVGKDKDEAERRRRELKDKLDRLREEHEEVKRRLEEELTRLRETHKKIEKELR |
| | | EALKRVRDRST |
| 58 | Peptide5_199 | SEAEDLEELIKELAELLKDVIRKLEKINRRLVKILEDIIRRLKEISKEAEEELRKGTVE |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | 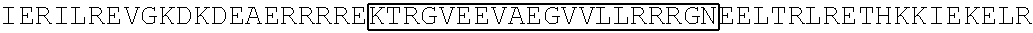 |
| | | EALKRVRDRST |
| 59 | Peptide5_35 | 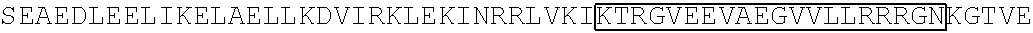 |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | IERILREVGKDKDEAERRRRELKDKLDRLREEHEEVKRRLEEELTRLRETHKKIEKELR |
| | | EALKRVRDRST |
| 60 | Peptide6_6 | 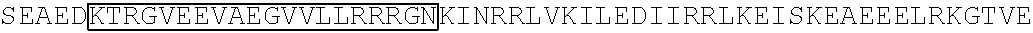 |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | IERILREVGKDKDEAERRRRELKDKLDRLREEHEEVKRRLEEELTRLRETHKKIEKELR |
| | | EALKRVRDRST |
| 61 | Peptide7_218 | DEDRIIEEIARLLEELLRELLELIKKLIETNRRLNEEHERAVRELARLLEELLDRLVKK |
| | | GISDEKLKRIRERLKRALDDLERLHREINKRLEDLVRELEKLVREILKELKDALEELRR |
| | | ASARAGGEEVLRRLEEIVKKLLDLVRRILERLKEIHKDNVRLLRELNERLTRIVEDLVR |
| | | 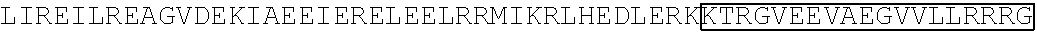 |
| | | 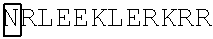 |
| 62 | Peptide7_3 | 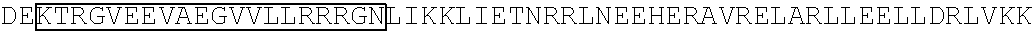 |
| | | GISDEKLKRIRERLKRALDDLERLHREINKRLEDLVRELEKLVREILKELKDALEELRR |
| | | ASARAGGEEVLRRLEEIVKKLLDLVRRILERLKEIHKDNVRLLRELNERLTRIVEDLVR |
| | | LIREILREAGVDEKIAEEIERELEELRRMIKRLHEDLERKLKESEDELREIEARLEEKI |
| | | RRLEEKLERKRR |
| 63 | Peptide8_200 | SKKELLEEVVRRAIELLKRHLEKLKRILEEIVRLLEEHLEKVERVLEAILSLLDDLLRR |
| | | GGDERAIRTLEDVKRRLREILERLADENAKAIKRLADLLDKLEKRNKEAIERLEEILEE |
| | | LKRVRRDEELLRVLETLLKIIEDILRENTKVLEDLLRLVEEILEANLRVVEELLRLARE |
| | | 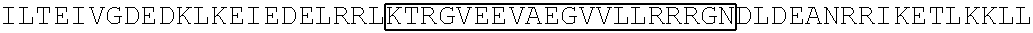 |
| | | REVEK |
| 64 | Peptide9_3 | 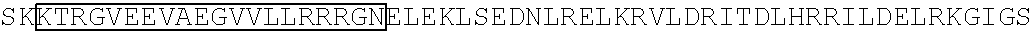 |
| | | EELLRRVEKVLKDNLDLLRKLVEEHKESSERDLKRVEDLVREIKEVLRKLLELEDRGTD |
| | | IRKIEEEIERLLRKIRKAVEESKDLNRRNSERIEEVARRSEELARRLLKEIRERGDSKA |
| | | AEDILRVLEKLVKVSREAIKLILELSEHHVRVSTRIARLLLDVARKLAEVIKEAER |
| 65 | Peptide9_214 | SKEEKLKDDVRAVLEDLDRVLKELEKLSEDNLRELKRVLDRITDLHRRILDELRKGIGS |
| | | EELLRRVEKVLKDNLDLLRKLVEEHKESSERDLKRVEDLVREIKEVLRKLLELEDRGTD |
| | | IRKIEEEIERLLRKIRKAVEESKDLNRRNSERIEEVARRSEELARRLLKEIRERGDSKA |
| | | 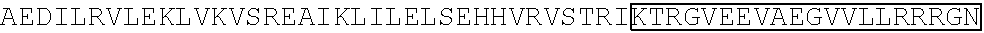 |
| 66 | Peptide9_6 | 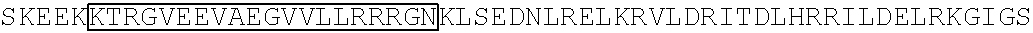 |
| | | EELLRRVEKVLKDNLDLLRKLVEEHKESSERDLKRVEDLVREIKEVLRKLLELEDRGTD |
| | | IRKIEEEIERLLRKIRKAVEESKDLNRRNSERIEEVARRSEELARRLLKEIRERGDSKA |
| | | AEDILRVLEKLVKVSREAIKLILELSEHHVRVSTRIARLLLDVARKLAEVIKEAER |
| 67 | Peptide9_13 | 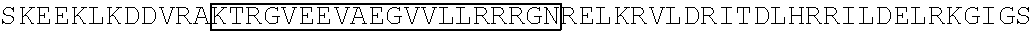 |
| | | EELLRRVEKVLKDNLDLLRKLVEEHKESSERDLKRVEDLVREIKEVLRKLLELEDRGTD |
| | | IRKIEEEIERLLRKIRKAVEESKDLNRRNSERIEEVARRSEELARRLLKEIRERGDSKA |
| | | AEDILRVLEKLVKVSREAIKLILELSEHHVRVSTRIARLLLDVARKLAEVIKEAER |
| 68 | Peptide10_219 | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | 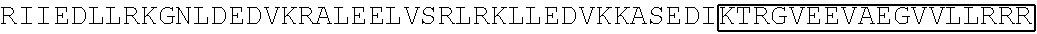 |
| | | 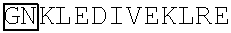 |
| 69 | Peptide10_222 | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | 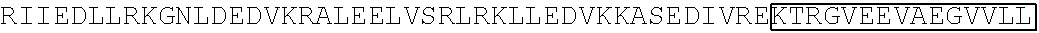 |
| | | 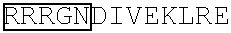 |
| 70 | Peptide10_190 | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | 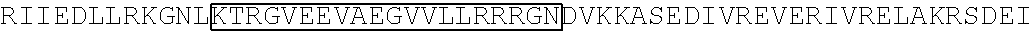 |
| | | LKKLEDIVEKLRE |
| 71 | Peptide10_13 | 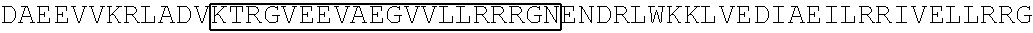 |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | RIIEDLLRKGNLDEDVKRALEELVSRLRKLLEDVKKASEDIVREVERIVRELAKRSDEI |
| | | LKKLEDIVEKLRE |
| 72 | Peptide10_6 | 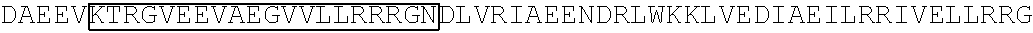 |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | RIIEDLLRKGNLDEDVKRALEELVSRLRKLLEDVKKASEDIVREVERIVRELAKRSDEI |
| | | LKKLEDIVEKLRE |
| 73 | Peptide10_193 | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | 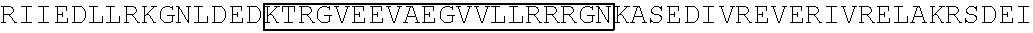 |
| | | LKKLEDIVEKLRE |
| 74 | Peptide10_38 | 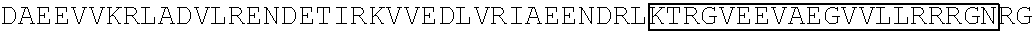 |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | RIIEDLLRKGNLDEDVKRALEELVSRLRKLLEDVKKASEDIVREVERIVRELAKRSDEI |
| | | LKKLEDIVEKLRE |
| 75 | Peptide11_13 | 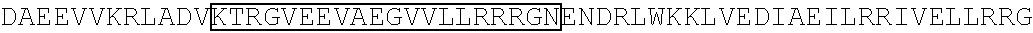 |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | RIIEDLLRKGNLDEDVKRALEELVSRLRKLLEDVKKASEDIVREVERIVRELAKRSDEI |
| | | LKKLEDIVEKLRE |
| 76 | Peptide12_206 | DEEETLRRLLERKVELAKEYLDVSKEVIDRTTKLLDEYLKTSKRIVDATVELLERGDLG |
| | | PDELIKRLAEELERSLRELEEEIKRLKRELEESLKKLKEIIDRLAEEAEKLLAVLKRGE |
| | | GSEEEALRALASLVRELIEVLRENDERLRDVLRRLIEALRKNNEILERVLRKLVRAAEE |
| | | 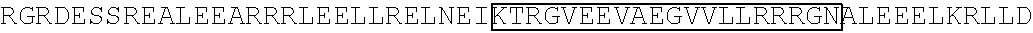 |
| | | ELKKRTD |

KEYS

| | | |
|---|---|---|
| 77 | Key1C | EDIVRKIERIVETIEREVRESVKKVEEIARDIRRKVDESVKNVEKLLRDVDKKARDRKK |
| 78 | Key1N | SVEELLRKLEEVLRKIREENERSLKELRDRAREIVKRNRETNRELEEVIKELEKRLSGA |
| 79 | Key2C | EEKLKDLIRKLRDILRRAAEAHKKLIDDARESLERAKREHEKLIDRLKKILEELER |
| 80 | Key2N | SESDDVIRKLRELLEELRTHVEKSIRDLRKILEDSTRHAKRSIEELERLLEEVRK |
| 81 | Key3C | DKALEEAKRVLAENNEVIKDIKRALDELLKELEKSVRESEERLRRLLEEVKRVIEEVAT |
| 82 | Key3N | SELEEIEKKVRELTKRHRELVERVRKTVKELIETNRRLLETLTERIKRVLEEVRDLERR |
| 83 | Key4C | SEDLKRVEERAREVSRRNEESMRRVKEDADRVSEANKEVLDRVREEVKRLIEEVRETLR |
| 84 | Key4N | DEIRKVVKEITDLLKASNDKNRKVVEEIRDLLRKSKKLADELVERLRALVEDLRRRIDK |
| 85 | Key 5C | KDEAERRRRELKDKLDRLREEHEEVKRRLEEELTRLRETHKKIEKELREALKRVRDRST |
| 86 | Key5N | SEAEDLEELIKELAELLKDVIRKLEKINRRLVKILEDIIRRLKEISKEAEEELRKG |
| 87 | Key6N | SDKEEIKRRVEKTARDLETEHDKIKKRLEDTVRDIKRELDELLEKYERVLRKIEKTLRE |
| | | G |
| 88 | Key7C | EKIAEEIERELEELRRMIKRLHEDLERKLKESEDELREIEARLEEKIRRLEEKLERKRR |
| 89 | Key7N | DEDRIIEEIARLLEELLRELLELIKKLIETNRRLNEEHERAVRELARLLEELLDRLVKK |
| | | G |
| 90 | Key8C | EDKLKEIEDELRRLLEELRRLDKAIKDRLRELKKDLDEANRRIKETLKKLLREVEK |
| 91 | Key9C | SKAAEDILRVLEKLVKVSREAIKLILELSEHHVRVSTRIARLLLDVARKLAEVIKEAER |
| 92 | Key9N | SKEEKLKDDVRAVLEDLDRVLKELEKLSEDNLRELKRVLDRITDLHRRILDELRKGI |
| 93 | Key10C | EDVKRALEELVSRLRKLLEDVKKASEDIVREVERIVRELAKRSDEILKKLEDIVEKLRE |
| 94 | Key10N | DAEEVVKRLADVLRENDETIRKVVEDLVRIAEENDRLVVKKLVEDIAEILRRIVELLRRG |
| 95 | Key11N | DAEEVVKRLADVLRENDETIRKVVEDLVRIAEENDRLVVKKLVEDIAEILRRIVELLRRG |
| 96 | Key12C | SREALEEARRRLEELLRELNEITKDLEAKLEKLLRDLNELTKALEEELKRLLDELKKRT |
| | | D |

CARs

| | | |
|---|---|---|
| 97 | CAR | MVLQTQVFISLLLWISGAYGQEQLVESGGRLVTPGGSLTLSCKASGFDFSAYYMSWVRQ |
| | MND-CD19t-hIgK- | APGKGLEWIATIYPSSGKTYYATSVNGRFTISSDNAQNTVDLQMNSLTAADRATYFCAR |
| | R12-BB-Z-_ | DSYADDGALFNISGPGTLVTISSGGGGSGGGGSGGGGSELVLTQSPSVSAALGSPAKIT |
| | Peptide3_163 | CTLSSAHKTDTIDWYQQLQGEAPRYLMQVQSDGSYTKRPGVPDRFSGSSSGADRYLIIP |
| | | SVQADDEADYYCGADYIGGYVFGGGTQLTVTGESKYGPPCPPCPMFWVLVVVGGVLACY |
| | | SLLVTVAFIIFWVKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPRGSSELEEIEK |
| | | KVRELTKRHRELVERVRKTVKELIETNRRLLETLTERIKRVLEEVRDLERRGEPKEDLK |
| | | ARLEDIIRRLAEDIERVKRETDKLLEELARIIEEVEKEMRETNKELDKEIKDGGLDDKA |
| | | 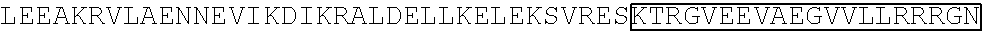 |
| 98 | CAR | ATGGTCTTGCAGACTCAAGTATTTATATCCCTTTTGCTCTGGATCTCTGGAGCTTACGG |
| | MND-CD19t-hIgK- | CCAGGAACAGCTCGTCGAAAGCGGCGGCAGACTGGTGACACCTGGCGGCAGCCTGACCC |
| | R12-BB-Z- | TGAGCTGCAAGGCCAGCGGCTTCGACTTCAGCGCCTACTACATGAGCTGGGTCCGCCAG |
| | Peptide3_163 | GCCCCTGGCAAGGGACTGGAATGGATCGCCACCATCTACCCCAGCAGCGGCAAGACCTA |
| | | CTACGCCACCTGGGTGAACGGACGGTTCACCATCTCCAGCGACAACGCCCAGAACACCG |
| | | TGGACCTGCAGATGAACAGCCTGACAGCCGCCGACCGGGCCACCTACTTTTGCGCTCGG |
| | | GACAGCTACGCCGACGACGGCGCCCTGTTCAACATCTGGGGCCCTGGCACCCTGGTGAC |
| | | AATCTCTAGCGGCGGAGGCGGATCTGGTGGCGGAGGAAGTGGCGGCGGAGGATCTGAGC |
| | | TGGTGCTGACCCAGAGCCCCTCTGTGTCTGCTGCCCTGGGAAGCCCTGCCAAGATCACC |
| | | TGTACCCTGAGCAGCGCCCACAAGACCGACACCATCGACTGGTATCAGCAGCTGCAGGG |
| | | CGAGGCCCCCAGATACCTGATGCAGGTGCAGAGCGACGGCAGCTACACCAAGAGGCCAG |
| | | GCGTGCCCGACAGGTTCAGCGGATCTAGCTCTGGCGCCGACCGCTACCTGATCATCCCC |
| | | AGCGTGCAGGCCGATGACGAGGCCGATTACTACTGTGGCGCCGACTACATCGGCGGCTA |
| | | CGTGTTCGGCGGAGGCACCCAGCTGACCGTGACCGGTGAAAGCAAATACGGACCGCCCT |
| | | GCCCCCCTTGCCCTATGTTCTGGGTGCTGGTGGTGGTCGGAGGCGTGCTGGCCTGCTAC |
| | | AGCCTGCTGGTCACCGTGGCCTTCATCATCTTTTGGGTGAAACGGGGCAGAAAGAAACT |
| | | CCTGTATATATTCAAACAACCATTTATGAGACCAGTACAAACTACTCAAGAGGAAGATG |
| | | GCTGTAGCTGCCGATTTCCAGAAGAAGAAGAAGGAGGATGTGAACTGCGGGTGAAGTTC |
| | | AGCAGAAGCGCCGACGCCCCTGCCTACCAGCAGGGCCAGAATCAGCTGTACAACGAGCT |
| | | GAACCTGGGCAGAAGGGAAGAGTACGACGTCCTGGATAAGCGGAGAGGCCGGGACCCTG |
| | | AGATGGGCGGCAAGCCTCGGCGGAAGAACCCCCAGGAAGGCCTGTATAACGAACTGCAG |
| | | AAAGACAAGATGGCCGAGGCCTACAGCGAGATCGGCATGAAGGGCGAGCGGAGGCGGGG |
| | | CAAGGGCCACGACGGCCTGTATCAGGGCCTGTCCACCGCCACCAAGGATACCTACGACG |
| | | CCCTGCACATGCAGGCCCTGCCCCCAAGAGGATCCTCCGAGCTGGAGGAGATTGAGAAG |
| | | AAGGTGAGGGAGCTGACCAAGCGCCACCGCGAACTGGTGGAGCGAGTGCGCAAGACCGT |
| | | GAAGGAGCTCATTGAGACTAACCGCCGGCTGCTCGAGACCCTGACGGAGCGTATCAAGC |
| | | GTGTCCTCGAGGAGGTGCGCGACCTGGAGCGGCGCGGGGAGCCCAAGGAAGATCTCAAG |
| | | GCCCGCCTGGAAGACATCATCCGCCGCCTGGCAGAGGACATTGAGCGCGTCAAGCGCGA |
| | | GACCGACAAGCTGTTGGAAGAGCTTGCTCGCATCATCGAGGAGGTGGAGAAAGAGATGC |
| | | GTGAAACCAACAAGGAGTTGGATAAAGAGATCAAAGACGGCGGCCTTGATGACAAGGCC |
| | | CTGGAGGAGGCCAAGCGCGTGCTGGCCGAGAACAACGAGGTGATTAAGGACATCAAGAG |
| | | AGCTCTGGACGAGCTGCTGAAGGAGCTGGAGAAGTCCGTGAGGGAGAGCAAGACTCGCG |
| | | GTGTGGAGGAAGTGGCGGAGGGCGTGGTCCTGCTGCGCCGTCGCGGCAAC |
| 99 | CAR | MVLQTQVFISLLLWISGAYGQEQLVESGGRLVTPGGSLTLSCKASGFDFSAYYMSWVRQ |
| | MND-CD19t- | APGKGLEWIATIYPSSGKTYYATWVNGRFTISSDNAQNTVDLQMNSLTAADRATYFCAR |
| | hIgK- | DSYADDGALFNIWGPGTLVTISSGGGGSGGGGSGGGGSELVLTQSPSVSAALGSPAKIT |
| | R12-BB-Z- | CTLSSAHKTDTIDWYQQLQGEAPRYLMQVQSDGSYTKRPGVPDRFSGSSSGADRYLIIP |
| | Peptide10_193 | SVQADDEADYYCGADYIGGYVFGGGTQLTVTGESKYGPPCPPCPMFWVLVVVGGVLACY |
| | | SLLVTVAFIIFWVKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPRGSDAEEVVKR |
| | | LADVLRENDETIRKVVEDLVRIAEENDRLWKKLVEDIAEILRRIVELLRRGGVPEELLD |
| | | RLAKVVKSIVEKAEKILERLNRVSKAIAEKLKTIVDELNEVSKEIVKRAEDILRKGKDK |
| | | ETVLRALRTLVKEYADLSKEVLERVERIVREYVKLSDEVVKSLAEIVEELIRIIEDLLR |
| | | 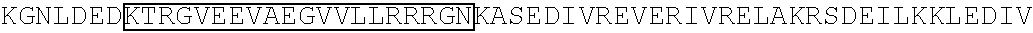 |
| | | EKLRE |
| 100 | CAR | ATGGTCTTGCAGACTCAAGTATTTATATCCCTTTTGCTCTGGATCTCTGGAGCTTACGG |
| | MND-CD19t-hIgK- | CCAGGAACAGCTCGTCGAAAGCGGCGGCAGACTGGTGACACCTGGCGGCAGCCTGACCC |
| | R12-BB-Z- | TGAGCTGCAAGGCCAGCGGCTTCGACTTCAGCGCCTACTACATGAGCTGGGTCCGCCAG |
| | Peptide10_193 | GCCCCTGGCAAGGGACTGGAATGGATCGCCACCATCTACCCCAGCAGCGGCAAGACCTA |
| | | CTACGCCACCTGGGTGAACGGACGGTTCACCATCTCCAGCGACAACGCCCAGAACACCG |
| | | TGGACCTGCAGATGAACAGCCTGACAGCCGCCGACCGGGCCACCTACTTTTGCGCTCGG |
| | | GACAGCTACGCCGACGACGGCGCCCTGTTCAACATCTGGGGCCCTGGCACCCTGGTGAC |
| | | AATCTCTAGCGGCGGAGGCGGATCTGGTGGCGGAGGAAGTGGCGGCGGAGGATCTGAGC |
| | | TGGTGCTGACCCAGAGCCCCTCTGTGTCTGCTGCCCTGGGAAGCCCTGCCAAGATCACC |
| | | TGTACCCTGAGCAGCGCCCACAAGACCGACACCATCGACTGGTATCAGCAGCTGCAGGG |
| | | CGAGGCCCCCAGATACCTGATGCAGGTGCAGAGCGACGGCAGCTACACCAAGAGGCCAG |
| | | GCGTGCCCGACAGGTTCAGCGGATCTAGCTCTGGCGCCGACCGCTACCTGATCATCCCC |
| | | AGCGTGCAGGCCGATGACGAGGCCGATTACTACTGTGGCGCCGACTACATCGGCGGCTA |
| | | CGTGTTCGGCGGAGGCACCCAGCTGACCGTGACCGGTGAAAGCAAATACGGACCGCCCT |
| | | GCCCCCCTTGCCCTATGTTCTGGGTGCTGGTGGTGGTCGGAGGCGTGCTGGCCTGCTAC |
| | | AGCCTGCTGGTCACCGTGGCCTTCATCATCTTTTGGGTGAAACGGGGCAGAAAGAAACT |
| | | CCTGTATATATTCAAACAACCATTTATGAGACCAGTACAAACTACTCAAGAGGAAGATG |
| | | GCTGTAGCTGCCGATTTCCAGAAGAAGAAGAAGGAGGATGTGAACTGCGGGTGAAGTTC |
| | | AGCAGAAGCGCCGACGCCCCTGCCTACCAGCAGGGCCAGAATCAGCTGTACAACGAGCT |
| | | GAACCTGGGCAGAAGGGAAGAGTACGACGTCCTGGATAAGCGGAGAGGCCGGGACCCTG |
| | | AGATGGGCGGCAAGCCTCGGCGGAAGAACCCCCAGGAAGGCCTGTATAACGAACTGCAG |
| | | AAAGACAAGATGGCCGAGGCCTACAGCGAGATCGGCATGAAGGGCGAGCGGAGGCGGGG |
| | | CAAGGGCCACGACGGCCTGTATCAGGGCCTGTCCACCGCCACCAAGGATACCTACGACG |
| | | CCCTGCACATGCAGGCCCTGCCCCCAAGAGGATCCGACGCCGAGGAGGTGGTCAAGCGT |
| | | CTAGCTGATGTGCTGCGAGAGAACGACGAGACTATCCGCAAGGTGGTCGAAGATCTGGT |
| | | GCGCATCGCTGAGGAGAATGACAGGCTTTGGAAGAAGCTGGTAGAAGACATCGCCGAGA |
| | | TTCTGCGTCGCATCGTGGAGCTGCTTCGCCGCGGCGGCGTGCCCGAGGAGCTGCTGGAC |
| | | CGCCTGGCGAAAGTGGTTAAGTCTATCGTGGAGAAAGCCGAGAAGATACTGGAGCGCCT |
| | | GAACCGCGTGTCGAAGGCCATCGCGGAAAAGCTCAAGACCATCGTCGACGAGCTCAACG |
| | | AGGTGTCCAAAGAGATCGTAAAGCGCGCCGAAGACATCCTGCGCAAGGGCAAGGACAAG |
| | | GAGACCGTGCTGCGCGCGCTGCGCACCCTGGTGAAGGAGTACGCGGACCTGTCCAAGGA |
| | | GGTATTGGAGCGAGTGGAGCGCATTGTCAGGGAGTACGTGAAGCTTTCCGACGAGGTGG |
| | | TGAAGAGCCTGGCAGAGATCGTGGAGGAACTCATCCGTATCATTGAGGACCTGCTGCGC |
| | | AAAGGCAACCTGGATGAAGATAAAACCCGCGGTGTGGAAGAGGTGGCGGAGGGCGTCGT |
| | | CCTGCTCCGTCGTCGGGGCAACAAGGCTTCCGAGGACATTGTGAGGGAGGTGGAGCGGA |
| | | TCGTGCGCGAGCTGGCCAAGCGCAGCGATGAGATCCTGAAGAAGCTGGAGGACATCGTG |
| | | GAGAAGCTGCGGGAG |

Linkers/Spacers (e.g., connecting helices)

| | | |
|---|---|---|
| 101 | Spacer1 | (GSGGS)n |
| 102 | Spacer2 | (GGGS)n |
| 103 | Spacer3 | GGSG |
| 104 | Spacer4 | GGSGG |
| 105 | Spacer5 | GSGSG |
| 106 | Spacer6 | GSGGG |
| 107 | Spacer7 | GGGSG |
| 109 | Spacer8 | GSSSG |

Degron peptides integrated in latches
(fragments of SEQ ID NO:1)

| | | |
|---|---|---|
| 110 | DegronPeptide1 | RRRG |
| 111 | DegronPeptide2 | RRRGN |
| 112 | DegronPeptide3 | LRRRG |
| 113 | DegronPeptide4 | LRRRGN |
| 114 | DegronPeptide5 | LLRRRG |
| 115 | DegronPeptide6 | LLRRRGN |
| 116 | DegronPeptide7 | VLLRRRG |
| 117 | DegronPeptide8 | VLLRRRGN |
| 118 | DegronPeptide9 | VVLLRRRG |
| 119 | DegronPeptide10 | VVLLRRRGN |
| 120 | DegronPeptide11 | GVVLLRRRG |
| 121 | DegronPeptide12 | GVVLLRRRGN |
| 122 | DegronPeptide13 | EGVVLLRRRG |
| 123 | DegronPeptide14 | EGVVLLRRRGN |
| 124 | DegronPeptide15 | AEGVVLLRRRG |
| 125 | DegronPeptide16 | AEGVVLLRRRGN |
| 126 | DegronPeptide17 | VAEGVVLLRRRG |
| 127 | DegronPeptide18 | VAEGVVLLRRRGN |
| 128 | DegronPeptide19 | EVAEGVVLLRRRG |
| 129 | DegronPeptide20 | EVAEGVVLLRRRGN |
| 130 | DegronPeptide21 | EEVAEGVVLLRRRG |
| 131 | DegronPeptide22 | EEVAEGVVLLRRRGN |
| 132 | DegronPeptide23 | VEEVAEGVVLLRRRG |
| 133 | DegronPeptide24 | VEEVAEGVVLLRRRGN |
| 134 | DegronPeptide25 | GVEEVAEGVVLLRRRG |
| 135 | DegronPeptide26 | GVEEVAEGVVLLRRRGN |
| 136 | DegronPeptide27 | RGVEEVAEGVVLLRRRG |
| 137 | DegronPeptide28 | RGVEEVAEGVVLLRRRGN |
| 138 | DegronPeptide29 | TRGVEEVAEGVVLLRRRG |
| 139 | DegronPeptide30 | TRGVEEVAEGVVLLRRRGN |
| 140 | DegronPeptide31 | KTRGVEEVAEGVVLLRRRG |
| 141 | DegronPeptide32 | KTRGVEEVAEGVVLLRRRGN |

Cage structural regions (SR) and latches

| | | |
|---|---|---|
| 142 | Cage1_N_SR | KEKVEELVRRIRRIVERVVEEDRRTVEEIEKIAREVVKRDRDSADRVRRTVEDVLRKAT |
| | | GSEDIVRKIERIVETIEREVRESVKKVEEIARDIRRKVDESVKNVEKLLRDVDKKARDR |
| | | KK |
| 143 | Cage1_C_SR | SVEELLRKLEEVLRKIREENERSLKELRDRAREIVKRNRETNRELEEVIKELEKRLSGA |
| | | DKEKVEELVRRIRRIVERVVEEDRRTVEEIEKIAREVVKRDRDSADRVRRTVEDVLRKA |
| | | TG |
| 144 | Cage2_N_SR | EVRKTVEEISRRVAENVKRLEDLYRRMEEEVKKNLDRLRKRVEDIIREVEEARKKGVDE |
| | | EKLKDLIRKLRDILRRAAEAHKKLIDDARESLERAKREHEKLIDRLKKILEELER |
| 145 | Cage2_C_SR | SESDDVIRKLRELLEELRTHVEKSIRDLRKILEDSTRHAKRSIEELERLLEEVRKKPGD |
| | | EEVRKTVEEISRRVAENVKRLEDLYRRMEEEVKKNLDRLRKRVEDIIREVEEARK |
| 146 | Cage3_N_SR | EPKEDLKARLEDIIRRLAEDIERVKRETDKLLEELARIIEEVEKEMRETNKELDKEIKD |
| | | GGLDDKALEEAKRVLAENNEVIKDIKRALDELLKELEKSVRESEERLRRLLEEVKRVIE |
| | | EVAT |
| 147 | Cage3_C_SR | SELEEIEKKVRELTKRHRELVERVRKTVKELIETNRRLLETLTERIKRVLEEVRDLERR |
| | | GEPKEDLKARLEDIIRRLAEDIERVKRETDKLLEELARIIEEVEKEMRETNKELDKEIK |
| | | DGGL |
| 148 | Cage4_N_SR | GDKETAEDIVRRIIEELKRILKEIEDLARRINREIERLVEEVERDNRDVNRAIEELLKD |
| | | IARRGGSEDLKRVEERAREVSRRNEESMRRVKEDADRVSEANKEVLDRVREEVKRLIEE |
| | | VRETLR |
| 149 | Cage4_C_SR | DEIRKVVKEITDLLKASNDKNRKVVEEIRDLLRKSKKLADELVERLRALVEDLRRRIDK |
| | | SGDKETAEDIVRRIIEELKRILKEIEDLARRINREIERLVEEVERDNRDVNRAIEELLK |
| | | DIARRG |
| 150 | Cage5_N_SR | KDILRDLERRLREILEESDRLLEELKRRLEEILRKSKELLRRLEEVLREILKRAEEVKR |
| | | SNLPKEELIKEIVKLLEELLRVIEKILEDNIRLLEELVEVIKEILEKHLRLLEELVRVI |
| | | ERILREVGKDKDEAERRRRELKDKLDRLREEHEEVKRRLEEELTRLRETHKKIEKELRE |
| | | ALKRVRDRST |
| 151 | Cage5_C_SR | SEAEDLEELIKELAELLKDVIRKLEKINRRLVKILEDIIRRLKEISKEAEEELRKGTVE |
| | | DKDILRDLERRLREILEESDRLLEELKRRLEEILRKSKELLRRLEEVLREILKRAEEVK |
| | | RSNLPKEELIKEIVKLLEELLRVIEKILEDNIRLLEELVEVIKEILEKHLRLLEELVRV |
| | | IERILREVGK |
| 152 | Cage6_N_SR | KDILRDLERRLREILEESDRLLEELKRRLEEILRKSKELLRRLEEVLREILKRAEEVKR |
| | | SNLPKEELIKEIVKLLEELLRVIEKILEDNIRLLEELVEVIKEILEKHLRLLEELVRVI |
| | | ERILREVGKDKDEAERRRRELKDKLDRLREEHEEVKRRLEEELTRLRETHKKIEKELRE |
| | | ALKRVRDRST |
| 153 | Cage6_C_SR | SEAEDLEELIRELAELLRDVIRRLERINRRLVRILEDIIRRLREISREAEEELRRGTVE |
| | | DKDILRDLERRLREILEESDRLLEELKRRLEEILRKSKELLRRLEEVLREILKRAEEVK |
| | | RSNLPREELIREIVRLLEELLRVIERILEDNIRLLEELVEVIREILERHLRLLEELVRV |
| | | IERILREVGR |
| 154 | Cage7_N_SR | ISDEKLKRIRERLKRALDDLERLHREINKRLEDLVRELEKLVREILKELKDALEELRRA |
| | | SARAGGEEVLRRLEEIVKKLLDLVRRILERLKEIHKDNVRLLRELNERLTRIVEDLVRL |
| | | IREILREAGVDERIAEEIERELEELRRMIRRLHEDLERRLRESEDELREIEARLEERIR |
| | | RLEKLERKRR |
| 155 | Cage7_C_SR | DEDRIIEEIARLLEELLRELLELIKKLIETNRRLNEEHERAVRELARLLEELLDRLVKK |
| | | GISDEKLKRIRERLKRALDDLERLHREINKRLEDLVRELEKLVREILKELKDALEELRR |
| | | ASARAGGEEVLRRLEEIVRRLLDLVRRILERLREIHRDNVRLLRELNERLTRIVEDLVR |
| | | LIREILREAG |
| 156 | Cage8_N_SR | GDERAIRTLEDVKRRLREILERLADENAKAIKRLADLLDKLEKRNKEAIERLEEILEEL |
| | | KRVRRDEELLRVLETLLKIIEDILRENTKVLEDLLRLVEEILEANLRVVEELLRLAREI |
| | | LTEIVGDEDKLKEIEDELRRLLEELRRLDKAIKDRLRELKKDLDEANRRIKETLKKLLR |
| | | EVER |
| 157 | Cage8_C_SR | SKKELLEEVVRRAIELLKRHLEKLKRILEEIVRLLEEHLEKVERVLEAILSLLDDLLRR |
| | | GGDERAIRTLEDVRRRLREILERLADENARAIRRLADLLDRLERRNREAIERLEEILEE |
| | | LKRVRRDEELLRVLETLLKIIEDILRENTKVLEDLLRLVEEILEANLRVVEELLRLARE |
| | | ILTE |
| 158 | Cage9_N_SR | ELLRRVERVLRDNLDLLRRLVEEHRESSERDLRRVEDLVREIREVLRRLLELEDRGTDI |
| | | RRIEEEIERLLRRIRRAVEESRDLNRRNSERIEEVARRSEELARRLLREIRE |
| | | RGDSRAAEDILRVLERLVRVSREAIRLILELSEHHVRVSTRIARLLLDVARRLAEVIRE |
| | | AER |
| 159 | Cage9_C_SR | SREERLRDDVRAVLEDLDRVLRELERLSEDNLRELRRVLDRITDLHRRILDELRRGIGS |
| | | EELLRRVERVLRDNLDLLRRLVEEHRESSERDLRRVEDLVREIREVLRRLLELEDRGTD |
| | | IRRIEEEIERLLRRIRRAVEESRDLNRRNSERIEEVARRSEELARRLLREIRE |
| | | RG |
| 160 | Cage10_N_SR | VPEELLDRLAKVVKSIVEKAEKILERLNRVSKAIAEKLKTIVDELNEVSKEIVKRAEDI |
| | | LRKGKDKETVLRALRTLVKEYADLSKEVLERVERIVREYVKLSDEVVKSLAE |
| | | IVEELIRIIEDLLRRGNLDEDVRRALEELVSRLRRLLEDVRRASEDIVREVERIVRELA |
| | | RRSDEILRRLEDIVERLRE |
| 161 | Cage10_C_SR | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAE |
| | | IVEELIRIIEDLLRRGNL |
| 162 | Cage11_N_SR | VPEELLDRLAKVVKSIVEKAEKILERLNRVSKAIAEKLKTIVDELNEVSKEIVKRAEDI |
| | | LRKGKDKETVLRALRTLVKEYADLSKEVLERVERIVREYVKLSDEVVKSLAEIVEELIR |
| | | IIEDLLRRGNLDEDVRRALEELVSRLRRLLEDVRRASEDIVREVERIVRELARRSDEIL |
| | | RRLEDIVERLRE |
| 163 | Cage11_C_SR | DAEEVVKRLADVLRENDETIRKVVEDLVRIAEENDRLWKKLVEDIAEILRRIVELLRRG |
| | | GVPEELLDRLAKVVKSIVEKAEKILERLNRVSKAIAEKLKTIVDELNEVSKEIVKRAED |
| | | ILRKGKDKETVLRALRTLVKEYADLSKEVLERVERIVREYVKLSDEVVKSLAEIVEELI |
| | | RIIEDLLRRGNL |
| 164 | Cage12_N_SR | DELIRRLAEELERSLRELEEEIRRLRRELEESLRRLREIIDRLAEEAERLLAVLRRGEG |
| | | SEEEALRALASLVRELIEVLRENDERLRDVLRRLIEALRRNNEILERVLRRLVRAAEER |
| | | GRDESSREALEEARRRLEELLRELNEITRDLEARLERLLRDLNELTRALEEELRRLLDE |
| | | LRRRTD |
| 165 | Cage12_C_SR | DEEETLRRLLERRVELAREYLDVSREVIDRTTRLLDEYLRTSRRIVDATVELLERGDLG |
| | | PDELIRRLAEELERSLRELEEEIRRLRRELEESLRRLREIIDRLAEEAERLLAVLRRGE |
| | | GSEEEALRALASLVRELIEVLRENDERLRDVLRRLIEALRRNNEILERVLRRLVRAAEE |
| | | RGRDES |
| 166 | Cage1_N_Latch | SVEELLRKLEEVLRKIREENERSLKELRDRAREIVKRNRETNRELEEVIKELEKRLSGA |
| | | D |
| 167 | Cage1_C_Latch | SEDIVRKIERIVETIEREVRESVKKVEEIARDIRRKVDESVKNVEKLLRDVDKKARDRK |
| | | K |
| 168 | Cage2_N_Latch | SESDDVIRKLRELLEELRTHVEKSIRDLRKILEDSTRHAKRSIEELERLLEEVRKKPGD |
| | | E |
| 169 | Cage2_C_Latch | KGVDEEKLKDLIRKLRDILRRAAEAHKKLIDDARESLERAKREHEKLIDRLKKILEELE |
| | | R |
| 170 | Cage3_N_Latch | SELEEIEKKVRELTKRHRELVERVRKTVKELIETNRRLLETLTERIKRVLEEVRDLERR |
| | | G |
| 171 | Cage3_C_Latch | DDKALEEAKRVLAENNEVIKDIKRALDELLKELEKSVRESEERLRRLLEEVKRVIEEVA |
| | | T |
| 172 | Cage4_N_Latch | DEIRKVVKEITDLLKASNDKNRKVVEEIRDLLRKSKKLADELVERLRALVEDLRRRIDK |
| | | S |
| 173 | Cage4_C_Latch | GSEDLKRVEERAREVSRRNEESMRRVKEDADRVSEANKEVLDRVREEVKRLIEEVRETL |
| | | R |
| 174 | Cage5_N_Latch | SEAEDLEELIKELAELLKDVIRKLEKINRRLVKILEDIIRRLKEISKEAEEELRKGTVE |
| | | D |
| 175 | Cage5_C_Latch | DKDEAERRRRELKDKLDRLREEHEEVKRRLEEELTRLRETHKKIEKELREALKRVRDRS |
| | | T |
| 176 | Cage6_N_Latch | SEAEDLEELIKELAELLKDVIRKLEKINRRLVKILEDIIRRLKEISKEAEEELRKGTVE |
| | | D |
| 177 | Cage6_C_Latch | DKDEAERRRRELKDKLDRLREEHEEVKRRLEEELTRLRETHKKIEKELREALKRVRDRS |
| | | T |
| 178 | Cage7_N_Latch | DEDRIIEEIARLLEELLRELLELIKKLIETNRRLNEEHERAVRELARLLEELLDRLVKK |
| | | G |
| 179 | Cage7_C_Latch | VDEKIAEEIERELEELRRMIKRLHEDLERKLKESEDELREIEARLEEKIRRLEKLERKR |
| | | R |
| 180 | Cage8_N_Latch | SKKELLEEVVRRAIELLKRHLEKLKRILEEIVRLLEEHLEKVERVLEAILSLLDDLLRR |
| | | G |
| 181 | Cage8_C_Latch | IVGDEDKLKEIEDELRRLLEELRRLDKAIKDRLRELKKDLDEANRRIKETLKKLLREVE |
| | | K |
| 182 | Cage9_N_Latch | SKEEKLKDDVRAVLEDLDRVLKELEKLSEDNLRELKRVLDRITDLHRRILDELRKGIGS |
| | | E |
| 183 | Cage9_C_Latch | DSKAAEDILRVLEKLVKVSREAIKLILELSEHHVRVSTRIARLLLDVARKLAEVIKEAE |
| | | R |
| 184 | Cage10_N_Latch | DAEEVVKRLADVLRENDETIRKVVEDLVRIAEENDRLWKKLVEDIAEILRRIVELLRRG |
| | | G |
| 185 | Cage10_N_CLatch | DEDVKRALEELVSRLRKLLEDVKKASEDIVREVERIVRELAKRSDEILKKLEDIVEKLR |
| | | E |
| 186 | Cage11_N_Latch | DAEEVVKRLADVLRENDETIRKVVEDLVRIAEENDRLWKKLVEDIAEILRRIVELLRRG |
| | | G |
| 187 | Cage11_C_Latch | DEDVKRALEELVSRLRKLLEDVKKASEDIVREVERIVRELAKRSDEILKKLEDIVEKLR |
| | | E |
| 188 | Cage12_N_Latch | DEEETLRRLLERKVELAKEYLDVSKEVIDRTTKLLDEYLKTSKRIVDATVELLERGDLG |
| | | P |
| 189 | Cage12_C_Latch | SREALEEARRRLEELLRELNEITKDLEAKLEKLLRDLNELTKALEEELKRLLDELKKRT |
| | | D |

Base Design Helices

| | | |
|---|---|---|
| 190 | Cage1_helix_1 | EELLRKLEEVLRKIREENERSLKELRDRAREIVKRNRETNRELEEVIKELEKRLSGA |
| 191 | Cage1_helix_2 | EKVEELVRRIRRIVERVVEEDRRTVEEIEKIAREVVKRDRDSADRVRRTVEDVLRKA |
| 192 | Cage1_helix_3 | DIVRKIERIVETIEREVRESVKKVEEIARDIRRKVDESVKNVEKLLRDVDKKARDRKK |
| 193 | Cage2_helix_1 | SDDVIRKLRELLEELRTHVEKSIRDLRKILEDSTRHAKRSIEELERLLEEVRK |
| 194 | Cage2_helix_2 | EVRKTVEEISRRVAENVKRLEDLYRRMEEEVKKNLDRLRKRVEDIIREVEEARKK |
| 195 | Cage2_helix_3 | EKLKDLIRKLRDILRRAAEAHKKLIDDARESLERAKREHEKLIDRLKKILEELER |
| 196 | Cage3_helix_1 | LEEIEKKVRELTKRHRELVERVRKTVKELIETNRRLLETLTERIKRVLEEVRDLERR |
| 197 | Cage3_helix_2 | EDLKARLEDIIRRLAEDIERVKRETDKLLEELARIIEEVEKEMRETNKELDKEIKDG |
| 198 | Cage3_helix_3 | KALEEAKRVLAENNEVIKDIKRALDELLKELEKSVRESEERLRRLLEEVKRVIEEVAT |
| 199 | Cage4_helix_1 | IRKVVKEITDLLKASNDKNRKVVEEIRDLLRKSKKLADELVERLRALVEDLRRRIDK |
| 200 | Cage4_helix_2 | ETAEDIVRRIIEELKRILKEIEDLARRINREIERLVEEVERDNRDVNRAIEELLKDIAR |
| | | RG |
| 201 | Cage4_helix_3 | EDLKRVEERAREVSRRNEESMRRVKEDADRVSEANKEVLDRVREEVKRLIEEVRETLR |
| 202 | Cage5_helix_1 | AEDLEELIKELAELLKDVIRKLEKINRRLVKILEDIIRRLKEISKEAEEELRKG |
| 203 | Cage5_helix_2 | DILRDLERRLREILEESDRLLEELKRRLEEILRKSKELLRRLEEVLREILKRAEEVKRS |
| 204 | Cage5_helix_3 | EELIKEIVKLLEELLRVIEKILEDNIRLLEELVEVIKEILEKHLRLLEELVRVIERILR |
| | | EVG |
| 205 | Cage5_helix_4 | DEAERRRRELKDKLDRLREEHEEVKRRLEEELTRLRETHKKIEKELREALKRVRDRST |
| 206 | Cage6_helix_1 | AEDLEELIKELAELLKDVIRKLEKINRRLVKILEDIIRRLKEISKEAEEELRKG |
| 207 | Cage6_helix_2 | DILRDLERRLREILEESDRLLEELKRRLEEILRKSKELLRRLEEVLREILKRAEEVKRS |
| 208 | Cage6_helix_3 | EELIKEIVKLLEELLRVIEKILEDNIRLLEELVEVIKEILEKHLRLLEELVRVIERILR |
| | | EVG |
| 209 | Cage6_helix_4 | DEAERRRRELKDKLDRLREEHEEVKRRLEEELTRLRETHKKIEKELREALKRVRDRST |
| 210 | Cage7_helix_1 | DRIIEEIARLLEELLRELLELIKKLIETNRRLNEEHERAVRELARLLEELLDRLVKKG |
| 211 | Cage7_helix_2 | KLKRIRERLKRALDDLERLHREINKRLEDLVRELEKLVREILKELKDALEELRRASAR |
| 212 | Cage7_helix_3 | EVLRRLEEIVKKLLDLVRRILERLKEIHKDNVRLLRELNERLTRIVEDLVRLIREILRE |
| | | AG |
| 213 | Cage7_helix_4 | KIAEEIERELEELRRMIKRLHEDLERKLKESEDELREIEARLEEKIRRLEEKLERKRR |
| 214 | Cage8_helix_1 | KELLEEVVRRAIELLKRHLEKLKRILEEIVRLLEEHLEKVERVLEAILSLLDDLLRRG |
| 215 | Cage8_helix_2 | RAIRTLEDVKRRLREILERLADENAKAIKRLADLLDKLEKRNKEAIERLEEILEELKRV |
| 216 | Cage8_helix_3 | EELLRVLETLLKIIEDILRENTKVLEDLLRLVEEILEANLRVVEELLRLAREILTEIV |
| 217 | Cage8_helix_4 | DKLKEIEDELRRLLEELRRLDKAIKDRLRELKKDLDEANRRIKETLKKLLREVEK |
| 218 | Cage9_helix_1 | EEKLKDDVRAVLEDLDRVLKELEKLSEDNLRELKRVLDRITDLHRRILDELRKGI |
| 219 | Cage9_helix_2 | ELLRRVEKVLKDNLDLLRKLVEEHKESSERDLKRVEDLVREIKEVLRKLLELEDR |
| 220 | Cage9_helix_3 | RKIEEEIERLLRKIRKAVEESKDLNRRNSERIEEVARRSEELARRLLKEIRERG |
| 221 | Cage9_helix_4 | KAAEDILRVLEKLVKVSREAIKLILELSEHHVRVSTRIARLLLDVARKLAEVIKEAER |
| 222 | Cage10_helix_1 | EEVVKRLADVLRENDETIRKVVEDLVRIAEENDRLVVKKLVEDIAEILRRIVELLRRG |
| 223 | Cage10_helix_2 | ELLDRLAKVVKSIVEKAEKILERLNRVSKAIAEKLKTIVDELNEVSKEIVKRAEDILRK |
| | | G |
| 224 | Cage10_helix_3 | ETVLRALRTLVKEYADLSKEVLERVERIVREYVKLSDEVVKSLAEIVEELIRIIEDLLR |
| | | KGN |
| 225 | Cage10_helix_4 | DVKRALEELVSRLRKLLEDVKKASEDIVREVERIVRELAKRSDEILKKLEDIVEKLRE |
| 226 | Cage11_helix_1 | EEVVKRLADVLRENDETIRKVVEDLVRIAEENDRLVVKKLVEDIAEILRRIVELLRRG |
| 227 | Cage11_helix_2 | ELLDRLAKVVKSIVEKAEKILERLNRVSKAIAEKLKTIVDELNEVSKEIVKRAEDILRK |
| | | G |
| 228 | Cage11_helix_3 | ETVLRALRTLVKEYADLSKEVLERVERIVREYVKLSDEVVKSLAEIVEELIRIIEDLLR |
| | | KG |
| 229 | Cage11_helix_4 | DVKRALEELVSRLRKLLEDVKKASEDIVREVERIVRELAKRSDEILKKLEDIVEKLRE |
| 230 | Cage12_helix_1 | EETLRRLLERKVELAKEYLDVSKEVIDRTTKLLDEYLKTSKRIVDATVELLERGD |
| 231 | Cage12_helix_2 | ELIKRLAEELERSLRELEEEIKRLKRELEESLKKLKEIIDRLAEEAEKLLAVLKRG |
| 232 | Cage12_helix_3 | EEALRALASLVRELIEVLRENDERLRDVLRRLIEALRKNNEILERVLRKLVRAAEERG |
| 233 | Cage12_helix_4 | REALEEARRRLEELLRELNEITKDLEAKLEKLLRDLNELTKALEEELKRLLDELKKRTD |

Helices in Chimeric Peptides with degrons

| | | |
|---|---|---|
| 234 | Peptide_1_10_helix_1 | 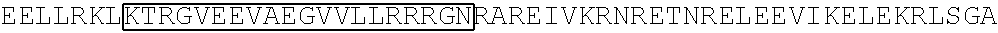 |
| 235 | Peptide_1_10_helix_2 | EKVEELVRRRRIVERVVEEDRRTVEEIEKIAREVVKRDRDSADRVRRTVEDVLRKA |
| 236 | Peptide_1_10_helix_3 | DIVRKIERIVETIEREVRESVKKVEEIARDIRRKVDESVKNVEKLLRDVDKKARDRKK |
| 237 | Peptide_1_121_helix_1 | EELLRKLEEVLRKIREENERSLKELRDRAREIVKRNRETNRELEEVIKELEKRLSGA |
| 238 | Peptide_1_121_helix_2 | EKVEELVRRIRRIVERVVEEDRRTVEEIEKIAREVVKRDRDSADRVRRTVEDVLRKA |
| 239 | Peptide_1_121_helix_3 | RGVEEVAEGVVLLRRRGNESVKKVEEIARDIRRKVDESVKNVEKLLRDVDKKARDRKK |
| 240 | Peptide_1_122_helix_1 | EELLRKLEEVLRKIREENERSLKELRDRAREIVKRNRETNRELEEVIKELEKRLSGA |
| 241 | Peptide_1_122_helix_2 | EKVEELVRRIRRIVERVVEEDRRTVEEIEKIAREVVKRDRDSADRVRRTVEDVLRKA |
| 242 | Peptide_1_122_helix_3 | TRGVEEVAEGVVLLRRRGNSVKKVEEIARDIRRKVDESVKNVEKLLRDVDKKARDRKK |
| 243 | Peptide_1_124_helix_1 | EELLRKLEEVLRKIREENERSLKELRDRAREIVKRNRETNRELEEVIKELEKRLSGA |
| 244 | Peptide_1_124_helix_2 | EKVEELVRRIRRIVERVVEEDRRTVEEIEKIAREVVKRDRDSADRVRRTVEDVLRKA |
| 245 | Peptide_1_124_helix_3 | 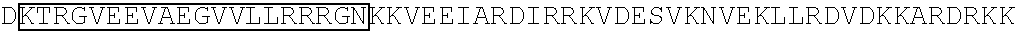 |
| 246 | Peptide1_125_helix_1 | EELLRKLEEVLRKIREENERSLKELRDRAREIVKRNRETNRELEEVIKELEKRLSGA |
| 247 | Peptide1_125_helix_2 | EKVEELVRRIRRIVERVVEEDRRTVEEIEKIAREVVKRDRDSADRVRRTVEDVLRKA |
| 248 | Peptide1_125_helix_3 | 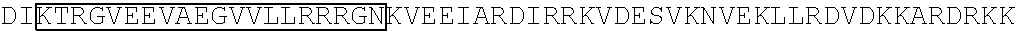 |
| 249 | Peptide1_128_helix_1 | EELLRKLEEVLRKIREENERSLKELRDRAREIVKRNRETNRELEEVIKELEKRLSGA |
| 250 | Peptide1_128_helix_2 | EKVEELVRRIRRIVERVVEEDRRTVEEIEKIAREVVKRDRDSADRVRRTVEDVLRKA |
| 251 | Peptide1_128_helix_3 | 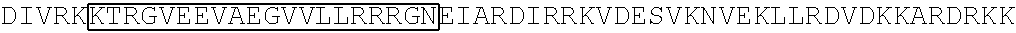 |
| 252 | Peptide1_13_helix_1 | 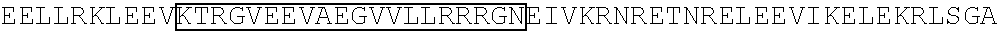 |
| 253 | Peptide1_13_helix_2 | EKVEELVRRIRRIVERVVEEDRRTVEEIEKIAREVVKRDRDSADRVRRTVEDVLRKA |
| 254 | Peptide1_13_helix_3 | DIVRKIERIVETIEREVRESVKKVEEIARDIRRKVDESVKNVEKLLRDVDKKARDRKK |
| 255 | Peptide1_139_helix_1 | EELLRKLEEVLRKIREENERSLKELRDRAREIVKRNRETNRELEEVIKELEKRLSGA |
| 256 | Peptide1_139_helix_2 | EKVEELVRRIRRIVERVVEEDRRTVEEIEKIAREVVKRDRDSADRVRRTVEDVLRKA |
| 257 | Peptide1_139_helix_3 | 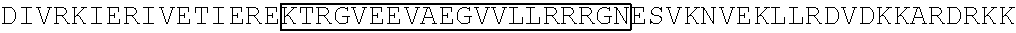 |
| 258 | Peptide1_157_helix_1 | EELLRKLEEVLRKIREENERSLKELRDRAREIVKRNRETNRELEEVIKELEKRLSGA |
| 259 | Peptide1_157_helix_2 | EKVEELVRRIRRIVERVVEEDRRTVEEIEKIAREVVKRDRDSADRVRRTVEDVLRKA |
| 260 | Peptide1_157_helix_3 | 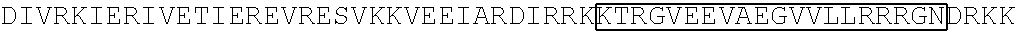 |
| 261 | Peptide1_2_helix_1 | TRGVEEVAEGVVLLRRRGNRSLKELRDRAREIVKRNRETNRELEEVIKELEKRLSGA |
| 262 | Peptide1_2_helix_2 | EKVEELVRRIRRIVERVVEEDRRTVEEIEKIAREVVKRDRDSADRVRRTVEDVLRKA |
| 263 | Peptide1_2_helix_3 | DIVRKIERIVETIEREVRESVKKVEEIARDIRRKVDESVKNVEKLLRDVDKKARDRKK |
| 264 | Peptide1_24_helix_1 | 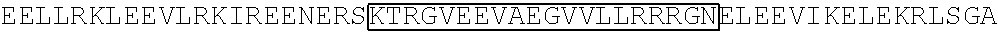 |
| 265 | Peptide1_24_helix_2 | EKVEELVRRIRRIVERVVEEDRRTVEEIEKIAREVVKRDRDSADRVRRTVEDVLRKA |
| 266 | Peptide1_24_helix_3 | DIVRKIERIVETIEREVRESVKKVEEIARDIRRKVDESVKNVEKLLRDVDKKARDRKK |
| 267 | Peptide1_6_helix_1 | 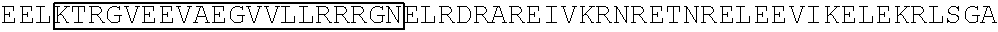 |
| 268 | Peptide1_6_helix_2 | EKVEELVRRIRRIVERVVEEDRRTVEEIEKIAREVVKRDRDSADRVRRTVEDVLRKA |
| 269 | Peptide1_6_helix_3 | DIVRKIERIVETIEREVRESVKKVEEIARDIRRKVDESVKNVEKLLRDVDKKARDRKK |
| 270 | Peptide2_10_helix_1 | 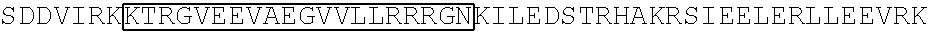 |
| 271 | Peptide2_10_helix_2 | EVRKTVEEISRRVAENVKRLEDLYRRMEEEVKKNLDRLRKRVEDIIREVEEARKK |
| 272 | Peptide2_10_helix_3 | EKLKDLIRKLRDILRRAAEAHKKLIDDARESLERAKREHEKLIDRLKKILEELER |
| 273 | Peptide2_118_helix_1 | SDDVIRKLRELLEELRTHVEKSIRDLRKILEDSTRHAKRSIEELERLLEEVRK |
| 274 | Peptide2_118_helix_2 | EVRKTVEEISRRVAENVKRLEDLYRRMEEEVKKNLDRLRKRVEDIIREVEEARKK |
| 275 | Peptide2_118_helix_3 | RGVEEVAEGVVLLRRRGNEAHKKLIDDARESLERAKREHEKLIDRLKKILEELER |
| 276 | Peptide2_118_helix_1 | SDDVIRKLRELLEELRTHVEKSIRDLRKILEDSTRHAKRSIEELERLLEEVRK |
| 277 | Peptide2_130_helix_2 | EVRKTVEEISRRVAENVKRLEDLYRRMEEEVKKNLDRLRKRVEDIIREVEEARKK |
| 278 | Peptide2_130_helix_3 | 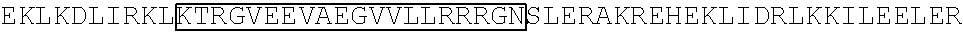 |
| 279 | Peptide2_133_helix_1 | SDDVIRKLRELLEELRTHVEKSIRDLRKILEDSTRHAKRSIEELERLLEEVRK |
| 280 | Peptide2_133_helix_2 | EVRKTVEEISRRVAENVKRLEDLYRRMEEEVKKNLDRLRKRVEDIIREVEEARKK |
| 281 | Peptide2_133_helix_3 | 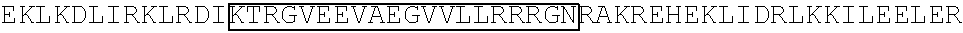 |
| 282 | Peptide2_140_helix_1 | SDDVIRKLRELLEELRTHVEKSIRDLRKILEDSTRHAKRSIEELERLLEEVRK |
| 283 | Peptide2_140_helix_2 | EVRKTVEEISRRVAENVKRLEDLYRRMEEEVKKNLDRLRKRVEDIIREVEEARKK |
| 284 | Peptide2_140_helix_3 | 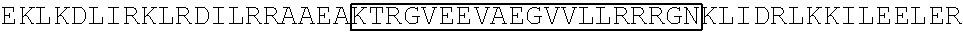 |
| 285 | Peptide2_147_helix_1 | SDDVIRKLRELLEELRTHVEKSIRDLRKILEDSTRHAKRSIEELERLLEEVRK |
| 286 | Peptide2_147_helix_2 | EVRKTVEEISRRVAENVKRLEDLYRRMEEEVKKNLDRLRKRVEDIIREVEEARKK |
| 287 | Peptide2_147_helix_3 | 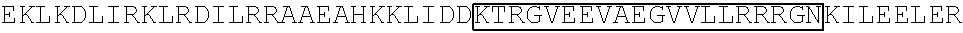 |
| 288 | Peptide2_151_helix_1 | SDDVIRKLRELLEELRTHVEKSIRDLRKILEDSTRHAKRSIEELERLLEEVRK |
| 289 | Peptide2_151_helix_2 | EVRKTVEEISRRVAENVKRLEDLYRRMEEEVKKNLDRLRKRVEDIIREVEEARKK |
| 290 | Peptide2_151_helix_3 | 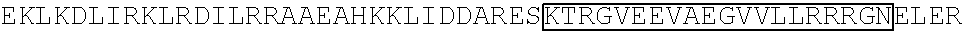 |
| 291 | Peptide2_154_helix_1 | SDDVIRKLRELLEELRTHVEKSIRDLRKILEDSTRHAKRSIEELERLLEEVRK |
| 292 | Peptide2_154_helix_2 | EVRKTVEEISRRVAENVKRLEDLYRRMEEEVKKNLDRLRKRVEDIIREVEEARKK |
| 293 | Peptide2_154_helix_3 | 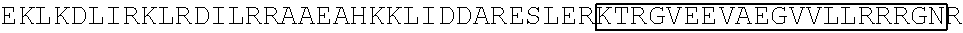 |
| 294 | Peptide2_25_helix_1 | 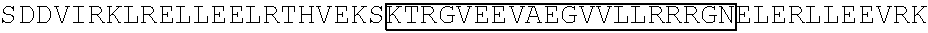 |
| 295 | Peptide2_25_helix_2 | EVRKTVEEISRRVAENVKRLEDLYRRMEEEVKKNLDRLRKRVEDIIREVEEARKK |
| 296 | Peptide2_25_helix_3 | EKLKDLIRKLRDILRRAAEAHKKLIDDARESLERAKREHEKLIDRLKKILEELER |
| 297 | Peptide2_28_helix_1 | 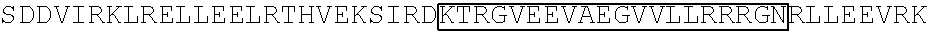 |
| 298 | Peptide2_28_helix_2 | EVRKTVEEISRRVAENVKRLEDLYRRMEEEVKKNLDRLRKRVEDIIREVEEARKK |
| 299 | Peptide2_28_helix_3 | EKLKDLIRKLRDILRRAAEAHKKLIDDARESLERAKREHEKLIDRLKKILEELER |
| 300 | Peptide2_32_helix_1 | 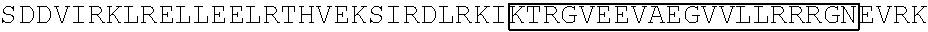 |
| 301 | Peptide2_32_helix_2 | EVRKTVEEISRRVAENVKRLEDLYRRMEEEVKKNLDRLRKRVEDIIREVEEARKK |
| 302 | Peptide2_32_helix_3 | EKLKDLIRKLRDILRRAAEAHKKLIDDARESLERAKREHEKLIDRLKKILEELER |
| 303 | Peptide2_35_helix_1 | 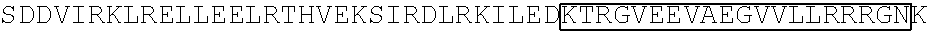 |
| 304 | Peptide2_35_helix_2 | EVRKTVEEISRRVAENVKRLEDLYRRMEEEVKKNLDRLRKRVEDIIREVEEARKK |
| 305 | Peptide2_35_helix_3 | EKLKDLIRKLRDILRRAAEAHKKLIDDARESLERAKREHEKLIDRLKKILEELER |
| 306 | Peptide3_10_helix_1 | 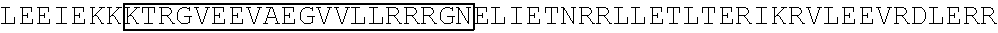 |
| 307 | Peptide3_10_helix_2 | EDLKARLEDIIRRLAEDIERVKRETDKLLEELARIIEEVEKEMRETNKELDKEIKDG |
| 308 | Peptide3_10_helix_3 | KALEEAKRVLAENNEVIKDIKRALDELLKELEKSVRESEERLRRLLEEVKRVIEEVAT |
| 309 | Peptide3_11_helix_1 | 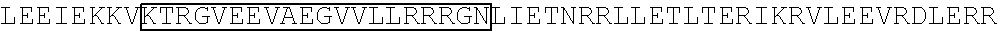 |
| 310 | Peptide3_11_helix_2 | EDLKARLEDIIRRLAEDIERVKRETDKLLEELARIIEEVEKEMRETNKELDKEIKDG |
| 311 | Peptide3_11_helix_3 | KALEEAKRVLAENNEVIKDIKRALDELLKELEKSVRESEERLRRLLEEVKRVIEEVAT |
| 312 | Peptide3_123_helix_1 | LEEIEKKVRELTKRHRELVERVRKTVKELIETNRRLLETLTERIKRVLEEVRDLERR |
| 313 | Peptide3_123_helix_2 | EDLKARLEDIIRRLAEDIERVKRETDKLLEELARIIEEVEKEMRETNKELDKEIKDGG |
| 314 | Peptide3_123_helix_3 | RGVEEVAEGVVLLRRRGNDIKRALDELLKELEKSVRESEERLRRLLEEVKRVIEEVAT |
| 315 | Peptide3_127_helix_1 | LEEIEKKVRELTKRHRELVERVRKTVKELIETNRRLLETLTERIKRVLEEVRDLERR |
| 316 | Peptide3_127_helix_2 | EDLKARLEDIIRRLAEDIERVKRETDKLLEELARIIEEVEKEMRETNKELDKEIKDG |
| 317 | Peptide3_127_helix_3 | 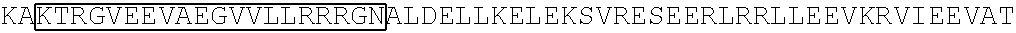 |
| 318 | Peptide3_13_helix_1 | 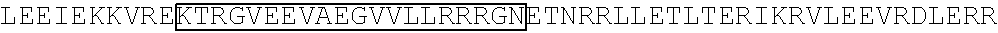 |
| 319 | Peptide3_13_helix_2 | EDLKARLEDIIRRLAEDIERVKRETDKLLEELARIIEEVEKEMRETNKELDKEIKDG |
| 320 | Peptide3_13_helix_3 | KALEEAKRVLAENNEVIKDIKRALDELLKELEKSVRESEERLRRLLEEVKRVIEEVAT |
| 321 | Peptide3_130_helix_1 | LEEIEKKVRELTKRHRELVERVRKTVKELIETNRRLLETLTERIKRVLEEVRDLERR |
| 322 | Peptide3_130_helix_2 | EDLKARLEDIIRRLAEDIERVKRETDKLLEELARIIEEVEKEMRETNKELDKEIKDGG |
| 323 | Peptide3_130_helix_3 | 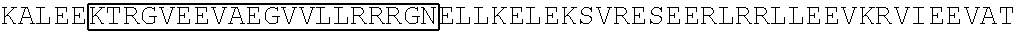 |
| 324 | Peptide3_14_helix_1 | 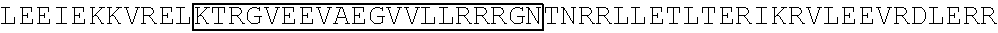 |
| 325 | Peptide3_14_helix_2 | EDLKARLEDIIRRLAEDIERVKRETDKLLEELARIIEEVEKEMRETNKELDKEIKDG |
| 326 | Peptide3_14_helix_3 | KALEEAKRVLAENNEVIKDIKRALDELLKELEKSVRESEERLRRLLEEVKRVIEEVAT |
| 327 | Peptide3_144_helix_1 | LEEIEKKVRELTKRHRELVERVRKTVKELIETNRRLLETLTERIKRVLEEVRDLERR |
| 328 | Peptide3_144_helix_2 | EDLKARLEDIIRRLAEDIERVKRETDKLLEELARIIEEVEKEMRETNKELDKEIKDG |
| 329 | Peptide3_144_helix_3 | 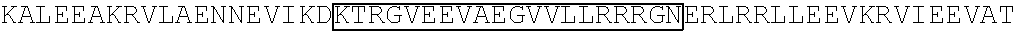 |
| 330 | Peptide3_148_helix_1 | LEEIEKKVRELTKRHRELVERVRKTVKELIETNRRLLETLTERIKRVLEEVRDLERR |
| 331 | Peptide3_148_helix_2 | EDLKARLEDIIRRLAEDIERVKRETDKLLEELARIIEEVEKEMRETNKELDKEIKDG |
| 332 | Peptide3_148_helix_3 | 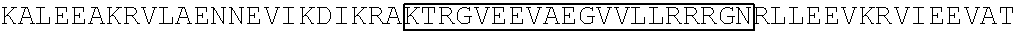 |
| 333 | Peptide3_152_helix_1 | LEEIEKKVRELTKRHRELVERVRKTVKELIETNRRLLETLTERIKRVLEEVRDLERR |
| 334 | Peptide3_152_helix_2 | EDLKARLEDIIRRLAEDIERVKRETDKLLEELARIIEEVEKEMRETNKELDKEIKDG |
| 335 | Peptide3_152_helix_3 | 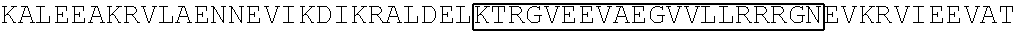 |
| 336 | Peptide3_159_helix_1 | LEEIEKKVRELTKRHRELVERVRKTVKELIETNRRLLETLTERIKRVLEEVRDLERR |
| 337 | Peptide3_159_helix_2 | EDLKARLEDIIRRLAEDIERVKRETDKLLEELARIIEEVEKEMRETNKELDKEIKDG |
| 338 | Peptide3_159_helix_3 | 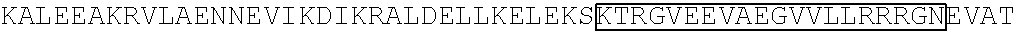 |
| 339 | Peptide3_160_helix_1 | LEEIEKKVRELTKRHRELVERVRKTVKELIETNRRLLETLTERIKRVLEEVRDLERR |
| 340 | Peptide3_160_helix_2 | EDLKARLEDIIRRLAEDIERVKRETDKLLEELARIIEEVEKEMRETNKELDKEIKDG |
| 341 | Peptide3_160_helix_3 | 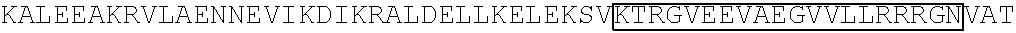 |
| 342 | Peptide3_162_helix_1 | LEEIEKKVRELTKRHRELVERVRKTVKELIETNRRLLETLTERIKRVLEEVRDLERR |
| 343 | Peptide3_162_helix_2 | EDLKARLEDIIRRLAEDIERVKRETDKLLEELARIIEEVEKEMRETNKELDKEIKDG |
| 344 | Peptide3_162_helix_3 | 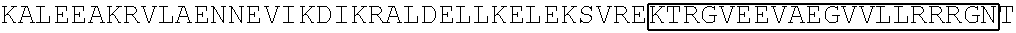 |
| 345 | Peptide3_163_helix_1 | LEEIEKKVRELTKRHRELVERVRKTVKELIETNRRLLETLTERIKRVLEEVRDLERR |
| 346 | Peptide3_163_helix_2 | EDLKARLEDIIRRLAEDIERVKRETDKLLEELARIIEEVEKEMRETNKELDKEIKDG |
| 347 | Peptide3_163_helix_3 | 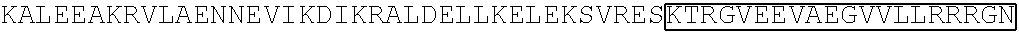 |
| 348 | Peptide3_17_helix_1 | 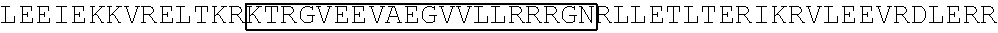 |
| 349 | Peptide3_17_helix_2 | EDLKARLEDIIRRLAEDIERVKRETDKLLEELARIIEEVEKEMRETNKELDKEIKDG |
| 350 | Peptide3_17_helix_3 | KALEEAKRVLAENNEVIKDIKRALDELLKELEKSVRESEERLRRLLEEVKRVIEEVAT |
| 351 | Peptide3_21_helix_1 | 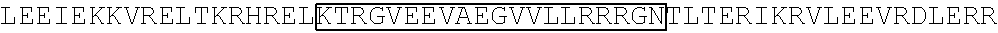 |
| 352 | Peptide3_21_helix_2 | EDLKARLEDIIRRLAEDIERVKRETDKLLEELARIIEEVEKEMRETNKELDKEIKDG |
| 353 | Peptide3_21_helix_3 | KALEEAKRVLAENNEVIKDIKRALDELLKELEKSVRESEERLRRLLEEVKRVIEEVAT |
| 354 | Peptide3_24_helix_1 | 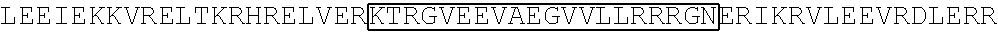 |
| 355 | Peptide3_24_helix_2 | EDLKARLEDIIRRLAEDIERVKRETDKLLEELARIIEEVEKEMRETNKELDKEIKDG |
| 356 | Peptide3_24_helix_3 | KALEEAKRVLAENNEVIKDIKRALDELLKELEKSVRESEERLRRLLEEVKRVIEEVAT |
| 357 | Peptide3_28_helix_1 | 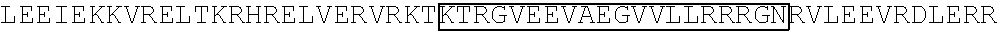 |
| 358 | Peptide3_28_helix_2 | EDLKARLEDIIRRLAEDIERVKRETDKLLEELARIIEEVEKEMRETNKELDKEIKDG |
| 359 | Peptide3_28_helix_3 | KALEEAKRVLAENNEVIKDIKRALDELLKELEKSVRESEERLRRLLEEVKRVIEEVAT |
| 360 | Peptide3_29_helix_1 | 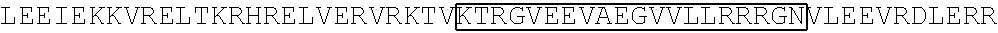 |
| 361 | Peptide3_29_helix_2 | EDLKARLEDIIRRLAEDIERVKRETDKLLEELARIIEEVEKEMRETNKELDKEIKDG |
| 362 | Peptide3_29_helix_3 | KALEEAKRVLAENNEVIKDIKRALDELLKELEKSVRESEERLRRLLEEVKRVIEEVAT |
| 363 | Peptide3_3_helix_1 | 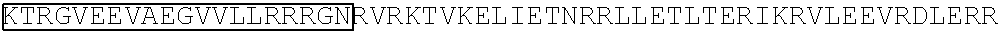 |
| 364 | Peptide3_3_helix_2 | EDLKARLEDIIRRLAEDIERVKRETDKLLEELARIIEEVEKEMRETNKELDKEIKDG |
| 365 | Peptide3_3_helix_3 | KALEEAKRVLAENNEVIKDIKRALDELLKELEKSVRESEERLRRLLEEVKRVIEEVAT |
| 366 | Peptide3_32_helix_1 | 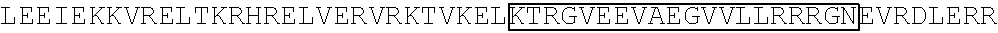 |
| 367 | Peptide3_32_helix_2 | EDLKARLEDIIRRLAEDIERVKRETDKLLEELARIIEEVEKEMRETNKELDKEIKDG |
| 368 | Peptide3_32_helix_3 | KALEEAKRVLAENNEVIKDIKRALDELLKELEKSVRESEERLRRLLEEVKRVIEEVAT |
| 369 | Peptide3_35_helix_1 | 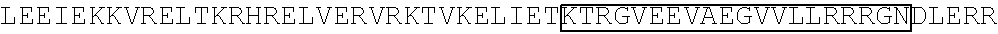 |
| 370 | Peptide3_35_helix_2 | EDLKARLEDIIRRLAEDIERVKRETDKLLEELARIIEEVEKEMRETNKELDKEIKDG |
| 371 | Peptide3_35_helix_3 | KALEEAKRVLAENNEVIKDIKRALDELLKELEKSVRESEERLRRLLEEVKRVIEEVAT |
| 372 | Peptide3_39_helix_1 | 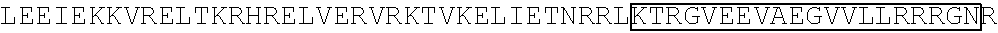 |
| 373 | Peptide3_39_helix_2 | EDLKARLEDIIRRLAEDIERVKRETDKLLEELARIIEEVEKEMRETNKELDKEIKDG |
| 374 | Peptide3_39_helix_3 | KALEEAKRVLAENNEVIKDIKRALDELLKELEKSVRESEERLRRLLEEVKRVIEEVAT |
| 375 | Peptide3_7_helix_1 | 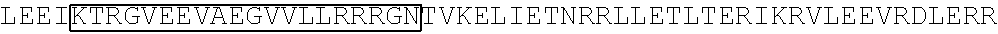 |
| 376 | Peptide3_7_helix_2 | EDLKARLEDIIRRLAEDIERVKRETDKLLEELARIIEEVEKEMRETNKELDKEIKDG |
| 377 | Peptide3_7_helix_3 | KALEEAKRVLAENNEVIKDIKRALDELLKELEKSVRESEERLRRLLEEVKRVIEEVAT |
| 378 | Peptide4_10_helix_1 | 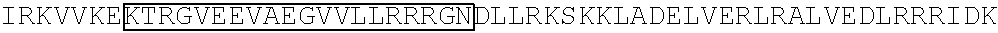 |
| 379 | Peptide4_10_helix_2 | ETAEDIVRRIIEELKRILKEIEDLARRINREIERLVEEVERDNRDVNRAIEELLKDIAR |
| | | RG |
| 380 | Peptide4_10_helix_3 | EDLKRVEERAREVSRRNEESMRRVKEDADRVSEANKEVLDRVREEVKRLIEEVRETLR |
| 381 | Peptide4_125_helix_1 | IRKVVKEITDLLKASNDKNRKVVEEIRDLLRKSKKLADELVERLRALVEDLRRRIDK |
| 382 | Peptide4_125_helix_2 | ETAEDIVRRIIEELKRILKEIEDLARRINREIERLVEEVERDNRDVNRAIEELLKDIAR |
| | | RG |
| 383 | Peptide4_125_helix_3 | RGVEEVAEGVVLLRRRGNESMRRVKEDADRVSEANKEVLDRVREEVKRLIEEVRETLR |
| 384 | Peptide4_129_helix_1 | IRKVVKEITDLLKASNDKNRKVVEEIRDLLRKSKKLADELVERLRALVEDLRRRIDK |
| 385 | Peptide4_129_helix_2 | ETAEDIVRRIIEELKRILKEIEDLARRINREIERLVEEVERDNRDVNRAIEELLKDIAR |
| | | RG |
| 386 | Peptide4_129_helix_3 | 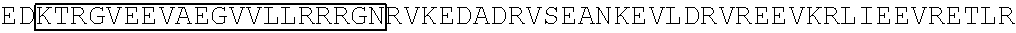 |
| 387 | Peptide4_132_helix_1 | IRKVVKEITDLLKASNDKNRKVVEEIRDLLRKSKKLADELVERLRALVEDLRRRIDK |
| 388 | Peptide4_132_helix_2 | ETAEDIVRRIIEELKRILKEIEDLARRINREIERLVEEVERDNRDVNRAIEELLKDIAR |
| | | RG |
| 389 | Peptide4_132_helix_3 | 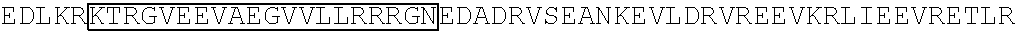 |
| 390 | Peptide4_133_helix_1 | IRKVVKEITDLLKASNDKNRKVVEEIRDLLRKSKKLADELVERLRALVEDLRRRIDK |
| 391 | Peptide4_133_helix_2 | ETAEDIVRRIIEELKRILKEIEDLARRINREIERLVEEVERDNRDVNRAIEELLKDIAR |
| | | RG |
| 392 | Peptide4_133_helix_3 | 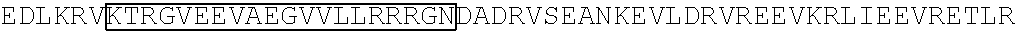 |
| 393 | Peptide4_135_helix_1 | IRKVVKEITDLLKASNDKNRKVVEEIRDLLRKSKKLADELVERLRALVEDLRRRIDK |
| 394 | Peptide4_135_helix_2 | ETAEDIVRRIIEELKRILKEIEDLARRINREIERLVEEVERDNRDVNRAIEELLKDIAR |
| | | RG |
| 395 | Peptide4_135_helix_3 | 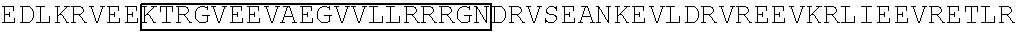 |
| 396 | Peptide4_136_helix_1 | IRKVVKEITDLLKASNDKNRKVVEEIRDLLRKSKKLADELVERLRALVEDLRRRIDK |
| 397 | Peptide4_136_helix_2 | ETAEDIVRRIIEELKRILKEIEDLARRINREIERLVEEVERDNRDVNRAIEELLKDIAR |
| | | RG |
| 398 | Peptide4_136_helix_3 | 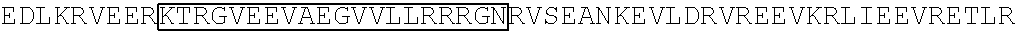 |
| 399 | Peptide4_139_helix_1 | IRKVVKEITDLLKASNDKNRKVVEEIRDLLRKSKKLADELVERLRALVEDLRRRIDK |
| 400 | Peptide4_139_helix_2 | ETAEDIVRRIIEELKRILKEIEDLARRINREIERLVEEVERDNRDVNRAIEELLKDIAR |
| | | RG |
| 401 | Peptide4_139_helix_3 | 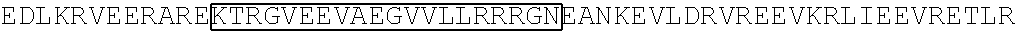 |
| 402 | Peptide4_140_helix_1 | IRKVVKEITDLLKASNDKNRKVVEEIRDLLRKSKKLADELVERLRALVEDLRRRIDK |
| 403 | Peptide4_140_helix_2 | ETAEDIVRRIIEELKRILKEIEDLARRINREIERLVEEVERDNRDVNRAIEELLKDIAR |
| | | RG |
| 404 | Peptide4_140_helix_3 | 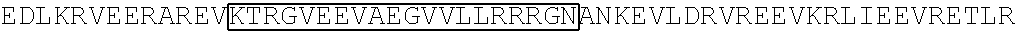 |
| 405 | Peptide4_141_helix_1 | IRKVVKEITDLLKASNDKNRKVVEEIRDLLRKSKKLADELVERLRALVEDLRRRIDK |
| 406 | Peptide4_141_helix_2 | ETAEDIVRRIIEELKRILKEIEDLARRINREIERLVEEVERDNRDVNRAIEELLKDIAR |
| | | RG |
| 407 | Peptide4_141_helix_3 | 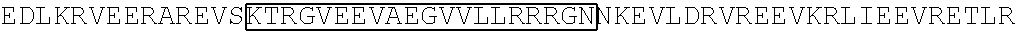 |
| 408 | Peptide4_144_helix_1 | IRKVVKEITDLLKASNDKNRKVVEEIRDLLRKSKKLADELVERLRALVEDLRRRIDK |
| 409 | Peptide4_144_helix_2 | ETAEDIVRRIIEELKRILKEIEDLARRINREIERLVEEVERDNRDVNRAIEELLKDIAR |
| | | RG |
| 410 | Peptide4_144_helix_3 | 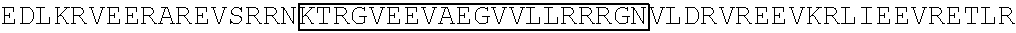 |
| 411 | Peptide4_147_helix_1 | IRKVVKEITDLLKASNDKNRKVVEEIRDLLRKSKKLADELVERLRALVEDLRRRIDK |
| 412 | Peptide4_147_helix_2 | ETAEDIVRRIIEELKRILKEIEDLARRINREIERLVEEVERDNRDVNRAIEELLKDIAR |
| | | RG |
| 413 | Peptide4_147_helix_3 | 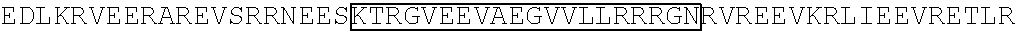 |
| 414 | Peptide4_150_helix_1 | IRKVVKEITDLLKASNDKNRKVVEEIRDLLRKSKKLADELVERLRALVEDLRRRIDK |
| 415 | Peptide4_150_helix_2 | ETAEDIVRRIIEELKRILKEIEDLARRINREIERLVEEVERDNRDVNRAIEELLKDIAR |
| | | RG |
| 416 | Peptide4_150_helix_3 | 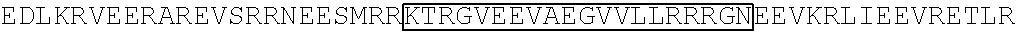 |
| 417 | Peptide4_151_helix_1 | IRKVVKEITDLLKASNDKNRKVVEEIRDLLRKSKKLADELVERLRALVEDLRRRIDK |
| 418 | Peptide4_151_helix_2 | ETAEDIVRRIIEELKRILKEIEDLARRINREIERLVEEVERDNRDVNRAIEELLKDIAR |
| | | RG |
| 419 | Peptide4_151_helix_3 | 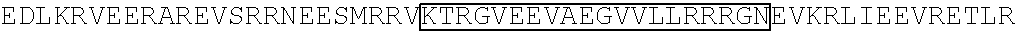 |
| 420 | Peptide4_154_helix_1 | IRKVVKEITDLLKASNDKNRKVVEEIRDLLRKSKKLADELVERLRALVEDLRRRIDK |
| 421 | Peptide4_154_helix_2 | ETAEDIVRRIIEELKRILKEIEDLARRINREIERLVEEVERDNRDVNRAIEELLKDIAR |
| | | RG |
| 422 | Peptide4_154_helix_3 | 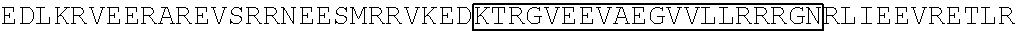 |
| 423 | Peptide4_155_helix_1 | IRKVVKEITDLLKASNDKNRKVVEEIRDLLRKSKKLADELVERLRALVEDLRRRIDK |
| 424 | Peptide4_155_helix_2 | ETAEDIVRRIIEELKRILKEIEDLARRINREIERLVEEVERDNRDVNRAIEELLKDIAR |
| | | RG |
| 425 | Peptide4_155_helix_3 | 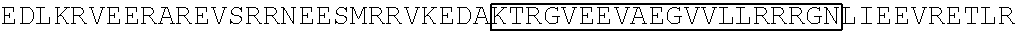 |
| 426 | Peptide4_157_helix_1 | IRKVVKEITDLLKASNDKNRKVVEEIRDLLRKSKKLADELVERLRALVEDLRRRIDK |
| 427 | Peptide4_157_helix_2 | ETAEDIVRRIIEELKRILKEIEDLARRINREIERLVEEVERDNRDVNRAIEELLKDIAR |
| | | RG |
| 428 | Peptide4_157_helix_3 | 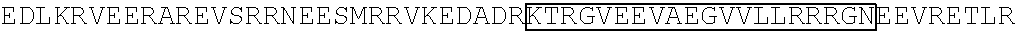 |
| 429 | Peptide4_158_helix_1 | IRKVVKEITDLLKASNDKNRKVVEEIRDLLRKSKKLADELVERLRALVEDLRRRIDK |
| 430 | Peptide4_158_helix_2 | ETAEDIVRRIIEELKRILKEIEDLARRINREIERLVEEVERDNRDVNRAIEELLKDIAR |
| | | RG |
| 431 | Peptide4_158_helix_3 | 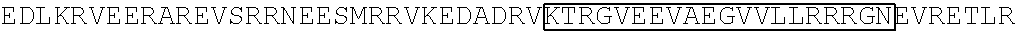 |
| 432 | Peptide4_161_helix_1 | IRKVVKEITDLLKASNDKNRKVVEEIRDLLRKSKKLADELVERLRALVEDLRRRIDK |
| 433 | Peptide4_161_helix_2 | ETAEDIVRRIIEELKRILKEIEDLARRINREIERLVEEVERDNRDVNRAIEELLKDIAR |
| | | RG |
| 434 | Peptide4_161_helix_3 | 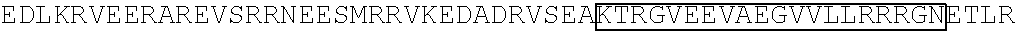 |
| 435 | Peptide4_162_helix_1 | IRKVVKEITDLLKASNDKNRKVVEEIRDLLRKSKKLADELVERLRALVEDLRRRIDK |
| 436 | Peptide4_162_helix_2 | ETAEDIVRRIIEELKRILKEIEDLARRINREIERLVEEVERDNRDVNRAIEELLKDIAR |
| | | RG |
| 437 | Peptide4_162_helix_3 | 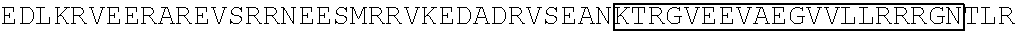 |
| 438 | Peptide4_164_helix_1 | IRKVVKEITDLLKASNDKNRKVVEEIRDLLRKSKKLADELVERLRALVEDLRRRIDK |
| 439 | Peptide4_164_helix_2 | ETAEDIVRRIIEELKRILKEIEDLARRINREIERLVEEVERDNRDVNRAIEELLKDIAR |
| | | RG |
| 440 | Peptide4_164_helix_3 | 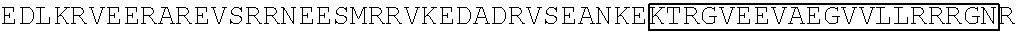 |
| 441 | Peptide4_165_helix_1 | IRKVVKEITDLLKASNDKNRKVVEEIRDLLRKSKKLADELVERLRALVEDLRRRIDK |
| 442 | Peptide4_165_helix_2 | ETAEDIVRRIIEELKRILKEIEDLARRINREIERLVEEVERDNRDVNRAIEELLKDIAR |
| | | RG |
| 443 | Peptide4_165_helix_3 | 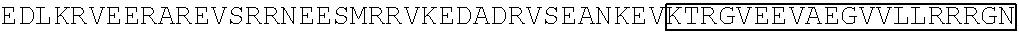 |
| 444 | Peptide4_18_helix_1 | 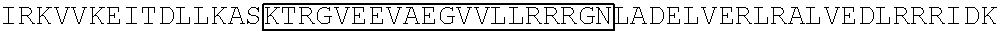 |
| 445 | Peptide4_18_helix_2 | ETAEDIVRRIIEELKRILKEIEDLARRINREIERLVEEVERDNRDVNRAIEELLKDIAR |
| | | RG |
| 446 | Peptide4_18_helix_3 | EDLKRVEERAREVSRRNEESMRRVKEDADRVSEANKEVLDRVREEVKRLIEEVRETLR |
| 447 | Peptide4_21_helix_1 | 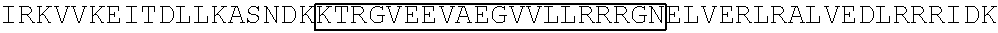 |
| 448 | Peptide4_21_helix_2 | ETAEDIVRRIIEELKRILKEIEDLARRINREIERLVEEVERDNRDVNRAIEELLKDIAR |
| | | RG |
| 449 | Peptide4_21_helix_3 | EDLKRVEERAREVSRRNEESMRRVKEDADRVSEANKEVLDRVREEVKRLIEEVRETLR |
| 450 | Peptide4_24_helix_1 | 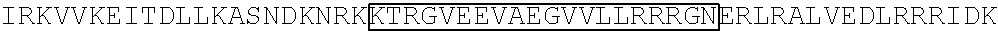 |
| 451 | Peptide4_24_helix_2 | ETAEDIVRRIIEELKRILKEIEDLARRINREIERLVEEVERDNRDVNRAIEELLKDIAR |
| | | RG |
| 452 | Peptide4_24_helix_3 | EDLKRVEERAREVSRRNEESMRRVKEDADRVSEANKEVLDRVREEVKRLIEEVRETLR |
| 453 | Peptide4_25_helix_1 | 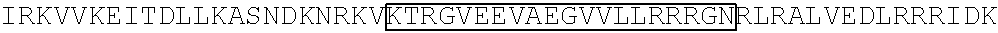 |
| 454 | Peptide4_25_helix_2 | ETAEDIVRRIIEELKRILKEIEDLARRINREIERLVEEVERDNRDVNRAIEELLKDIAR |
| | | RG |
| 455 | Peptide4_25_helix_3 | EDLKRVEERAREVSRRNEESMRRVKEDADRVSEANKEVLDRVREEVKRLIEEVRETLR |
| 456 | Peptide4_36_helix_1 | 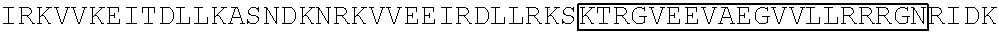 |
| 457 | Peptide4_36_helix_2 | ETAEDIVRRIIEELKRILKEIEDLARRINREIERLVEEVERDNRDVNRAIEELLKDIAR |
| | | RG |
| 458 | Peptide4_36_helix_3 | EDLKRVEERAREVSRRNEESMRRVKEDADRVSEANKEVLDRVREEVKRLIEEVRETLR |
| 459 | Peptide4_39_helix_1 | 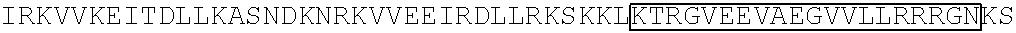 |
| 460 | Peptide4_39_helix_2 | ETAEDIVRRIIEELKRILKEIEDLARRINREIERLVEEVERDNRDVNRAIEELLKDIAR |
| | | RG |
| 461 | Peptide4_39_helix_3 | EDLKRVEERAREVSRRNEESMRRVKEDADRVSEANKEVLDRVREEVKRLIEEVRETLR |
| 462 | Peptide4_6_helix_1 | 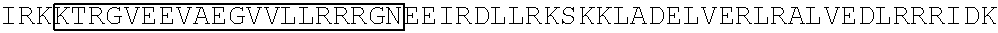 |
| 463 | Peptide4_6_helix_2 | ETAEDIVRRIIEELKRILKEIEDLARRINREIERLVEEVERDNRDVNRAIEELLKDIAR |
| | | RG |
| 464 | Peptide4_6_helix_3 | EDLKRVEERAREVSRRNEESMRRVKEDADRVSEANKEVLDRVREEVKRLIEEVRETLR |
| 465 | Peptide4_7_helix_1 | 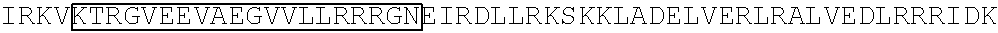 |
| 466 | Peptide4_7_helix_2 | ETAEDIVRRIIEELKRILKEIEDLARRINREIERLVEEVERDNRDVNRAIEELLKDIAR |
| | | RG |
| 467 | Peptide4_7_helix_3 | EDLKRVEERAREVSRRNEESMRRVKEDADRVSEANKEVLDRVREEVKRLIEEVRETLR |
| 468 | Peptide5_10_helix_1 | 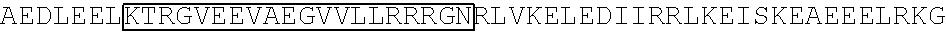 |
| 469 | Peptide5_10_helix_2 | DILRDLERRLREILEESDRLLEELKRRLEEILRKSKELLRRLEEVLREILKRAEEVKRS |
| 470 | Peptide5_10_helix_3 | EELIKEIVKLLEELLRVIEKILEDNIRLLEELVEVIKEILEKHLRLLEELVRVIERILR |
| | | EVG |
| 471 | Peptide5_10_helix_4 | DEAERRRRELKDKLDRLREEHEEVKRRLEEELTRLRETHKKIEKELREALKRVRDRST |
| 472 | Peptide5_190_helix_1 | AEDLEELIKELAELLKDVIRKLEKINRRLVKILEDIIRRLKEISKEAEEELRKG |
| 473 | Peptide5_190_helix_2 | DILRDLERRLREILEESDRLLEELKRRLEEILRKSKELLRRLEEVLREILKRAEEVKRS |
| 474 | Peptide5_190_helix_3 | EELIKEIVKLLEELLRVIEKILEDNIRLLEELVEVIKEILEKHLRLLEELVRVIERILR |
| | | EVG |
| 475 | Peptide5_190_helix_4 | 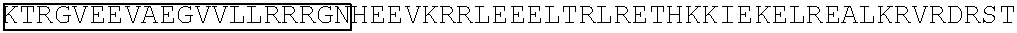 |
| 476 | Peptide5_192_helix_1 | AEDLEELIKELAELLKDVIRKLEKINRRLVKILEDIIRRLKEISKEAEEELRKG |
| 477 | Peptide5_192_helix_2 | DILRDLERRLREILEESDRLLEELKRRLEEILRKSKELLRRLEEVLREILKRAEEVKRS |
| 478 | Peptide5_192_helix_3 | EELIKEIVKLLEELLRVIEKILEDNIRLLEELVEVIKEILEKHLRLLEELVRVIERILR |
| | | EVG |
| 479 | Peptide5_192_helix_4 | 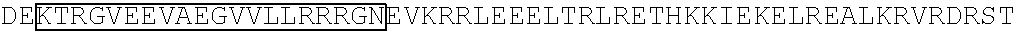 |
| 480 | Peptide5_196_helix_1 | AEDLEELIKELAELLKDVIRKLEKINRRLVKILEDIIRRLKEISKEAEEELRKG |
| 481 | Peptide5_196_helix_2 | DILRDLERRLREILEESDRLLEELKRRLEEILRKSKELLRRLEEVLREILKRAEEVKRS |
| 482 | Peptide5_196_helix_3 | EELIKEIVKLLEELLRVIEKILEDNIRLLEELVEVIKEILEKHLRLLEELVRVIERILR |
| | | EVG |
| 483 | Peptide5_196_helix_4 | 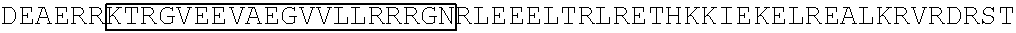 |
| 484 | Peptide5_199_helix_1 | AEDLEELIKELAELLKDVIRKLEKINRRLVKILEDIIRRLKEISKEAEEELRKG |
| 485 | Peptide5_199_helix_2 | DILRDLERRLREILEESDRLLEELKRRLEEILRKSKELLRRLEEVLREILKRAEEVKRS |
| 486 | Peptide5_199_helix_3 | EELIKEIVKLLEELLRVIEKILEDNIRLLEELVEVIKEILEKHLRLLEELVRVIERILR |
| | | EVG |
| 487 | Peptide5_199_helix_4 | 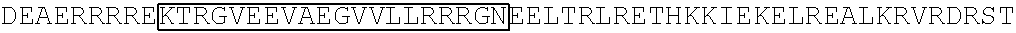 |
| 488 | Peptide5_203_helix_1 | AEDLEELIKELAELLKDVIRKLEKINRRLVKILEDIIRRLKEISKEAEEELRKG |
| 489 | Peptide5_203_helix_2 | DILRDLERRLREILEESDRLLEELKRRLEEILRKSKELLRRLEEVLREILKRAEEVKRS |
| 490 | Peptide5_203_helix_3 | EELIKEIVKLLEELLRVIEKILEDNIRLLEELVEVIKEILEKHLRLLEELVRVIERILR |
| | | EVG |
| 491 | Peptide5_203_helix_4 | 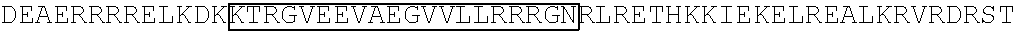 |
| 492 | Peptide5_210_helix_1 | AEDLEELIKELAELLKDVIRKLEKINRRLVKILEDIIRRLKEISKEAEEELRKG |
| 493 | Peptide5_210_helix_2 | DILRDLERRLREILEESDRLLEELKRRLEEILRKSKELLRRLEEVLREILKRAEEVKRS |
| 494 | Peptide5_210_helix_3 | EELIKEIVKLLEELLRVIEKILEDNIRLLEELVEVIKEILEKHLRLLEELVRVIERILR |
| | | EVG |
| 495 | Peptide5_210_helix_4 | 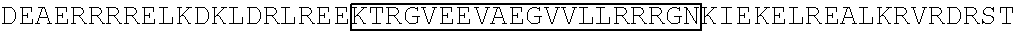 |
| 496 | Peptide5_221_helix_1 | AEDLEELIKELAELLKDVIRKLEKINRRLVKILEDIIRRLKEISKEAEEELRKG |
| 497 | Peptide5_221_helix_2 | DILRDLERRLREILEESDRLLEELKRRLEEILRKSKELLRRLEEVLREILKRAEEVKRS |
| 498 | Peptide5_221_helix_3 | EELIKEIVKLLEELLRVIEKILEDNIRLLEELVEVIKEILEKHLRLLEELVRVIERILR |
| | | EVG |
| 499 | Peptide5_221_helix_4 | 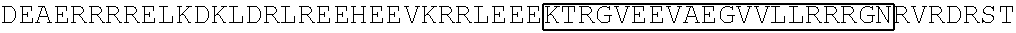 |
| 500 | Peptide5_224_helix_1 | AEDLEELIKELAELLKDVIRKLEKINRRLVKILEDIIRRLKEISKEAEEELRKG |
| 501 | Peptide5_224_helix_2 | DILRDLERRLREILEESDRLLEELKRRLEEILRKSKELLRRLEEVLREILKRAEEVKRS |
| 502 | Peptide5_224_helix_3 | EELIKEIVKLLEELLRVIEKILEDNIRLLEELVEVIKEILEKHLRLLEELVRVIERILR |
| | | EVG |
| 503 | Peptide5_224_helix_4 | 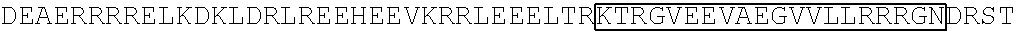 |
| 504 | Peptide5_228_helix_1 | AEDLEELIKELAELLKDVIRKLEKINRRLVKILEDIIRRLKEISKEAEEELRKG |
| 505 | Peptide5_228_helix_2 | DILRDLERRLREILEESDRLLEELKRRLEEILRKSKELLRRLEEVLREILKRAEEVKRS |
| 506 | Peptide5_228_helix_3 | EELIKEIVKLLEELLRVIEKILEDNIRLLEELVEVIKEILEKHLRLLEELVRVIERILR |
| | | EVG |
| 507 | Peptide5_228_helix_4 | 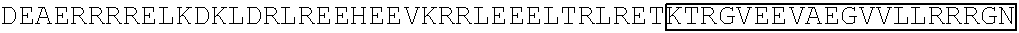 |
| 508 | Peptide5_3_helix_1 | 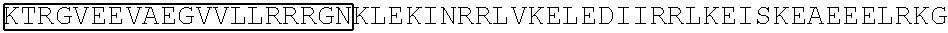 |
| 509 | Peptide5_3_helix_2 | DILRDLERRLREILEESDRLLEELKRRLEEILRKSKELLRRLEEVLREILKRAEEVKRS |
| 510 | Peptide5_3_helix_3 | EELIKEIVKLLEELLRVIEKILEDNIRLLEELVEVIKEILEKHLRLLEELVRVIERILR |
| | | EVG |
| 511 | Peptide5_3_helix_4 | DEAERRRRELKDKLDRLREEHEEVKRRLEEELTRLRETHKKIEKELREALKRVRDRST |
| 512 | Peptide5_35_helix_1 | 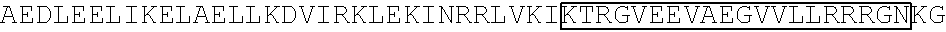 |
| 513 | Peptide5_35_helix_2 | DILRDLERRLREILEESDRLLEELKRRLEEILRKSKELLRRLEEVLREILKRAEEVKRS |
| 514 | Peptide5_35_helix_3 | EELIKEIVKLLEELLRVIEKILEDNIRLLEELVEVIKEILEKHLRLLEELVRVIERILR |
| | | EVG |
| 515 | Peptide5_35_helix_4 | DEAERRRRELKDKLDRLREEHEEVKRRLEEELTRLRETHKKIEKELREALKRVRDRST |
| 516 | Peptide5_6_helix_1 | 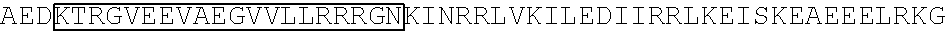 |
| 517 | Peptide5_6_helix_2 | DILRDLERRLREILEESDRLLEELKRRLEEILRKSKELLRRLEEVLREILKRAEEVKRS |
| 518 | Peptide5_6_helix_3 | EELIKEIVKLLEELLRVIEKILEDNIRLLEELVEVIKEILEKHLRLLEELVRVIERILR |
| | | EVG |
| 519 | Peptide5_6_helix_4 | DEAERRRRELKDKLDRLREEHEEVKRRLEEELTRLRETHKKIEKELREALKRVRDRST |
| 520 | Peptide6_206_helix_1 | AEDLEELIKELAELLKDVIRKLEKINRRLVKILEDIIRRLKEISKEAEEELRKG |
| 521 | Peptide6_206_helix_2 | DILRDLERRLREILEESDRLLEELKRRLEEILRKSKELLRRLEEVLREILKRAEEVKRS |
| 522 | Peptide6_206_helix_3 | EELIKEIVKLLEELLRVIEKILEDNIRLLEELVEVIKEILEKHLRLLEELVRVIERILR |
| | | EVG |
| 523 | Peptide6_206_helix_4 | 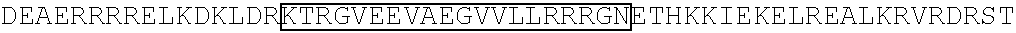 |
| 524 | Peptide6_6_helix_1 | 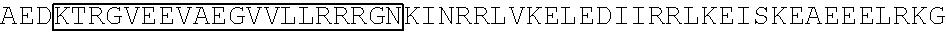 |
| 525 | Peptide6_6_helix_2 | DILRDLERRLREILEESDRLLEELKRRLEEILRKSKELLRRLEEVLREILKRAEEVKRS |
| 526 | Peptide6_6_helix_3 | EELIKEIVKLLEELLRVIEKILEDNIRLLEELVEVIKEILEKHLRLLEELVRVIERILR |
| | | EVG |
| 527 | Peptide6_6_helix_4 | DEAERRRRELKDKLDRLREEHEEVKRRLEEELTRLRETHKKIEKELREALKRVRDRST |
| 528 | Peptide7_218_helix_1 | DRIIEEIARLLEELLRELLELIKKLIETNRRLNEEHERAVRELARLLEELLDRLVKKG |
| 529 | Peptide7_218_helix_2 | KLKRIRERLKRALDDLERLHREINKRLEDLVRELEKLVREILKELKDALEELRRASAR |
| 530 | Peptide7_218_helix_3 | EVLRRLEEIVKKLLDLVRRILERLKEIHKDNVRLLRELNERLTRIVEDLVRLIREILRE |
| | | AG |
| 531 | Peptide7_218_helix_4 | 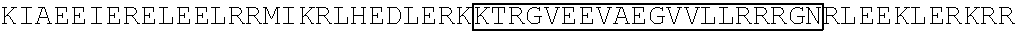 |
| 532 | Peptide7_3_helix_1 | 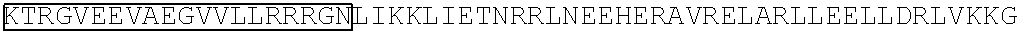 |
| 533 | Peptide7_3_helix_2 | KLKRIRERLKRALDDLERLHREINKRLEDLVRELEKLVREILKELKDALEELRRASAR |
| 534 | Peptide7_3_helix_3 | EVLRRLEEIVKKLLDLVRRILERLKEIHKDNVRLLRELNERLTRIVEDLVRLIREILRE |
| | | AG |
| 535 | Peptide7_3_helix_4 | KIAEEIERELEELRRMIKRLHEDLERKLKESEDELREIEARLEEKIRRLEEKLERKRR |
| 536 | Peptides_196_helix_1 | KELLEEVVRRAIELLKRHLEKLKRILEEIVRLLEEHLEKVERVLEAILSLLDDLLRRG |
| 537 | Peptides_196_helix_2 | RAIRTLEDVKRRLREILERLADENAKAIKRLADLLDKLEKRNKEAIERLEEILEELKRV |
| 538 | Peptides_196_helix_3 | EELLRVLETLLKIIEDILRENTKVLEDLLRLVEEILEANLRVVEELLRLAREILTEIV |
| 539 | Peptides_196_helix_4 | 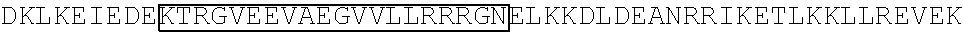 |
| 540 | Peptides_200_helix_1 | KELLEEVVRRAIELLKRHLEKLKRILEEIVRLLEEHLEKVERVLEAILSLLDDLLRRG |
| 541 | Peptides_200_helix_2 | RAIRTLEDVKRRLREILERLADENAKAIKRLADLLDKLEKRNKEAIERLEEILEELKRV |
| 542 | Peptides_200_helix_3 | EELLRVLETLLKIIEDILRENTKVLEDLLRLVEEILEANLRVVEELLRLAREILTEIV |
| 543 | Peptides_200_helix_4 | 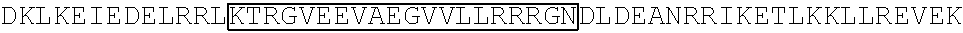 |
| 544 | Peptides_203_helix_1 | KELLEEVVRRAIELLKRHLEKLKRILEEIVRLLEEHLEKVERVLEAILSLLDDLLRRG |
| 545 | Peptides_203_helix_2 | RAIRTLEDVKRRLREILERLADENAKAIKRLADLLDKLEKRNKEAIERLEEILEELKRV |
| 546 | Peptides_203_helix_3 | EELLRVLETLLKIIEDILRENTKVLEDLLRLVEEILEANLRVVEELLRLAREILTEIV |
| 547 | Peptides_203_helix_4 | 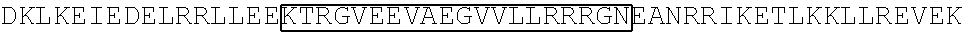 |
| 548 | Peptides_218_helix_1 | KELLEEVVRRAIELLKRHLEKLKRILEEIVRLLEEHLEKVERVLEAILSLLDDLLRRG |
| 549 | Peptides_218_helix_2 | RAIRTLEDVKRRLREILERLADENAKAIKRLADLLDKLEKRNKEAIERLEEILEELKRV |
| 550 | Peptides_218_helix_3 | EELLRVLETLLKIIEDILRENTKVLEDLLRLVEEILEANLRVVEELLRLAREILTEIV |
| 551 | Peptides_218_helix_4 | 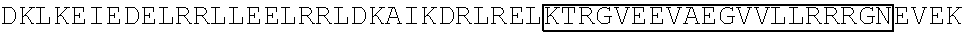 |
| 552 | Peptide9_10_helix_1 | 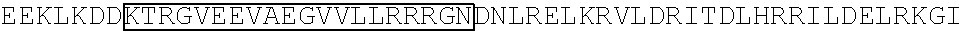 |
| 553 | Peptide9_10_helix_2 | ELLRRVEKVLKDNLDLLRKLVEEHKESSERDLKRVEDLVREIKEVLRKLLELEDR |
| 554 | Peptide9_10_helix_3 | RKIEEEIERLLRKIRKAVEESKDLNRRNSERIEEVARRSEELARRLLKEIRERG |
| 555 | Peptide9_10_helix_4 | KAAEDILRVLEKLVKVSREAIKLILELSEHHVRVSTRIARLLLDVARKLAEVIKEAER |
| 556 | Peptide9_13_helix_1 | 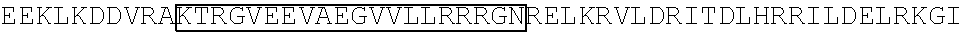 |
| 557 | Peptide9_13_helix_2 | ELLRRVEKVLKDNLDLLRKLVEEHKESSERDLKRVEDLVREIKEVLRKLLELEDR |
| 558 | Peptide9_13_helix_3 | RKIEEEIERLLRKIRKAVEESKDLNRRNSERIEEVARRSEELARRLLKEIRERG |
| 559 | Peptide9_13_helix_4 | KAAEDILRVLEKLVKVSREAIKLILELSEHHVRVSTRIARLLLDVARKLAEVIKEAER |
| 560 | Peptide9_174_helix_1 | EEKLKDDVRAVLEDLDRVLKELEKLSEDNLRELKRVLDRITDLHRRILDELRKGI |
| 561 | Peptide9_174_helix_2 | ELLRRVEKVLKDNLDLLRKLVEEHKESSERDLKRVEDLVREIKEVLRKLLELEDR |
| 562 | Peptide9_174_helix_3 | RKIEEEIERLLRKIRKAVEESKDLNRRNSERIEEVARRSEELARRLLKEIRERG |
| 563 | Peptide9_174_helix_4 | RGVEEVAEGVVLLRRRGNEAIKLILELSEHHVRVSTRIARLLLDVARKLAEVIKEAER |
| 564 | Peptide9_214_helix_1 | EEKLKDDVRAVLEDLDRVLKELEKLSEDNLRELKRVLDRITDLHRRILDELRKGI |
| 565 | Peptide9_214_helix_2 | ELLRRVEKVLKDNLDLLRKLVEEHKESSERDLKRVEDLVREIKEVLRKLLELEDR |
| 566 | Peptide9_214_helix_3 | RKIEEEIERLLRKIRKAVEESKDLNRRNSERIEEVARRSEELARRLLKEIRERG |
| 567 | Peptide9_214_helix_4 | 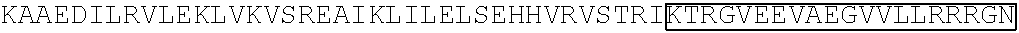 |
| 568 | Peptide9_3_helix_1 | 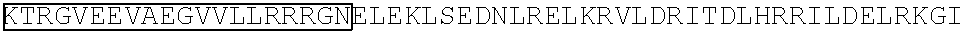 |
| 569 | Peptide9_3_helix_2 | ELLRRVEKVLKDNLDLLRKLVEEHKESSERDLKRVEDLVREIKEVLRKLLELEDR |
| 570 | Peptide9_3_helix_3 | RKIEEEIERLLRKIRKAVEESKDLNRRNSERIEEVARRSEELARRLLKEIRERG |
| 571 | Peptide9_3_helix_4 | KAAEDILRVLEKLVKVSREAIKLILELSEHHVRVSTRIARLLLDVARKLAEVIKEAER |
| 572 | Peptide9_6_helix_1 | 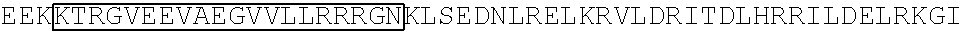 |
| 573 | Peptide9_6_helix_2 | ELLRRVEKVLKDNLDLLRKLVEEHKESSERDLKRVEDLVREIKEVLRKLLELEDR |
| 574 | Peptide9_6_helix_3 | RKIEEEIERLLRKIRKAVEESKDLNRRNSERIEEVARRSEELARRLLKEIRERG |
| 575 | Peptide9_6_helix_4 | KAAEDILRVLEKLVKVSREAIKLILELSEHHVRVSTRIARLLLDVARKLAEVIKEAER |
| 576 | Peptide10_13_helix_1 | 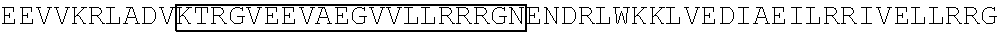 |
| 577 | Peptide10_13_helix_2 | ELLDRLAKVVKSIVEKAEKILERLNRVSKAIAEKLKTIVDELNEVSKEIVKRAEDILRK |
| | | G |
| 578 | Peptide10_13_helix_3 | ETVLRALRTLVKEYADLSKEVLERVERIVREYVKLSDEVVKSLAEIVEELIRIIEDLLR |
| | | KGN |
| 579 | Peptide10_13_helix_4 | DVKRALEELVSRLRKLLEDVKKASEDIVREVERIVRELAKRSDEILKKLEDIVEKLRE |
| 580 | Peptide10_190_helix_ | EEVVKRLADVLRENDETIRKVVEDLVRIAEENDRLVVKKLVEDIAEILRRIVELLRRG |
| | 1 | |
| 581 | Peptide10_190_helix_ | ELLDRLAKVVKSIVEKAEKILERLNRVSKAIAEKLKTIVDELNEVSKEIVKRAEDILRK |
| | 2 | G |
| 582 | Peptide10_190_helix_ | ETVLRALRTLVKEYADLSKEVLERVERIVREYVKLSDEVVKSLAEIVEELIRIIEDLLR |
| | 3 | KGN |
| 583 | Peptide10_190_helix_ | RGVEEVAEGVVLLRRRGNDVKKASEDIVREVERIVRELAKRSDEILKKLEDIVEKLRE |
| | 4 | |
| 584 | Peptide10_193_helix_ | EEVVKRLADVLRENDETIRKVVEDLVRIAEENDRLVVKKLVEDIAEILRRIVELLRRG |
| | 1 | |
| 585 | Peptide10_193_helix_ | ELLDRLAKVVKSIVEKAEKILERLNRVSKAIAEKLKTIVDELNEVSKEIVKRAEDILRK |
| | 2 | G |
| 586 | Peptide10_193_helix_ | ETVLRALRTLVKEYADLSKEVLERVERIVREYVKLSDEVVKSLAEIVEELIRIIEDLLR |
| | 3 | KGN |
| 587 | Peptide10_193_helix_ | 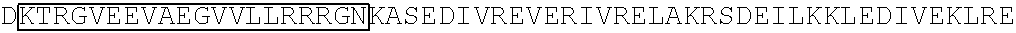 |
| | 4 | |
| 588 | Peptide10_197_helix_ | EEVVKRLADVLRENDETIRKVVEDLVRIAEENDRLVVKKLVEDIAEILRRIVELLRRG |
| | 1 | |
| 589 | Peptide10_197_helix_ | ELLDRLAKVVKSIVEKAEKILERLNRVSKAIAEKLKTIVDELNEVSKEIVKRAEDILRK |
| | 2 | G |
| 590 | Peptide10_197_helix_ | ETVLRALRTLVKEYADLSKEVLERVERIVREYVKLSDEVVKSLAEIVEELIRIIEDLLR |
| | 3 | KG |
| 591 | Peptide10_197_helix_ | 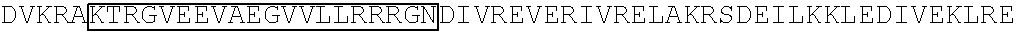 |
| | 4 | |
| 592 | Peptide10_211_helix_ | EEVVKRLADVLRENDETIRKVVEDLVRIAEENDRLVVKKLVEDIAEILRRIVELLRRG |
| | 1 | |
| 593 | Peptide10_211_helix_ | ELLDRLAKVVKSIVEKAEKILERLNRVSKAIAEKLKTIVDELNEVSKEIVKRAEDILRK |
| | 2 | G |
| 594 | Peptide10_211_helix_ | ETVLRALRTLVKEYADLSKEVLERVERIVREYVKLSDEVVKSLAEIVEELIRIIEDLLR |
| | 3 | KGN |
| 595 | Peptide10_211_helix_ | 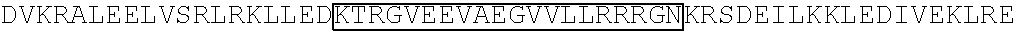 |
| | 4 | |
| 596 | Peptide10_219_helix_ | EEVVKRLADVLRENDETIRKVVEDLVRIAEENDRLVVKKLVEDIAEILRRIVELLRRG |
| | 1 | |
| 597 | Peptide10_219_helix_ | ELLDRLAKVVKSIVEKAEKILERLNRVSKAIAEKLKTIVDELNEVSKEIVKRAEDILRK |
| | 2 | G |
| 598 | Peptide10_219_helix_ | ETVLRALRTLVKEYADLSKEVLERVERIVREYVKLSDEVVKSLAEIVEELIRIIEDLLR |
| | 3 | KGN |
| 599 | Peptide10_219_helix_ | 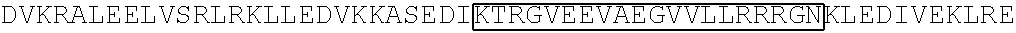 |
| | 4 | |
| 600 | Peptide10_222_helix_ | EEVVKRLADVLRENDETIRKVVEDLVRIAEENDRLVVKKLVEDIAEILRRIVELLRRG |
| | 1 | |
| 601 | Peptide10_222_helix_ | ELLDRLAKVVKSIVEKAEKILERLNRVSKAIAEKLKTIVDELNEVSKEIVKRAEDILRK |
| | 2 | G |
| 602 | Peptide10_222_helix_ | ETVLRALRTLVKEYADLSKEVLERVERIVREYVKLSDEVVKSLAEIVEELIRIIEDLLR |
| | 3 | KGN |
| 603 | Peptide10_222_helix_ | 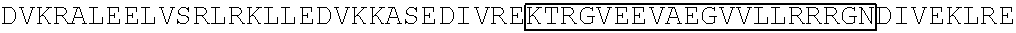 |
| | 4 | |
| 604 | Peptide10_229_helix_ | EEVVKRLADVLRENDETIRKVVEDLVRIAEENDRLVVKKLVEDIAEILRRIVELLRRG |
| | 1 | |
| 605 | Peptide10_229_helix_ | ELLDRLAKVVKSIVEKAEKILERLNRVSKAIAEKLKTIVDELNEVSKEIVKRAEDILRK |
| | 2 | G |
| 606 | Peptide10_229_helix_ | ETVLRALRTLVKEYADLSKEVLERVERIVREYVKLSDEVVKSLAEIVEELIRIIEDLLR |
| | 3 | KGN |
| 607 | Peptide10_229_helix_ | 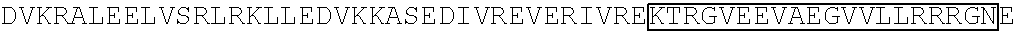 |
| | 4 | |
| 608 | Peptide10_230_helix_ | EEVVKRLADVLRENDETIRKVVEDLVRIAEENDRLVVKKLVEDIAEILRRIVELLRRG |
| | 1 | |
| 609 | Peptide10_230_helix_ | ELLDRLAKVVKSIVEKAEKILERLNRVSKAIAEKLKTIVDELNEVSKEIVKRAEDILRK |
| | 2 | G |
| 610 | Peptide10_230_helix_ | ETVLRALRTLVKEYADLSKEVLERVERIVREYVKLSDEVVKSLAEIVEELIRIIEDLLR |
| | 3 | KGN |
| 611 | Peptide10_230_helix_ | 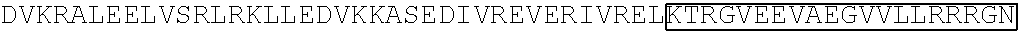 |
| | 4 | |
| 612 | Peptide10_31_helix_1 | 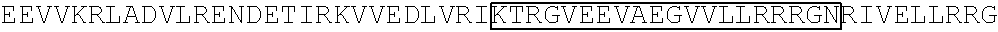 |
| 613 | Peptide10_31_helix_2 | ELLDRLAKVVKSIVEKAEKILERLNRVSKAIAEKLKTIVDELNEVSKEIVKRAEDILRK |
| | | G |
| 614 | Peptide10_31_helix_3 | ETVLRALRTLVKEYADLSKEVLERVERIVREYVKLSDEVVKSLAEIVEELIRIIEDLLR |
| | | KGN |
| 615 | Peptide10_31_helix_4 | DVKRALEELVSRLRKLLEDVKKASEDIVREVERIVRELAKRSDEILKKLEDIVEKLRE |
| 616 | Peptide10_38_helix_1 | 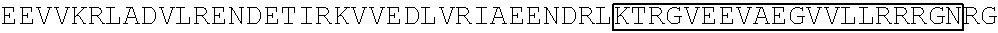 |
| 617 | Peptide10_38_helix_2 | ELLDRLAKVVKSIVEKAEKILERLNRVSKAIAEKLKTIVDELNEVSKEIVKRAEDILRK |
| | | G |
| 618 | Peptide10_38_helix_3 | ETVLRALRTLVKEYADLSKEVLERVERIVREYVKLSDEVVKSLAEIVEELIRIIEDLLR |
| | | KGN |
| 619 | Peptide10_38_helix_4 | DVKRALEELVSRLRKLLEDVKKASEDIVREVERIVRELAKRSDEILKKLEDIVEKLRE |
| 620 | Peptide10_6_helix_1 | 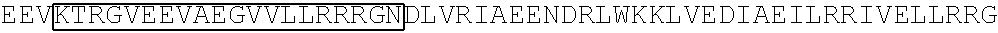 |
| 621 | Peptide10_6_helix_2 | ELLDRLAKVVKSIVEKAEKILERLNRVSKAIAEKLKTIVDELNEVSKEIVKRAEDILRK |
| | | G |
| 622 | Peptide10_6_helix_3 | ETVLRALRTLVKEYADLSKEVLERVERIVREYVKLSDEVVKSLAEIVEELIRIIEDLLR |
| | | KGN |
| 623 | Peptide10_6_helix_4 | DVKRALEELVSRLRKLLEDVKKASEDIVREVERIVRELAKRSDEILKKLEDIVEKLRE |
| 624 | Peptide10_9_helix_1 | 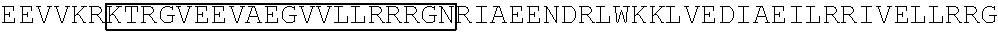 |
| 625 | Peptide10_9_helix_2 | ELLDRLAKVVKSIVEKAEKILERLNRVSKAIAEKLKTIVDELNEVSKEIVKRAEDILRK |
| | | G |
| 626 | Peptide10_9_helix_3 | ETVLRALRTLVKEYADLSKEVLERVERIVREYVKLSDEVVKSLAEIVEELIRIIEDLLR |
| | | KGN |
| 627 | Peptide10_9_helix_4 | DVKRALEELVSRLRKLLEDVKKASEDIVREVERIVRELAKRSDEILKKLEDIVEKLRE |
| 628 | Peptide_11_13_helix_1 | 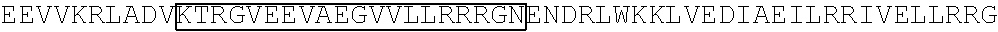 |
| 629 | Peptide_11_13_helix_2 | ELLDRLAKVVKSIVEKAEKILERLNRVSKAIAEKLKTIVDELNEVSKEIVKRAEDILRK |
| | | G |
| 630 | Peptide_11_13_helix_3 | ETVLRALRTLVKEYADLSKEVLERVERIVREYVKLSDEVVKSLAEIVEELIRIIEDLLR |
| | | KG |
| 631 | Peptide_11_13_helix_4 | DVKRALEELVSRLRKLLEDVKKASEDIVREVERIVRELAKRSDEILKKLEDIVEKLRE |
| 632 | Peptide11_190_helix_ | EEVVKRLADVLRENDETIRKVVEDLVRIAEENDRLVVKKLVEDIAEILRRIVELLRRG |
| | 1 | |
| 633 | Peptide11_190_helix_ | ELLDRLAKVVKSIVEKAEKILERLNRVSKAIAEKLKTIVDELNEVSKEIVKRAEDILRK |
| | 2 | G |
| 634 | Peptide11_190_helix_ | ETVLRALRTLVKEYADLSKEVLERVERIVREYVKLSDEVVKSLAEIVEELIRIIEDLLR |
| | 3 | KG |
| 635 | Peptide11_190_helix_ | RGVEEVAEGVVLLRRRGNDVKKASEDIVREVERIVRELAKRSDEILKKLEDIVEKLRE |
| | 4 | |
| 636 | Peptide11_20_helix_1 | 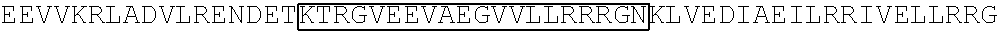 |
| 637 | Peptide11_20_helix_2 | ELLDRLAKVVKSIVEKAEKILERLNRVSKAIAEKLKTIVDELNEVSKEIVKRAEDILRK |
| | | G |
| 638 | Peptide11_20_helix_3 | ETVLRALRTLVKEYADLSKEVLERVERIVREYVKLSDEVVKSLAEIVEELIRIIEDLLR |
| | | KG |
| 639 | Peptide11_20_helix_4 | DVKRALEELVSRLRKLLEDVKKASEDIVREVERIVRELAKRSDEILKKLEDIVEKLRE |
| 640 | Peptide_11_201_helix_ | EEVVKRLADVLRENDETIRKVVEDLVRIAEENDRLVVKKLVEDIAEILRRIVELLRRG |
| | 1 | |
| 641 | Peptide_11_201_helix_ | ELLDRLAKVVKSIVEKAEKILERLNRVSKAIAEKLKTIVDELNEVSKEIVKRAEDILRK |
| | 2 | G |
| 642 | Peptide_11_201_helix_ | ETVLRALRTLVKEYADLSKEVLERVERIVREYVKLSDEVVKSLAEIVEELIRIIEDLLR |
| | 3 | KG |
| 643 | Peptide_11_201_helix_ | 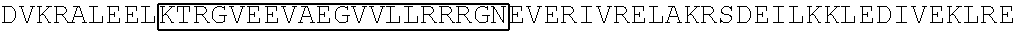 |
| | 4 | |
| 644 | Peptide11_219_helix_ | EEVVKRLADVLRENDETIRKVVEDLVRIAEENDRLVVKKLVEDIAEILRRIVELLRRG |
| | 1 | |
| 645 | Peptide11_219_helix_ | ELLDRLAKVVKSIVEKAEKILERLNRVSKAIAEKLKTIVDELNEVSKEIVKRAEDILRK |
| | 2 | G |
| 646 | Peptide11_219_helix_ | ETVLRALRTLVKEYADLSKEVLERVERIVREYVKLSDEVVKSLAEIVEELIRIIEDLLR |
| | 3 | KG |
| 647 | Peptide11_219_helix_ | 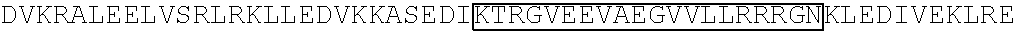 |
| | 4 | |
| 648 | Peptide_12_13helix_1 | 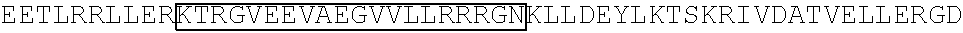 |
| 649 | Peptide_12_13helix_2 | ELIKRLAEELERSLRELEEEIKRLKRELEESLKKLKEIIDRLAEEAEKLLAVLKRG |
| 650 | Peptide_12_13helix_3 | EEALRALASLVRELIEVLRENDERLRDVLRRLIEALRKNNEILERVLRKLVRAAEERG |
| 651 | Peptide_12_13helix_4 | REALEEARRRLEELLRELNEITKDLEAKLEKLLRDLNELTKALEEELKRLLDELKKRTD |
| 652 | Peptide_12_188_helix_ | EETLRRLLERKVELAKEYLDVSKEVIDRTTKLLDEYLKTSKRIVDATVELLERGD |
| | 1 | |
| 653 | Peptide_12_188_helix_ | ELIKRLAEELERSLRELEEEIKRLKRELEESLKKLKEIIDRLAEEAEKLLAVLKRG |
| | 2 | |
| 654 | Peptide_12_188_helix_ | EEALRALASLVRELIEVLRENDERLRDVLRRLIEALRKNNEILERVLRKLVRAAEERG |
| | 3 | |
| 655 | Peptide_12_188_helix_ | 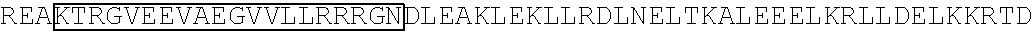 |
| | 4 | |
| 656 | Peptide_12_196_helix_ | EETLRRLLERKVELAKEYLDVSKEVIDRTTKLLDEYLKTSKRIVDATVELLERGD |
| | 1 | |
| 657 | Peptide_12_196_helix_ | ELIKRLAEELERSLRELEEEIKRLKRELEESLKKLKEIIDRLAEEAEKLLAVLKRG |
| | 2 | |
| 658 | Peptide_12_196_helix_ | EEALRALASLVRELIEVLRENDERLRDVLRRLIEALRKNNEILERVLRKLVRAAEERG |
| | 3 | |
| 659 | Peptide_12_196_helix_ | 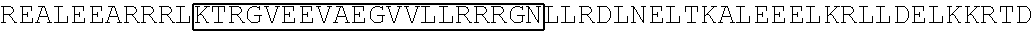 |
| | 4 | |
| 660 | Peptide_12_199_helix_ | EETLRRLLERKVELAKEYLDVSKEVIDRTTKLLDEYLKTSKRIVDATVELLERGD |
| | 1 | |
| 661 | Peptide_12_199_helix_ | ELIKRLAEELERSLRELEEEIKRLKRELEESLKKLKEIIDRLAEEAEKLLAVLKRG |
| | 2 | |
| 662 | Peptide_12_199_helix_ | EEALRALASLVRELIEVLRENDERLRDVLRRLIEALRKNNEILERVLRKLVRAAEERG |
| | 3 | |
| 663 | Peptide_12_199_helix_ | 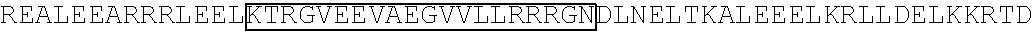 |
| | 4 | |
| 664 | Peptide_12_206_helix_ | EETLRRLLERKVELAKEYLDVSKEVIDRTTKLLDEYLKTSKRIVDATVELLERGD |
| | 1 | |
| 665 | Peptide_12_206_helix_ | ELIKRLAEELERSLRELEEEIKRLKRELEESLKKLKEIIDRLAEEAEKLLAVLKRG |
| | 2 | |
| 666 | Peptide_12_206_helix_ | EEALRALASLVRELIEVLRENDERLRDVLRRLIEALRKNNEILERVLRKLVRAAEERG |
| | 3 | |
| 667 | Peptide_12_206_helix_ | 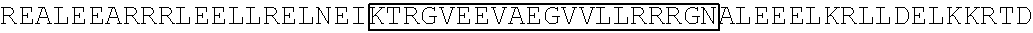 |
| | 4 | |
| 668 | Peptide_12_213_helix_ | EETLRRLLERKVELAKEYLDVSKEVIDRTTKLLDEYLKTSKRIVDATVELLERGD |
| | 1 | |
| 669 | Peptide_12_213_helix_ | ELIKRLAEELERSLRELEEEIKRLKRELEESLKKLKEIIDRLAEEAEKLLAVLKRG |
| | 2 | |
| 670 | Peptide_12_213_helix_ | EEALRALASLVRELIEVLRENDERLRDVLRRLIEALRKNNEILERVLRKLVRAAEERG |
| | 3 | |
| 671 | Peptide_12_213_helix_ | 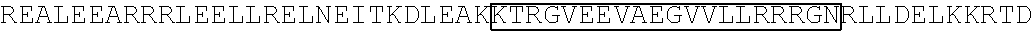 |
| | 4 | |
| 672 | Peptide_12_217_helix_ | EETLRRLLERKVELAKEYLDVSKEVIDRTTKLLDEYLKTSKRIVDATVELLERGD |
| | 1 | |
| 673 | Peptide_12_217_helix_ | ELIKRLAEELERSLRELEEEIKRLKRELEESLKKLKEIIDRLAEEAEKLLAVLKRG |
| | 2 | |
| 674 | Peptide_12_217_helix_ | EEALRALASLVRELIEVLRENDERLRDVLRRLIEALRKNNEILERVLRKLVRAAEERG |
| | 3 | |
| 675 | Peptide_12_217_helix_ | 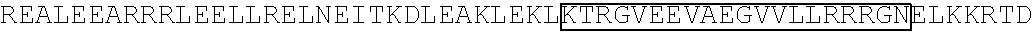 |
| | 4 | |
| 676 | Peptide_12_6_helix_1 | 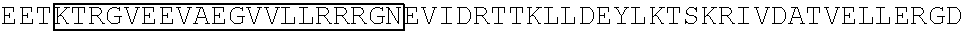 |
| 677 | Peptide_12_6_helix_2 | ELIKRLAEELERSLRELEEEIKRLKRELEESLKKLKEIIDRLAEEAEKLLAVLKRG |
| 678 | Peptide_12_6_helix_3 | EEALRALASLVRELIEVLRENDERLRDVLRRLIEALRKNNEILERVLRKLVRAAEERG |
| 679 | Peptide_12_6_helix_4 | REALEEARRRLEELLRELNEITKDLEAKLEKLLRDLNELTKALEEELKRLLDELKKRTD |

Other Degron peptides integrated in latches
(fragments of SEQ ID NO:1)

| | | |
|---|---|---|
| 680 | DegronPeptide33 | KTRGVEEVAEGVVLLRRRGN |
| 681 | DegronPeptide34 | KTRGVEEVAEGVVLLRRR |
| 682 | DegronPeptide35 | KTRGVEEVAEGVVLLRR |
| 683 | DegronPeptide36 | KTRGVEEVAEGVVLLR |
| 684 | DegronPeptide37 | KTRGVEEVAEGVVLL |
| 685 | DegronPeptide38 | KTRGVEEVAEGVVL |
| 686 | DegronPeptide39 | KTRGVEEVAEGVV |
| 687 | DegronPeptide40 | KTRGVEEVAEGV |
| 688 | DegronPeptide41 | KTRGVEEVAEG |
| 689 | DegronPeptide42 | KTRGVEEVAE |
| 690 | DegronPeptide43 | KTRGVEEVA |
| 691 | DegronPeptide44 | KTRGVEEV |
| 692 | DegronPeptide45 | KTRGVEE |
| 693 | DegronPeptide46 | KTRGVE |
| 694 | DegronPeptide47 | KTRGV |
| 695 | DegronPeptide48 | KTRG |
| 696 | DegronPeptide49 | TRGV |
| 697 | DegronPeptide50 | RGVE |
| 698 | DegronPeptide51 | GVEE |
| 699 | DegronPeptide52 | VEEV |
| 700 | DegronPeptide53 | EEVA |
| 701 | DegronPeptide54 | VAEG |
| 702 | DegronPeptide55 | AEGV |
| 703 | DegronPeptide56 | EGVV |
| 704 | DegronPeptide57 | GVVL |
| 705 | DegronPeptide58 | VVLL |
| 706 | DegronPeptide59 | VLLR |
| 707 | DegronPeptide60 | LLRR |
| 708 | DegronPeptide61 | LRRR |
| 709 | DegronPeptide62 | RRGN |
| 710 | DegronPeptide63 | TRGVE |
| 711 | DegronPeptide64 | RGVEE |
| 712 | DegronPeptide65 | GVEEV |
| 713 | DegronPeptide66 | VEEVA |
| 714 | DegronPeptide67 | EEVAE |
| 715 | DegronPeptide68 | EVAEG |
| 716 | DegronPeptide69 | VAEGV |
| 717 | DegronPeptide70 | AEGVV |
| 718 | DegronPeptide71 | EGVVL |
| 719 | DegronPeptide72 | GVVLL |
| 720 | DegronPeptide73 | VVLLR |
| 721 | DegronPeptide74 | VLLRR |
| 722 | DegronPeptide75 | LLRRR |
| 723 | DegronPeptide76 | TRGVEE |
| 724 | DegronPeptide77 | RGVEEV |
| 725 | DegronPeptide78 | GVEEVA |
| 726 | DegronPeptide79 | VEEVAE |
| 727 | DegronPeptide80 | EEVAEG |
| 728 | DegronPeptide81 | EVAEGV |
| 729 | DegronPeptide82 | VAEGVV |
| 730 | DegronPeptide83 | AEGVVL |
| 731 | DegronPeptide84 | EGVVLL |
| 732 | DegronPeptide85 | GVVLLR |
| 733 | DegronPeptide86 | VVLLRR |
| 734 | DegronPeptide87 | VLLRRR |
| 735 | DegronPeptide88 | TRGVEEV |
| 736 | DegronPeptide89 | RGVEEVA |
| 737 | DegronPeptide90 | GVEEVAE |
| 738 | DegronPeptide91 | VEEVAEG |
| 739 | DegronPeptide92 | EEVAEGV |
| 740 | DegronPeptide93 | EVAEGVV |
| 741 | DegronPeptide94 | VAEGVVL |
| 742 | DegronPeptide95 | AEGVVLL |
| 743 | DegronPeptide96 | EGVVLLR |
| 744 | DegronPeptide97 | GVVLLRR |
| 745 | DegronPeptide98 | VVLLRRR |
| 746 | DegronPeptide99 | TRGVEEVA |
| 747 | DegronPeptide100 | RGVEEVAE |
| 748 | DegronPeptide101 | GVEEVAEG |
| 749 | DegronPeptide102 | VEEVAEGV |
| 750 | DegronPeptide103 | EEVAEGVV |
| 751 | DegronPeptide104 | EVAEGVVL |
| 752 | DegronPeptide105 | VAEGVVLL |
| 753 | DegronPeptide106 | AEGVVLLR |
| 754 | DegronPeptide107 | EGVVLLRR |
| 755 | DegronPeptide108 | GVVLLRRR |
| 756 | DegronPeptide109 | TRGVEEVAE |
| 757 | DegronPeptide110 | RGVEEVAEG |
| 758 | DegronPeptide111 | GVEEVAEGV |
| 759 | DegronPeptide112 | VEEVAEGVV |
| 760 | DegronPeptide113 | EEVAEGVVL |
| 761 | DegronPeptide114 | EVAEGVVLL |
| 762 | DegronPeptide115 | VAEGVVLLR |
| 763 | DegronPeptide116 | AEGVVLLRR |
| 764 | DegronPeptide117 | EGVVLLRRR |
| 765 | DegronPeptide118 | TRGVEEVAEG |
| 766 | DegronPeptide119 | RGVEEVAEGV |
| 767 | DegronPeptide120 | GVEEVAEGVV |
| 768 | DegronPeptide121 | VEEVAEGVVL |
| 769 | DegronPeptide122 | EEVAEGVVLL |
| 770 | DegronPeptide123 | EVAEGVVLLR |
| 771 | DegronPeptide124 | VAEGVVLLRR |
| 772 | DegronPeptide125 | AEGVVLLRRR |
| 773 | DegronPeptide126 | TRGVEEVAEGV |
| 774 | DegronPeptide127 | RGVEEVAEGVV |
| 775 | DegronPeptide128 | GVEEVAEGVVL |
| 776 | DegronPeptide129 | VEEVAEGVVLL |
| 777 | DegronPeptide130 | EEVAEGVVLLR |
| 778 | DegronPeptide131 | EVAEGVVLLRR |
| 779 | DegronPeptide132 | VAEGVVLLRRR |
| 780 | DegronPeptide133 | TRGVEEVAEGVV |
| 781 | DegronPeptide134 | RGVEEVAEGVVL |
| 782 | DegronPeptide135 | GVEEVAEGVVLL |
| 783 | DegronPeptide136 | VEEVAEGVVLLR |
| 784 | DegronPeptide137 | EEVAEGVVLLRR |
| 785 | DegronPeptide138 | EVAEGVVLLRRR |
| 786 | DegronPeptide139 | TRGVEEVAEGVVL |
| 787 | DegronPeptide140 | RGVEEVAEGVVLL |
| 788 | DegronPeptide141 | GVEEVAEGVVLLR |
| 789 | DegronPeptide142 | VEEVAEGVVLLRR |
| 790 | DegronPeptide143 | EEVAEGVVLLRRR |
| 791 | DegronPeptide144 | TRGVEEVAEGVVLL |
| 792 | DegronPeptide145 | RGVEEVAEGVVLLR |
| 793 | DegronPeptide146 | GVEEVAEGVVLLRR |
| 794 | DegronPeptide147 | VEEVAEGVVLLRRR |
| 795 | DegronPeptide148 | TRGVEEVAEGVVLLR |
| 796 | DegronPeptide149 | RGVEEVAEGVVLLRR |
| 797 | DegronPeptide150 | GVEEVAEGVVLLRRR |
| 798 | DegronPeptide151 | TRGVEEVAEGVVLLRR |
| 799 | DegronPeptide152 | RGVEEVAEGVVLLRRR |
| 800 | DegronPeptide153 | TRGVEEVAEGVVLLRRR |

TABLE 4
Additional sequences.
Underlined amino acids indicate a position that is mutable

| | | |
|---|---|---|
| SEQ | | |
| ID | | |
| NO | Description | Sequence |

CAGES WITH DEGRON

| | | |
|---|---|---|
| 801 | Degron A* | KTRGVEEVAEGWLLRRRGNK |
| | (degron A, SEQ ID | |
| | NO: 1, extended by one | |
| | C-terminal K) | |
| 802 | DegronA*_Truncation1 | KEEVAEGWLLRRRGNK |
| 803 | DegronA*_Truncation2 | KTRGVEEVAEGWLLRK |
| 804 | DegronA*_Truncation3 | EGWLLRRRGNK |
| 805 | DegronA*_Truncation4 | KTRGVEEV |
| 806 | DegronA*_Truncation5 | KTRGVEEVAEGV |
| 807 | Peptide3_147 | KALEEAKRVLAENNEVIKDIKRALDELLKELEKSVRESEERLRRLLEEVKRVIEEVAG |
| | [[degronSwitch_degronA_ | SGSPLKARLEDIIRRLAEDIERVKRETDKLLEELARIIEEVEKEMRETNKELDKEIKD |
| | 2plus1_5419_circ_thread- | AESQNGSPEEIEKKVRELTKRHRELKTRGVEEVAEGVVLLRRRGNTLTERIKRVLEEV |
| | 147]] | RDLER |
| 808 | Peptide3_147_shortened_ | LAENNEVIKDIKRALDELLKELEKSVRESEERLRRLLEEVKRVIEEVATKASKSNDHD |
| | 9N [[degronSwitch_ | LKARLEDIIRRLAEDIERVKRETDKLLEELARIIEEVEKEMRETNKELDGSGSPELTK |
| | degronA_2plus1_5419_ | RHRELKTRGVEEVAEGVVLLRRRGNTLTERIKRVLEEVRDLER |
| | circ_thread-147 | |
| | shortened_9N]] | |
| 809 | Peptide3_147_ | KALEEAKRVLAENNEVIKDIKRALDELLKELEKSVRESEERLRRLLEEYKVGSPEDIE |
| | shortened_9C | RVKRETDKLLEELARIIEEVEKEMRETNKELDKEIKDAESQNQSLEEIEKKVRELTKR |
| | [[degronSwitch_degronA_ | HRELKTRGVEEVAEGVVLLRRRGNTLTERIKR |
| | 2plus1_5419_circ_thread- | |
| | 147_shortened_9C]] | |
| 810 | Peptide3_147_ | LAENNEVIKDIKRALDELLKELEKSVRESEERLRRLLEEYKVGSPEDIERVKRETDKL |
| | shortened_9C9N | LEELARIIEEVEKEMRETNKELDGSGSPELTKRHRELKTRGVEEVAEGVVLLRRRGNT |
| | [[degronSwitch_degronA_ | LTERIKR |
| | 2plus1_5419_circ_thread- | |
| | 147_shortened_9C9N]] | |
| 811 | Peptide3_21_ | ARELTKRHRELKTRGVEEVAEGVVLLRRRGNTLTERIKRVLEEVRDLERRGEPKEDLK |
| | shortened_9N | ARLEDIIRRLAEDIERVKRETDKLLEELARIIEEVEKEMRETNKELEEAAKGSPALAE |
| | [[degronSwtich_degronA_ | NNEVIKDIKRALDELLKELEKSVRESEERLRRLLEEVKRVIEEVAT |
| | 2plus1_5419_thread- | |
| | 21_shortened_9N]] | |
| 812 | Peptide4_154_medium_ | ATDLLKASNDKNRKVVEEIRDLLRKSKKLADELVERLRALVEDLRRRIDKSGDKETAE |
| | degron | DIVRRIIEELKRILKEIEDLARRINREIERLVEEVERDNRDVNRKAGDEEARRRAEES |
| | [[medium_degronSwitch_ | MRRVKEDKTRGVEEVAEGVVLLRRRGNRLIEEVRETLR |
| | degronA_2plus1_5432_ | |
| | thread-154]] | |
| 813 | Peptide4_154_short_ | NRKVVEEMRDLLRKSKKLADELVERLRALVEDLRRRIDKSGDKETAEDIVRRIIEELK |
| | degron [[short_ | RILKEIEDLARRINREIERAGADDSAKTRGVEEVAEGWLLRRRGNRLIEEVRETLR |
| | degronSwitch_degronA_ | |
| | 2plus1_5432_thread-154]] | |
| 814 | Peptide3_21_medium_ | SELEEIEKKVRELTKRHRELKTRGVEEVAEGVVLLRRRGNTLTEKIKNGKDERKLAED |
| | degron [[medium_ | IERVKRETDKLLEELARIIEEVEKEMRETNKELDKEIKDGGLDDKALEEAKRVLAENN |
| | degronSwitch_degronA_ | EVIKDIKRALDELLKELEKSVRESEERLRRLLEE |
| | 2plus1_5419_thread-21]] | |
| 815 | Peptide3_21_short_ | TKRIRELKTRGVEEVAEGVVLLRRRGNTLTEKIKNGKDERKLAEDIERVKRETDKLLE |
| | degron [[short_ | ELARIIEELKKGNVDDDVSRALDELLKELEKSVRESEERLRRLLEE |
| | degronSwitch_degronA_ | |
| | 2plus1_5419_thread-21]] | |
| 816 | Peptide10_38_medium | ADVLRENDETIRKVVEDLVRIAEENDRLKTRGVEEVAEGVVLLRRRGNRGGVPEELLD |
| | degron | RLAKVVKSIVEKAEKILERLNRVSKAIAEKLKTIVDELNEVSKSGASQKEFADLSKEV |
| | [[mediumdegronSwitch_ | LERVERIVREYVKLSDEVVKSLAEIVEELIRIIEDLLRKGNLDEDVKRALEELVSRLR |
| | degronA_3plus1_646_ | KLLEDVKKASEDIVREVERIVRELAKRSDEILKK |
| | thread-38]] | |
| 817 | Peptide10_38_short_ | TARKVVEDLVRIAEENDRLKTRGVEEVAEGVVLLRRRGNRGGVPEELLDRLAKVVKSI |
| | degron | VEKAEKILERLNRVSKAIAEKLKSTGNKEEVERIVREYVKLSDEVVKSLAEIVEELIR |
| | [[short_degronSwitch_ | IIEDLLRKGNLDEDVKRALEELVSRLRKLLEDVKKASEDIVREVERIVREL |
| | degronA_3plus1_646_ | |
| | thread-38]] | |
| 818 | Peptide2_35_medium_ | LREEKEEARTHVEKSIRDLRKILEDKTRGVEEVAEGVVLLRRRGNKKPGDEEVRKTVE |
| | degron [[medium_ | EISRRVAENVKRLEDLYRRMEEEVKKNLDRLRKEGVDEDILRRAAEAHKKLIDDARES |
| | degronSwitch_degronA_ | LERAKREHEKLIDRLKKILEELER |
| | 2plus1_5404_thread-35]] | |
| 819 | Peptide2_35_short_ | THVEKSIRDLRKILEDKTRGVEEVAEGVVLLRRRGNKKPGDEEVRKTVEEISRRVAEN |
| | degron [[short_ | VKRLEDLYRRMEEEAGSESELADDARESLERAKREHEKLIDRLKKILEELER |
| | degronSwitch_degronA_ | |
| | 2plus1_5404_thread-35]] | |
| 820 | Peptide7_218_medium_ | DEDRIIEEIARLLEELLRELLELIKKLIETNRRLNEEHERAVRELARLGTDKALDDLE |
| | degron [[medium_ | RLHREINKRLEDLVRELEKLVREILKELKDALEELRRASARAGGEEVLRRLEEIVKKL |
| | degronSwitch_degronA_ | LDLVRRILERLKEIHKDNVRLLRELNERLTRILGNADEEIRRMVKRLHEDLERKKTRG |
| | 3plus1_534_thread-218]] | VEEVAEGVVLLRRRGNRLEEKLERKRR |
| 821 | Peptide7_218_short_ | DEDRIIEEIARLLEELLRELLELIKKLIETNRRLAEEANGDSKVNKRLEDLVRELEKL |
| | degron [[short_ | VREILKELKDALEELRRASARAGGEEVLRRLEEIVKKLLDLVRRILERLKEIHKDNSV |
| | degronSwitch_degronA_ | FGDKRKKTRGVEEVAEGVVLLRRRGNRLEEKLERKRR |
| | 3plus1_534_thread-218]] | |
| 822 | C terminal degron | SGGGGSGGGGSLPMSCAQESGMDRHPAACASARINV |
| | sequence of mouse | |
| | ornithine | |
| | decarboxylase | |

CAGES

| | | |
|---|---|---|
| 823 | Peptide3_147/Cage13 | KALEEAKRVLAENNEVIKDIKRALDELLKELEKSVRESEERLRRLLEEVKRVIEEVAG |
| | | SGSPLKARLEDIIRRLAEDIERVKRETDKLLEELARIIEEVEKEMRETNKELDKEIKD |
| | | AESQNGSPEEIEKKVRELTKRHRELVERVRKTVKELIETNRRLLETLTERIKRVLEEV |
| | | RDLER |

| | | |
|---|---|---|
| 824 | Peptide3_147_ | LAENNEVIKDIKRALDELLKELEKSVRESEERLRRLLEEVKRVIEEVATKASKSNDHD |
| | shortened_9N/Cage14 | LKARLEDIIRRLAEDIERVKRETDKLLEELARIIEEVEKEMRETNKELDGSGSPELTK |
| | | RHRELVERVRKTVKELIETNRRLLETLTERIKRVLEEVRDLER |
| 825 | Peptide3_147_ | KALEEAKRVLAENNEVIKDIKRALDELLKELEKSVRESEERLRRLLEEYKVGSPEDIE |
| | shortened_9C/Cage15 | RVKRETDKLLEELARIIEEVEKEMRETNKELDKEIKDAESQNQSLEEIEKKVRELTKR |
| | | HRELVERVRKTVKELIETNRRLLETLTERIKR |
| 826 | Peptide3_147_ | LAENNEVIKDIKRALDELLKELEKSVRESEERLRRLLEEYKVGSPEDIERVKRETDKL |
| | shortened_9C9N/ | LEELARIIEEVEKEMRETNKELDGSGSPELTKRHRELVERVRKTVKELIETNRRLLET |
| | Cage16 | LTERIKR |
| 827 | Peptide3_21_ | ARELTKRHRELVERVRKTVKELIETNRRLLETLTERIKRVLEEVRDLERRGEPKEDLK |
| | shortened_9N/ | ARLEDIIRRLAEDIERVKRETDKLLEELARIIEEVEKEMRETNKELEEAAKGSPALAE |
| | Cage17 | NNEVIKDIKRALDELLKELEKSVRESEERLRRLLEEVKRVIEEVAT |
| 828 | Peptide3_21_medium_ | SELEEIEKKVRELTKRHRELVERVRKTVKELIETNRRLLETLTEKIKNGKDERKLAED |
| | degron/Cage18 | IERVKRETDKLLEELARIIEEVEKEMRETNKELDKEIKDGGLDDKALEEAKRVLAENN |
| | | EVIKDIKRALDELLKELEKSVRESEERLRRLLEE |
| 829 | Peptide3_21_short_ | TKRIRELVERVRKTVKELIETNRRLLETLTEKIKNGKDERKLAEDIERVKRETDKLLE |
| | degron/Cage19 | ELARIIEELKKGNVDDDVSRALDELLKELEKSVRESEERLRRLLEE |
| 830 | Peptide4_154_medium_ | ATDLLKASNDKNRKVVEEIRDLLRKSKKLADELVERLRALVEDLRRRIDKSGDKETAE |
| | degron/Cage20 | DIVRRIIEELKRILKEIEDLARRINREIERLVEEVERDNRDVNRKAGDEEARRRAEES |
| | | MRRVKEDADRVSEANKEVLDRVREEVKRLIEEVRETLR |
| 831 | Peptide4_154_short_ | NRKVVEEMRDLLRKSKKLADELVERLRALVEDLRRRIDKSGDKETAEDIVRRIIEELK |
| | degron/Cage21 | RILKEIEDLARRINREIERAGADDSAADRVSEANKEVLDRVREEVKRLIEEVRETLR |
| 832 | Peptide10_38_medium_ | ADVLRENDETIRKVVEDLVRIAEENDRLWKKLVEDIAEILRRIVELLRRGGVPEELLD |
| | degron/Cage22 | RLAKVVKSIVEKAEKILERLNRVSKAIAEKLKTIVDELNEVSKSGASQKEFADLSKEV |
| | | LERVERIVREYVKLSDEVVKSLAEIVEELIRIIEDLLRKGNLDEDVKRALEELVSRLR |
| | | KLLEDVKKASEDIVREVERIVRELAKRSDEILKK |
| 833 | Peptide10_38_short_ | TARKVVEDLVRIAEENDRLWKKLVEDIAEILRRIVELLRRGGVPEELLDRLAKVVKSI |
| | degron/Cage23 | VEKAEKILERLNRVSKAIAEKLKSTGNKEEVERIVREYVKLSDEVVKSLAEIVEELIR |
| | | IIEDLLRKGNLDEDVKRALEELVSRLRKLLEDVKKASEDIVREVERIVREL |
| 834 | Peptide2_35_medium_ | LREEKEEARTHVEKSIRDLRKILEDSTRHAKRSIEELERLLEEVRKKPGDEEVRKTVE |
| | degron/Cage24 | EISRRVAENVKRLEDLYRRMEEEVKKNLDRLRKEGVDEDILRRAAEAHKKLIDDARES |
| | | LERAKREHEKLIDRLKKILEELER |
| 835 | Peptide2_35_short_ | THVEKSIRDLRKILEDSTRHAKRSIEELERLLEEVRKKPGDEEVRKTVEEISRRVAEN |
| | degron/Cage25 | VKRLEDLYRRMEEEAGSESELADDARESLERAKREHEKLIDRLKKILEELER |
| 836 | Peptide7_218_medium_ | DEDRIIEEIARLLEELLRELLELIKKLIETNRRLNEEHERAVRELARLGTDKALDDLE |
| | degron/Cage26 | RLHREINKRLEDLVRELEKLVREILKELKDALEELRRASARAGGEEVLRRLEEIVKKL |
| | | LDLVRRILERLKEIHKDNVRLLRELNERLTRILGNADEEIRRMVKRLHEDLERKLKES |
| | | EDELREIEARLEEKIRRLEEKLERKRR |
| 837 | Peptide7_218_short_ | DEDRIIEEIARLLEELLRELLELIKKLIETNRRLAEEANGDSKVNKRLEDLVRELEKL |
| | degron/Cage27 | VREILKELKDALEELRRASARAGGEEVLRRLEEIVKKLLDLVRRILERLKEIHKDNSV |
| | | FGDKRKLKESEDELREIEARLEEKIRRLEEKLERKRR |

STRUCTURAL REGIONS (SR) AND LATCHES

| | | |
|---|---|---|
| 838 | Peptide3_147/ | KALEEAKRVLAENNEVIKDIKRALDELLKELEKSVRESEERLRRLLEEVKRVIEEVAG |
| | Cage13_SR | SGSPLKARLEDIIRRLAEDIERVKRETDKLLEELARIIEEVEKEMRETNKELDKEIKD |
| | | AESQNGSPEEIEKKVR |
| 839 | Peptide3_147/ | ELTKRHRELVERVRKTVKELIETNRRLLETLTERIKRVLEEVRDLER |
| | Cage13_Latch | |
| 840 | Peptide3_147_ | LAENNEVIKDIKRALDELLKELEKSVRESEERLRRLLEEVKRVIEEVATKASKSNDHD |
| | shortened_9N/ | LKARLEDIIRRLAEDIERVKRETDKLLEELARIIEEVEKEMRETNKELDGSGSP |
| | Cage14_SR | |
| 841 | Peptide3_147_ | ELTKRHRELVERVRKTVKELIETNRRLLETLTERIKRVLEEVRDLER |
| | shortened_9N/ | |
| | Cage14_Latch | |
| 842 | Peptide3_147_ | KALEEAKRVLAENNEVIKDIKRALDELLKELEKSVRESEERLRRLLEEYKVGSPEDIE |
| | shortened_9C/ | RVKRETDKLLEELARIIEEVEKEMRETNKELDKEIKDAESQNQSLEEIEKKVR |
| | Cage15_SR | |
| 843 | Peptide3_147_ | ELTKRHRELVERVRKTVKELIETNRRLLETLTERIKR |
| | shortened_9C/ | |
| | Cage15_Latch | |
| 844 | Peptide3_147_ | LAENNEVIKDIKRALDELLKELEKSVRESEERLRRLLEEYKVGSPEDIERVKRETDKL |
| | shortened_9C9N/ | LEELARIIEEVEKEMRETNKELDGSGSP |
| | Cage16_SR | |
| 845 | Peptide3_147_ | ELTKRHRELVERVRKTVKELIETNRRLLETLTERIKR |
| | shortened_9C9N/ | |
| | Cage16_Latch | |
| 846 | Peptide3_21_ | RIKRVLEEVRDLERRGEPKEDLKARLEDIIRRLAEDIERVKRETDKLLEELARIIEEV |
| | shortened_9N/ | EKEMRETNKELEEAAKGSPALAENNEVIKDIKRALDELLKELEKSVRESEERLRRLLE |
| | Cage17_SR | EVKRVIEEVAT |
| 847 | Peptide3_21_ | ARELTKRHRELVERVRKTVKELIETNRRLLETLTE |
| | shortened_9N/ | |
| | Cage17_Latch | |
| 848 | Peptide3_21_medium_ | KIKNGKDERKLAEDIERVKRETDKLLEELARIIEEVEKEMRETNKELDKEIKDGGLDD |
| | degron/Cage18_SR | KALEEAKRVLAENNEVIKDIKRALDELLKELEKSVRESEERLRRLLEE |
| 849 | Peptide3_21_medium_ | SELEEIEKKVRELTKRHRELVERVRKTVKELIETNRRLLETLTE |
| | degron/Cage18_Latch | |
| 850 | Peptide3_21_short_ | KIKNGKDERKLAEDIERVKRETDKLLEELARIIEELKKGNVDDDVSRALDELLKELEK |
| | degron/Cage19_SR | SVRESEERLRRLLEE |
| 851 | Peptide3_21_short_ | TKRIRELVERVRKTVKELIETNRRLLETLTE |
| | degron/Cage19_Latch | |
| 852 | Peptide4_154_medium_ | ATDLLKASNDKNRKVVEEIRDLLRKSKKLADELVERLRALVEDLRRRIDKSGDKETAE |
| | degron/Cage20_SR | DIVRRIIEELKRILKEIEDLARRINREIERLVEEVERDNRDVNRKAG |
| 853 | Peptide4_154_medium_ | DEEARRRAEESMRRVKED ADRVSEANKEVLDRVREEVK RLIEEVRETLR |
| | degron/Cage20_Latch | |
| 854 | Peptide4_154_short_ | NRKVVEEMRDLLRKSKKLADELVERLRALVEDLRRRIDKSGDKETAEDIVRRIIEELK |
| | degron/Cage21_SR | RILKEIEDLARRINREIERA |
| 855 | Peptide4_154_short_ | GADDSA ADRVSEANKEVLDRVREEVK RLIEEVRETLR |
| | degron/Cage21_Latch | |
| 856 | Peptide10_38_medium_ | PEELLDRLAKVVKSIVEKAEKILERLNRVSKAIAEKLKTIVDELNEVSKSGASQKEFA |
| | degron/Cage22_SR | DLSKEVLERVERIVREYVKLSDEVVKSLAEIVEELIRIIEDLLRKGNLDEDVKRALEE |
| | | LVSRLRKLLEDVKKASEDIVREVERIVRELAKRSDEILKK |
| 857 | Peptide10_38_medium_ | ADVLRENDETIRKVVEDLVRIAEENDRLWKKLVEDIAEILRRIVELLRRGGV |
| | degron/Cage22_Latch | |
| 858 | Peptide10_38_short_ | PEELLDRLAKVVKSIVEKAEKILERLNRVSKAIAEKLKSTGNKEEVERIVREYVKLSD |
| | degron/Cage23_SR | EVVKSLAEIVEELIRIIEDLLRKGNLDEDVKRALEELVSRLRKLLEDVKKASEDIVRE |
| | | VERIVREL |
| 859 | Peptide10_38_short_ | TARKVVEDLVRIAEENDRLWKKLVEDIAEILRRIVELLRRGGV |
| | degron/Cage23_Latch | |
| 860 | Peptide2_35_medium_ | PGDEEVRKTVEEISRRVAENVKRLEDLYRRMEEEVKKNLDRLRKEGVDEDILRRAAEA |
| | degron/Cage24_SR | HKKLIDDARESLERAKREHEKLIDRLKKILEELER |
| 861 | Peptide2_35_medium_ | LREEKEEARTHVEKSIRDLRKILEDSTRHAKRSIEELERLLEEVRKK |
| | degron/Cage24_Latch | |
| 862 | Peptide2_35_short_ | PGDEEVRKTVEEISRRVAENVKRLEDLYRRMEEEAGSESELADDARESLERAKREHEK |
| | degron/Cage25_SR | LIDRLKKILEELER |
| 863 | Peptide2_35_short_ | THVEKSIRDLRKILEDSTRHAKRSIEELERLLEEVRKK |
| | degron/Cage25_Latch | |
| 864 | Peptide7_218_medium_ | DEDRIIEEIARLLEELLRELLELIKKLIETNRRLNEEHERAVRELARLGTDKALDDLE |
| | degron/Cage26_SR | RLHREINKRLEDLVRELEKLVREILKELKDALEELRRASARAGGEEVLRRLEEIVKKL |
| | | LDLVRRILERLKEIHKDNVRLLRELNERLTRILGNA |
| 865 | Peptide7_218_medium_ | DEEIRRMVKRLHEDLERKLKESEDELREIEARLEEKIRRLEEKLERKRR |
| | degron/Cage26_Latch | |
| 866 | Peptide7_218_short_ | DEDRIIEEIARLLEELLRELLELIKKLIETNRRLAEEANGDSKVNKRLEDLVRELEKL |
| | degron/Cage27_SR | VREILKELKDALEELRRASARAGGEEVLRRLEEIVKKLLDLVRRILERLKEIHKDNS |
| 867 | Peptide7_218_short_ | VFGDKRKLKESEDELREIEARLEEKIRRLEEKLERKRR |
| | degron/Cage27_Latch | |

SR BASE HELICES

| | | |
|---|---|---|
| 868 | Cage 13 Base helix 1 | KALEEAKRVLAENNEVIKDIKRALDELLKELEKSVRESEERLRRLL |
| 869 | Cage 13 Base helix 2 | EEVKRVIEEVAGSGSPLKARLEDIIRRLAEDIERVKRETDKLL |
| 870 | Cage 13 Base helix 3 | EELARIIEEVEKEMRETNKELDKEIKDAESQNGSPEEIEKKVR |
| 871 | Cage 14 Base helix 1 | LAENNEVIKDIKRALDELLKELEKSVRESEERLRRLLEEVKRVIEEVATKASKSND |
| 872 | Cage 14 Base helix 2 | HDLKARLEDIIRRLAEDIERVKRETDKLLEELARIIEEVEKEMRETNKELDGSGSP |
| 873 | Cage 15 Base helix 1 | KALEEAKRVLAENNEVIKDIKRALDELLKELEKSVRESEERLRRLLEEYKVGSPED |
| 874 | Cage 15 Base helix 2 | IERVKRETDKLLEELARIIEEVEKEMRETNKELDKEIKDAESQNQSLEEIEKKVR |
| 875 | Cage 16 Base helix 1 | LAENNEVIKDIKRALDELLKELEKSVRESEERLRRLLEEYKVG |
| 876 | Cage 16 Base helix 2 | SPEDIERVKRETDKLLEELARIIEEVEKEMRETNKELDGSGSP |
| 877 | Cage 17 Base helix 1 | RIKRVLEEVRDLERRGEPKEDLKARLEDIIRRLAEDIERVKRETDKLLEELARIIEEV |
| | | EKEMR |
| 878 | Cage 17 Base helix 2 | ETNKELEEAAKGSPALAENNEVIKDIKRALDELLKELEKSVRESEERLRRLLEEVKRV |
| | | IEEVAT |
| 879 | Cage 18 Base helix 1 | KIKNGKDERKLAEDIERVKRETDKLLEELARIIEEVEKEMRETNKELDKEIKD |
| 880 | Cage 18 Base helix 2 | GGLDDKALEEAKRVLAENNEVIKDIKRALDELLKELEKSVRESEERLRRLLEE |
| 881 | Cage 19 Base helix 1 | KIKNGKDERKLAEDIERVKRETDKLLEELARIIEEL |
| 882 | Cage 19 Base helix 2 | KKGNVDDDVSRALDELLKELEKSVRESEERLRRLLEE |
| 883 | Cage 20 Base helix 1 | ATDLLKASNDKNRKVVEEIRDLLRKSKKLADELVERLRALVEDLRRRIDKSG |
| 884 | Cage 20 Base helix 2 | DKETAEDIVRRIIEELKRILKEIEDLARRINREIERLVEEVERDNRDVNRKAG |
| 885 | Cage 21 Base helix 1 | NRKVVEEMRDLLRKSKKLADELVERLRALVEDLRRRIDK |
| 886 | Cage 21 Base helix 2 | SGDKETAEDIVRRIIEELKRILKEIEDLARRINREIERA |
| 887 | Cage 22 Base helix 1 | PEELLDRLAKVVKSIVEKAEKILERLNRVSKAIAEKLKTIVDELNEVSKSGA |
| 888 | Cage 22 Base helix 2 | SQKEFADLSKEVLERVERIVREYVKLSDEVVKSLAEIVEELIRIIEDLLRKG |
| 889 | Cage 22 Base helix 3 | NLDEDVKRALEELVSRLRKLLEDVKKASEDIVREVERIVRELAKRSDEILKK |
| 890 | Cage 23 Base helix 1 | PEELLDRLAKVVKSIVEKAEKILERLNRVSKAIAEKLKSTGN |
| 891 | Cage 23 Base helix 2 | KEEVERIVREYVKLSDEVVKSLAEIVEELIRIIEDLLRKGN |
| 892 | Cage 23 Base helix 3 | LDEDVKRALEELVSRLRKLLEDVKKASEDIVREVERIVREL |
| 893 | Cage 24 Base helix 1 | PGDEEVRKTVEEISRRVAENVKRLEDLYRRMEEEVKKNLDRLRKEGV |
| 894 | Cage 24 Base helix 2 | DEDILRRAAEAHKKLIDDARESLERAKREHEKLIDRLKKILEELER |
| 895 | Cage 25 Base helix 1 | PGDEEVRKTVEEISRRVAENVKRLEDLYRRMEEEAG |
| 896 | Cage 25 Base helix 2 | SESELADDARESLERAKREHEKLIDRLKKILEELER |
| 897 | Cage 26 Base helix 1 | DEDRIIEEIARLLEELLRELLELIKKLIETNRRLNEEHERAVRELARLGTD |
| 898 | Cage 26 Base helix 2 | KALDDLERLHREINKRLEDLVRELEKLVREILKELKDALEELRRASARAG |
| 899 | Cage 26 Base helix 3 | GEEVLRRLEEIVKKLLDLVRRILERLKEIHKDNVRLLRELNERLTRILGNA |
| 900 | Cage 27 Base helix 1 | DEDRIIEEIARLLEELLRELLELIKKLIETNRRLAEEA |
| 901 | Cage 27 Base helix 2 | NGDSKVNKRLEDLVRELEKLVREILKELKDALEELRRA |
| 902 | Cage 27 Base helix 3 | SARAGGEEVLRRLEEIVKKLLDLVRRILERLKEIHKDNS |

LATCH BASE HELICES

| | | |
|---|---|---|
| 903 | Cage 13 Latch helix | ELTKRHRELVERVRKTVKELIETNRRLLETLTERIKRVLEEVRDLER |
| 904 | Cage 14 Latch helix | ELTKRHRELVERVRKTVKELIETNRRLLETLTERIKRVLEEVRDLER |
| 905 | Cage 15 Latch helix | ELTKRHRELVERVRKTVKELIETNRRLLETLTERIKR |
| 906 | Cage 16 Latch helix | ELTKRHRELVERVRKTVKELIETNRRLLETLTERIKR |
| 907 | Cage 17 Latch helix | ARELTKRHRELVERVRKTVKELIETNRRLLETLTE |
| 908 | Cage 18 Latch helix | SELEEIEKKVRELTKRHRELVERVRKTVKELIETNRRLLETLTE |
| 909 | Cage 19 Latch helix | TKRIRELVERVRKTVKELIETNRRLLETLTE |
| 910 | Cage 20 Latch helix | DEEARRRAEESMRRVKEDADRVSEANKEVLDRVREEVKRLIEEVRETLR |
| 911 | Cage 21 Latch helix | GADDSAADRVSEANKEVLDRVREEVKRLIEEVRETLR |
| 912 | Cage 22 Latch helix | ADVLRENDETIRKVVEDLVRIAEENDRLWKKLVEDIAEILRRIVELLRRGGV |
| 913 | Cage 23 Latch helix | TARKVVEDLVRIAEENDRLWKKLVEDIAEILRRIVELLRRGGV |
| 914 | Cage 24 Latch helix | LREEKEEARTHVEKSIRDLRKILEDSTRHAKRSIEELERLLEEVRKK |
| 915 | Cage 25 Latch helix | THVEKSIRDLRKILEDSTRHAKRSIEELERLLEEVRKK |
| 916 | Cage 26 Latch helix | DEEIRRMVKRLHEDLERKLKESEDELREIEARLEEKIRRLEEKLERKRR |
| 917 | Cage 27 Latch helix | VFGDKRKLKESEDELREIEARLEEKIRRLEEKLERKRR |

DEGRON A TRUNCATIONS

| | | |
|---|---|---|
| 918 | DegronA*_Truncation6 | TRGVEEVAEGWLLRRRGNK |
| 919 | DegronA*_Truncation7 | RGVEEVAEGVVLLRRRGNK |
| 920 | DegronA*_Truncation8 | GVEEVAEGVVLLRRRGNK |
| 921 | DegronA*_Truncation9 | VEEVAEGVVLLRRRGNK |
| 922 | DegronA*_Truncation10 | EEVAEGVVLLRRRGNK |
| 923 | DegronA*_Truncation11 | EVAEGVVLLRRRGNK |
| 924 | DegronA*_Truncation12 | VAEGVVLLRRRGNK |
| 925 | DegronA*_Truncation13 | AEGVVLLRRRGNK |
| 926 | DegronA*_Truncation14 | GVVLLRRRGNK |
| 927 | DegronA*_Truncation15 | VVLLRRRGNK |
| 928 | DegronA*_Truncation16 | VLLRRRGNK |
| 929 | DegronA*_Truncation17 | LLRRRGNK |
| 930 | DegronA*_Truncation18 | LRRRGNK |
| 931 | DegronA*_Truncation19 | RRRGNK |
| 932 | DegronA*_Truncation20 | KTEVAEGVVLLRRRGNK |
| 933 | DegronA*_Truncation21 | KTRGAEGVVLLRRRGNK |
| 934 | DegronA*_Truncation22 | KTRGVEGVVLLRRRGNK |
| 935 | DegronA*_Truncation23 | KTRGVEEVVLLRRRGNK |
| 936 | DegronA*_Truncation24 | KTRGVEEVAELRRRGNK |
| 937 | DegronA*_Truncation25 | KTVLLRRRGNK |
| 938 | DegronA*_Truncation26 | KTRGLRRRGNK |

DEGRON A MUTATIONS

| | | |
|---|---|---|
| 939 | DegronA*_Mutation1 | CTRGVEEVAEGVVLLRRRGNK |
| 940 | DegronA*_Mutation2 | FTRGVEEVAEGVVLLRRRGNK |
| 941 | DegronA*_Mutation3 | KIRGVEEVAEGVVLLRRRGNK |
| 942 | DegronA*_Mutation4 | KLRGVEEVAEGVVLLRRRGNK |
| 943 | DegronA*_Mutation5 | KNRGVEEVAEGVVLLRRRGNK |
| 944 | DegronA*_Mutation6 | KRRGVEEVAEGVVLLRRRGNK |
| 945 | DegronA*_Mutation7 | KTDGVEEVAEGVVLLRRRGNK |
| 946 | DegronA*_Mutation8 | KTFGVEEVAEGVVLLRRRGNK |
| 947 | DegronA*_Mutation9 | KTHGVEEVAEGVVLLRRRGNK |
| 948 | DegronA*_Mutation10 | KTPGVEEVAEGVVLLRRRGNK |
| 949 | DegronA*_Mutation11 | KTRGKEEVAEGVVLLRRRGNK |
| 950 | DegronA*_Mutation12 | KTRGMEEVAEGVVLLRRRGNK |
| 951 | DegronA*_Mutation13 | KTRGTEEVAEGVVLLRRRGNK |
| 952 | DegronA*_Mutation14 | KTRGVAEVAEGVVLLRRRGNK |
| 953 | DegronA*_Mutation15 | KTRGVEAVAEGVVLLRRRGNK |
| 954 | DegronA*_Mutation16 | KTRGVEDVAEGVVLLRRRGNK |
| 955 | DegronA*_Mutation17 | KTRGVEECAEGVVLLRRRGNK |
| 956 | DegronA*_Mutation18 | KTRGVEENAEGVVLLRRRGNK |
| 957 | DegronA*_Mutation19 | KTRGVEETAEGVVLLRRRGNK |
| 958 | DegronA*_Mutation20 | KTRGVEEVACGVVLLRRRGNK |
| 959 | DegronA*_Mutation21 | KTRGVEEVAEEVVLLRRRGNK |
| 960 | DegronA*_Mutation22 | KTRGVEEVAEGCVLLRRRGNK |
| 961 | DegronA*_Mutation23 | KTRGVEEVAEGVGLLRRRGNK |
| 962 | DegronA*_Mutation24 | KTRGVEEVAEGVVLLRHRGNK |
| 963 | DegronA*_Mutation25 | KTRGVEEVAEGVVLLRQRGNK |
| 964 | DegronA*_Mutation26 | KTRGVEEVAEGVVLLRRRGFK |
| 965 | DegronA*_Mutation27 | KTRGVEEVAEGVVLLRRRGNH |
| 966 | DegronA*_Mutation28 | KTRGVEEVAEGVVLTRRRGNK |
| 967 | DegronA*_Mutation29 | KTRGVEEVAEGWVLLRRRGNK |
| 968 | DegronA*_Mutation30 | KTRGVEEVAEPVVLLRRRGNK |
| 969 | DegronA*_Mutation31 | KTRGVEEVAHGVVLLRRRGNK |
| 970 | DegronA*_Mutation32 | KTRGVEEVAPGVVLLRRRGNK |
| 971 | DegronA*_Mutation33 | KTRGVEEVAQGVVLLRRRGNK |
| 972 | DegronA*_Mutation34 | KTRGVEEVATGVVLLRRRGNK |
| 973 | DegronA*_Mutation35 | KTRGVEEVHEGVVLLRRRGNK |
| 974 | DegronA*_Mutation36 | KTRGVEEVPEGVVLLRRRGNK |
| 975 | DegronA*_Mutation37 | KTRGVEEVREGVVLLRRRGNK |
| 976 | DegronA*_Mutation38 | KTRGVEHVAEGVVLLRRRGNK |
| 977 | DegronA*_Mutation39 | KTRGVEPVAEGVVLLRRRGNK |
| 978 | DegronA*_Mutation40 | KTRGVERVAEGVVLLRRRGNK |
| 979 | DegronA*_Mutation41 | KTRGVEVVAEGVVLLRRRGNK |
| 980 | DegronA*_Mutation42 | KTRGVQEVAEGVVLLRRRGNK |
| 981 | DegronA*_Mutation43 | KTRSVEEVAEGVVLLRRRGNK |
| 982 | DegronA*_Mutation44 | QTRGVEEVAEGVVLLRRRGNK |

TABLE 5
Degron A consensus sequences

| | | |
|---|---|---|
| 983 | DegronA*_Consensus1 | L-X1-R-X2-R-G-X3-X4, where X1 |
| | | is L or T; X2 is Q, R, or H; X3 is F |
| | | or N; and X4 is K or H |
| 984 | DegronA*_Consensus2 | X1-R-X2-R-X3-X4-X5, where X1 |
| | | is L, T, K, or C; X2 is Q, K, R, H, |
| | | or Y; X3 is N, G, Y, A, or C; X4 is |
| | | L, N, G, Q, P, K, or F; and X5 is K, |
| | | H, Q, S, W, or R |

### EXAMPLES

### 1. Methods

Cell Culture and Lentiviral Transduction

Jurkat, SupT1, and Jeko1 cell lines were obtained from American Type Culture Collection (Manassas Va.). SupT1 and Jeko1 cells were maintained in RPMI 1640 media with GLUTAMAX™ (Gibco) containing 10% fetal calf serum (Gibco). For lentiviral transduction, SupT1 and Jurkat cells were fed with fresh media 4-16 hours before transduction, then incubated with lentivirus in complete media supplemented with LENTIBOOST™ at the manufacturers recommended concentration (Sirion Biotech). 18 hours after transfection, lentivirus and LENTIBOOST™ were diluted by addition of 1 volume fresh media.

Pre-selected, cryopreserved primary human CD4 and CD8 T cells from normal donors were obtained from Bloodworks (Seattle, Wash.). Human T cells were cultured in OPTMIZER™ medium (Thermo Fisher) supplemented with Immune Cell Serum Replacement (Thermo Fisher), 2 mM L-glutamine (Gibco), 2 mM GLUTAMAX™ (Gibco), 200 IU/ml IL-2 (R&D systems), 120 IU/ml IL-7 (R&D systems), and 20 IU/ml IL-15 (R&D systems).

For lentiviral transduction, T cells were stimulated with a 1:100 dilution of T cell TRANSACT™ (Miltenyi) for 30 hours. Virus was then added to T cells for 18-24 hours. Stimulation and viral infection were then terminated by addition of 7 volumes of fresh media without TRANSACT™, and cells were cultured for 3-7 additional days before analysis.

Flow Cytometry

Flow cytometry was performed on a Ze5 cytometer (Biorad). To determine expression of surface markers, between 1×10⁵ and 2×10⁵ total cells were transferred to a V bottom 96-well culture dish (Corning). Cells were washed twice with flow cytometry staining buffer (eBioscience), then stained with the relevant reagents in a total volume of 50 ul flow cytometry staining buffer for 30 minutes on ice. After staining, cells were washed twice with flow cytometry staining buffer, fixed in FLUOROFIX™ Buffer (Biolegend) and kept at 4° C. in the dark until analysis.

TABLE 6
Antibodies used

| Target | Fluorochrome | Clone | Supplier |
|---|---|---|---|
| CD19 | BV421 | Hib19 | Biolegend |
| EGFR | BV421 | AY13 | Biolegend |
| CD3 | BUV805 | SK7 | ThermoFisher |
| CD69 | PE | FN50 | Biolegend |
| PD1 | PE-cy7 | J105 | ThermoFisher |

Purified recombinant Ror1 linked to human Ig Fc was conjugated to ALEXA FLUOR 647® dye for detection. EFLUOR 780® Fixable Viability dye (eBioscience) was included during primary antibody stain at a 1:800 dilution. Flow cytometry data were analyzed using FLOWJO™ 10 (Tree Star).

Jeko Co-Culture Experiments

For co-culture of Jeko target cells with CAR transduced SupT1 or primary T cells, cells were stained with VIASTAIN™ AOPI solution (Nexcelom Bioscience) to identify viable cells and counted on a CELLOMETER® automated cell counting system (Nexcelom Bioscience). An appropriate number of target and effector cells were collected by centrifugation at 300×g for 5 minutes and resuspended in complete media without cytokines at a final concentration of 1×10⁶/ml at the indicated effector:target ratios. Cells were co-cultured for 24 or 48 hours, followed by harvest of supernatant for cytokine secretion (primary T cells) or analysis of surface marker expression by flow cytometry (primary T cells and SupT1).

T Cell Killing Assay

To assess T cell cytotoxicity, CAR T cells were seeded at a 1:1 or 1:5 effector to target ratio (E:T) ratio with NUCLIGHT™ Red (NLR)-ROR1+ target or NLR Ror1KO Jeko1 target cells in a 48 well plate (Corning). Tumor cell killing was determined via incucyte measurement over time of total NLR+ cells/well compared to tumor cells alone, or via flow cytometry by measurement of the fraction of viable NLR+ cells remaining at the indicated time point relative to tumor cells alone.

Cytokine Secretion Assay

Cytokine secretion was measured with a custom V-PLEX® human proinflammatory kit (human IFNγ, IL-2, TNFα) (Meso Scale Discovery). Supernatant was collected from T cells cultured under the indicated conditions. Supernatant was centrifuged for 5 minutes at 300×g to remove cells and debris, then frozen at −80° C. For detection, supernatant was diluted 1:5 or 1:10 with MSD calibrator diluent and analyzed according to manufacturer's instructions.

Degron a Mutational Library Screening

A library of degron mutants containing (1) progressive truncations from each of the N- and C-termini, (2) sliding 4- and 10-amino acid deletions, and (3) saturation mutagenesis in which each position of the 21 amino acid degron was individually mutated to each of the 20 canonical amino acids was generated by PCR using an oligopool (IDT) containing a common priming region, each mutant degron sequence, and a homology region for Gibson assembly. The degron library was cloned via Gibson Assembly (NEB) immediately downstream of CD3z in a plasmid encoding a ROR1 CAR followed by P2A and an EGFR-based transduction marker. The assembly reaction was purified using Ampure Beads (Beckman), electroporated into NEB-10-beta electrocompetent cells and plated onto LB-Agar (+Carb) for overnight growth. Bacterial lawns were scraped and midiprepped (macherey-nagel). Purified plasmid was used to generate lentivirus, which was titered on primary human CD4+ T cells to identify the dilution of virus required to obtain ˜25% EGFR+ cells. Based on this titer, ˜20M primary human CD4+ T cells were transduced and expanded for 72 hrs. Cells were stained for EGFR and CAR (ROR1-Fc-AF647). EGFR+ cells were sorted into four bins based on CAR expression: high-high (hh), medium-high (mh), medium-low (ml), and low-low (11). In addition, 1M EGFR+ cells were sorted as an unselected control. Sorted cells were pelleted, gDNA was extracted, and sequenced using Illumina MiSeq. Sequencing reads were filtered for quality, extraneous sequence was removed (cutadapt), and the frequency of each sequence in a given bin was calculated by the number of reads for a given sequence divided by the total number of reads in that bin. The enrichment ratio (ER) for each sequence in each bin was calculated by the frequency of sequence in a given bin divided by the frequency of that sequence in the sample of unselected EGFR+ cells. These enrichment ratios are summarized in the following figures.

### 2. Introduction

A major driver of exhaustion is sustained signaling through the TCR or CAR. Transient downregulation of CAR surface expression could interrupt this process and prevent T cell exhaustion. (FIG. 1). Potential mechanisms for mediating CAR downregulation are, e.g., (1) mRNA transcription, (2) mRNA translation, and (3) protein stability. Regulation of membrane protein stability is largely via targeted internalization and trafficking to the endosome, which can occur via ubiquitin dependent and ubiquitin independent pathways (FIG. 2). Both ubiquitination and non-ubiquitin mediated pathways can involve recognition of short peptide motifs. The de novo protein engineering LOCKR system allows for programmable protein interactions driven by binding or release of a “latch” helix from a multi-helix bundle (FIG. 4). This latch can be engineered to contain a bioactive peptide, including those which modulate protein stability (FIG. 5), which can be selectively exposed in the presence of a matching key protein (FIG. 6).

### 3. Identification of Degron A as an Active Motif for Membrane Protein Regulation

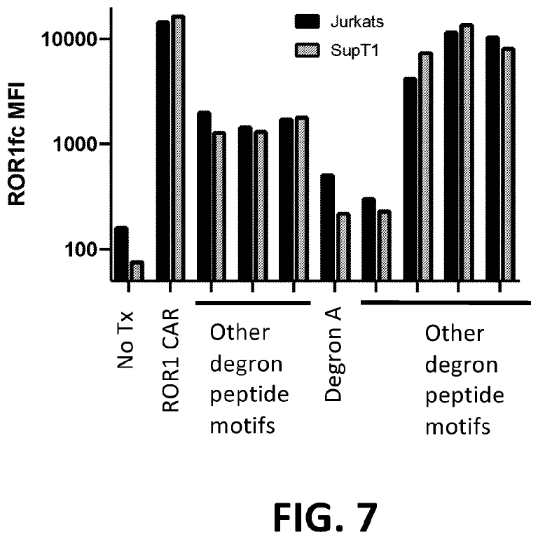

To identify peptide motifs capable of triggering internalization and degradation of CAR membrane proteins, candidate sequences were inserted between the 41BB and CD3 zeta signaling domains of a ROR1 CAR linked by a p2a sequence to the EGFRt transduction marker expressed from a 3rd generation lentiviral vector. The mean fluorescent intensity of Ror1-antigen binding by EGFRt+ transduced Jurkat and SupT1 T cell lines was then assessed. The Degron A sequence (FIG. 3) resulted in more than ten-fold lower CAR expression compared to the parental ROR1 CAR (FIG. 7).

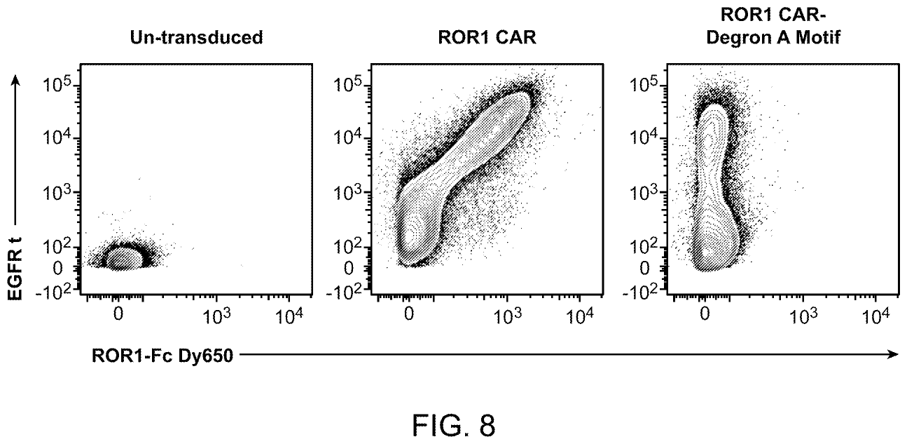

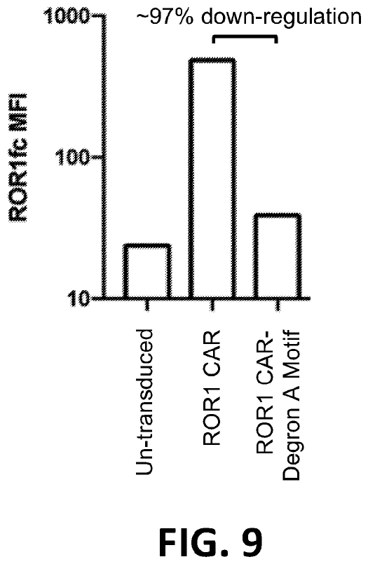

Transduction of primary human T cells revealed that the Degron A sequence led to almost complete loss of surface CAR expression, without affecting the surface expression of the p2a linked EGFRt transduction marker (FIGS. 8 and 9).

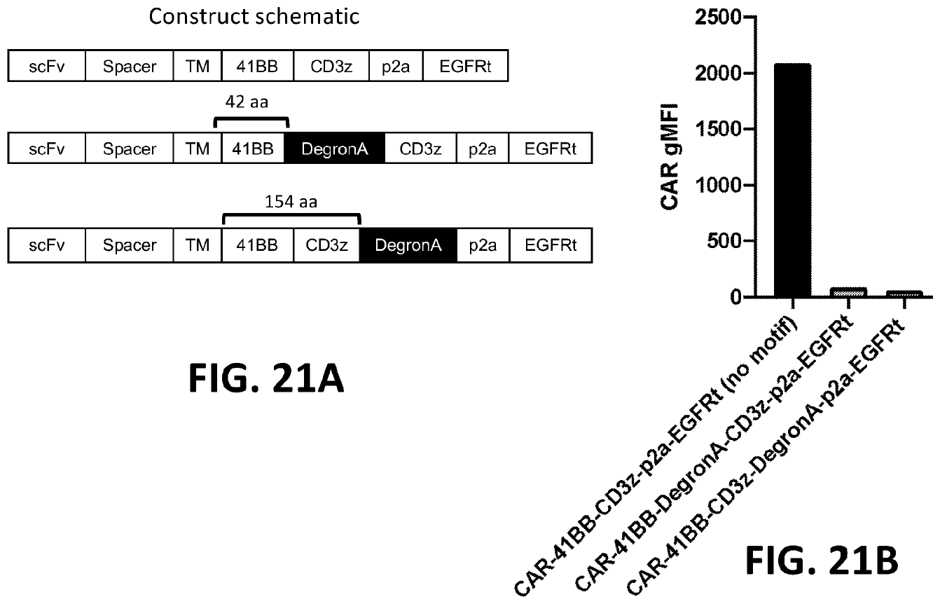

The activity of Degron A was not affected by altering the position of the motif relative to the cell membrane. Degron A inserted between the 41BB and CD3 zeta signaling domains of a ROR1 CAR, 42 amino acids from the transmembrane helix, and Degron A inserted after the CD3 zeta signaling domains, 154 amino acids from the transmembrane helix led to equivalent degron activity (FIGS. 21A and 21B).

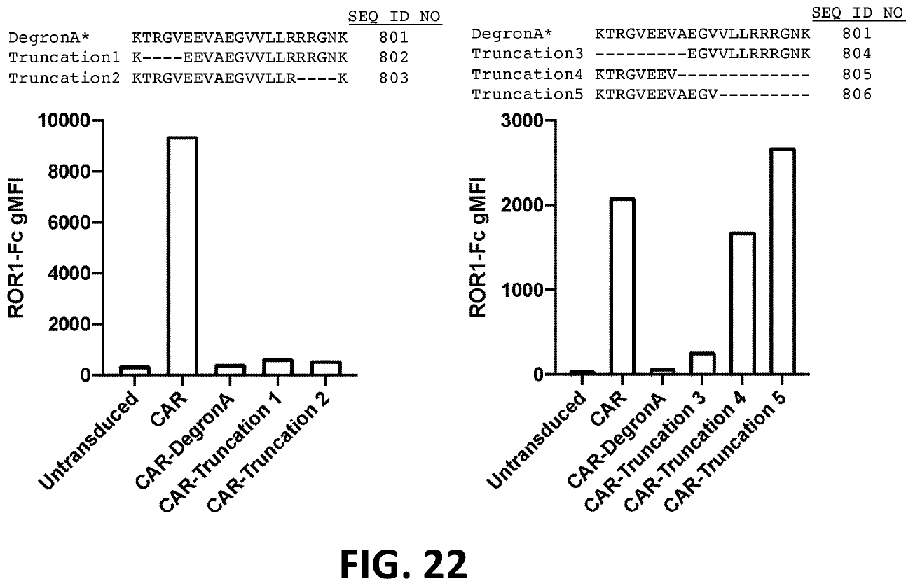

To assess sequence requirements for Degron A activity, SupT1 cells were transduced with constructs containing truncations of the Degron A sequence (FIG. 22). Sequences containing 4 amino acid truncations at the C and N terminal ends of the Degron A sequence (Truncations 1 and 2) while preserving the terminal lysine residues maintained potent activity. Truncation of 9 amino acids from the N terminal end of Degron A only modestly affected degron activity (Truncation 3), whereas truncations of 9 or 13 amino acids from the C terminal end of Degron A eliminated degron activity (Truncations 4 and 5).

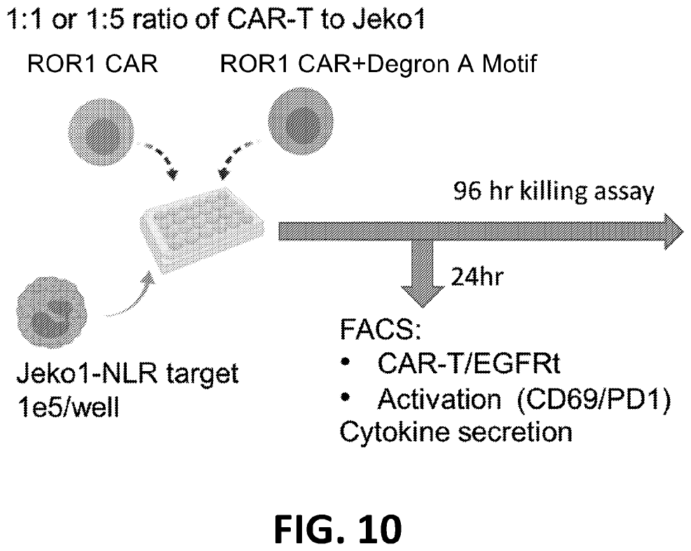

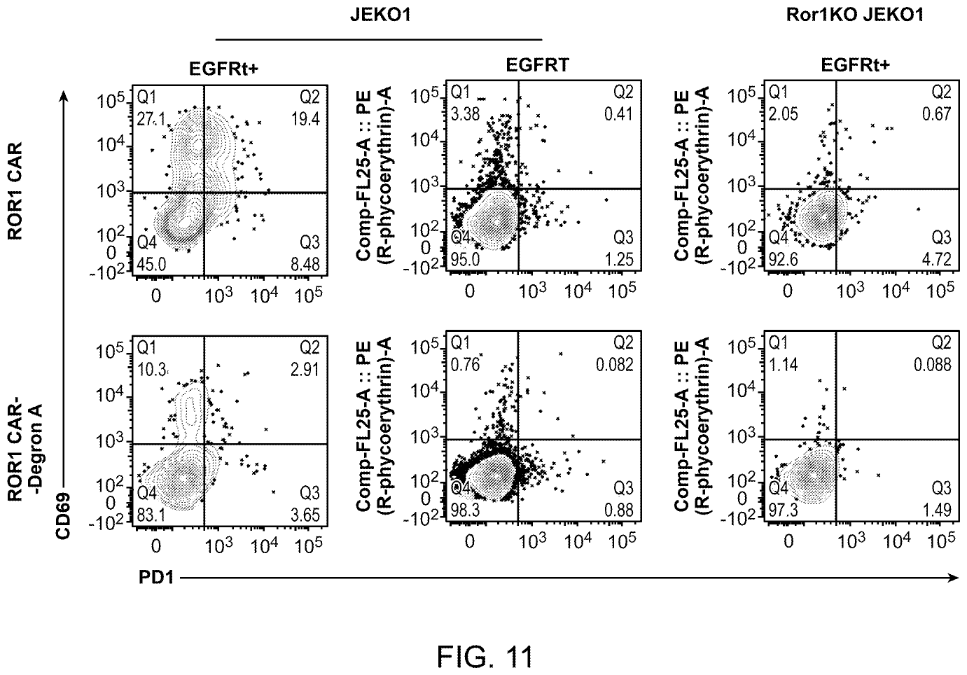

To assess the functional impact of Degron A on CAR signaling, T cells transduced with parental ROR1 CAR or Degron A containing ROR1 CAR were challenged with the Ror1-expressing Jeko1 tumor cell line (FIG. 10). Exposure to target antigen expressing cells lead to robust upregulation of the activation marker CD69 and the activation/exhaustion marker on ROR1 CAR-transduced cells, whereas Degron A linked ROR1 CAR induced less CD69, and no PD1 expression, (EGFRt+, left) (FIG. 11). Untransduced EGFRt− cells or transduced EGFRt+ cells exposed to Ror1-knockout Jeko1 cells did not upregulated CD69 or PD1.

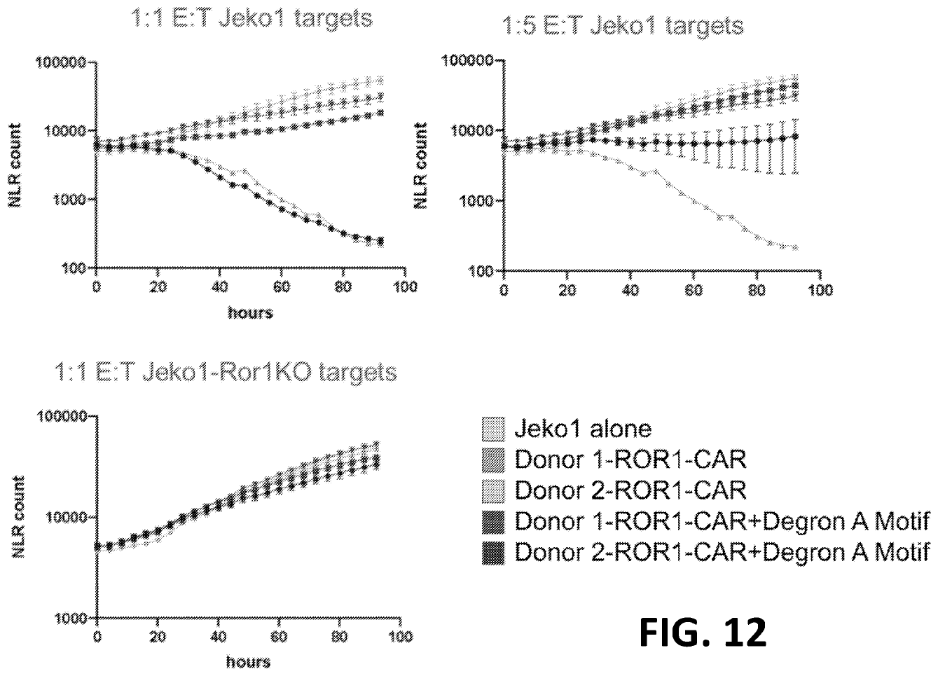

To further assess the functional impact of Degron A on CAR signaling, the ability of T cells transduced with parental ROR1 CAR or Degron A containing ROR1 CAR to kill Ror1-expressing Jeko1 cells was tested (FIG. 12). ROR1 CAR transduced T cells effectively killed Ror1 expressing Jeko1 cells at 1:1 and 1:5 effector to target cell ratios, while the Degron A containing CAR T cells failed to control Jeko1 cells (top). Jeko1 cells lacking the Ror1 antigen (Ror1 KO) were not affected by co-culture with either CAR (bottom).

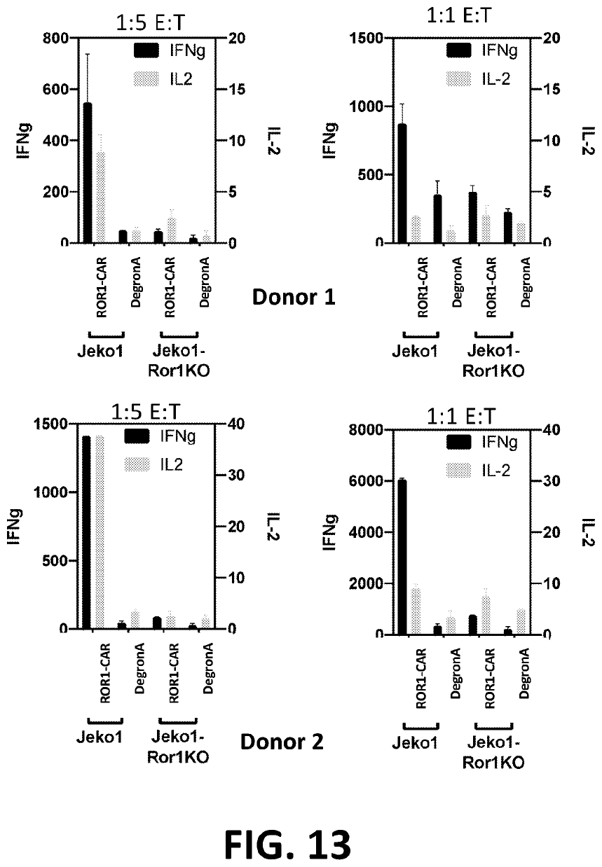

Another method to assess the functional impact of Degron A on CAR signaling, was determining the ability of T cells transduced with parental ROR1 CAR or Degron A containing ROR1 CAR to secrete cytokines after exposure to Ror1-expressing Jeko1 cells (FIG. 13). ROR1 CAR transduced T cells secreted cytokines in response to target cells, while the Degron A containing CAR T cells did not secrete either IFNg or IL-2.

### 4. Expression and Function of CAR-LOCKR Fusions

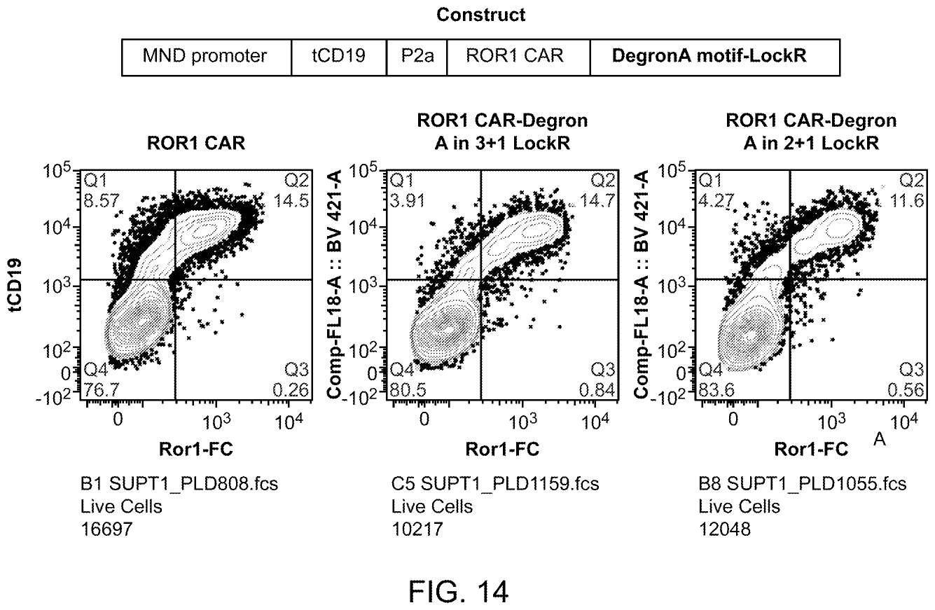

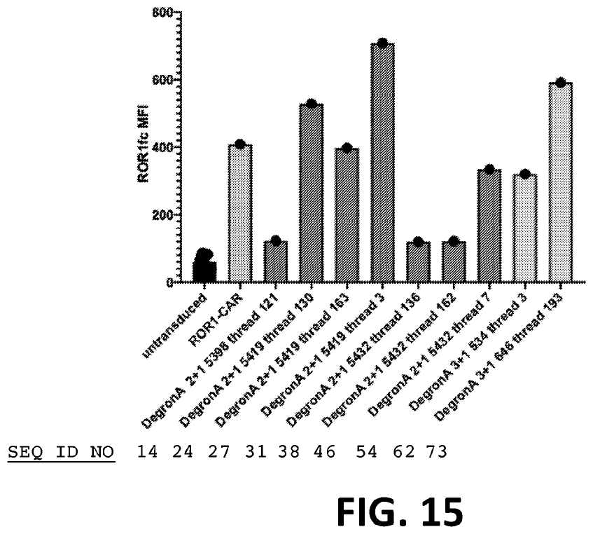

To test the ability of T cell lines to express CAR-LOCKR fusion proteins containing the Degron A motif, LOCKR constructs were fused to the C terminus of the ROR1 CAR, linked via p2a skip peptide to the truncated CD19 transduction marker. Representative FACS plots for transduction marker and Ror1 antigen binding for parental ROR1 CAR and two independent LOCKR fusions are shown in FIG. 14, and median fluorescence intensities for truncated CD19+SupT1 cells for 9 LOCKR combinations in SupT1 cells are shown in FIG. 15.

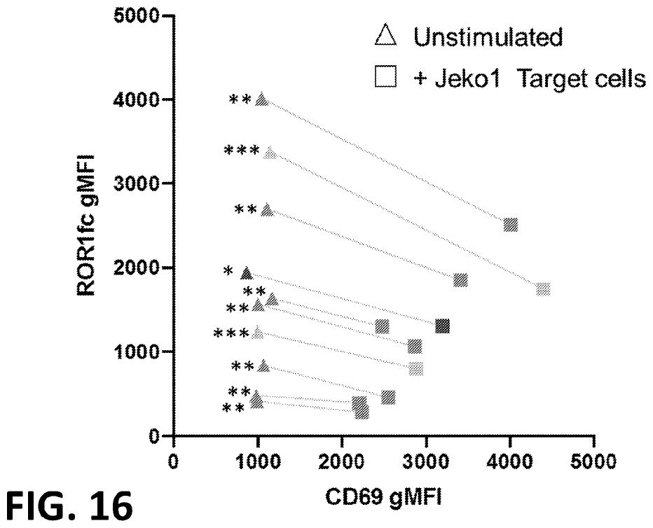

To test the ability of CAR-LOCKR fusion proteins to activate T cell lines, SupT1 cells transduced with the parental ROR1 CAR and nine independent CAR-LOCKR fusions containing Degron A were challenged with Ror1 expressing Jeko1 cells. After co-culture, the upregulation of the activation marker CD69 in unstimulated and Jeko1 conditions were compared (FIG. 16). The results demonstrated CD69 upregulation linked to total CAR surface expression, independent of the presence of LOCKR fusions.

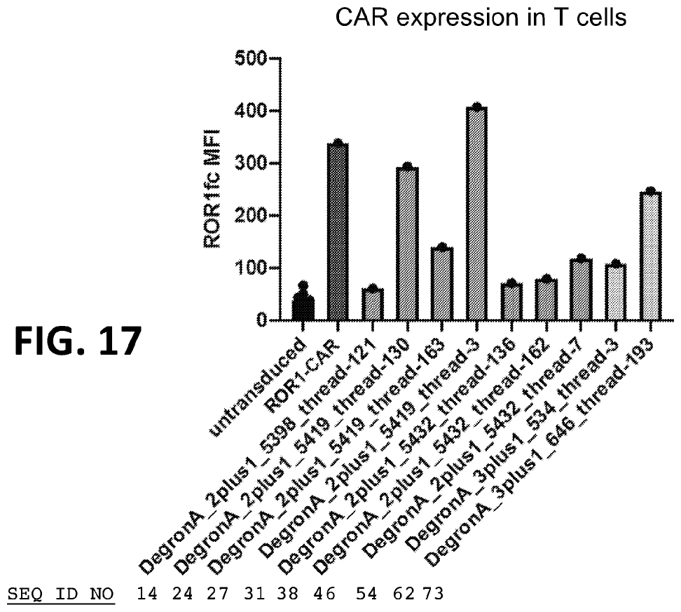

To test the ability of primary T cells to express CAR-LOCKR fusion proteins containing the Degron A motif, LOCKR constructs were fused to the C terminus of the ROR1 CAR, linked via p2a skip peptide to the truncated CD19 transduction marker. FIG. 17 shows median fluorescence intensity for truncated CD19+ cells corresponding to nine LOCKR combinations in primary T cells.

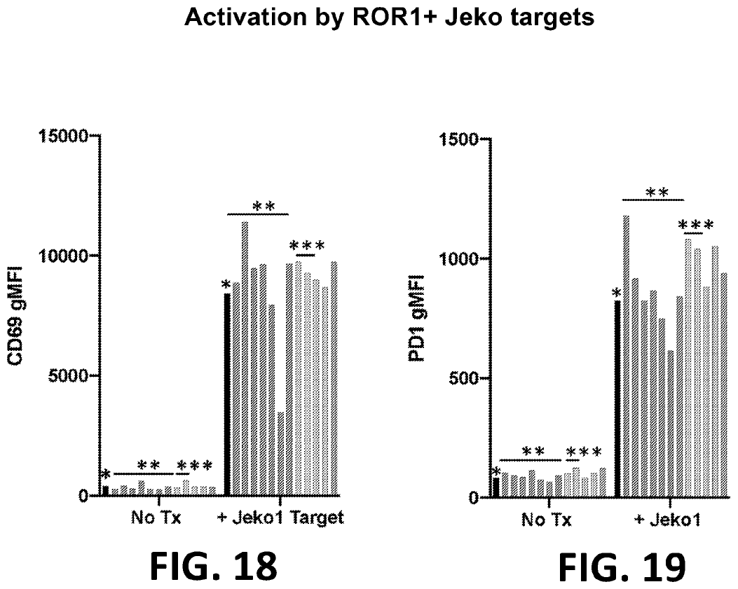

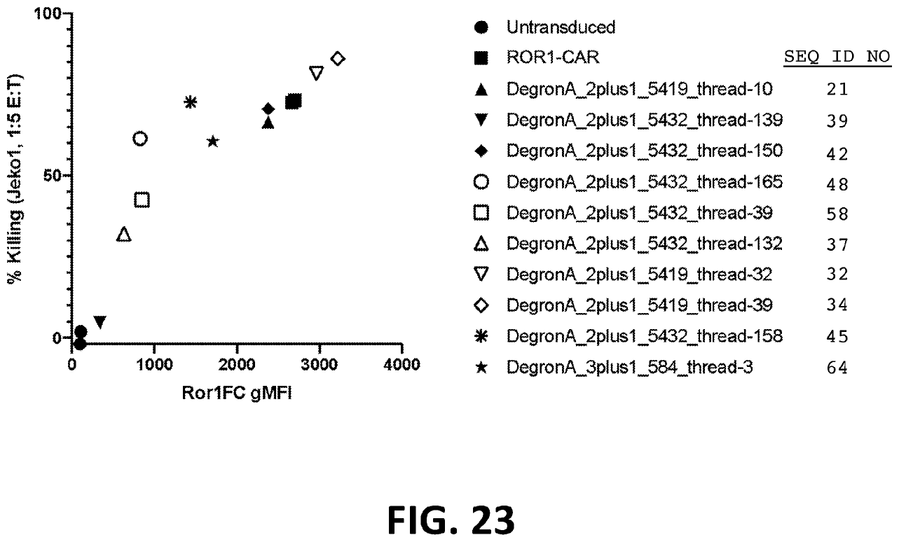

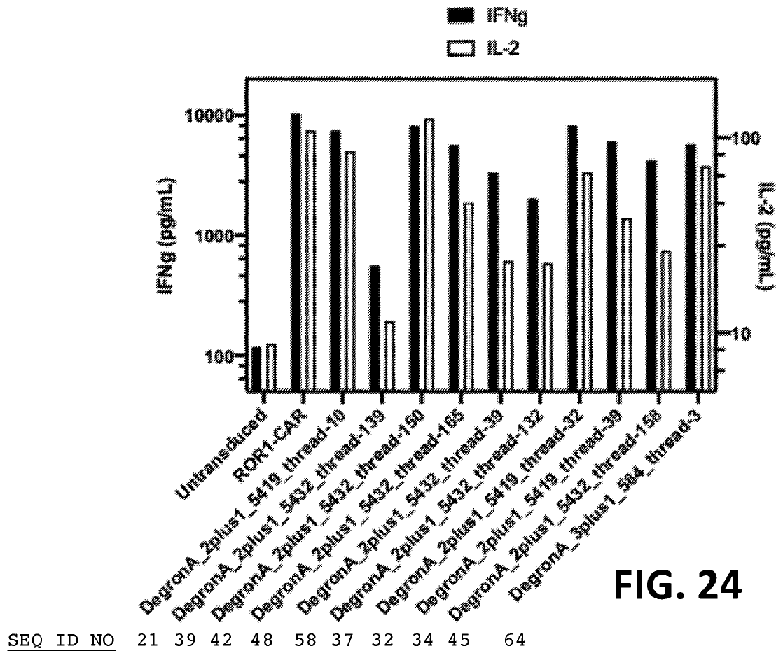

To test the ability of CAR-LOCKR fusion proteins to activate primary T cells, T cells transduced with the parental ROR1 CAR or nine independent CAR-LOCKR fusions containing Degron A were challenged with Ror1 expressing Jeko1 cells and compared the upregulation of the activation marker CD69 (FIG. 18) and the activation/exhaustion marker PD1 (FIG. 19) in unstimulated and Jeko1 conditions (FIG. 16). The results demonstrated activation induced CD69 and PD1 independent of the presence of LOCKR fusions. Consistent with these results, primary T cells transduced with the control CAR construct and CAR constructs expressing at the C-terminus a LOCKR protein containing Degron A were able to kill target cells, in a manner dependent on total cell surface expression, but independent of the presence of LOCKR fusions (see FIG. 23). CAR-LOCKR fusions were also able to specifically secrete cytokines in response to target cells (FIG. 24).

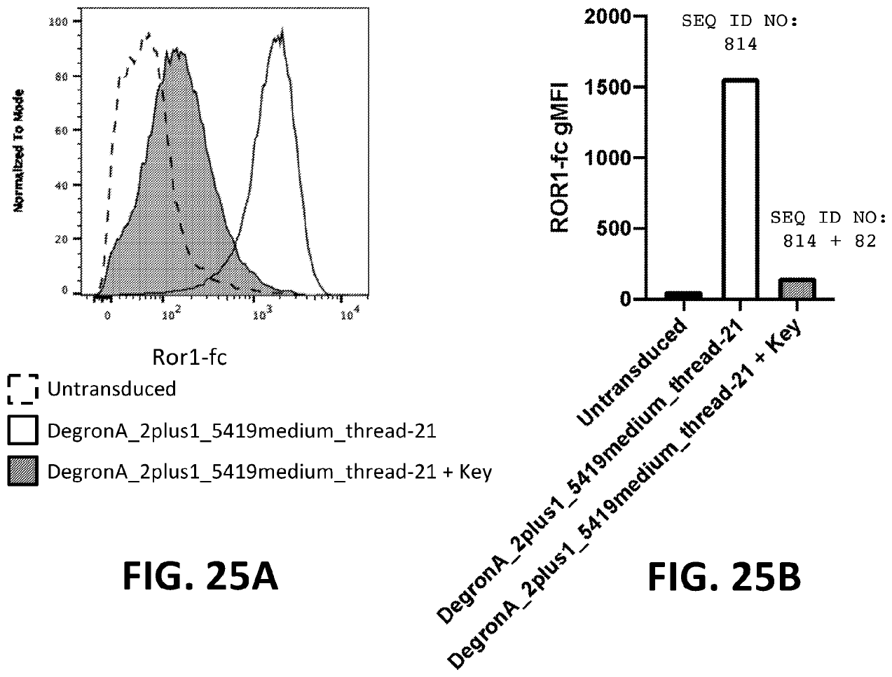

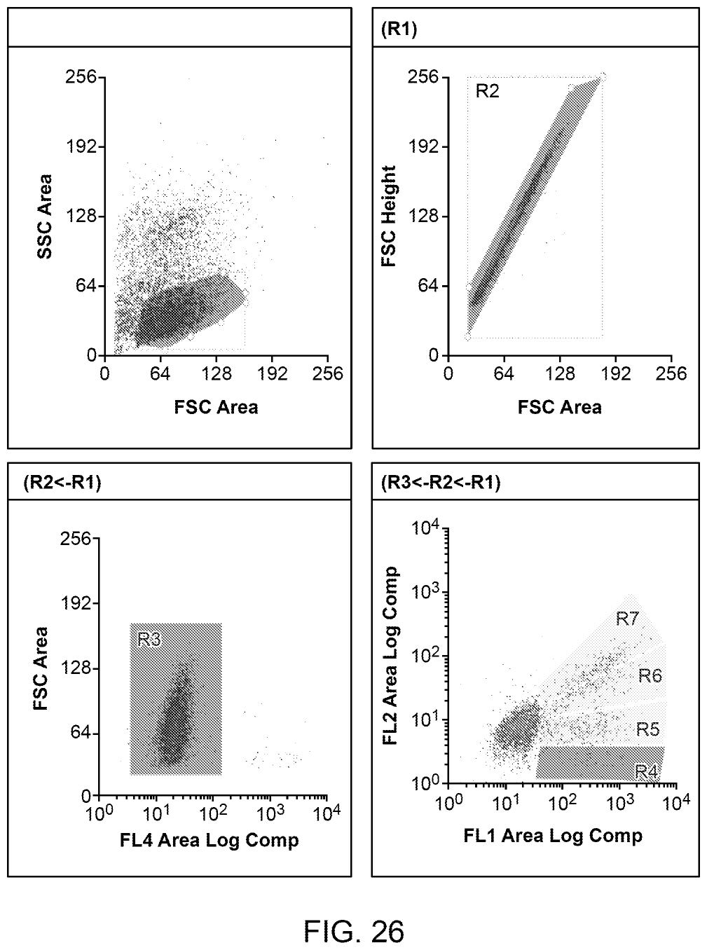

A critical aspect of Degron CAR-LOCKR function is switching between a close state with a sequestered degron sequence and an open state with an active degron motif driven by the binding of a key protein. To test the switching activity, SupT1 cells were transduced with either a DegronA containing CAR-LOCKR alone or transduced with both a CAR-LOCKR and its cognate key fused to the cytoplasmic portion of the RQR8 synthetic transmembrane protein (Philip B. et al, Blood, 2014), and expression of CAR measured by FACS. Surface expression of LOCKR-CAR was decreased in the presence of key, with detection just above background staining levels observed in untransduced cells (FIGS. 25A and 25B).

### Comprehensive Analysis of the Sequence Requirements for Degron a Activity.

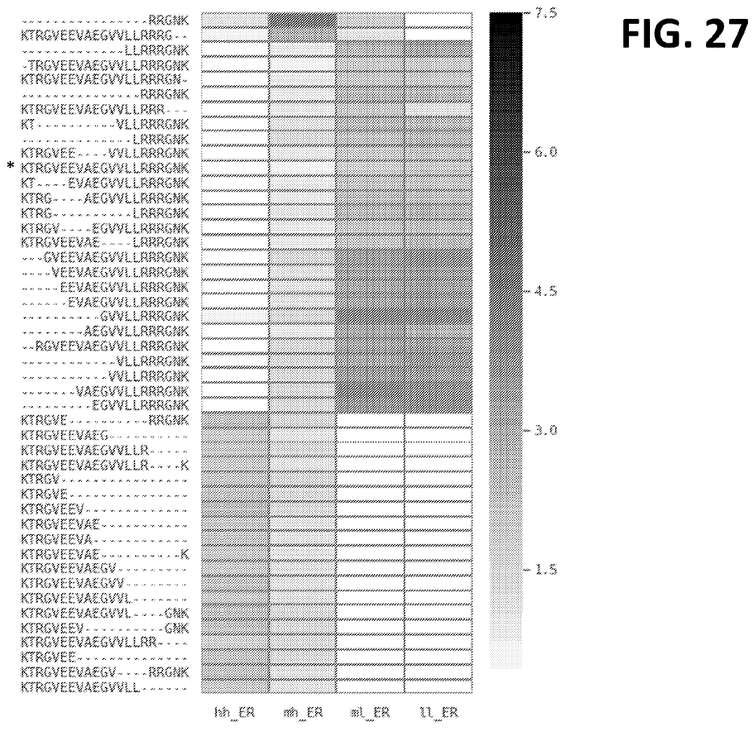

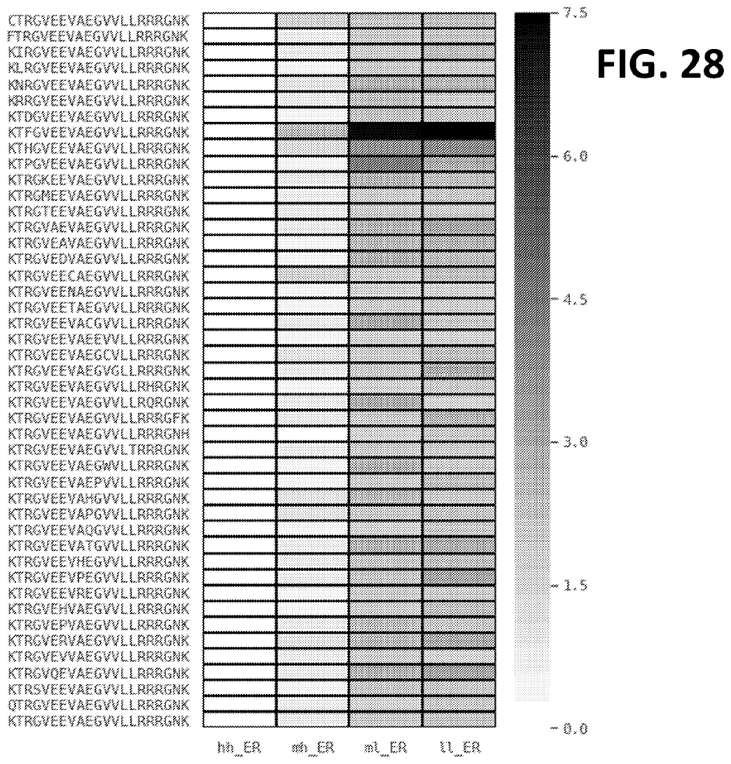

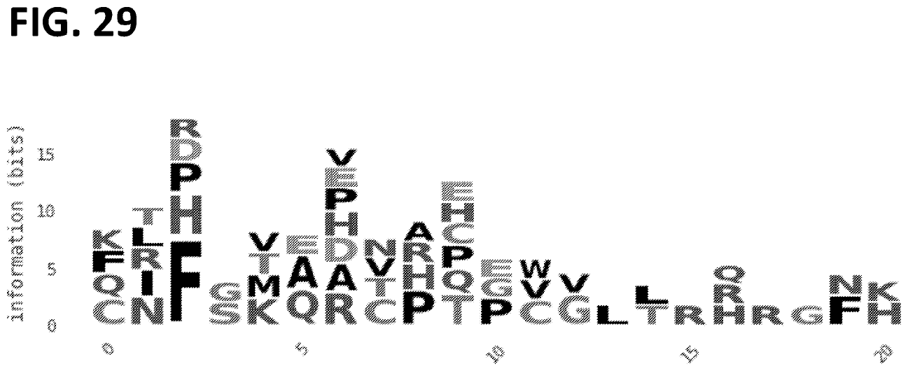

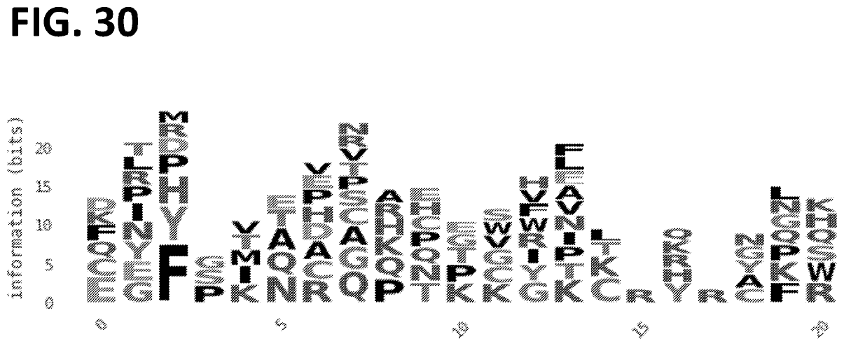

To determine the sequence requirements for Degron A activity on CAR membrane proteins, we generated a comprehensive lentiviral library of mutations, including progressive truncation progressive truncations from the N- and C-termini, sliding 4 and 10 amino acid deletions, and saturation mutagenesis of each position within the 21 amino acid degron. To minimize interactions between the mutational screen and CAR functional elements, the mutated Degron A sequences were positioned after CD3 zeta, as described for the CAR-41BB-CD3z-DegronA-p2a-EGFRt construct in FIG. 21. Primary T cells were transduced under conditions where the majority of transduced cells contained a single integration and sorted into four bins based on CAR expression: high-high (hh), medium-high (mh), medium-low (ml), and low-low (ll), as well as total EGFR+ cells as an unselected control. Mutational frequency in each bin were determined by sequencing of genomic DNA, and an enrichment ratio (ER) for each sequence in each bin was calculated by (frequency of sequence in a given bin/frequency of that sequence in the sample of unselected EGFR+ cells). Examination of sequences representing truncations revealed a critical LRRRGN sequence (SEQ ID NO: 113) towards the C-terminal end of Degron A (FIG. 27). Stringent filtering of point mutagenesis data identified a set of sequences with function (ER) similar to the original Degron A sequences (FIG. 28). Comparison of functional sequences to the original degron A sequence revealed that the N terminal portion of the degron sequence did not contribute as much to the degradation activity as did the C terminal portion. Furthermore, the core degron motif was mapped to LLRRRGN (SEQ ID NO: 115) (FIG. 29); the consensus sequence that captures these variants with function similar to the original Degron A sequences is DegronA*_Consensus1 (Table 5). Less stringent filtering of point mutagenesis data identified a more varied set of sequences which retained at least some degron function (FIG. 30); the consensus sequence that captures these variants identified by less stringent filtering is DegronA*_Consensus2 (Table 5).

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

1-125. (canceled)

126. A polynucleotide encoding a degron peptide comprising a sequence of X1-R-X2-R—X3-X4-X5, wherein
XI is L, T, K, or C; X2 is Q, K, R, H, or Y; X3 is N, G, Y, A, or C; X4 is L, N, G, Q, P, K, or F; and X5 is K, H, Q, S, W, or R.

127. The polynucleotide of claim 126, wherein the polynucleotide further encodes a latch region comprising the degron peptide, and encodes a structural region, wherein the latch region is capable of binding to the structural region.

128. The polynucleotide of claim 127, wherein the structural region comprises a peptide sequence having at least about 95%, at least about 96%, at least about 97%, at least about 98%, at least about 99% or about at least 100% sequence identity to the sequence of any one of SEQ ID of SEQ ID NOS: 142-165, 838, 840, 842, 844, 846, 848, 850, 852, 854, 856, 858, 860, 862, 864, and 866.

129. The polynucleotide of claim 127, wherein the latch region comprises a peptide sequence having at least about 95%, at least about 96%, at least about 97%, at least about 98%, at least about 99% or about at least 100% sequence identity to the sequence of any one of SEQ ID NOS: 166-189, 839, 841, 843, 845, 847, 849, 851, 853, 855, 857, 859, 861, 863, 865, 867.

130. The polynucleotide of claim 127, wherein the polynucleotide further encodes for one or more of a signaling domain, a co-stimulatory domain, an antigen binding domain, a chimeric antigen receptor (CAR), and a transmembrane domain.

131. The polynucleotide of claim 130, wherein the signaling domain comprises a domain derived from any one of CD3zeta, FcRgamma, FcR beta, CD3 gamma, CD3 delta, CD3 epsilon, CDS, CD22, CD79a, CD79b, and CD66d.

132. The polynucleotide of claim 130, wherein the co-stimulatory domain comprises a domain derived from any one of 2B4, HVEM, ICOS, LAG3, DAP10, DAP12, CD27, CD28, 4_1BB (CD137), OX40 (CD134), CD30, CD40, ICOS (CD278), glucocorticoid-induced tumor necrosis factor receptor (GITR), lymphocyte function-associated antigen_1 (LFA_1), CD2, CD7, LIGHT, NKG2C, or B7 H3.

133. The polynucleotide of claim 130, wherein the antigen binding domain comprises antibody or an antigen binding fragment thereof that specifically binds to an epitope on a tumor antigen, wherein antigen binding domain is an lg NAR, a Fab, a Fab′, a F(ab)′2, a F(ab)′3, an Fv, a single chain variable fragment (scFv), a bis-scFv, a (scFv)2, a minibody, a diabody, a triabody, a tetrabody, an intrabody, a disulfide stabilized Fv protein (dsFv), a unibody, or a nanobody.

134. The polynucleotide of claim 133, wherein the antigen binding domain specifically binds to any one of CD19, TRAC, TCR, BCMA, CLL_1, CSI, CD38, CD19, TSHR, CD123, CD22, CD30, CD70, CDI 71, CD33, EGFRvIII, GD2, GD3, Tn Ag, PSMA, ROR1, ROR2, GPCI, GPC2, FLT3, FAP, TAG72, CD44v6, CEA, EPCAM, B7H3, KIT, IL_13Ra2, Mesothelin, IL-1 1Ra, PSCA, PRSS21, VEGFR2, LewisY, CD24, PDGFR-beta, SSEA 4, CD20, Folate receptor alpha, ERBB2 (Her2/neu), MUCI, MUC16, EGFR, NCAM, Prostase, PAP, ELF2M, Ephrin B2, IGF_I receptor, CAIX, LMP2, gplOO, bcr-abl, tyrosinase, EphA2, Fucosyl GMI, sLe, GM3, TGS5, HMWMAA, o_acetyl_GD2, Folate receptor beta, TEM1/CD248, TEM7R, CLDN6, GPRC5D, CXORF61, CD97, CD179a, ALK, Polysialic acid, PLACI, GloboH, NY_BR_1, UPK2, HAVCRI, ADRB3, PANX3, GPR20, LY6K, OR51E2, TARP, WTI, NY_ESO_1, LAGE-la, MAGE-Al, legumain, HPV E6,E7, MAGE Al, ETV6_AML, sperm protein 17, XAGEI, Tie 2, MAD_CT_1, MAD_CT_2, Fas-related antigen 1, p53, p53 mutant, prostein, survivin and telomerase, PCTA_1/Galectin 8, MelanA/MARTI, Ras mutant, hTERT, sarcoma translocation breakpoints, ML_IAP, ERG (TMPRSS2 ETS fusion gene), NAI 7, PAX3, Androgen receptor, Cyclin B1, MYCN, RhoC, TRP_2, CYPIBI, BORIS, SART3, PAX5, OY_TESI, LCK, AKAP_4, SSX2, RAGE_1, human telomerase reverse transcriptase, RUI, RU2, intestinal carboxyl esterase, mut hsp70_2, CD79a, CD79b, CD72, LAIRI, FCAR, LILRA2, CD300LF, CLEC12A, BST2, EMR2, LY75, GPC3, FCRL5, and IGLLI, or any combination thereof.

135. The polynucleotide of claim 130, wherein the CAR is designed as a standard CAR, a split CAR, an off-switch CAR, an on-switch CAR, a first-generation CAR, a second-generation CAR, a third-generation CAR, or a fourth-generation CAR.

136. The polynucleotide of claim 135, wherein the CAR comprises:
(i) a chimeric polypeptide encoded by the polynucleotide of any one of claim 131 or claim 132;
(ii) an antigen-binding domain that binds to an epitope on a tumor antigen expressed on a target cell; and
(iii) a transmembrane domain.

137. The polynucleotide of claim 136, wherein the CAR further comprises a CAR spacer between the antigen binding domain and the transmembrane domain.

138. The polynucleotide of claim 126, wherein the polypeptide comprises a T cell receptor, induces IFNy and/or IL-2 expression in a cell, is capable of being degraded through a ubiquitin dependent pathway, is capable of being degraded when in contact with a key polypeptide, and is a DNA molecule, an RNA molecule, or any combination thereof.

139. The polynucleotide of claim 126, wherein the polypeptide encoding a degron peptide comprises a sequence of any one of SEQ ID NOS: 1, 801-804, and 918-982.

140. A polynucleotide set comprising a first polynucleotide comprising the polynucleotide of claim 127 and a second polynucleotide encoding a key polypeptide that is capable of inhibiting binding of a latch region to a structural region of the first polynucleotide when the key polypeptide of the second polynucleotide comes in contact with the first polynucleotide.

141. The polynucleotide set of claim 140, wherein the key polypeptide comprises an amino acid sequence that is at least about 50%, at least about 55%, at least about 60%, at least about 65%, at least about 70%, at least about 75%, at least about 80%, at least about 85%, at least about 90%, at least about 95%, or about 100% sequence identity to any sequence of SEQ ID NOS: 77-96, wherein the key polypeptide is capable of binding to the structural region of the chimeric polypeptide.

142. A vector comprising, a polynucleotide of claim 126, wherein the vector is a viral vector, a mammalian vector, or a bacterial vector.

143. A composition for treating a subject in need of a CAR therapy, comprising a polynucleotide of claim 136.

144. A kit comprising: the polynucleotide set of claim 140.

145. A cell comprising the polynucleotide of claim 127.

146. A method of making an engineered cell comprising transfecting the polynucleotide of claim 127.

147. A method of treating a tumor, comprising:
administering an immune cell expressing a polynucleotide of claim 127 in combination with one or more additional therapeutic agents to a subject.

148. The method of claim 147, wherein administering the immune cell expressing the polynucleotide of claim 1 further includes expressing a second polynucleotide encoding a key polypeptide that is capable of inhibiting binding of a latch region to a structural region of polynucleotide of claim 127 when the key polypeptide of the second polynucleotide comes in contact with the polynucleotide of claim 127.

149. A method of providing an anti-tumor immunity in a subject, comprising
administering a cell composition to the subject, the cell composition comprising the cell of claim 145.

150. The method of claim 149, wherein the cell composition further comprises a second polynucleotide encoding a key polypeptide that is capable of inhibiting binding of a latch region to a structural region of the polynucleotide of claim 127 when the key polypeptide of the second polynucleotide comes in contact with the first polynucleotide of claim 127.

151. A method of stimulating a T cell-mediated immune response to a target cell population or tissue comprising:
administering an effective amount of a cell composition to a subject wherein the cell composition comprises the cell of claim 145.

152. The method of claim 151, wherein the cell composition further includes a second polynucleotide encoding a key polypeptide that is capable of inhibiting binding of a latch region to a structural region of the polynucleotide of claim 127 when the key polypeptide of the second polynucleotide comes in contact with the polynucleotide of claim 127.
